(19) United States
(12) Patent Application Publication
(10) Pub. No.: US 2013/0317029 A1
(43) Pub. Date: Nov. 28, 2013

(54) OXYPYRIMIDINES AS SYK MODULATORS

(71) Applicants: Anjali Pandey, Fremont, CA (US); Qing Xu, Foster City, CA (US); Wolin Huang, Foster City, CA (US); Zhaozhong J. Jia, San Mateo, CA (US); Yonghong Song, Foster City, CA (US)

(72) Inventors: Anjali Pandey, Fremont, CA (US); Qing Xu, Foster City, CA (US); Wolin Huang, Foster City, CA (US); Zhaozhong J. Jia, San Mateo, CA (US); Yonghong Song, Foster City, CA (US)

(73) Assignee: PORTOLA PHARMACEUTICALS, INC., South San Francisco, CA (US)

(21) Appl. No.: 13/882,958

(22) PCT Filed: Nov. 1, 2011
(86) PCT No.: PCT/US11/58822

§ 371 Date: Jul. 8, 2013

Related U.S. Application Data
(60) Provisional application No. 61/409,060, filed on Nov. 1, 2010.

(51) Int. Cl. C07D 239/48; C07D 405/12; C07D 417/12; C07D 403/12; C07D 401/12

(52) U.S. Cl. CPC C07D 239/48; C07D 403/12; C07D 401/12; C07D 417/12; C07D 405/12
USPC 514/235.8; 544/321; 514/272; 435/184; 544/296; 544/295; 514/252.14; 544/123

(57) ABSTRACT

The present invention is directed to compounds of formula (I) and tautomers thereof or pharmaceutically acceptable salts, esters, and prodrugs thereof which arc inhibitor of Syk kinase. The present invention is also directed to intermediates used in making such compounds, the preparation of such a compound, pharmaceutical compositions containing such a compound, methods of inhibition Syk kinase activity, methods of inhibition the platelet aggregation, and methods to prevent or treat a number of conditions mediated at least in part by Syk kinase activity, such as undesired thrombosis and Non Hodgkin's Lymphoma.

### CROSS-REFERENCES TO RELATED APPLICATIONS

This application claims priority under 35 U.S.C. §119(e) from U.S. Provisional Application 61/409,060, filed Nov. 1, 2010, which is incorporated by reference in its entirety herewith.

### BACKGROUND OF THE INVENTION

This invention is directed to pyrimidine-5-carboxamide compounds which act as inhibitors of Spleen tyrosine kinase (Syk) kinases. This invention is also directed to pharmaceutical compositions containing the pyrimidine-5-carboxamide compounds and methods of using the compounds or compositions to treat a condition mediated at least in part by Syk activity. The invention is also directed to methods of making the compounds described herein.

Protein kinases constitute a large family of structurally related enzymes that are responsible for the control of a variety of signal transduction processes within cells (see, e.g., Hardie and Hanks, The Protein Kinase Facts Book, I and II, Academic Press, San Diego, Calif., 1995). Protein kinases are thought to have evolved from a common ancestral gene due to the conservation of their structure and catalytic function. Almost all kinases contain a similar 250-300 amino acid catalytic domain. The kinases can be categorized into families by the substrates they phosphorylate (e.g., protein-tyrosine, protein-serine/threonine, lipids, etc.). Sequence motifs have been identified that generally correspond to each of these families (see, e.g., Hanks & Hunter, (1995), FASEB J. 9:576-596; Knighton et al., (1991), Science 253:407-414; Hiles et al., (1992), Cell 70:419-429; Kunz et al., (1993), Cell 73:585-596; Garcia-Bustos et al., (1994), EMBO J. 13:2352-2361).

Many diseases are associated with abnormal cellular responses triggered by protein kinase-mediated events. These diseases include autoimmune diseases, inflammatory diseases, bone diseases, metabolic diseases, neurological and neurodegenerative diseases, cancer, cardiovascular diseases, allergies, asthma, Alzheimer's disease and hormone-related diseases. As a consequence, there has been substantial efforts in medicinal chemistry to find inhibitors of protein kinases for use as therapeutic agents.

Immunoreceptor tyrosine activation motif (ITAM)-mediated signaling has emerged as a primary event in signaling pathways responsible for human pathologies. ITAM-mediated signaling is responsible for relaying activation signals initiated at classical immune receptors such as T-cell receptors, B-cell receptors, Fc receptors in immune cells and at GPVI and FcγRIIa in platelets to downstream intracellular molecules such as Syk and ZAP-70 (Underhill, D. M and Goodridge, H. S., Trends Immunol., 28:66-73, 2007).

The binding of a ligand to an ITAM-containing receptor triggers signaling events which allows for the recruitment of proteins from a family of nonreceptor tyrosine kinases called the Src family. These kinases phosphorylate tyrosine residues within the ITAM sequence, a region with which the tandem SH2 domains on either Syk or ZAP-70 interact.

Syk, along with Zap-70, is a member of the Syk family of protein tyrosine kinases. The interaction of Syk or ZAP-70 with diphosphorylated ITAM sequences induces a conformation change in the kinases that allows for tyrosine phosphorylation of the kinase itself. Phosphorylated Syk family members activate a multitude of downstream signaling pathway proteins which include Src homology 2 (SH2) domain containing leukocyte-specific phosphoprotein of 76 kDa (SLP-76), Linker of Activation of T-cells (LAT) and PLC (phospholipase C)γ2.

Human pathologies attributed to dysfunctional ITAM-mediated signaling include autoimmune diseases such as rheumatoid arthritis, systemic lupus, multiple sclerosis, hemolytic anemia, immune-thrombocytopenia purpura, and heparin-induced thrombocytopenia and arteriosclerosis. Interestingly, many of the above mentioned diseases are thought to occur through crosslinking of Fc receptors by antibodies which, via Syk, activate a signaling cascade in mast, basophil and other immune cells that result in the release of cell mediators responsible for inflammatory reactions. The release of mediators and the production of cytokines in IgE stimulation-dependent allergic and inflammatory reactions from mast cells and basophiles can be controlled by inhibiting the tyrosine kinase activity of Syk (Rossi, A. B. et al., J Allergy Clin Immunol., 118:749-755, 2006). In immune-thrombocytopenia, antibody bound platelets are cleared by the spleen by an Fc receptor/ITAM/Syk-mediated process (Crow, A. R. et al., Blood, 106:abstract 2165, 2005). Drug-induced thrombocytopenia, caused by heparin-platelet factor 4 immune complexes that activate platelet FcγRIIa, also involve Syk signaling downstream of receptor engagement (Reilly, M. P., Blood, 98:2442-2447, 2001).

Platelet agonists induce inside-out integrin signaling resulting in fibrinogen binding and platelet aggregation. This initiates outside-in signaling which produces further stimulation of platelets. Syk is activated during both phases of integrin signaling, and inhibition of Syk is shown to inhibit platelet adhesion to immobilized proteins (Law, D. A. et al., Blood, 93:2645-2652, 1999). Release of arachidonic acid and serotonin and platelet aggregation induced by collagen are markedly inhibited in platelets derived from Syk deficient mouse (Poole, A. et al., EMBO J., 16:2333-2341, 1997). Thus Syk inhibitors may also possess anticoagulation action.

Because of the role Syk plays in Ig-induced platelet activations, it is likely to be important in arteriosclerosis and restenosis. Arteriosclerosis is a class of diseases characterized by the thickening and hardening of the arterial walls of blood vessels. Although all blood vessels are susceptible to this serious degenerative condition, the aorta and the coronary arteries serving the heart are most often affected. Arteriosclerosis is of profound clinical importance since it can increase the risk of heart attacks, myocardial infarctions, strokes, and aneurysms.

The traditional treatment for arteriosclerosis includes vascular recanalization procedures for less-serious blockages and coronary bypass surgery for major blockages. A serious shortcoming of intravascular procedures is that, in a significant number of treated individuals, some or all of the treated vessels restenose (i.e., re-narrow). For example, restenosis of an atherosclerotic coronary artery after PTCA occurs in 10-50% of patients undergoing this procedure and subsequently requires either further angioplasty or a coronary artery bypass graft. Furthermore, restenosis of an atherosclerotic coronary artery after stenting occurs in 10-20% of patients undergoing this procedure and subsequently requires repeat treatments to maintain adequate blood flow through the affected artery. Restenosis generally occurs in a relatively brief time period, e.g., roughly less than six months, after treatment.

While the exact hormonal and cellular processes promoting restenosis have not been determined, restenosis is thought to be due in part to mechanical injury to the walls of the blood vessels caused by the balloon catheter or other intravascular device. For example, the process of PTCA, in addition to opening the obstructed artery, also injures resident coronary arterial smooth muscle cells (SMCs). In response to this injury, adhering platelets, infiltrating macrophages, leukocytes, or the smooth muscle cells themselves release cell-derived growth factors such as platelet-derived growth factor (PDGF), with subsequent proliferation and migration of medial SMCs through the internal elastic lamina to the area of the vessel intima. Further proliferation and hyperplasia of intimal SMCs and, most significantly, production of large amounts of extracellular matrix over a period of three to six months results in the filling in and narrowing of the vascular space sufficient to significantly obstruct blood flow.

In addition to the role Syk plays in Ig-induced platelet activations, Syk plays a very important role in collagen-mediated signaling. The primary adhesive protein responsible for platelet adhesion and activation is collagen. Collagen is a filamentous protein contained within the fibrotic caps of atheromas which becomes exposed to blood during plaque rupture. Collagen functions initially by binding von Willebrand factor which tethers platelets through binding platelet membrane GPIb. Collagen functions secondarily by engaging the two collagen receptors on platelets, GPVI and integrin α2β1.

GPVI exists in platelet membranes as a complex with FcRγ, an interaction required for the expression of GPVI. Activation of FcγRIIa on platelets results in platelet shape change, secretion and thrombosis. Signaling by the GPVI/FcRγcomplex is initiated by tyrosine phosphorylation of the ITAM domain of FCRγ followed by the recruitment of Syk. Activation of GPVI leads to induction of multiple platelet functions including: activation of integrins α2β1 to achieve firm platelet adhesion, and GP IIb-IIIa which mediates platelet aggregation and thrombosis growth; platelet secretion, allowing for the delivery of inflammatory proteins such as CD40L, RANTES and TGFβ to the vessel wall; and the expression of P-selectin which allows for the recruitment of leukocytes. Therefore, it is believed that Syk inhibitors can inhibit thrombotic events mediated by platelet adhesion, activation and aggregation.

It has been reported that the tyrosine phosphorylation of intracellular protein (activation) induced by stimulation of a receptor for IgG antibody, FcγR, and the phagocytosis mediated by FcγR are considerably inhibited in macrophages derived from Syk deficient mouse (Crowley, M. T. et al., J. Exp. Med., 186:1027-1039, 1997). This suggests that Syk has a markedly important role in the FcγR-mediated phagocytosis of macrophages.

It has also been reported that an antisense oligonucleotide of Syk suppresses the apoptosis inhibition of eosinophils induced by GM-CSF (Yousefi, S. et al., J. E. Med., 183:1407-1414, 1996), showing that Syk is essential for the life extending signal of eosinophils caused by GM-CSF and the like. Since life extension of eosinophils is closely related to the transition of diseases into a chronic state in allergic disorders, such as asthma, Syk inhibitors can also serve as therapeutic agents for chronic eosinophilic inflammation.

Syk is important for the activation of B-cells via a B-cell antigen receptor and is involved in the phosphatidylinositol metabolism and increase in the intracellular calcium concentration caused by the antigen receptor stimulation (Hutchcroft, J E. et al., J. Biol. Chem., 267:8613-8619, 1992; and Takata, M. et al., EMBO J., 13:1341-1349, 1994). Thus, Syk inhibitors may be used to control the function of B-cells and are, therefore, expected to serve as therapeutic agents for antibody-related diseases.

Syk binds to a T-cell antigen receptor, quickly undergoes tyrosine phosphorylation through crosslinking of the receptor and synergistically acts upon intracellular signals mediated by Src tyrosine kinases such as Lck (Couture, C. et al., Proc. Natl. Acad. Sci. USA, 91:5301-5305, 1994; and Couture, C. et al., Mol. Cell. Biol., 14:5249-5258, 1994). Syk is present in mature T-cell populations, such as intraepithelial γδT-cells and naïve β T-cells, and has been reported to be capable of phosphorylation of multiple components of the TCR signaling cascade (Latour, S. et. al., Mol Cell Biol., 17:4434-4441, 1997). As a consequence, Syk inhibitors may serve as agents for inhibiting cellular immunity mediated by T-cell antigen receptor.

Recent comparative genomic hybridization studies have identified Syk as another gene important in the pathogenesis of Mantle Cell Lymphoma (MCL) (Chen, R. et al. Journal of Clinical Oncology, 2007 ASCO Annual Meeting Proceedings (Post-Meeting Edition). Vol 25, No 18S (June 20 Supplement), 2007: 8056). MCL represents 5-10% of all non-Hodgkins lymphomas and it is a difficult form of lymphoma to treat. It has the worst prognosis among the B cell lymphomas with median survival of three years. It has been reported that Syk is overexpressed in MCL (Rinaldi, A, et. al, Br. J. Haematol., 2006; 132:303-316) and that Syk mediates mTOR (mammalian target of Rapamycin) survival signals in follicular, mantel cell, Burkitt's, and diffuse large B-cell non-Hodgkin's lymphomas (Leseux, L., et. al, Blood, 2006; 108:4156-4162).

Several lines of evidence suggest that many B-cell lymphomas depend upon B-cell receptor (BCR)-mediated survival signals. BCR signaling induces receptor oligomerization and phosphorylation of Igα and β immunoreceptor tyrosine-based activated motifs by SRC family kinases. ITAM phosphorylation results in the recruitment and activation of Syk that initiates downstream events and amplifies the original BCR signal. Given the role of tonic BCR signaling in normal B cell and Syk-dependent survival of non-Hodgkins lymphoma cell lines in vitro (Chen, L., et. al, Blood, 2006; 108:3428-3433), Syk inhibition is a promising rational treatment target for certain B-cell lymphomas and chronic lymphocytic leukemia (CLL) (Stefania Gobessi, Luca Laurenti, Pablo Longo, Laura Carsetti, Giuseppe Leone, Dimitar G. Efremov, Constitutive activation of the protein tyrosine kinase Syk in Chronic Lymphocytic Leukemia B-cells, Blood, 2007, 110, Abstract 1123). Recent data shows that administration of a multikinase inhibitor which inhibits Syk, may have significant clinical activity in CLL patients (Friedberg J W et al, Blood 2008; 112(11), Abstract 3).

The oncogenic potential of the spleen tyrosine kinase (Syk) has been described in a number of different settings. Clinically, Syk over-expression is reported in Mantle Cell Lymphoma (Rinaldi, A, et. al, Br. J. Haematol., 2006; 132:303-316) and the TEL-Syk fusion protein (Translocated ETS Leukemia) generated by a chromosomal translocation (t(9; 12)(q22; p12)) leads to increased Syk activity and is associated with myelodysplastic syndrome (Kuno, Y., et. al, Blood, 2001; 97:1050-1055). Leukemia is induced in mice by adoptively transferring bone marrow cells that express human TEL-Syk (Wossning, T., JEM, 2006; 203:2829-2840). Further, in mouse primary bone marrow cells, over-expression of Syk results in IL-7 independent growth in culture (Wossning, T., et. al, JEM, 2006; 203:2829-2840).

Interestingly, Syk signaling appears to be required for B-cell development and survival in humans and mouse. Inducible loss of the B-cell receptor (Lam, K., et. al, Cell, 1997; 90:1073-1083) or Iga (Kraus, M., et. al, Cell, 2004; 117:787-800) results in loss of peripheral B-cells in mice. Over-expression of the protein tyrosine phosphatase PTP-RO, which is known to negatively regulate Syk activity, inhibits proliferation and induces apoptosis in cell lines derived from non-Hodgkin's lymphomas (Chen, L., et. al, Blood, 2006; 108:3428-3433). Finally, B-cell lymphomas rarely exhibit loss of BCR expression, and anti-idiotype therapy rarely leads to resistance (Kuppers, R. Nat Rev Cancer, 2005; 5:251-262).

Engagement of the antigen-specific B cell receptor (BCR) activates multiple signaling pathways that ultimately regulate the cells activation status, promoting survival and clonal expansion. Signaling through the BCR is made possible by its association with two other members of the immunoglobulin super-family; Igα and Igβ, each bearing an immuno-tyrosine based activation motif (ITAM) (Jumaa, Hendriks et al. Annu Rev Immunol 23: 415-45 (2005). The ITAM domain is directly phosphorylated by Src family kinases in response to BCR engagement. The spleen tyrosine kinase (Syk) docks with and phosphorylates the ITAM, a process that enhances its kinase activity, resulting in Syk autophosphorylation and tyrosine phosphorylation of multiple downstream substrates (Rolli, Gallwitz et al. Mol Cell 10(5): 1057-69 (2002). This signaling pathway is active in B cells beginning at the transition from pro- to pre-B cell stage of development, when the newly formed pre-BCR is expressed. In fact, B cell development arrests at the pro-B cell stage in Syk knockout mice (Cheng, Rowley et al. 1995; Turner, Mee et al. Nature 378(6554): 303-6 (1995). Inducible loss of the B cell receptor (Lam, Kuhn et al. Cell 90(6): 1073-83 (1997) or Iga (Kraus, Alimzhanov et al. Cell 117(6): 787-800 (2004) results in loss of peripheral B cells in mice. Human B cells also appear to require Syk for proliferation and survival. Over-expression of the protein tyrosine phosphatase PTP-RO, a negative regulator of Syk activity, inhibits proliferation and induces apoptosis in cell lines derived from non-Hodgkin's lymphomas (NHL) (Chen, Juszczynski et al. Blood 108(10): 3428-33 (2006). Knock down of Syk by siRNA in the NHL line SUDHL-4 led to a block in the Gl/S transition of the cell cycle (Gururajan, Dasu et al. J Immunol 178(1): 111-21 (2007). Together, these data suggest that Syk signaling is required for the development, proliferation, and even survival of human and mouse B cells.

Conversely, the oncogenic potential of Syk has been described in a number of different settings. Clinically, Syk over-expression is reported in Mantle Cell Lymphoma (Rinaldi, Kwee et al. Br J Haematol 132(3): 303-16 (2006) and the TEL-Syk fusion protein (Translocated ETS Leukemia) generated by a chromosomal translocation (t(9; 12)(q22; p12)) leads to increased Syk activity and is associated with myelodysplastic syndrome (Kuno, Abe et al. Blood 97(4): 1050-5 (2001). Leukemia is induced in mice by the adoptive transfer of bone marrow cells that express human TEL-Syk (Wossning, Herzog et al. J Exp Med 203(13): 2829-40 (2006). Further, in mouse primary bone marrow cells, over-expression of Syk results in IL-7 independent growth in culture (Wossning, Herzog et al. 2006). Consistently, Syk was reported to mediate mTOR (mammalian target of Rapamycin) survival signals in follicular, mantle cell, Burkitt's, and diffuse large B-cell NHL (Leseux, Hamdi et al. Blood 108(13): 4156-62 (2006). Additional recent studies also suggest that Syk-dependant survival signals may play a role in B-cell malignancies, including DLBCL, mantle cell lymphoma and follicular lymphoma (Gururajan, Jennings et al. 2006; Irish, Czerwinski et al. J Immunol 176(10): 5715-9 (2006). Given the role of tonic BCR signaling in normal B cells and Syk-dependent survival of NHL cell lines in vitro, the specific inhibition of Syk may prove promising for the treatment of certain B-cell lymphomas.

Recently, R406 (Rigel Pharmaceuticals) was reported to inhibit ITAM signaling in response to various stimuli, including FcεR1 and BCR induced Syk activation (Braselmann, Taylor et al. J Pharmacol Exp Ther 319(3): 998-1008 (2006). Interestingly, this ATP-competitive inhibitor of Syk was also active against Flt3, cKit, and JAK kinases, but not against Src kinsase (Braselmann, Taylor et al. 2006). Activating mutations to Flt3 are associated with AML and inhibition of this kinase is currently under clinical development (Burnett and Knapper Hematology Am Soc Hematol Educ Program 2007: 429-34 (2007). Over-activation of the tyrosine kinase cKit is also associated with hematologic malignancies, and a target for cancer therapy (Heinrich, Griffith et al. Blood 96(3): 925-32 (2000). Similarly, JAK3 signaling is implicated in leukemias and lymphomas, and is currently exploited as a potential therapeutic target (Heinrich, Griffith et al. 2000). Importantly, the multi-kinase inhibitory activity of R406 attenuates BCR signaling in lymphoma cell lines and primary human lymphoma samples, resulting in apoptosis of the former (Chen, Monti et al. Blood 111(4): 2230-7 (2008). Further, a phase II clinical trial reported favorable results by this compound in refractory NHL and chronic lymphocytic leukemia (Friedberg J W et al, Blood 2008; 112(11), Abstract 3). Although the precise mechanism of action is unclear for R406, the data suggest that inhibition of kinases that mediate survival signaling in lymphocytes is clinically beneficial.

Additional recent studies also suggest that Syk-dependant survival signals may play a role in B-cell malignancies, including DLBCL, mantle cell lymphoma and follicular lymphoma (see e.g., S. Linfengshen et al. Blood, February 2008; 111: 2230-2237; J. M. Irish et al. Blood, 2006; 108: 3135-3142; A. Renaldi et al. Brit J. Haematology, 2006; 132: 303-316; M. Guruoajan et al. J. Immunol, 2006; 176: 5715-5719; L. Laseux et al. Blood, 2006; 108: 4156-4162.

Patents and patent applications describing substituted pyrimidinediamine compounds include: U.S. application Ser. No. 10/355,543 filed Jan. 31, 2003 (US2004/0029902A1), international application Serial No. PCT/US03/03022 filed Jan. 31, 2003 (WO 03/063794), U.S. application Ser. No. 10/631,029 filed Jul. 29, 2003, international application Serial No. PCT/US03/24087 (WO 04/014382), U.S. application Ser. No. 10/903,263 filed Jul. 30, 2004, and international application Serial No. PCT/US2004/24716 (WO 05/016893), the disclosures of which are incorporated herein by reference. Substituted pyrimidinediamine compounds are also described in international patent application publication numbers: WO 02/059110, WO 03/074515, WO 03/106416, WO 03/066601, WO 03/063794, WO 04/046118, WO 05/016894, WO 05/122294, WO 05/066156, WO 03/002542, WO 03/030909, WO 00/39101, WO 05/037800 and U.S. Pat. Pub. No. 2003/0149064.

While progress has been made in this field, there remains a need in the art for compounds that inhibit Syk kinase, as well as for methods for treating conditions in a patient, such as restenosis, thrombosis, and/or inflammation that can benefit from such inhibition. Moreover, the availability of compounds that selectively inhibit one of these kinases as compared to other kinases would also be desirable. The present invention satisfies this and other needs.

### BRIEF SUMMARY OF THE INVENTION

The present invention provides novel compounds having activity as inhibitors of Syk activity (also referred to herein as “Syk inhibitors”), as well as to methods for their preparation and use, and to pharmaceutical compositions containing the same. Such compounds have the following structure (I):

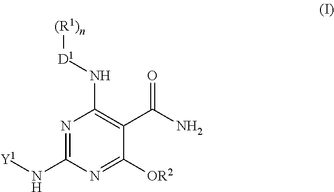

or a pharmaceutically acceptable salt thereof, wherein D¹, R¹, R², Y¹ and n are as defined below.

The present invention also provides a pharmaceutical composition comprising a therapeutically effective amount of a compound of formula I, or a pharmaceutical acceptable salt thereof, and a pharmaceutically acceptable carrier and/or diluent.

The compounds of the present invention have utility over a wide range of therapeutic applications, and may be used to treat a variety of conditions, mediated at least in part by Syk activity, in both men and women, as well as a mammal in general (also referred to herein as a “subject”). For example, such conditions include, but are not limited to, those associated with cardiovascular disease, inflammatory disease or autoimmune disease. More specifically, the compounds of the present invention have utility for treating conditions or disorders including, but not limited to: restenosis, thrombosis, inflammation, heparin induced thrombocytopenia, dilated cardiomyopathy, sickle cell disease, atherosclerosis, myocardial infarction, vascular inflammation, unstable angina, acute coronary syndromes, allergy, asthma, rheumatoid arthritis, B-cell mediated diseases such as Non Hodgkin's lymphoma, anti-phospholipid syndrome, lupus, psoriasis, multiple sclerosis, end stage renal disease, hemolytic anemia, immune thrombocytopenic purpura, and chronic lymphocytic leukemia. Thus, in one embodiment, methods are disclosed which include the administration of an effective amount of a compound of formula (I), typically in the form of a pharmaceutical composition, to a subject in need thereof.

The conditions associated with cardiovascular disease is selected from the group consisting of acute coronary syndrome, myocardial infarction, unstable angina, refractory angina, occlusive coronary thrombosis occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolism, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, and conditions requiring the fitting of prosthetic devices.

The present invention also provides a method for inhibiting the Syk kinase activity of a blood sample comprising contacting said sample with a compound of the present invention.

The present invention further provides compounds in purified forms, as well as chemical intermediates.

These and other aspects, objects, features and advantages of the invention will be apparent upon reference to the following detailed description and figures. To this end, various references are set forth herein which describe in more detail certain background information, procedures, compounds and/or compositions, and are each hereby incorporated by reference in their entirety.

### DETAILED DESCRIPTION OF THE INVENTION

As used herein, the below terms have the following meanings unless specified otherwise:

### 1. ABBREVIATIONS AND DEFINITIONS

The abbreviations used herein are conventional, unless otherwise defined. The following abbreviations are used: AcOH=acetic acid, AIBN=azobisisobutyronitrile (also azobisisobutylonitrile), aq.=aqueous, Boc=t-butylcarboxy, Bz—benzyl, BOP=benzotriazol-1-yloxytris(dimethylamino)-phosphonium hexafluorophosphate, BPO=benzoyl peroxide, nBuOH=n-butanol, CBr₄=tetrabromomethane, mCPBA=m-chloroperoxybenzoic acid, CH₂Cl₂ or DCM=dichloromethane, Cs₂CO₃=cesium carbonate, CuCl₂=copper chloride; DPPA=diphenyl phosphoryl azide; DIBAL=diisobutylaluminum hydride, DIEA=Hunig's base or diisopropyl ethylamine, DME=dimethyl ether, DMF=dimethyl formamide, DMSO=dimethyl sulfoxide, Et₃N=triethylamine, EtOAc=ethyl acetate, g=gram, HATU=2-(1H 7-Azabenzotriazol-1-yl)-1,1,3,3-tetramethyl uronium hexafluorophosphate, H₂=hydrogen; H₂O=water; HBr=hydrogen bromide; HCl=hydrogen chloride, HIV=human immunodeficiency virus, HPLC=high pressure liquid chromatography, h=hour, IgE=immunoglobulin E, IC₅₀=The concentration of an inhibitor that is required for 50% inhibition of an enzyme in vitro, IPA=isopropyl alcohol, kg=kilogram, KCN=potassium cyanide, KOH=potassium hydroxide, K₂PO₄=potassium phosphate, LDA=lithium diisopropylamine, LiAlH₄=lithium aluminum hydride=LiOH: lithium hydroxide; MeCN=acetonitrile; MS=Mass Spec, m/z=mass to charge ratio, MHz=Mega Hertz, MeOH=methanol, μM=micromolar, μL=microliter, mg=milligram, mm=millimeter, mM=millimolar, mmol=millimole, mL=milliliter, mOD/min=millioptical density units per minute, min=minute, M=molar, Na₂CO₃=sodium carbonate, ng=nanogram, NaHCO₃=sodium bicarbonate; NaNO₂=sodium nitrite; NaOH=sodium hydroxide; Na₂S₂O₃=sodium bisulfate; Na₂SO₄=sodium sulfate; NBS=N-bromosuccinamide; NH₄Cl=ammonium chloride; NH₄OAc=ammonium acetate; NaSMe=sodium methylthiolate, NBS=N-bromosuccinamide, n-BuLi=n-butyl lithium, nm=nanometer, nM=nanomolar, N=Normal, NMP=N-methylpyrrolidine, NMR=nuclear magnetic resonance, Pd/C=palladium on carbon, Pd(PPh₃)₄=Tetrakis-(triphenyl-phosphine)-palladium, pM=picomolar, Pin=pinacolato, PEG=polyethylene glycol, PPh₃ or Ph₃P=triphenyl phosphine, RLV=Raucher leukemia virus, Ra-Ni=Rainey Nickel, SOCl₂=thionyl chloride, RT=room temperature, TEA=triethylamine, THF=tetrahydrofuran, TFA=trifluoroacetic acid, TLC=thin layer chromatography, TMS=trimethylsilyl, Tf=trifluoromethylsulfonyl and TSC=trisodium citrate.

It is noted here that as used in this specification and the appended claims, the singular forms “a,” “an,” and “the” include plural reference unless the context clearly dictates otherwise.

“Alkyl,” by itself or as part of another substituent, means, unless otherwise stated, a straight or branched chain, fully saturated aliphatic hydrocarbon radical having the number of carbon atoms designated. For example, “C₁₋₈alkyl” refers to a hydrocarbon radical straight or branched, containing from 1 to 8 carbon atoms that is derived by the removal of one hydrogen atom from a single carbon atom of a parent alkane. The phrase “unsubstituted alkyl” refers to alkyl groups that do not contain groups other than fully saturated aliphatic hydrocarbon radicals. Thus the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups such as isopropyl, t-butyl, isobutyl, sec-butyl, and the like. Representative alkyl groups include straight and branched chain alkyl groups having 1, 2, 3, 4, 5, 6, 7, 8, 9, 10, 11 or 12 carbon atoms. Further representative alkyl groups include straight and branched chain alkyl groups having 1, 2, 3, 4, 5, 6, 7 or 8 carbon atoms.

“Alkylene” by itself or as part of another substituent means a divalent radical derived from an alkane, as exemplified by —CH₂CH₂CH₂CH₂—. Typically, an alkylene group will have from 1, 2, 3, 4, 5, 6, 7 or 8 carbon atoms that is derived by the removal of one hydrogen atom from a single carbon atom of a parent alkyl.

“Cycloalkyl” or “carbocycle”, by themselves or in combination with other terms, represent, unless otherwise stated, cyclic versions of “alkyl”, “alkenyl” and “alkynyl” in which all ring atoms are carbon. “Cycloalkyl” or “carbocycle” refers to a mono- or polycyclic group. When used in connection with cycloalkyl substituents, the term “polycyclic” refers herein to fused and non-fused alkyl cyclic structures. “Cycloalkyl” or “carbocycle” may form a bridged ring or a spiro ring. The cycloalkyl group may have one or more double or triple bond(s). The term “cycloalkenyl” refers to a cycloalkyl group that has at least one site of alkenyl unsaturation between the ring vertices. The term “cycloalkynyl” refers to a cycloalkyl group that has at least one site of alkynyl unsaturation between the ring vertices. When “cycloalkyl” is used in combination with “alkyl”, as in C₃₋₈cycloalkylC₃₋₈alkylene-, the cycloalkyl portion is meant to have the stated number of carbon atoms (e.g., from three to eight carbon atoms), while the alkylene portion has from one to eight carbon atoms. Typical cycloalkyl substituents have from 3 to 8 ring atoms. Examples of cycloalkyl include cyclopentyl, cyclohexyl, 1-cyclohexenyl, 3-cyclohexenyl, cycloheptyl, and the like.

“Aryl” by itself or as part of another substituent refers to a polyunsaturated, aromatic, hydrocarbon group containing from 6 to 14 carbon atoms, which can be a single ring or multiple rings (up to three rings) which are fused together or linked covalently. Thus the phrase includes, but is not limited to, groups such as phenyl, biphenyl, anthracenyl, naphthyl by way of example. Non-limiting examples of unsubstituted aryl groups include phenyl, 1-naphthyl, 2-naphthyl and 4-biphenyl. “Substituted aryl group” includes, for example, —CH₂OH (one carbon atom and one heteroatom replacing a carbon atom) and —CH₂SH. The term “heteroalkylene” by itself or as part of another substituent means a divalent radical derived from heteroalkyl, as exemplified by —CH₂₋CH₂₋S—CH₂CH₂₋— and —CH₂₋S—CH₂₋CH₂₋NH—CH₂₋. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied.

The terms “heterocycle”, “heterocyclyl” or “heterocyclic” refer to a saturated or unsaturated non-aromatic cyclic group containing at least one heteroatom. As used herein, the term “heteroatom” is meant to include oxygen (O), nitrogen (N), sulfur (S) and silicon (Si). Each heterocycle can be attached at any available ring carbon or heteroatom. Each heterocycle may have one or more rings. When multiple rings are present, they can be fused together or linked covalently. Each heterocycle typically contains 1, 2, 3, 4 or 5, independently selected heteroatoms. Preferably, these groups contain 1, 2, 3, 4, 5, 6, 7, 8, 9 or 10 carbon atoms, 0, 1, 2, 3, 4 or 5 nitrogen atoms, 0, 1 or 2 sulfur atoms and 0, 1 or 2 oxygen atoms. More preferably, these groups contain 1, 2 or 3 nitrogen atoms, 0-1 sulfur atoms and 0-1 oxygen atoms. Non-limiting examples of heterocycle groups include morpholin-3-one, piperazine-2-one, piperazin-1-oxide, pyridine-2-one, piperidine, morpholine, piperazine, isoxazoline, pyrazoline, imidazoline, pyrazol-5-one, pyrrolidine-2,5-dione, imidazolidine-2,4-dione, pyrrolidine, tetrahydroquinolinyl, decahydroquinolinyl, tetrahydrobenzooxazepinyl dihydrodibenzooxepin and the like.

“Heteroaryl” refers to a cyclic or polycyclic aromatic radical that contain from one to five heteroatoms selected from N, O, and S, wherein the nitrogen and sulfur atoms are optionally oxidized, and the nitrogen atom(s) are optionally quaternized. A heteroaryl group can be attached to the remainder of the molecule through a heteroatom or through a carbon atom and can contain 5 to 10 carbon atoms. Non-limiting examples of heteroaryl groups include 1-pyrrolyl, 2-pyrrolyl, 3-pyrrolyl, 1-pyrazolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl and 4-pyrimidyl. If not specifically stated, substituents for each of the above noted aryl and heteroaryl ring systems are selected from the group of acceptable substituents described herein. “Substituted heteroaryl” refers to a unsubstituted heteroaryl group as defined above in which one or more of the ring members is bonded to a non-hydrogen atom such as described above with respect to substituted alkyl groups and substituted aryl groups. Representative substituents include straight and branched chain alkyl groups-CH₃, —C₂H₅, —CH₂OH, —OH, —OCH₃, —OC₂H₅, —OCF₃, —OC(═O)CH₃, —OC(═O)NH₂, —OC(═O)N(CH₃)₂, —CN, —NO₂, —C(═O)CH₃, —CO₂H, —CO₂CH₃, —CONH₂, —NH₂, —N(CH₃)₂, —NHSO₂CH₃, —NHCOCH₃, —NHC(═O)OCH₃, —NHSO₂CH₃, —SO₂CH₃, —SO₂NH₂ and halo.

“Bicyclic heteroaryl” refers to bicyclic aromatic radical that contain from one to five heteroatoms selected from N, O, and S, wherein the nitrogen and sulfur atoms are optionally oxidized, and the nitrogen atom(s) are optionally quaternized. A bicyclic heteroaryl group can be attached to the remainder of the molecule through a heteroatom or through a carbon atom and can contain 5 to 10 carbon atoms. Non-limiting examples of bicyclic heteroaryl groups include 5-benzothiazolyl, purinyl, 2-benzimidazolyl, benzopyrazolyl, 5-indolyl, azaindole, 1-isoquinolyl, 5-isoquinolyl, 2-quinoxalinyl, 5-quinoxalinyl, 3-quinolyl and 6-quinolyl. If not specifically stated, substituents for each of the above noted aryl and heteroaryl ring systems are selected from the group of acceptable substituents described herein.

In each of the above embodiments designating a number of atoms e.g. “C₁₋₈” is meant to include all possible embodiments that have one fewer atom. Non-limiting examples include C₁₋₇, C₂₋₈, C₂₋₇, C₃₋₈, C₃₋₇ and the like.

Each of the terms herein (e.g., “alkyl,” “cycloalkyl,” “heteroalkyl,” “aryl” and “heteroaryl”) is meant to include both “unsubstituted” and optionally “substituted” forms of the indicated radical, unless otherwise indicated. Typically each radical is substituted with 0, 1, 2 3 4 or 5 substituents, unless otherwise indicated. Examples of substituents for each type of radical are provided below.

“Substituted” refers to a group as defined herein in which one or more bonds to a carbon(s) or hydrogen(s) are replaced by a bond to non-hydrogen and non-carbon atom “substituents” such as, but not limited to, a halogen atom such as F, Cl, Br, and I; an oxygen atom in groups such as hydroxyl groups, alkoxy groups, aryloxy, and acyloxy groups; a sulfur atom in groups such as thiol groups, alkyl and aryl sulfide groups, sulfone groups, sulfonyl groups, and sulfoxide groups; a nitrogen atom in groups such as amino, alkylamines, dialkylamines, arylamines, alkylarylamines, diarylamines, alkoxyamino, hydroxyamino, acylamino, sulfonylamino, N-oxides, imides, and enamines; and other heteroatoms in various other groups. “Substituents” also include groups in which one or more bonds to a carbon(s) or hydrogen(s) atom is replaced by a higher-order bond (e.g., a double- or triple-bond) to a heteroatom such as oxygen in oxo, acyl, amido, alkoxycarbonyl, aminocarbonyl, carboxyl, and ester groups; nitrogen in groups such as imines, oximes, hydrazones, and nitriles. “Substituents” further include groups in which one or more bonds to a carbon(s) or hydrogen(s) atoms is replaced by a bond to a cycloalkyl, heterocyclyl, aryl, and heteroaryl groups. Representative “substituents” include, among others, groups in which one or more bonds to a carbon or hydrogen atom is/are replaced by one or more bonds to fluoro, chloro, or bromo group. Another representative “substituent” is the trifluoromethyl group and other groups that contain the trifluoromethyl group. Other representative “substituents” include those in which one or more bonds to a carbon or hydrogen atom is replaced by a bond to an oxygen atom such that the substituted alkyl group contains a hydroxyl, alkoxy, or aryloxy group. Other representative “substituents” include alkyl groups that have an amine, or a substituted or unsubstituted alkylamine, dialkylamine, arylamine, (alkyl)(aryl)amine, diarylamine, heterocyclylamine, diheterocyclylamine, (alkyl)(heterocyclyl)amine, or (aryl)(heterocyclyl)amine group. Still other representative “substituents” include those in which one or more bonds to a carbon(s) or hydrogen(s) atoms is replaced by a bond to an alkyl, cycloalkyl, aryl, heteroaryl, or heterocyclyl group.

The herein-defined groups may include prefixes and/or suffixes that are commonly used in the art to create additional well-recognized substituent groups. As examples, “alkylamino” refers to a group of the formula —NR^{a}R^{b}. Unless stated otherwise, for the following groups containing R^{a}, R^{b}, R^{c}, R^{d} and R^{e}: R^{a}, and R^{b} are each independently selected from H, alkyl, alkoxy, thioalkoxy, cycloalkyl, aryl, heteroaryl, or heterocyclyl or are optionally joined together with the atom(s) to which they are attached to form a cyclic group. When R^{a} and R^{b} are attached to the same nitrogen atom, they can be combined with the nitrogen atom to form a 5-, 6- or 7-membered ring. For example, —NR^{a}R^{b} is meant to include 1-pyrrolidinyl and 4-morpholinyl.

R^{c}, R^{d}, R^{e} and R^{f} are each independently selected from alkyl, alkenyl, alkynyl, cycloalkyl, cycloalkenyl, aryl, heteroaryl, heterocyclyl or alkylenearyl as defined herein.

Typically, a particular radical will have 0, 1, 2 or 3 substituents, with those groups having two or fewer substituents being preferred in the present invention. More preferably, a radical will be unsubstituted or monosubstituted. Most preferably, a radical will be unsubstituted.

“Substituents” for the alkyl and heteroalkyl radicals (as well as those groups referred to as alkylene, alkenyl, heteroalkylene, heteroalkenyl, alkynyl, cycloalkyl, heterocyclyl) can be a variety of groups selected from, in a number ranging from zero to three, with those groups having zero, one or two substituents being particularly preferred.

In some embodiments, “substituents” for the alkyl and heteroalkyl radicals are selected from: —OR^{a}, ═O, =NR^{a}, ═N—OR^{a}, —NR^{a}R^{b}, —SR^{a}, halogen, —SiR^{a}R^{b}R^{c}, —OC(O)R^{a}, —C(O)R^{a}, —CO₂R^{a}, —CONR^{a}R^{b}, —OC(O)NR^{a}R^{b}, —NR^{b}C(O)R^{a}, —NR^{a}—C(O)NR^{b}R^{c}, —NR^{a}—SO₂ NR^{b}R^{c}, —NR^{b}CO₂R^{a}, —NH—C(NH₂)═NH, —NR^{a}C(NH₂)═NH, —NH—C(NH₂)═NR^{a}, —S(O)R^{a}, —SO₂R^{a}, SO₂NR^{a}R^{b}, —NR^{b}SO₂R, —CN and —NO₂, where R^{a} and R^{b} are as defined above. In some embodiments, substituents are selected from: —OR^{a}, ═O, —NR^{a}R^{b}, halogen, —OC(O) R^{a}, —CO₂R^{a}, —CONR^{a}R^{b}, —OC(O)NR^{a}R^{b}, —NR^{b}C(O)R^{a}, —NR^{b}CO₂R^{a}, —NR^{a}—SO₂NR^{b}R^{c}, —SO₂R^{a}, —SO₂NR^{a}R^{b}, —NR″SO₂R, —CN and —NO₂.

Examples of substituted alkyl are: —(CH₂)₃NH₂, —(CH₂)₃NH(CH₃), —(CH₂)₃NH (CH₃)₂, —CH₂C(═CH₂)CH₂NH₂, —CH₂C(═O)CH₂NH₂, —CH₂S(═O)₂CH₃, —CH₂OCH₂NH₂, CO₂H. Examples of substituents of substituted alkyl are: CH₂OH, —OH, —OCH₃, —OC₂H₅, —OCF₃, —OC(═O)CH₃, —OC(═O)NH₂, —OC(═O)N(CH₃)₂, —CN, —NO₂, —C(═O)CH₃, —CO₂H, —CO₂CH₃, —CONH₂, —NH₂, —N(CH₃)₂, —NHSO₂CH₃, —NHCOCH₃, —NHC(═O)OCH₃, —NHSO—₂CH₃, —SO₂CH₃, —SO₂NH₂, and halo.

Similarly, “substituents” for the aryl and heteroaryl groups are varied and are selected from: -halogen, —OR^{a}, —OC(O)R^{a}, —NR^{a}R^{b}, —SR^{a}, —CN, —NO₂, —CO₂R^{a}, —CON R^{a}R^{b}, —C(O)R^{a}, —OC(O)NR^{a}R^{b}, —NR^{b}C(O)R^{a}, —NR^{b}C(O)₂R^{a}, —NR^{a}—C(O)NR^{b}R^{c}, —NH—C(NH₂)═NH, —NR^{a}C(NH₂)═NH, —NH—C(NH₂)═NR^{a}, —S(O)R^{a}, —S(O)₂R^{a}, —S(O)₂NR^{a}′R^{b}, —N₃, —CH(Ph)₂, perfluoroC₁₋₈alkoxy, and perfluoroC₁₋₈alkyl, in a number ranging from zero to the total number of open valences on the aromatic ring system; and where R^{a}, R^{b} and R^{c} are independently selected from hydrogen, C₁₋₆alkyl and heteroalkyl, unsubstituted aryl and heteroaryl, (unsubstituted aryl)-C₁₋₈alkyl, and (unsubstituted aryl)oxy-C₁₋₈alkyl.

Two of the “substituents” on adjacent atoms of the aryl or heteroaryl ring may optionally be replaced with a substituent of the formula -T-C(O)—(CH₂)q-U—, wherein T and U are independently —NH—, —O—, —CH₂₋ or a single bond, and q is 0, 1 or 2. Alternatively, two of the substituents on adjacent atoms of the aryl or heteroaryl ring may optionally be replaced with a substituent of the formula -A-(CH₂)ᵣ₋B—, wherein A and B are independently —CH₂₋, —O—, —NH—, —S—, —S(O)—, —S(O)₂₋, —S(O)₂NR^{a}— or a single bond, and r is 1, 2 or 3. One of the single bonds of the new ring so formed may optionally be replaced with a double bond. Alternatively, two of the substituents on adjacent atoms of the aryl or heteroaryl ring may optionally be replaced with a substituent of the formula —(CH₂)ₛ₋X—(CH₂)ₜ₋—, where s and t are independently integers of 0 to 3, and X is —O—, —S—, —S(O)—, —S(O)₂₋, or —S(O) 2NR^{a}—. The substituent R^{a} in —NR^{a}— and —S(O)₂NR^{a}— is selected from hydrogen or unsubstituted C₁₋₆alkyl. Otherwise, R′ is as defined above.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

The term “acyl” refers to the group —C(═O)R^{c} where R^{c} is alkyl, alkenyl, alkynyl, cycloalkyl, cycloalkenyl, aryl, heteroaryl or heterocyclyl. Acyl includes the “acetyl” group —C(═O)CH₃.

“Acylamino-” refers to the group —NR^{a}C(═O)R^{c} where R^{c} is alkyl, alkenyl, alkynyl, cycloalkyl, cycloalkenyl, aryl, heteroaryl or heterocyclyl.

“Acyloxy” refers to —OC(═O)—R^{c} where R^{c} is alkyl, alkenyl, alkynyl, cycloalkyl, cycloalkenyl, aryl, heteroaryl or heterocyclyl.

“Alkoxy” refers to —OR^{d} wherein R^{d} is alkyl as defined herein. Representative examples of alkoxy groups include methoxy, ethoxy, t-butoxy, trifluoromethoxy, and the like.

“Alkoxyamino” refers to the group —NHOR^{d} where R^{d} is alkyl.

“Alkoxycarbonyl” refers to —C(═O)OR^{d} wherein R^{d} is alkyl. Representative alkoxycarbonyl groups include, for example, those shown below.

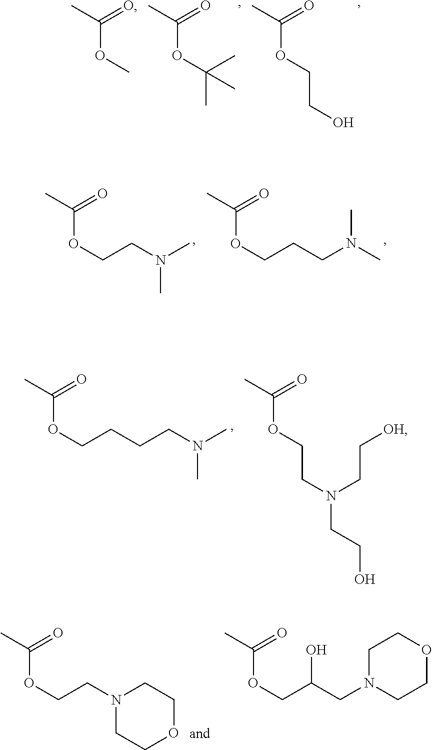

These alkoxycarbonyl groups can be further substituted as will be apparent to those having skill in the organic and medicinal chemistry arts in conjunction with the disclosure herein.

“Alkoxycarbonylamino” refers to to —NR^{a}C(═O)OR^{d} wherein R^{d} is alkyl.

“Alkoxysulfonylamino” refers to the group —NR^{a}S(═O)₂—OR^{d} where R^{d} is alkyl.

“Alkylcarbonyl” refers to the group —C(═O)R^{c} where R^{c} is alkyl.

“Alkylcarbonyloxy” refers to —OC(═O)—R^{c} where R^{c} is alkyl.

“Alkylcarbonylamino” refers to —NR^{a}C(═O)R^{c} wherein R^{c} is alkyl. Representative alkylcarbonylamino groups include, for example, —NHC(═O)CH₃, —NHC(═O)CH₂CH₃, —NHC(═O)CH₂NH(CH₃), —NHC(═O)CH₂N(CH₃)₂, or —NHC(═O)(CH₂)₃OH.

“Alkylsulfanyl”, “alkylthio”, or “thioalkoxy” refers to the group S—R^{d}. where R^{d} is alkyl.

“Alkylsulfonyl” refers to —S(═O)₂R^{e} where R^{e} is alkyl. Alkylsulfonyl groups employed in compounds of the present invention are typically C₁₋₆alkylsulfonyl groups.

“Alkylsulfonylamino” refers to —NR^{a}S(═O)₂—R^{e} wherein R^{e} is alkyl.

“Alkynyloxy” refers to the group —O-alkynyl, wherein alkynyl is as defined herein. Alkynyloxy includes, by way of example, ethynyloxy, propynyloxy, and the like.

“Amidino” refers to the group —C(═NR^{a})NR^{b}R^{c}, wherein R^{b} and R^{e} independently are selected from the group consisting of hydrogen, alkyl, substituted alkyl, alkenyl, alkynyl, aryl, cycloalkyl, cycloalkenyl, heteroaryl, heterocyclic, and where R^{b} and R^{e} are optionally joined together with the nitrogen bound thereto to form a heterocyclic or substituted heterocyclic group. R^{a} is selected from the group consisting of hydrogen, alkyl, alkenyl, alkynyl, cycloalkynyl, aryl, cycloalkyl, cycloalkenyl, heteroaryl, heterocyclic, substituted heterocyclic, nitro, nitroso, hydroxy, alkoxy, cyano, —N═N—N-alkyl, —N(alkyl)SO₂-alkyl, —N═N═N-alkyl, acyl and —SO₂-alkyl.

“Amino” refers to a monovalent radical —NR^{a}R^{b} or divalent radical —NR^{a}—. The term “alkylamino” refers to the group —NR^{a}R^{b} where R^{a} is alkyl and R^{b} is H or alkyl. The term “arylamino” refers to the group —NR^{a}R^{b} where at least one R^{a} or R^{b} is aryl. The term “(alkyl)(aryl)amino” refers to the group —NR^{a}R^{b} where R^{a} is alkyl and R^{b} is aryl. Additionally, for dialkylamino groups, the alkyl portions can be the same or different and can also be combined to form a 3-7 membered ring with the nitrogen atom to which each is attached. Accordingly, a group represented as —NR^{a}R^{b} is meant to include piperidinyl, pyrrolidinyl, morpholinyl, azetidinyl and the like.

“Aminocarbonyl” or “aminoacyl” refers to the amide —C(═O)—NR^{a}R^{b}. The term “alkylaminocarbonyl” refers herein to the group —C(═O)—NR^{a}R^{b} where R^{a} is alkyl and R^{b} is H or alkyl. The term “arylaminocarbonyl” refers herein to the group —C(═O)—NR^{a}R^{b} where R^{a} or R^{b} is aryl. Representative aminocarbonyl groups include, for example, those shown below. These aminocarbonyl group can be further substituted as will be apparent to those having skill in the organic and medicinal chemistry arts in conjunction with the disclosure herein.

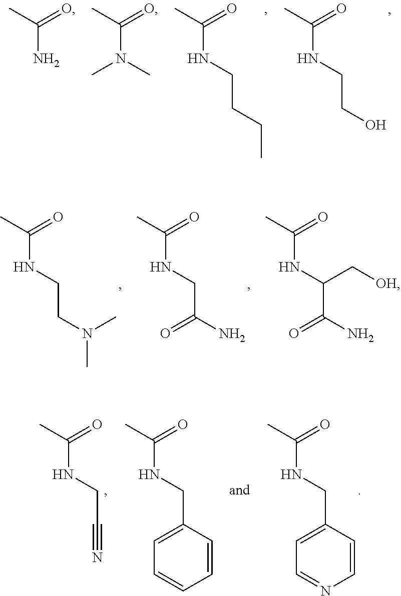

“Aminocarbonylamino” refers to the group —NR^{a}(O)NR^{a}R^{b}, wherein R^{a} is hydrogen or alkyl and R^{a} and R^{b} independently are selected from the group consisting of hydrogen, alkyl, alkenyl, alkynyl, aryl, cycloalkyl, cycloalkenyl, heteroaryl, heterocyclic, and where R^{a} and R^{b} are optionally joined together with the nitrogen bound thereto to form a heterocyclic or substituted heterocyclic group.

“Aminosulfonyl” refers to —S(O)₂NR^{a}R^{b} where R is independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R^{a} and R^{b} are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminosulfonyloxy” refers to the group —O—SO₂NR^{a}R^{b}, wherein R^{a} and R^{b} independently are selected from the group consisting of hydrogen, alkyl, alkenyl, alkynyl, aryl, cycloalkyl, cycloalkenyl, heteroaryl and heterocyclic; R^{a} and R^{b} are optionally joined together with the nitrogen bound thereto to form a heterocyclic or substituted heterocyclic group.

“Aminosulfonylamino” refers to the group —NR^{a}—SO₂NR^{b}R^{c}, wherein R^{a} is hydrogen or alkyl and R^{b} and R^{c} independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R^{b} and R^{c} are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Aminothiocarbonyl” refers to the group —C(S)NR^{a}R^{b}, wherein R^{a} and R^{b} independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R^{a} and R^{b} are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NR^{a}C(S)NR^{a}R^{b}, wherein R^{a} is hydrogen or alkyl and R^{b} and R^{c} are optionally joined together with the nitrogen bound thereto to form a heterocyclic or substituted heterocyclic group.

“Arylcarbonyl” refers to the group —C(═O)R^{c} where R^{c} is aryl.

“Arylcarbonylamino” refers to —NR^{a}C(═O)R^{c} wherein R^{c} is aryl.

“Arylcarbonyloxy” refers to —OC(═O)—R^{c} where R^{c} is aryl.

“Aryloxy” refers to —OR^{d} where R^{d} is aryl. Representative examples of aryloxy groups include phenoxy, naphthoxy, and the like.

“Aryloxycarbonyl” refers to —C(═O)OR^{d} wherein R^{d} is aryl.

“Aryloxycarbonylamino” refers to —NR^{a}C(═O)OR^{d} wherein R^{d} is aryl.

“Arylsulfanyl”, “arylthio”, or “thioaryloxy” refers to the group S—R^{d}. where R^{d} is aryl.

“Arylsulfonyl” refers to —S(═O)₂R^{e} where R^{e} is aryl.

“Arylsulfonylamino” refers to —NR^{a}S(═O)₂—R^{e} wherein R^{e} is aryl.

“Arylthio” refers to the group —S-aryl, wherein aryl is as defined herein. In other embodiments, sulfur may be oxidized to —S(O)— or —SO₂— moieties. The sulfoxide may exist as one or more stereoisomers.

“Bond” when used a element in a Markush group means that the corresponding group does not exist, and the groups of both sides are directly linked.

“Carbonyl” refers to the divalent group —C(═O)—.

“Carboxy” or “carboxyl” refers to the group —CO₂H.

“Carboxyl ester” or “carboxy ester” refers to the groups —C(═O)OR^{c}.

“Cyano” refers to —CN.

“Cycloalkylalkylene” refers to a radical —R^{x}R^{y} wherein R^{x} is an alkylene group and R^{y} is a cycloalkyl group as defined herein, e.g., cyclopropylmethyl, cyclohexenylpropyl, 3-cyclohexyl-2-methylpropyl, and the like.

“Ester” refers to —C(═O)OR^{d} wherein R^{d} is alkyl, cycloalkyl, aryl, heteroaryl, or heterocyclyl.

“Halo” or “halogen” by themselves or as part of another substituent, mean, unless otherwise stated, a fluorine, chlorine, bromine, or iodine atom. Additionally, terms such as “haloalkyl”, are meant to include alkyl in which one or more hydrogen is substituted with halogen atoms which can be the same or different, in a number ranging from one up to the maximum number of halogens permitted e.g. for alkyl, (2 m′+1), where m′ is the total number of carbon atoms in the alkyl group. For example, the term “haloC₁₋₈alkyl” is meant to include trifluoromethyl, 2,2,2-trifluoroethyl, 4-chlorobutyl, 3-bromopropyl, and the like. The term “perhaloalkyl” means, unless otherwise stated, alkyl substituted with (2 m′+1) halogen atoms, where m′ is the total number of carbon atoms in the alkyl group. For example, the term “perhaloC₁₋₈alkyl”, is meant to include trifluoromethyl, pentachloroethyl, 1,1,1-trifluoro-2-bromo-2-chloroethyl, and the like. Additionally, term “haloalkoxy” refers to an alkoxy radical substituted with one or more halogen atoms.

“Heterocyclylcarbonyl” refers to the —C(═O)R^{c} where R^{e} is heterocyclyl.

“Hydroxy” or “hydroxyl” refers to the group —OH.

“Hydroxyamino” refers to the group —NHOH.

“Nitro” refers to —NO₂.

“Nitroso” refers to the group —NO.

The terms “optional” or “optionally” as used throughout the specification means that the subsequently described event or circumstance may but need not occur, and that the description includes instances where the event or circumstance occurs and instances in which it does not. For example, “heterocyclo group optionally mono- or di-substituted with an alkyl group means that the alkyl may but need not be present, and the description includes situations where the heterocyclo group is mono- or disubstituted with an alkyl group and situations where the heterocyclo group is not substituted with the alkyl group.

“Optionally substituted” means a ring which is optionally substituted independently with substituents. A site of a group that is unsubstituted may be substituted with hydrogen.

“Oxo” refers to the divalent group ═O.

“Sulfanyl” refers to the group —SR^{f} where R^{f} is as defined herein.

“Sulfinyl” refers to the group —S(═O)—R^{e} where R^{e} is as defined herein.

“Sulfonic acid” refers to the group —S(O)₂—OH.

“Sulfonyl” refers to the group —S(O)₂—R^{e} where R^{e} is as defined herein.

“Sulfonylamino” refers to —NR^{a}S(═O)₂—R^{e} where R^{a} is selected from the group consisting of hydrogen, alkyl, alkenyl, alkynyl, aryl, cycloalkyl, cycloalkenyl, heteroaryl and heterocyclyl and R^{e} is as defined herein.

“Sulfonyloxy” refers to the group —OSO₂—R^{e}.

Compounds that have the same molecular formula but differ in the nature or sequence of bonding of their atoms or the arrangement of their atoms in space are termed “isomers”. Isomers that differ in the arrangement of their atoms in space are termed “stereoisomers”. “Stereoisomer” and “stereoisomers” refer to compounds that exist in different stereoisomeric forms if they possess one or more asymmetric centers or a double bond with asymmetric substitution and, therefore, can be produced as individual stereoisomers or as mixtures. Stereoisomers include enantiomers and diastereomers. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Unless otherwise indicated, the description is intended to include individual stereoisomers as well as mixtures. The methods for the determination of stereochemistry and the separation of stereoisomers are well-known in the art (see discussion in Chapter 4 of ADVANCED ORGANIC CHEMISTRY, 4th edition J. March, John Wiley and Sons, New York, 1992) differ in the chirality of one or more stereocenters.

“Thioacyl” refers to the groups R^{a}—C(S)—.

“Thiol” refers to the group —SH.

“Tautomer” refers to alternate forms of a molecule that differ in the position of a proton, such as enol-keto, hydroxyimine-amide and imine-enamine tautomers, or the tautomeric forms of heteroaryl groups containing a —N═C(H)—NH— ring atom arrangement, such as pyrazoles, imidazoles, benzimidazoles, triazoles, and tetrazoles. One specific tautomeric form of the compounds of the present invention may be represented as follows:

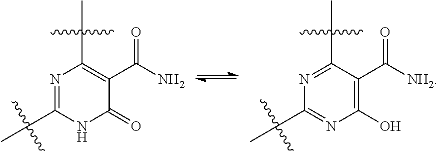 [SMILES: *c1nc(*)c(C(N)=O)c(=O)[nH]1 ; *c1nc(*)c(C(N)=O)c(O)n1]

A person of ordinary skill in the art would recognize that other tautomeric ring atom or chain atom arrangements are possible.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups are limited to -substituted aryl-(substituted aryl)-substituted aryl.

“Protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed., 1999, John Wiley & Sons, NY and Harrison et al., Compendium of Synthetic Organic Methods, Vols. 1-8, 1971-1996, John Wiley & Sons, NY. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl and trityl ethers, as well as alkyl ethers, tetrahydropyranyl ethers, trialkylsilyl ethers (e.g., TMS or TIPPS groups) and allyl ethers.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge, S. M. et al., “Pharmaceutical Salts,” Journal of Pharmaceutical Science, 66:1-19, 1977). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug ester form. “Prodrug”s of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are frequently, but not necessarily, pharmacologically inactive until converted into the active drug. Prodrugs are typically obtained by masking a functional group in the drug believed to be in part required for activity with a progroup (defined below) to form a promoiety which undergoes a transformation, such as cleavage, under the specified conditions of use to release the functional group, and hence the active drug. The cleavage of the promoiety may proceed spontaneously, such as by way of a hydrolysis reaction, or it may be catalyzed or induced by another agent, such as by an enzyme, by light, by acid or base, or by a change of or exposure to a physical or environmental parameter, such as a change of temperature. The agent may be endogenous to the conditions of use, such as an enzyme present in the cells to which the prodrug is administered or the acidic conditions of the stomach, or it may be supplied exogenously.

“Progroup” refers to a type of protecting group that, when used to mask a functional group within an active drug to form a promoiety, converts the drug into a prodrug. Progroups are typically attached to the functional group of the drug via bonds that are cleavable under specified conditions of use. Thus, a progroup is that portion of a promoiety that cleaves to release the functional group under the specified conditions of use. As a specific example, an amide promoiety of the formula —NH—C(O)CH₃ comprises the progroup —C(O)CH₃.

A wide variety of progroups, as well as the resultant promoieties, suitable for masking functional groups in the active Syk selective inhibitory compounds to yield prodrugs are well-known in the art. For example, a hydroxyl functional group may be masked as a sulfonate, ester (such as acetate or maleate) or carbonate promoiety, which may be hydrolyzed in vivo to provide the hydroxyl group. An amino functional group may be masked as an amide, carbamate, imine, urea, phosphenyl, phosphoryl or sulfenyl promoiety, which may be hydrolyzed in vivo to provide the amino group. A carboxyl group may be masked as an ester (including methyl, ethyl, pivaloyloxymethyl, silyl esters and thioesters), amide or hydrazide promoiety, which may be hydrolyzed in vivo to provide the carboxyl group. The invention includes those esters and acyl groups known in the art for modifying the solubility or hydrolysis characteristics for use as sustained-release or prodrug formulations. Other specific examples of suitable progroups and their respective promoieties will be apparent to those of skill in the art.

Certain compounds of the present invention can exist in unsolvated forms as well as solvated forms, including hydrated forms. “Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention. These isomers can be resolved or asymmetrically synthesized using conventional methods to render the isomers “optically pure”, i.e., substantially free of its other isomers. If, for instance, a particular enantiomer of a compound of the present invention is desired, it may be prepared by asymmetric synthesis, or by derivation with a chrial auxilliary, where the resulting diastereomeric mixture is separated and the auxilliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diasteromers thus formed by fractional crystallization or chromatographic means well known in the art, and subsequent recovery of the pure enantiomers.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

An “agonist” or “activator” refers to an agent or molecule that binds to a receptor of the invention, stimulates, increases, opens, activates, facilitates, enhances activation or enzymatic activity, sensitizes or up regulates the activity of a receptor of the invention.

An “antagonist” or “inhibitor” refers to an agent or molecule that inhibits or binds to, partially or totally blocks stimulation or activity, decreases, closes, prevents, delays activation or enzymatic activity, inactivates, desensitizes, or down regulates the activity of a receptor of the invention. As used herein, “antagonist” also includes a reverse or inverse agonist.

As used herein, the term “condition or disorder responsive to modulation of Syk” and related terms and phrases refer to a condition or disorder associated with inappropriate, e.g., less than or greater than normal, activity of Syk and at least partially responsive to or affected by modulation of Syk (e.g., Syk antagonist or agonist results in some improvement in patient well-being in at least some patients). Inappropriate functional activity of Syk might arise as the result of expression of Syk in cells which normally do not express the receptor, greater than normal production of Syk, or slower than normal metabolic inactivation or elimination of Syk or its active metabolites, increased expression of Syk or degree of intracellular activation (leading to, e.g., inflammatory and immune-related disorders and conditions) or decreased expression of Syk A condition or disorder associated with Syk may include a “Syk-mediated condition or disorder”.

As used herein, the phrases “a condition or disorder mediated at least in part by Syk kinase activity”, and related phrases and terms refer to a condition or disorder characterized by inappropriate, e.g., greater than normal, Syk activity. Inappropriate Syk functional activity might arise as the result of Syk expression in cells which normally do not express Syk or increased Syk expression or degree of intracellular activation (leading to, e.g., inflammatory and immune-related disorders and conditions). A condition or disorder mediated at least in part by Syk kinase activity may be completely or partially mediated by inappropriate Syk functional activity. However, a condition or disorder mediated at least in part by Syk kinase activity is one in which modulation of Syk results in some effect on the underlying condition or disorder (e.g., an Syk antagonist results in some improvement in patient well-being in at least some patients).

The term “inflammation” as used herein refers to infiltration of white blood cells (e.g., leukocytes, monocytes, etc.) into the area being treated for restenosis.

The term “intervention” refers to an action that produces an effect or that is intended to alter the course of a disease process. For example, “vascular intervention” refers to the use of an intravascular procedure such as angioplasty or a stent to open an obstructed blood vessel.

The term “intravascular device” refers to a device useful for a vascular recanalization procedure to restore blood flow through an obstructed blood vessel. Examples of intravascular devices include, without limitation, stents, balloon catheters, autologous venous/arterial grafts, prosthetic venous/arterial grafts, vascular catheters, and vascular shunts.

As used herein, the term “JAK” refers to a Janus kinase (RefSeq Accession No. P-43408) or a variant thereof that is capable of mediating gene expression in vitro or in vivo. JAK variants include proteins substantially homologous to native JAK, i.e., proteins having one or more naturally or non-naturally occurring amino acid deletions, insertions or substitutions (e.g., JAK derivatives, homologs and fragments). The amino acid sequence of JAK variant preferably is at least about 80% identical to a native JAK, more preferably at least about 90% identical, and most preferably at least about 95% identical.

The term “leukocyte” refers to any of the various blood cells that have a nucleus and cytoplasm, separate into a thin white layer when whole blood is centrifuged, and help protect the body from infection and disease. Examples of leukocytes include, without limitation, neutrophils, eosinophils, basophils, lymphocytes, and monocytes.

The term “mammal” includes, without limitation, humans, domestic animals (e.g., dogs or cats), farm animals (cows, horses, or pigs), monkeys, rabbits, mice, and laboratory animals.

The terms “modulate”, “modulation” and the like refer to the ability of a compound to increase or decrease the function and/or expression of Syk, where such function may include transcription regulatory activity and/or protein-binding. Modulation may occur in vitro or in vivo. Modulation, as described herein, includes the inhibition, antagonism, partial antagonism, activation, agonism or partial agonism of a function or characteristic associated with Syk, either directly or indirectly, and/or the upregulation or downregulation of the expression of Syk, either directly or indirectly. In a preferred embodiment, the modulation is direct. Inhibitors or antagonists are compounds that, e.g., bind to, partially or totally block stimulation, decrease, prevent, inhibit, delay activation, inactivate, desensitize, or downregulate signal transduction. Activators or agonists are compounds that, e.g., bind to, stimulate, increase, open, activate, facilitate, enhance activation, activate, sensitize or upregulate signal transduction. The ability of a compound to inhibit the function of Syk can be demonstrated in a biochemical assay, e.g., binding assay, or a cell-based assay, e.g., a transient transfection assay.

“Modulators” of activity are used to refer to “ligands”, “antagonists” and “agonists” identified using in vitro and in vivo assays for activity and their homologs and mimetics. Modulators include naturally occurring and synthetic ligands, antagonists, agonists, molecules and the like. Assays to identify antagonists and agonists include, e.g., applying putative modulator compounds to cells, in the presence or absence of a receptor of the invention and then determining the functional effects on a receptor of the invention activity. Samples or assays comprising a receptor of the invention that are treated with a potential activator, inhibitor, or modulator are compared to control samples without the inhibitor, activator, or modulator to examine the extent of effect. Control samples (untreated with modulators) are assigned a relative activity value of 100%. Inhibition is achieved when the activity value of a receptor of the invention relative to the control is about 80%, optionally 50% or 25-1%. Activation is achieved when the activity value of a receptor of the invention relative to the control is 110%, optionally 150%, optionally 200-500%, or 1000-3000% higher.

“Patient” refers to human and non-human animals, especially mammals. Examples of patients include, but are not limited to, humans, cows, dogs, cats, goats, sheep, pigs and rabbits.

Turning next to the compositions of the invention, the term “pharmaceutically acceptable carrier or excipient” means a carrier or excipient that is useful in preparing a pharmaceutical composition that is generally safe, non-toxic and neither biologically nor otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

The terms “pharmaceutically effective amount”, “therapeutically effective amount” or “therapeutically effective dose” refers to the amount of the subject compound that will elicit the biological or medical response of a tissue, system, animal or human that is being sought by the researcher, veterinarian, medical doctor or other clinician. The term “therapeutically effective amount” includes that amount of a compound that, when administered, is sufficient to prevent development of, or alleviate to some extent, one or more of the symptoms of the condition or disorder being treated. The therapeutically effective amount will vary depending on the compound, the disorder or condition and its severity and the age, weight, etc., of the mammal to be treated.

The term “platelet” refers to a minute, normucleated, disklike cell found in the blood plasma of mammals that functions to promote blood clotting.

The terms “prevent”, “preventing”, “prevention” and grammatical variations thereof as used herein, refers to a method of partially or completely delaying or precluding the onset or recurrence of a disorder or condition and/or one or more of its attendant symptoms or barring a subject from acquiring or reacquiring a disorder or condition or reducing a subject's risk of acquiring or reaquiring a disorder or condition or one or more of its attendant symptoms.

The term “recanalization” refers to the process of restoring flow to or reuniting an interrupted channel of the body, such as a blood vessel.

The term “restenosis” refers to a re-narrowing or blockage of an artery at the same site where treatment, such as an angioplasty or a stent procedure, has been performed.

The phrase “selectively” or “specifically” when referring to binding to a receptor, refers to a binding reaction that is determinative of the presence of the receptor, often in a heterogeneous population of receptors and other biologics. Thus, under designated conditions, the compounds bind to a particular receptor at least two times the background and more typically more than 10 to 100 times background. Specific binding of a compound under such conditions requires a compound that is selected for its specificity for a particular receptor. For example, small organic molecules can be screened to obtain only those compounds that specifically or selectively bind to a selected receptor and not with other receptors or proteins. A variety of assay formats may be used to select compounds that are selective for a particular receptor. For example, High-throughput screening assays are routinely used to select compounds that are selective for a particular a receptor.

As used herein, the term “Sickle cell anemia” refers to an inherited disorder of the red blood cells in which both hemoglobin alleles encode the sickle hemoglobin (S) protein, i.e., the S/S genotype. The presence of abnormal hemoglobin results in the production of unusually shaped cells, which do not survive the usual length of time in the blood circulation. Thus, anemia results. “Anemia” refers to a decrease in the number of red blood cells and/or hemoglobin in the blood.

The term “Sickle cell disease” refers to an inherited disorder of the red blood cells in which one hemoglobin allele encodes the sickle hemoglobin (S) protein, and the other allele encodes another unusual hemoglobin protein, such as hemoglobin (S), (C), (D), (E), and (βThal). Examples of sickle cell disease genotypes include, without limitation, the S/S, S/C, S/D, S/E, and S/βThal genotypes. The most common types of sickle cell disease include sickle cell anemia, sickle-hemoglobin C disease, sickle beta-plus thalassemia, and sickle beta-zero thalassemia.

The “subject” is defined herein to include animals such as mammals, including, but not limited to, primates (e.g., humans), cows, sheep, goats, horses, dogs, cats, rabbits, rats, mice and the like. In preferred embodiments, the subject is a human.

As used herein, the term “Syk” refers to a spleen tyrosine kinase (RefSeq Accession No. P-043405) or a variant thereof that is capable of mediating a cellular response to T-cell receptors in vitro or in vivo. Syk variants include proteins substantially homologous to native Syk, i.e., proteins having one or more naturally or non-naturally occurring amino acid deletions, insertions or substitutions (e.g., Syk derivatives, homologs and fragments). The amino acid sequence of Syk variant preferably is at least about 80% identical to a native Syk, more preferably at least about 90% identical, and most preferably at least about 95% identical.

The term “Syk inhibitor” refers to any agent that inhibits the catalytic activity of spleen tyrosine kinase.

The term “thrombosis” refers to the blockage or clotting of a blood vessel caused by a clumping of cells, resulting in the obstruction of blood flow. The term “thrombosis” refers to the clot that is formed within the blood vessel.

The terms “treat”, “treating”, “treatment” and grammatical variations thereof as used herein, includes partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, pallatively or remedially.

The term “vessel” refers to any channel for carrying a fluid, such as an artery or vein. For example, a “blood vessel” refers to any of the vessels through which blood circulates in the body. The lumen of a blood vessel refers to the inner open space or cavity of the blood vessel.

### 2. EMBODIMENTS OF THE INVENTION

a. Compounds

The present invention provides in one group of embodiments, a compound having formula (I):

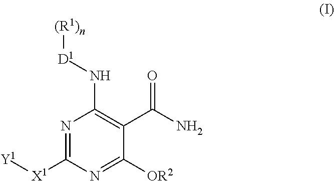

or a tautomer thereof or a pharmaceutically acceptable salt or hydrate thereof, wherein:

D¹ is selected from the group consisting of:
(a) aryl substituted with a group, R⁵,
R⁵ is selected from the group consisting of:
   (i) heteroaryl;
   (ii) C₁₋₈alkyl;
   (iii) C₁₋₈alkoxy;
   (iv) C₁₋₈alkylcarbonylamino;
   (v) aminosulfonyl;
   (vi) heterocyclyl;
   (vii) halo;
   (viii) haloalkoxy;
each R⁵ is optionally further substituted with from 1 to 3 substituents independently selected from the group consisting of C₁₋₈alkyl, C₂₋₈ alkenyl, C₂₋₈alkynyl, hydroxyl, C₁₋₈alkoxy, oxo, halo, C₁₋₈alkylcarbonyl, aminocarbonyl, C₁₋₈ alkylcarbonylamino; C₁₋₈ alkylaminocarbonyl; aminosulfonyl, heteroaryl and heterocyclyl;
   (b) C₃₋₈cycloalkyl;
   (c) heteroaryl; and

each R¹ is independently selected from the group consisting of C₁₋₈alkyl, C₂₋₈ alkenyl, C₂₋₈alkynyl, hydroxyl, C₁₋₈alkoxy, oxo, halo, C₁₋₈alkylcarbonyl, aminocarbonyl, C₁₋₈ alkylcarbonylamino; C₁₋₈ alkylaminocarbonyl; aminosulfonyl, heteroaryl and heterocyclyl;

X¹ is NH or S;

Y¹ is selected from the group consisting of:

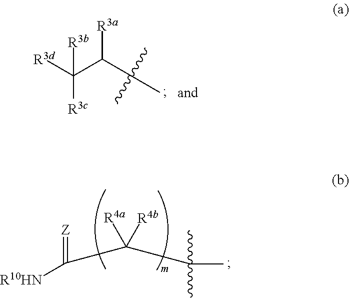

Z is O or S; and
(c) phenyl; substituted with a group, R^{6a}, selected from the group consisting of:
   (i) heterocyclyl;
   (ii) C₁₋₈alkylcarbonylamino;
   (iii) C₁₋₈alkoxy;
   (iv) heteroaryl;
   (v) aminosulfonyl;
each R^{6a} is optionally further substituted with from 1 to 2 substituents independently selected from the group consisting of C₁₋₈alkyl, C₂₋₈ alkenyl, C₂₋₈alkynyl, hydroxyl, C₁₋₈alkoxy, oxo, halo, C₁₋₈alkylcarbonyl, aminocarbonyl, C₁₋₈ alkylcarbonylamino; C₁₋₈ alkylaminocarbonyl; aminosulfonyl, heteroaryl and heterocyclyl;

R² is H or C₁₋₈alkyl, optionally substituted with from 1 to 2 hydroxy, C₁₋₈ alkoxy or amino groups;

each R^{3a}, R^{4a} and R^{4b} is independently selected from the group consisting of: H, C₁₋₈alkyl, hydroxyC₁₋₈alkyl, C₁₋₈haloalkyl, amino, C₁₋₈alkylamino, C₁₋₈ alkoxycarbonylaminoC₁₋₈ alkylene, C₃₋₈cycloalkyl, heteroaryl, C₁₋₈ alkylC₃₋₈cycloalkyl, C₁₋₈alkylthioC₁₋₈ alkyl, C₁₋₈alkylsulfonylC₁₋₈ alkylene, aminocarbonyl, C₁₋₈alkoxyC₁₋₈alkyl, haloC₁₋₈alkyl, aryl and heterocyclyl; wherein the aryl is optionally substituted by hydroxyl, C₁₋₈alkoxy, halo or haloC₁₋₈alkyl; or taken together with R^{3b} and the atoms to which they are attached to form a C₃₋₈ cycloalkyl or heterocycloalkyl ring;

R^{3b} is selected from the group consisting of H, C₁₋₈alkyl, C₁₋₈alkylamino, amino aminoC₁₋₈alkyl, carboxy, C₁₋₈alkylaminoC₁₋₈alkyl, C₁₋₈alkoxyC₁₋₈alkyl, hydroxyC₁₋₈alkyl; carboxyC₁₋₈alkyl, C₃₋₈cycloalkylC₁₋₈alkyl, aryloxyC₁₋₈alkyl, arylC₁₋₈alkyl, heteroarylC₁₋₈alkyl, and hydroxyC₁₋₈alkoxy and hydroxyC₁₋₈alkoxy; or may be combined with R^{3a} or R^{3d} and the atoms to which they are attached to form a C₃₋₈ cycloalkyl or heterocyclyl ring;

R^{3c} is selected from the group consisting of H, amino, C₁₋₈alkylamino, hydroxycarbonylamino C₁₋₈alkoxycarbonylamino, arylC₁₋₈alkoxycarbonylamino and hydroxyl;

R^{3d} is H or alkyl or may be combined with R^{3b} and the atoms to which they are attached to form a C₃₋₈ cycloalkyl or heterocyclyl ring;

R¹⁰ is H or C₁₋₈alkyl;

the subscript n is 0, 1, 2, 3 or 4; and

the subscript m is an integer of 1, 2 or 3; and the wavy line indicates the point of attachment to the rest of the molecule.

The present invention provides in another group of embodiments, a compound wherein R² is H. The present invention provides in another group of embodiments, a compound wherein R² is C₁₋₈alkyl.

The present invention provides in another group of embodiments, a compound wherein Y¹ is:

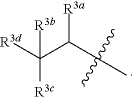

The present invention provides in another group of embodiments, a compound wherein Y¹ is:

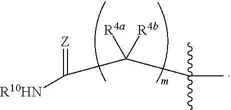

The present invention provides in another group of embodiments, a compound wherein Z is S. The present invention provides in another group of embodiments, a compound wherein Z is O.

The present invention provides in another group of embodiments, a compound wherein Y¹ is phenyl.

The present invention provides in another group of embodiments, a compound wherein D¹ is aryl. The present invention provides in another group of embodiments, a compound wherein D¹ is phenyl.

The present invention provides in another group of embodiments, a compound,

wherein R⁵ is heteroaryl. The present invention provides in another group of embodiments, a compound, wherein R⁵ is C₁₋₈alkyl. The present invention provides in another group of embodiments, a compound, wherein R⁵ is C₁₋₈alkoxy. The present invention provides in another group of embodiments, a compound, wherein R⁵ is C₁₋₈alkylcarbonylamino. The present invention provides in another group of embodiments, a compound, wherein R⁵ is aminosulfonyl. The present invention provides in another group of embodiments, a compound, wherein R⁵ is heterocyclyl. The present invention provides in another group of embodiments, a compound, wherein R⁵ is halo. The present invention provides in another group of embodiments, a compound, wherein R⁵ is haloalkoxy.

The present invention provides in another group of embodiments, a compound wherein D¹ is C₃₋₈cycloalkyl. The present invention provides in another group of embodiments, a compound, wherein D¹ is selected from the group consisting of cyclopropyl, cyclobutyl, cyclopentyl and cyclohexyl. The present invention provides in another group of embodiments, a compound wherein D¹ is heteroaryl.

The present invention provides in another group of embodiments, a compound wherein X¹ is NH. The present invention provides in another group of embodiments, a compound, wherein X¹ is S.

The present invention provides in another group of embodiments, a compound wherein the heteroaryl group, alone or when part of a group containing a heteroaryl moiety is selected from the group consisting of:

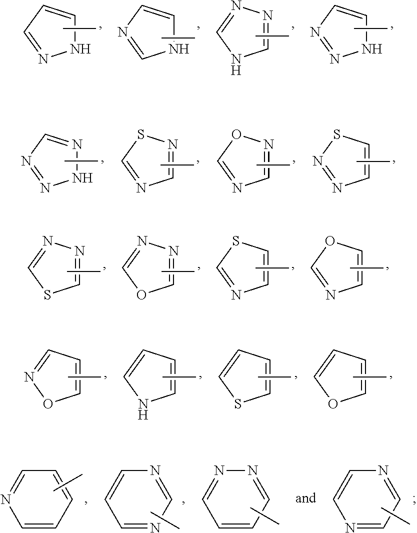

each of which is optionally substituted with from 1 to 2 substituents independently selected from the group consisting of: C₁₋₈ alkyl, amino, hydroxyl, oxo, halo, C₁₋₈alkoxy, hydroxyC₁₋₈alkyl-, aminoC₁₋₈alkyl, C₁₋₈alkylcarbonyl, haloC₁₋₈alkyl, C₃₋₈cycloalkyl, C₁₋₈-aminocycloalkyl, aminoC₁₋₈alkylenecarbonyl, aminocarbonyl, C₁₋₈alkyleneaminoC₁₋₈alkylenecarbonyl, C₁₋₈alkoxyC₁₋₈alkylenecarbonyl, hydroxyC₁₋₈alkylenecarbonyl, hydroxyC₁₋₈alkoxycarbonyl, C₁₋₈alkoxycarbonylamino, aryl, arylC₁₋₈alkoxycarbonylamino, C₁₋₈alkylsulfonyl, aminoC₁₋₈alkylenesulfonyl, aminosulfonyl, C₁₋₈alkyleneaminoC₁₋₈alkylenesulfonyl, C₁₋₈alkoxyC₁₋₈alkylenesulfonyl, hydroxyC₁₋₈alkylenesulfonyl, hydroxyC₁₋₈alkoxysulfonyl, aminosulfonyl, and C₁₋₈alkylheterocyclyl.

The present invention provides in another group of embodiments, a compound wherein the heteroaryl group, alone or when part of a group containing a heteroaryl moiety is selected from the group consisting of:

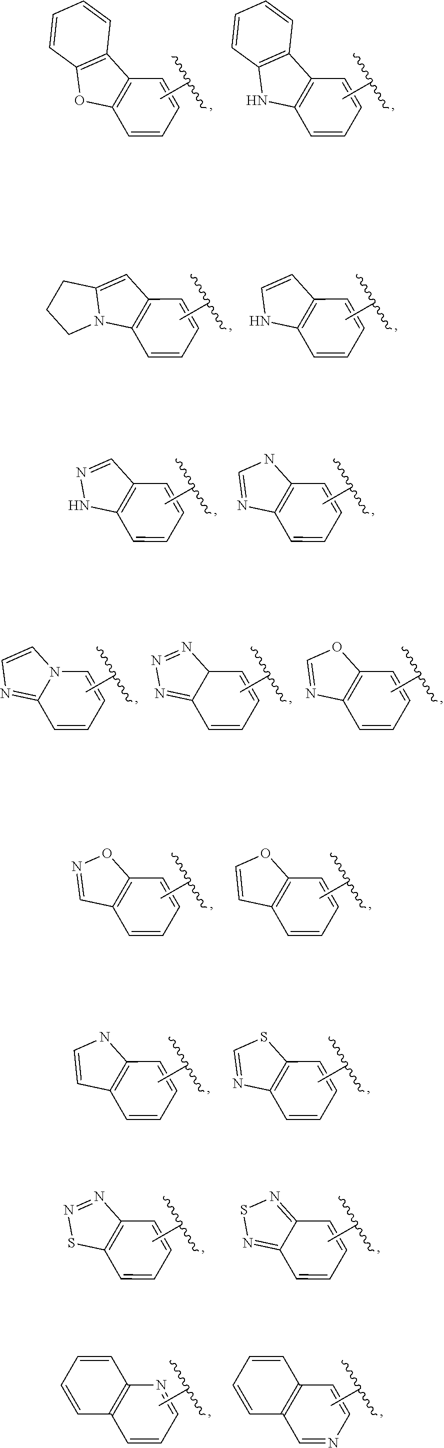

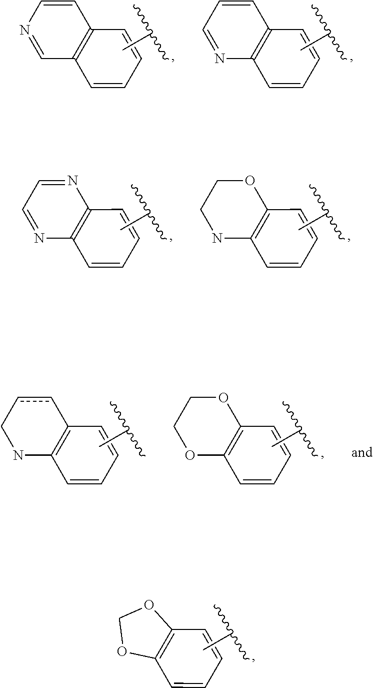

optionally substituted with from 1 to 3 R⁷ substituents independently selected from the group consisting of: C₁₋₈ alkyl, C₁₋₈alkylcarbonyl, C₁₋₈-aminocycloalkyl, aminoC₁₋₈alkylenecarbonyl, aminocarbonyl, C₁₋₈alkyleneaminoC₁₋₈alkylenecarbonyl, C₁₋₈alkoxyC₁₋₈alkylenecarbonyl, hydroxyC₁₋₈alkylenecarbonyl, hydroxyC₁₋₈alkoxycarbonyl, aminocarbonyl, amino, C₁₋₈ alkoxycarbonylamino, aryl, arylC₁₋₈ alkoxycarbonylamino, hydroxyl, C₁₋₈ alkoxy, C₁₋₈alkylsulfonyl, aminoC₁₋₈alkylenesulfonyl, aminosulfonyl, C₁₋₈alkyleneaminoC₁₋₈alkylenesulfonyl, C₁₋₈alkoxyC₁₋₈alkylenesulfonyl, hydroxyC₁₋₈alkylenesulfonyl, hydroxyC₁₋₈alkoxysulfonyl, aminosulfonyl, oxo, halo, phenyl and C₁₋₈alkylheterocyclyl; and the wavy line indicates the point of attachment to the rest of the molecule.

The present invention provides in another group of embodiments, a compound, wherein R⁵ is selected from the group consisting of

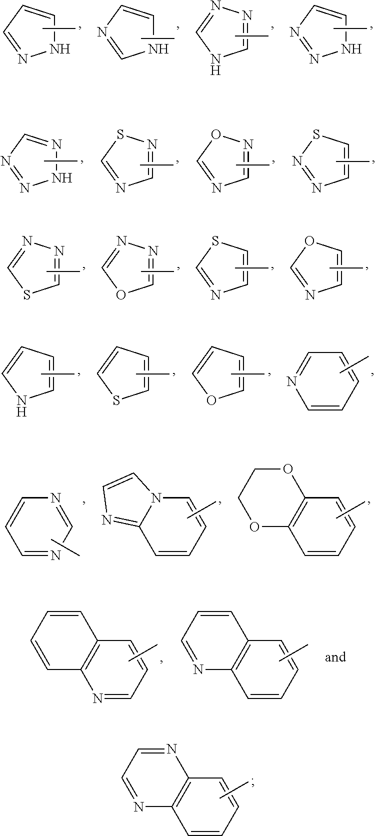

each of which is optionally substituted with from 1 to 2 substituents independently selected from the group consisting of C₁₋₈alkyl, C₂₋₈ alkenyl, C₂₋₈alkynyl, hydroxyl, C₁₋₈alkoxy, oxo, halo, C₁₋₈alkylcarbonyl, aminocarbonyl, C₁₋₈ alkylcarbonylamino; C₁₋₈ alkylaminocarbonyl; aminosulfonyl, heteroaryl and heterocyclyl.

The present invention provides in another group of embodiments, a compound wherein R⁵ is selected from the group consisting of

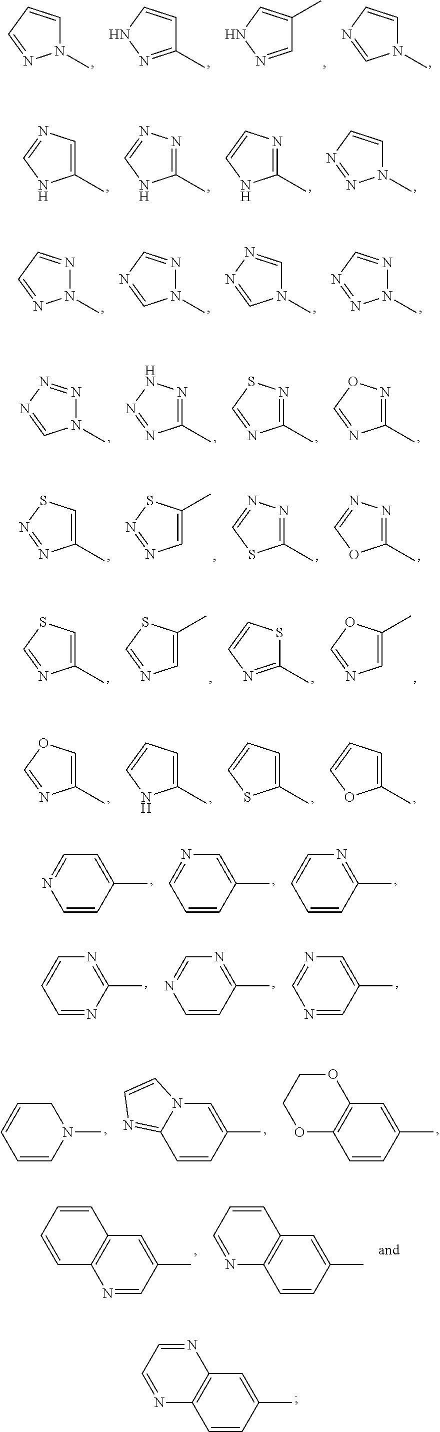

each of which is optionally substituted with from 1 to 2 substituents independently selected from the group consisting of C₁₋₈alkyl, C₂₋₈ alkenyl, C₂₋₈alkynyl, hydroxyl, C₁₋₈alkoxy, oxo, halo, C₁₋₈alkylcarbonyl, aminocarbonyl, C₁₋₈ alkylcarbonylamino; C₁₋₈ alkylaminocarbonyl; aminosulfonyl, heteroaryl and heterocyclyl.

The present invention provides in another group of embodiments, a compound wherein R⁵ is selected from the group consisting of

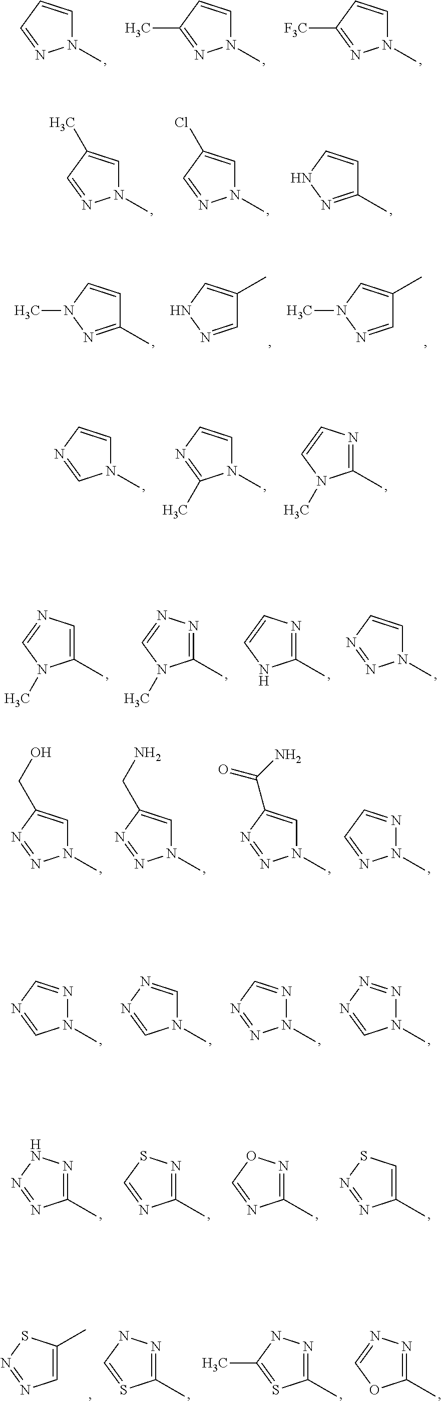

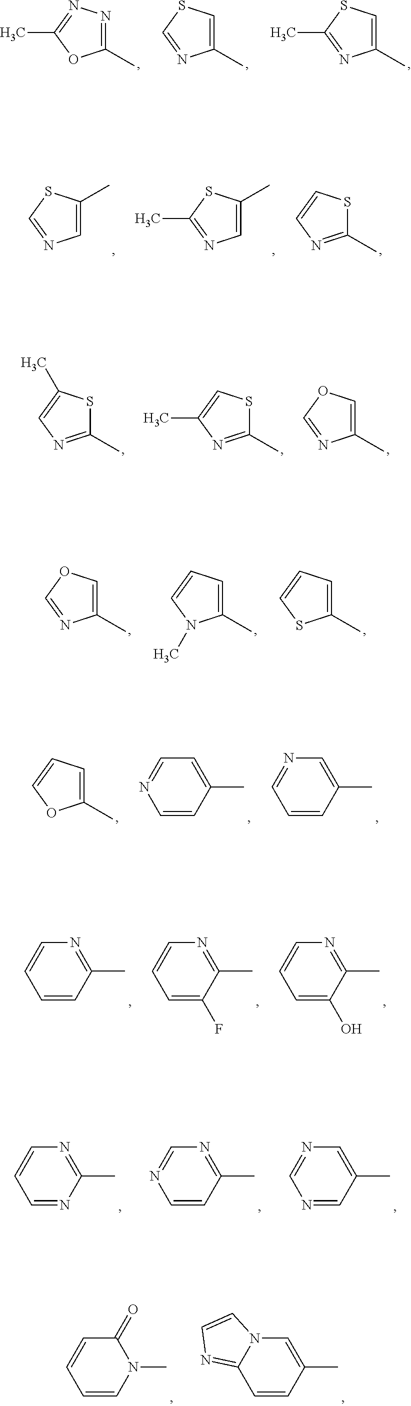

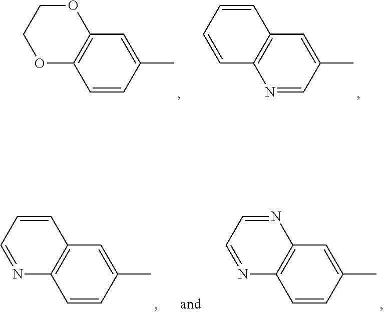

The present invention provides in another group of embodiments, a compound, wherein: D¹ is bicyclic heteroaryl. The present invention provides in another group of embodiments, a compound, wherein D¹ is selected from the group consisting of:

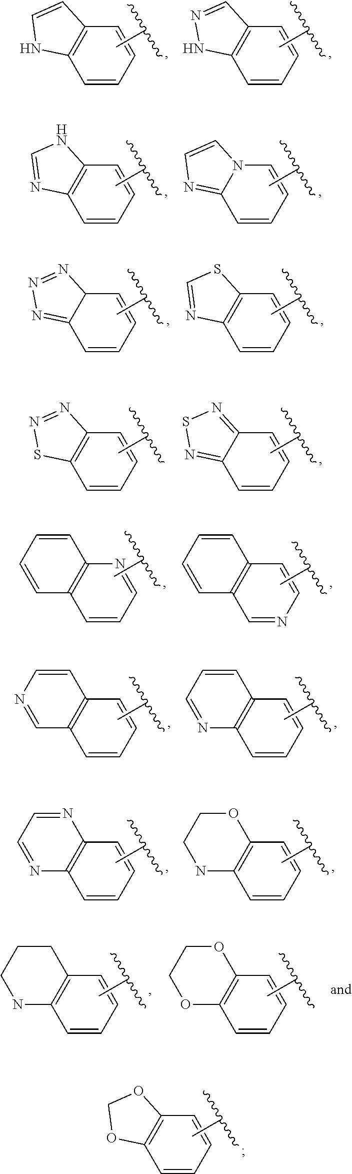

optionally substituted with from 1 to 3 R⁷ substituents independently selected from the group consisting of: C₁₋₈ alkyl, C₁₋₈alkylcarbonyl, aminoC₁₋₈alkylenecarbonyl, aminocarbonyl, C₁₋₈ alkyleneaminoC₁₋₈ alkylenecarbonyl, C₁₋₈ alkoxyC₁₋₈ alkylenecarbonyl, hydroxyC₁₋₈ alkylenecarbonyl, hydroxyC₁₋₈ alkoxycarbonyl, aminocarbonyl, amino, C₁₋₈ alkoxycarbonylamino, arylC₁₋₈ alkoxycarbonylamino, hydroxyl, C₁₋₈ alkoxy, C₁₋₈ alkylsulfonyl, aminoC₁₋₈ alkylenesulfonyl, aminosulfonyl, C₁₋₈ alkyleneaminoC₁₋₈ alkylenesulfonyl, C₁₋₈ alkoxyC₁₋₈ alkylenesulfonyl, hydroxyC₁₋₈alkylenesulfonyl, hydroxyC₁₋₈alkoxysulfonyl, aminosulfonyl, oxo, halo, phenyl and C₁₋₈alkylheterocyclyl; and the wavy line indicates the point of attachment to the rest of the molecule.

The present invention provides in another group of embodiments, a compound, wherein D¹ is selected from the group consisting of:

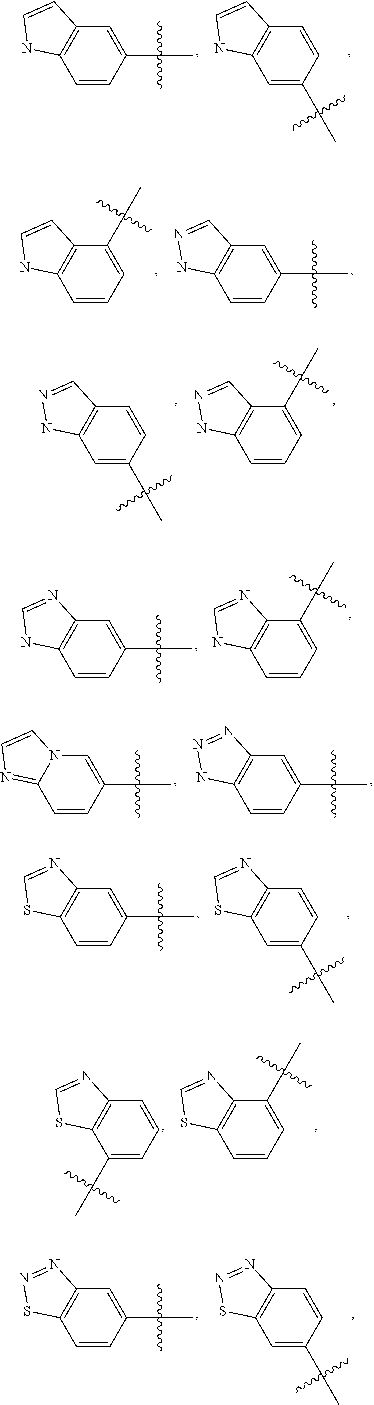

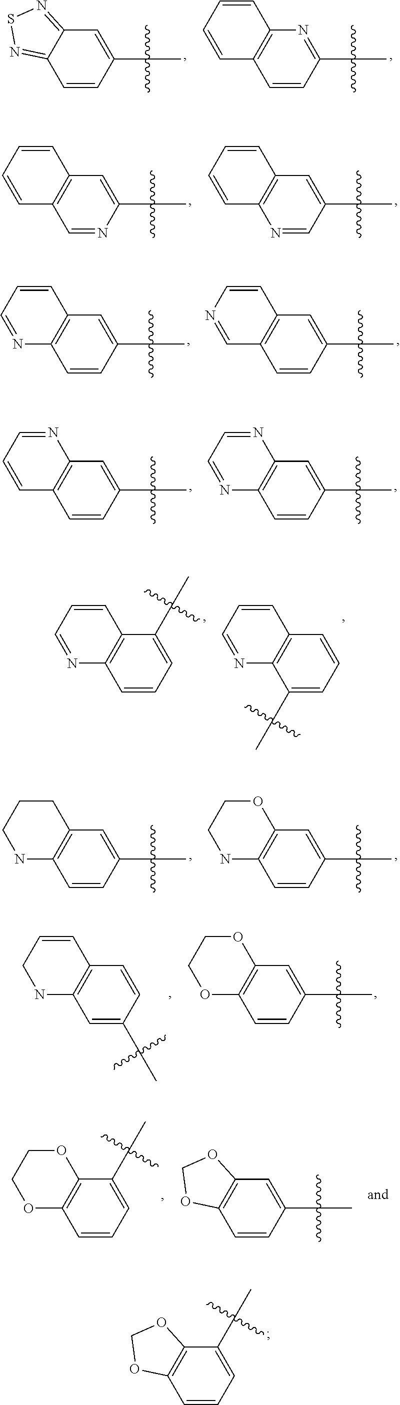

and the wavy line indicates the point of attachment to the rest of the molecule.

The present invention provides in another group of embodiments, a compound, wherein D¹ is selected from the group consisting of:

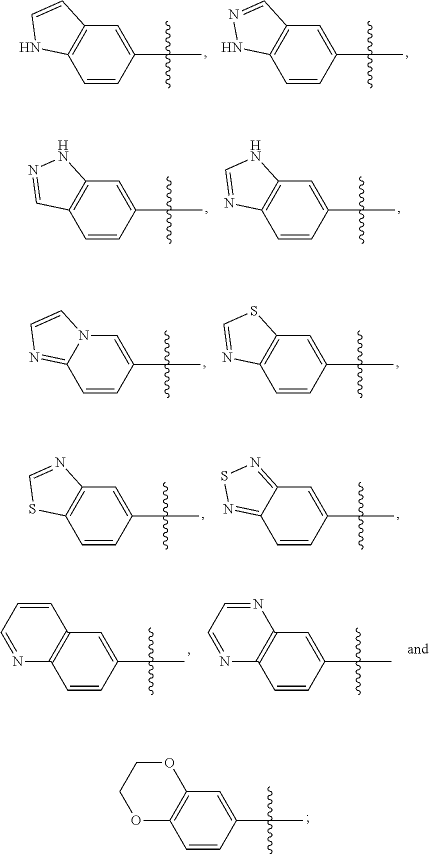

and the wavy line indicates the point of attachment to the rest of the molecule.

The present invention provides in another group of embodiments, a compound, wherein D¹ is selected from the group consisting of:

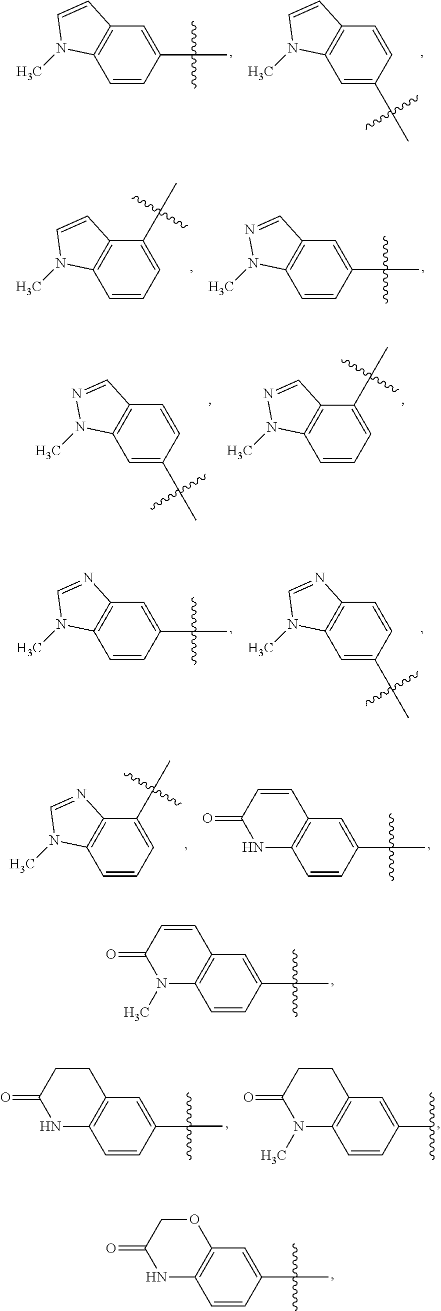

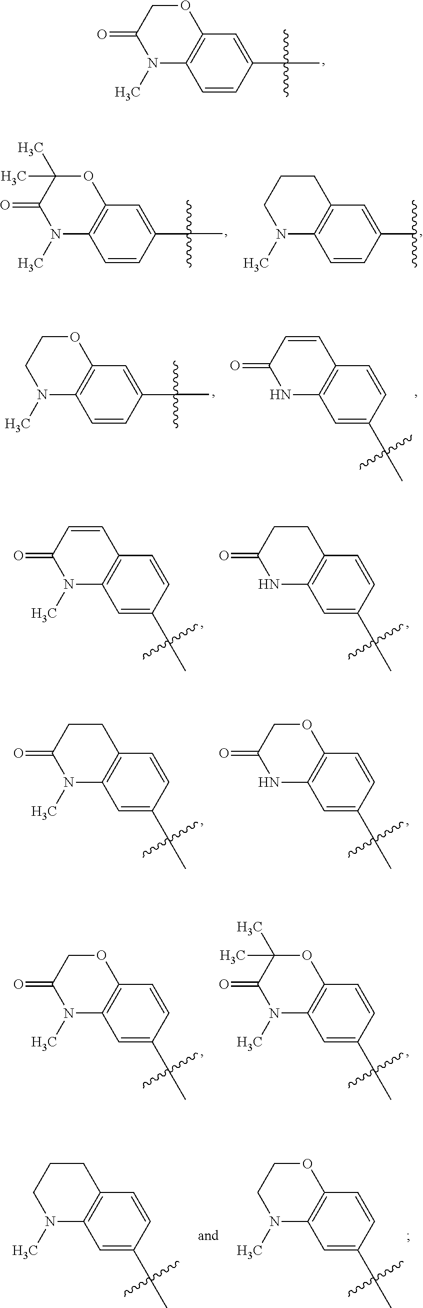

and the wavy line indicates the point of attachment to the rest of the molecule.

The present invention provides in another group of embodiments, a compound, wherein each R¹ is independently selected from the group consisting of C₁₋₈alkyl, C₂₋₈ alkenyl, C₂₋₈alkynyl, hydroxyl, C₁₋₈alkoxy, oxo, halo, C₁₋₈alkylcarbonyl, aminocarbonyl, C₁₋₈ alkylcarbonylamino; C₁₋₈ alkylaminocarbonyl; aminosulfonyl, heteroaryl and heterocyclyl.

The present invention provides in another group of embodiments, a compound, wherein R^{3c} is amino.

The present invention provides in another group of embodiments, a compound, wherein: the moiety:

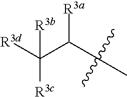

is

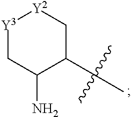

wherein Y² and Y³ is each independently selected from the group consisting of: CH₂, NH, NCOCH₃ and S.

The present invention provides in another group of embodiments, a compound, wherein the moiety:

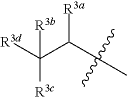

is selected from the group consisting of:

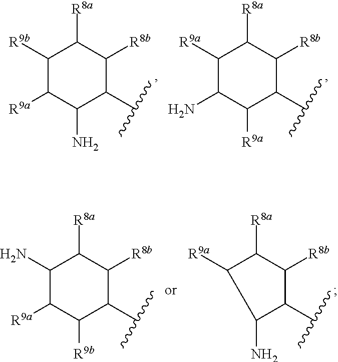

wherein each R^{8a} and R^{8b} is independently H, hydroxyl, halo or if on adjacent carbon atoms, may be combined with the atoms to which they are attached to form a fused benzene ring; and
each R^{9a} and R^{9b} is independently H, hydroxyl, halo or, if on adjacent carbon atoms, may be combined with the atoms to which they are attached to form a fused benzene ring; and the wavy line indicates the point of attachment to the rest of the molecule.

The present invention provides in another group of embodiments, a compound, wherein: the moiety:

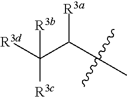

is

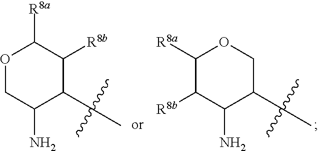

wherein each R^{8a} and R^{8b} is independently H or may be combined with the atoms to which they are attached to form a fused benzene ring; and the wavy line indicates the point of attachment to the rest of the molecule.

The present invention provides in another group of embodiments, a compound wherein the compound has the formula:

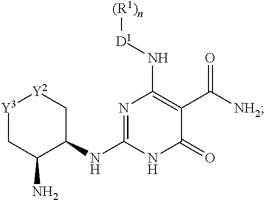

wherein each Y² and Y³ is independently selected from the group consisting of: CH₂, NH, NCOCH₃ and S.

The present invention provides in another group of embodiments, a compound wherein the compound has the formula:

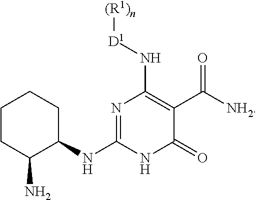

The present invention provides in another group of embodiments, a compound wherein the compound has the formula:

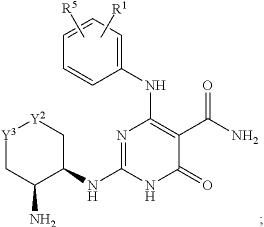

wherein each Y² and Y³ is independently selected from the group consisting of: CH₂, NH, NCOCH₃ and S.

The present invention provides in another group of embodiments, a compound wherein the compound has the formula:

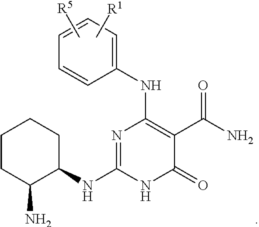

The present invention provides in another group of embodiments, a compound wherein the compound has the formula:

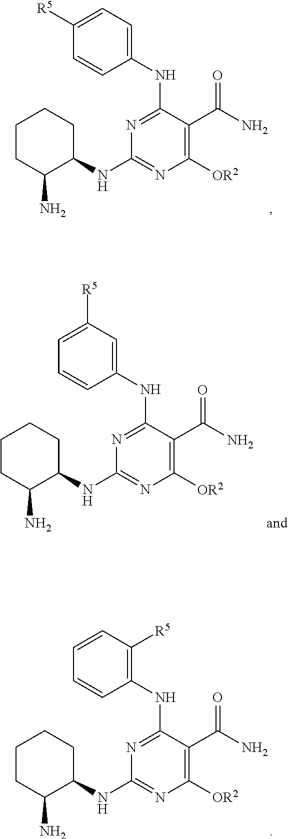

The present invention provides in another group of embodiments, a compound wherein the compound has the formula selected from the group consisting of:

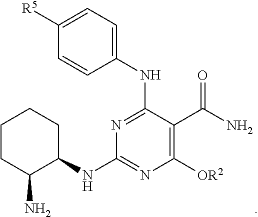

The present invention provides in another group of embodiments, a compound wherein the compound has the formula selected from the group consisting of:

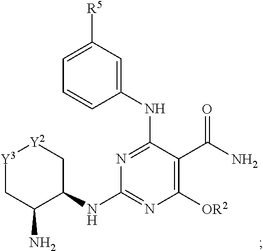

wherein each Y² and Y³ is independently selected from the group consisting of: CH₂, NH, NCOCH₃ and S.

The present invention provides in another group of embodiments, a compound wherein the compound has the formula selected from the group consisting of:

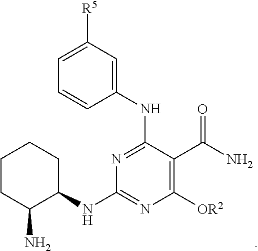

The present invention provides in another group of embodiments, a compound wherein R⁵ is:

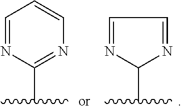

The present invention provides in another group of embodiments, a compound, wherein the moiety:

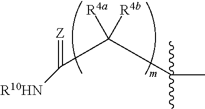

is

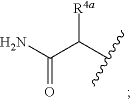

and the wavy line indicates the point of attachment to the rest of the molecule.

The present invention provides in another group of embodiments, a compound having the formula:

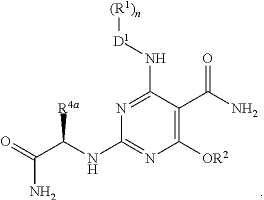

The present invention provides in another group of embodiments, a compound having the formula:

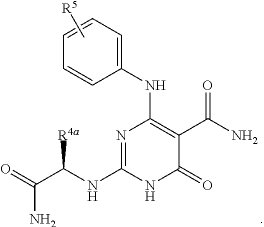

The present invention provides in another group of embodiments, a compound wherein D¹ has a formula selected from the group consisting of:

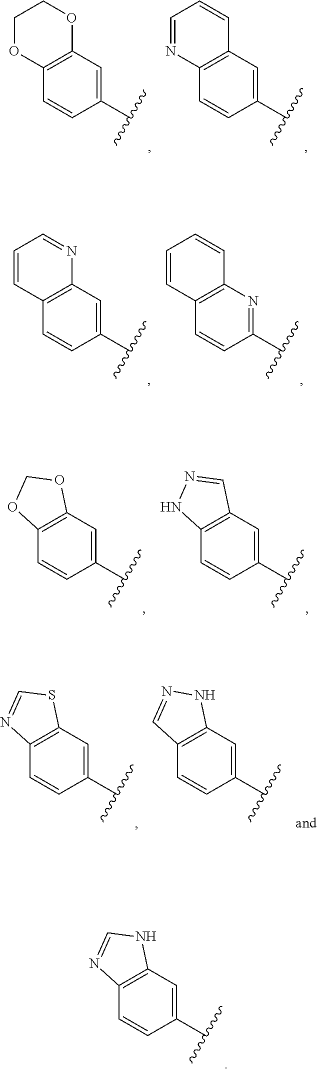

The present invention provides in another group of embodiments, a compound wherein the compound has a formula selected from the group consisting of:

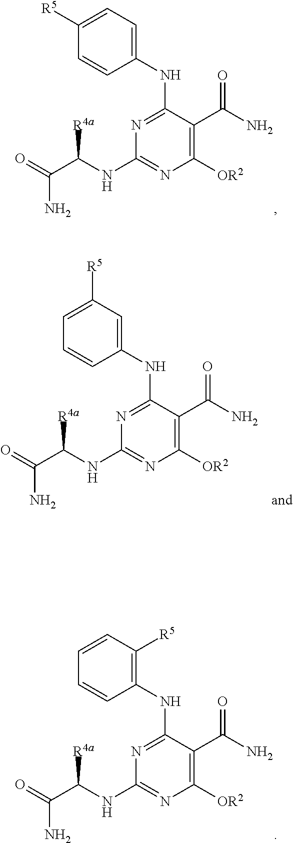

The present invention provides in another group of embodiments, a compound wherein R⁵ has a formula selected from the group consisting of —OCH₃, —OCHF₂, —OCF₃, —OCH₂CH₂OCH₃,

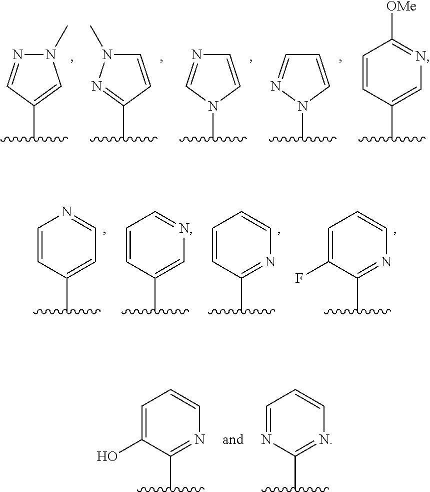

The present invention provides in another group of embodiments, a compound wherein R^{4a} is selected from the group consisting of C₁₋₈alkyl, C₃₋₈cycloalkyl, aryl, heteroaryl and heterocyclyl. The present invention provides in another group of embodiments, a compound, wherein R^{4a} is C₁₋₈alkyl. The present invention provides in another group of embodiments, a compound, wherein R^{4a} is methyl, ethyl, isopropyl, isobutyl, cyclopropylmethyl or phenyl.

The present invention provides in another group of embodiments, a compound, wherein the moiety:

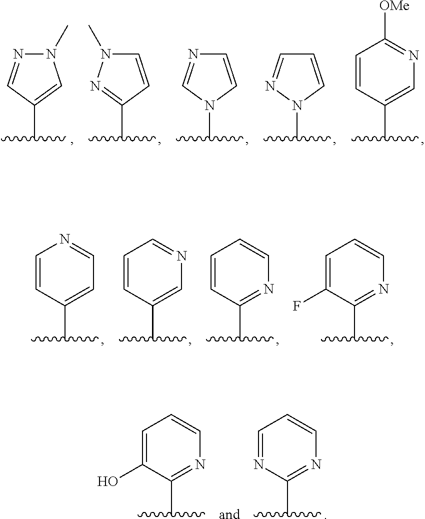

is selected from the group consisting of:

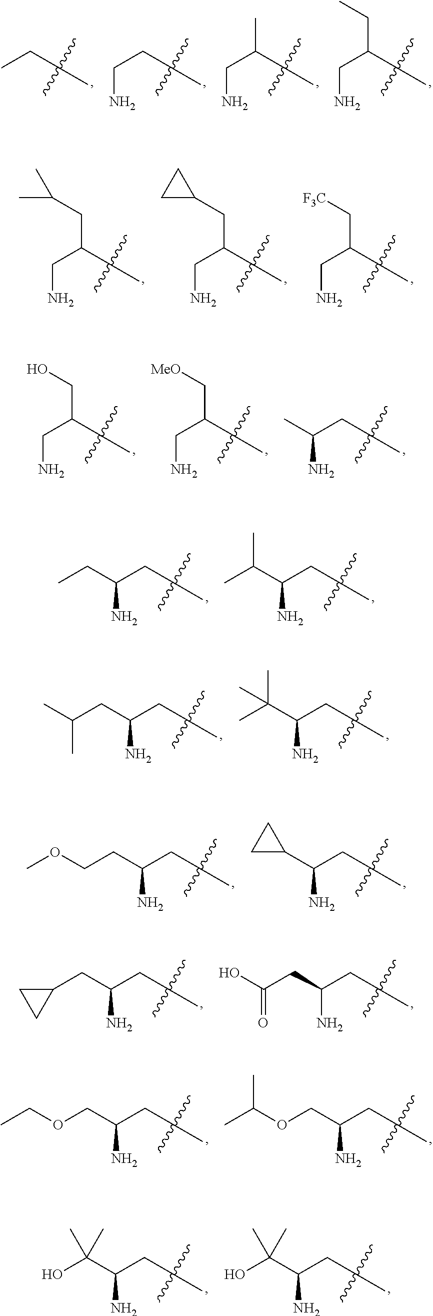

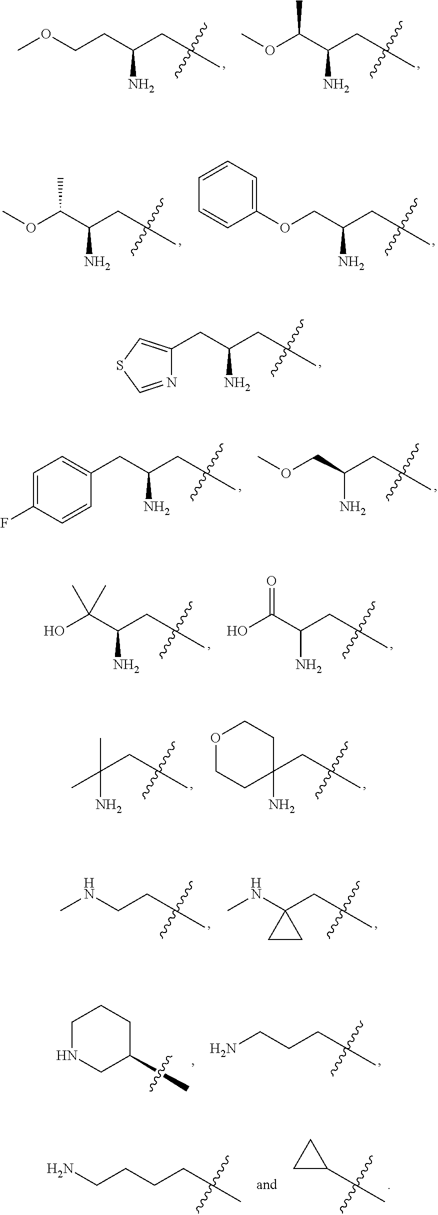

The present invention provides in another group of embodiments, a compound, wherein D² is selected from the group consisting of: phenyl, 3-methylphenyl, 4-methylphenyl, 3-methoxyphenyl, 4-methoxyphenyl, 4-chlorophenyl, 4-aminosulfonylphenyl,

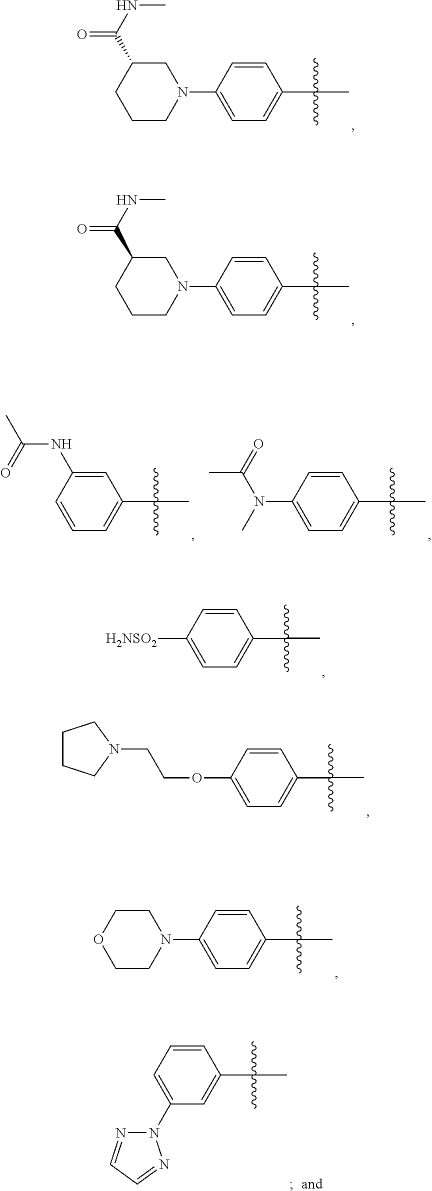

the wavy line indicates the point of attachment to the rest of the molecule.

The present invention provides in another embodiment, a compound selected from the group consisting of: (R)-2-(1-amino-3-methyl-1-oxobutan-2-ylamino)-4-(3,5-dimethylphenylamino)-6-methoxypyrimidine-5-carboxamide; (R)-2-(1-amino-3-methyl-1-oxobutan-2-ylamino)-4-(3,5-dimethylphenylamino)-6-oxo-1,6-dihydropyrimidine-5-carboxamide; 2-((1R,2 S)-2-aminocyclohexylamino)-4-(3-(2H-1,2,3-triazol-2-yl)phenylamino)-6-methoxypyrimidine-5-carboxamide; (R)-2-(1-amino-3-methyl-1-oxobutan-2-ylamino)-4-(3,5-dimethylphenylamino)-6-ethoxypyrimidine-5-carboxamide; 4-(3-(2H-1,2,3-triazol-2-yl)phenylamino-2-((1R,2S)-2-aminocyclohexyl)-6-oxo-1,6-dihydropyrimidine-5-carboxamide; (R)-4-(3-(2H-1,2,3-triazol-2-yl)phenylamino)-2-(1-amino-3-methyl-1-oxobutan-2-ylamino)-6-oxo-1,6-dihydropyrimidine-5-carboxamide; (R)-4-(3-(2H-1,2,3-triazol-2-yl)phenylamino)-2-(1-amino-4-methyl-1-oxopentan-2-ylamino)-6-oxo-1,6-dihydropyrimidine-5-carboxamide; (R)-4-(3-(2H-1,2,3-triazol-2-yl)phenylamino)-2-(1-amino-1-oxobutan-2-ylamino)-6-oxo-1,6-dihydropyrimidine-5-carboxamide; (R)-2-(1-amino-4-methyl-1-oxopentan-2-ylamino)-6-oxo-4-(m-tolylamino)-1,6-dihydropyrimidine-5-carboxamide; (R)-2-(1-amino-3-methyl-1-oxobutan-2-ylamino)-6-oxo-4-(m-tolylamino)-1,6-dihydropyrimidine-5-carboxamide; (R)-2-(1-amino-1-oxobutan-2-ylamino)-6-oxo-4-(m-tolylamino)-1,6-dihydropyrimidine-5-carboxamide; (R)-2-(1-amino-3-methylbutan-2-ylamino)-4-(3,5-dimethylphenylamino)-6-oxo-1,6-dihydropyrimidine-5-carboxamide; (R)-2-(1-amino-4-methyl-1-oxopentan-2-ylamino)-6-oxo-4-(quinolin-6-ylamino)-1,6-dihydropyrimidine-5-carboxamide; 2-(2-(methylamino)ethylamino-6-oxo-4-(m-tolyl)-1,6-dihydropyrimidine-5-carboxamide; (R)-2-(1-amino-3-methyl-1-oxobutan-2-ylamino)-4-methoxy-6-(quinolin-6-ylamino)pyrimidine-5-carboxamide; (R)-2-(1-amino-1-oxobutan-2-ylamino)-4-methoxy-6-(quinolin-6-ylamino)pyrimidine-5-carboxamide; (R)-6-(2-(1-amino-1-oxobutan-2-ylamino)-5-carbamoyl-6-methoxypyrimidin-4-ylamino)quinoline 1-oxide; (R)-4-(4-(1H-pyrazol-1ylamino)phenylamino)-2-(1-amino-4-methyl-1-oxopentan-2-ylamino)-6-oxo-1,6-dihydropyrimidine-5-carboxamide; (R)-4-(4-(1H-pyrazol-1ylamino)phenylamino)-2-(1-amino-3-methyl-1-oxobutan-2-ylamino)-6-oxo-1,6-dihydropyrimidine-5-carboxamide; (R)-2-(1-amino-3-methyl-1-oxobutan-2-ylamino)-6-oxo-4-(quinolin-6-ylamino)-1,6-dihydropyrimidine-5-carboxamide; (R)-2-(1-amino-1-oxobutan-2-ylamino)-6-oxo-4-(quinolin-6-ylamino)-1,6-dihydropyrimidine-5-carboxamide; (R)-4-(3-(2H-1,2,3-triazol-2-yl)phenylamino)-2-(1-amino-3-methylbutan-2-ylamino)-6-oxo-1,6-dihydropyrimidine-5-carboxamide; 4-(3-(2H-1,2,3-triazol-2-yl)phenylamino)-2-(2-(methylamino)ethylamino)-6-oxo-1,6-dihydropyrimidine-5-carboxamide; (R)-4-(4-(1H-pyrazol-1ylamino)phenylamino)-2-(1-amino-1-oxobutan-2-ylamino)-6-oxo-1,6-dihydropyrimidine-5-carboxamide; (R)-4-(1H-indazol-5-ylamino)-2-(1-amino-4-methyl-1-oxopentan-2-ylamino)-6-oxo-1,6-dihydropyrimidine-5-carboxamide; (R)-4-(1H-indazol-5-ylamino)-2-(1-amino-3-methyl-1-oxobutan-2-ylamino)-6-oxo-1,6-dihydropyrimidine-5-carboxamide; (R)-2-(1-amino-4-methyl-1-oxopentan-2-ylamino)-6-oxo-4-(3-(pyrimidin-2-yl)phenylamino)-1,6-dihydropyrimidine-5-carboxamide; (R)-2-(1-amino-3-methyl-1-oxobutan-2-ylamino)-6-oxo-4-(3-(pyrimidin-2-yl)phenylamino)-1,6-dihydropyrimidine-5-carboxamide; (R)-2-(1-amino-4-methyl-1-oxopentan-2-ylamino)-6-oxo-4-(3-(pyrimidin-2-yl)phenylamino)-1,6-dihydropyrimidine-5-carboxamide; (R)-2-(1-amino-3-methyl-1-oxobutan-2-ylamino)-6-oxo-4-(3-(pyrimidin-2-yl)phenylamino)-1,6-dihydropyrimidine-5-carboxamide; (R)-2-(1-amino-1-oxobutan-2-ylamino)-6-oxo-4-(3-(pyrimidin-2-yl)phenylamino)-1,6-dihydropyrimidine-5-carboxamide; (R)-4-(3-(1H-pyrazol-1ylamino)phenylamino)-2-(1-amino-4-methyl-1-oxopentan-2-ylamino)-6-oxo-1,6-dihydropyrimidine-5-carboxamide; (R)-4-(3-(1H-pyrazol-1ylamino)phenylamino)-2-(1-amino-3-methyl-1-oxobutan-2-ylamino)-6-oxo-1,6-dihydropyrimidine-5-carboxamide; (R)-4-(3-(1H-pyrazol-1ylamino)phenylamino)-2-(1-amino-1-oxobutan-2-ylamino)-6-oxo-1,6-dihydropyrimidine-5-carboxamide; (R)-4-(1H-indazol-6-ylamino)-2-(1-amino-4-methyl-1-oxopentan-2-ylamino)-6-oxo-1,6-dihydropyrimidine-5-carboxamide; (R)-4-(1H-indazol-6-ylamino)-2-(1-amino-1-oxobutan-2-ylamino)-6-oxo-1,6-dihydropyrimidine-5-carboxamide; (R)-4-(3-(2H-1,2,3-triazol-2-yl)phenylamino)-2-(1-amino-3-methylbutan-2-ylamino)-6-oxo-1,6-dihydropyrimidine-5-carboxamide; 4-(3-(pyrimidin-2-yl)phenylamino)-2-((1R,2S)-2-aminocyclohexyl)-6-oxo-1,6-dihydropyrimidine-5-carboxamide; (R)-2-(1-amino-1-oxobutan-2-ylamino)-4-(3-(methoxy)phenylamino)-6-oxo-1,6-dihydropyrimidine-5-carboxamide; (R)-2-(1-amino-4-methyl-1-oxopentan-2-ylamino)-6-oxo-4-(3-(pyridin-2-yl)phenylamino)-1,6-dihydropyrimidine-5-carboxamide; (R)-2-(1-amino-1-oxobutan-2-ylamino)-6-oxo-4-(3-(pyridin-2-yl)phenylamino)-1,6-dihydropyrimidine-5-carboxamide; 2-(cyclopropylamino)-6-oxo-4-(4-sulfamoylphenylamino)-1,6-dihydropyrimidine-5-carboxamide; 2-(cyclopropylamino)-4-(4-(N-methylacetamido)phenylamino)-6-oxo-1,6-dihydropyrimidine-5-carboxamide; 4-(cyclopropylamino)-6-oxo-2-(4-sulfamoylphenylamino)-1,6-dihydropyrimidine-5-carboxamide; 4-(cyclobutylamino)-6-oxo-2-(4-sulfamoylphenylamino)-1,6-dihydropyrimidine-5-carboxamide; 2,4-bis(4-(4-acetylpiperazin-1-yl)phenylamino)-6-oxo-1,6-dihydropyrimidine-5-carboxamide; 2-(4-(4-acetylpiperazin-1-yl)phenylamino)-4-(cyclobutylamino)-6-oxo-1,6-dihydropyrimidine-5-carboxamide; 2-(2-aminocyclohexylamino)-4-(cyclopropylamino)-6-hydroxypyrimidine-5-carboxamide and 2-(2-amino-2-oxoethylamino)-4-(3,5-dimethylphenylamino)-6-oxo-1,6-dihydropyrimidine-5-carboxamide; 2-(2-amino-2-oxoethylamino)-4-(3,5-dimethylphenylamino)-6-methoxypyrimidine-5-carboxamide; (S)-2-(2-aminopropylthio)-6-oxo-4-(quinolin-6-ylamino)-1,6-dihydropyrimidine-5-carboxamide; 4-(m-toluidino)-6-(3-aminopropoxy)-2-(ethylamino)pyrimidine-5-carboxamide; (S)-4-(m-toluidino)-2-(2-aminopropylamino)-6-(2-hydroxyethoxy)pyrimidine-5-carboxamide; (S)-2-(2-aminopropylamino)-4-(3,5-dimethylphenylamino)-6-oxo-1,6-dihydropyrimidine-5-carboxamide; (R)-2-(1-amino-4-methyl-1-oxopentan-2-ylamino)-4-(3,5-dimethylphenylamino)-6-oxo-1,6-dihydropyrimidine-5-carboxamide; (R)-2-(1-amino-1-oxopropan-2-ylamino)-4-(3,5-dimethylphenylamino)-6-oxo-1,6-dihydropyrimidine-5-carboxamide; 2-(2-aminocyclohexylamino)-4-(3,5-dimethylphenylamino)-6-oxo-1,6-dihydropyrimidine-5-carboxamide; 4-(cyclopropylamino)-2-(4-(N-methylacetamido)phenylamino)-6-oxo-1,6-dihydropyrimidine-5-carboxamide; 2-(4-(4-acetylpiperazin-1-yl)phenylamino)-4-(3,5-dimethylphenylamino)-6-oxo-1,6-dihydropyrimidine-5-carboxamide; 2-(cyclopropylamino)-4-(3,5-dimethylphenylamino)-6-oxo-1,6-dihydropyrimidine-5-carboxamide; 4-(4-(3,5-dimethylphenylamino)-5-carboxamide-6-oxo-1,6-dihydropyrimidin-2-ylamino)benzenesulfonamide; 2-(4-(4-acetylpiperazin-1-yl)phenylamino)-4-(cyclopropylamino)-6-oxo-1,6-dihydropyrimidine-5-carboxamide; 4-(4-(3-methylphenylamino)-5-carboxamide-6-oxo-1,6-dihydropyrimidin-2-ylamino)benzenesulfonamide; 4-(m-toluidino)-2-(4-(4-acetylpiperazin-1-yl)phenylamino)-6-hydroxypyrimidine-5-carboxamide; 2-(2-aminocyclohexylamino)-4-(3,5-dimethylphenylamino)-6-methoxypyrimidine-5-carboxamide; (R)-2-(1-amino-1-oxopropan-2-ylamino)-4-(3,5-dimethylphenylamino)-6-methoxypyrimidine-5-carboxamide; (R)-2-(1-amino-4-methyl-1-oxopentan-2-ylamino)-4-(3,5-dimethylphenylamino)-6-methoxypyrimidine-5-carboxamide; (S)-2-(2-aminopropylamino)-4-(3,5-dimethylphenylamino)-6-methoxypyrimidine-5-carboxamide; 2-(4-(4-acetylpiperazin-1-yl)phenylamino)-4-(cyclopropylamino)-6-methoxypyrimidine-5-carboxamide; 4-(m-toluidino)-6-methoxy-2-(4-morpholinophenylamino)pyrimidine-5-carboxamide; (R)-4-(m-toluidino)-2-(2-amino-2-oxo-1-phenylethylamino)-6-methoxypyrimidine-5-carboxamide; (R)-2-(2-amino-2-oxo-1-phenylethylamino)-4-(3,5-dimethylphenylamino)-6-methoxypyrimidine-5-carboxamide; 4-(cyclopropylamino)-6-methoxy-2-(4-morpholinophenylamino)pyrimidine-5-carboxamide; 4-(m-toluidino)-2-(4-(4-acetylpiperazin-1-yl)phenylamino)-6-methoxypyrimidine-5-carboxamide; 4-(m-toluidino)-2-(ethylamino)-6-(2-hydroxyethoxy)pyrimidine-5-carboxamide; (R)-2-(1-amino-1-oxobutan-2-ylamino)-4-(benzo[d][1,3]dioxol-5-ylamino)-6-oxo-1,6-dihydropyrimidine-5-carboxamide; (R)-2-(1-amino-4-methyl-1-oxopentan-2-ylamino)-4-(3-methoxyphenylamino)-6-oxo-1,6-dihydropyrimidine-5-carboxamide; (R)-2-(1-amino-4-methyl-1-oxopentan-2-ylamino)-4-(2,3-dihydrobenzo[b][1,4]dioxin-6-ylamino)-6-oxo-1,6-dihydropyrimidine-5-carboxamide; (R)-2-(1-amino-4-methyl-1-oxopentan-2-ylamino)-4-(benzo[d][1,3]dioxol-5-ylamino)-6-oxo-1,6-dihydropyrimidine-5-carboxamide; (R)-2-(1-amino-4-methyl-1-oxopentan-2-ylamino)-6-oxo-4-(3-(3-fluoropyridin-2-yl)phenylamino)-1,6-dihydropyrimidine-5-carboxamide; (R)-2-(1-amino-1-oxobutan-2-ylamino)-6-oxo-4-(3-(3-fluoropyridin-2-yl)phenylamino)-1,6-dihydropyrimidine-5-carboxamide; (R)-2-(1-amino-1-oxobutan-2-ylamino)-6-oxo-4-(3-(3-hydroxypyridin-2-yl)phenylamino)-1,6-dihydropyrimidine-5-carboxamide; (R)-2-(1-amino-4-methyl-1-oxopentan-2-ylamino)-4-(3,4-dimethoxyphenylamino)-6-oxo-1,6-dihydropyrimidine-5-carboxamide; (R)-2-(1-amino-1-oxobutan-2-ylamino)-4-(2,3-dihydrobenzo[b][1,4]dioxin-6-ylamino)-6-oxo-1,6-dihydropyrimidine-5-carboxamide; (R)-2-(1-amino-4-methyl-1-oxopentan-2-ylamino)-4-(3-(difluoromethoxy)phenylamino)-6-oxo-1,6-dihydropyrimidine-5-carboxamide; (R)-2-(1-amino-1-oxobutan-2-ylamino)-4-(3-(difluoromethoxy)phenylamino)-6-oxo-1,6-dihydropyrimidine-5-carboxamide; (R)-2-(1-amino-4-methyl-1-oxopentan-2-ylamino)-4-(4-methoxyphenylamino)-6-oxo-1,6-dihydropyrimidine-5-carboxamide; (R)-2-(1-amino-1-oxobutan-2-ylamino)-4-(4-methoxyphenylamino)-6-oxo-1,6-dihydropyrimidine-5-carboxamide; (R)-2-(1-amino-4-methyl-1-oxopentan-2-ylamino)-4-(4-(trifluoromethoxy)phenylamino)-6-oxo-1,6-dihydropyrimidine-5-carboxamide; (R)-2-(1-amino-1-oxobutan-2-ylamino)-4-(4-(trifluoromethoxy)phenylamino)-6-oxo-1,6-dihydropyrimidine-5-carboxamide; (R)-2-(1-amino-4-methyl-1-oxopentan-2-ylamino)-4-(4-(2-methoxyethoxy)phenylamino)-6-oxo-1,6-dihydropyrimidine-5-carboxamide; (R)-2-(1-amino-1-oxobutan-2-ylamino)-4-(4-(2-methoxyethoxy)phenylamino)-6-oxo-1,6-dihydropyrimidine-5-carboxamide; (R)-2-(1-amino-3-cyclopropyl-1-oxopropan-2-ylamino)-6-oxo-4-(3-(pyridin-2-yl)phenylamino)-1,6-dihydropyrimidine-5-carboxamide; (R)-2-(1-amino-3-methyl-1-oxobuan-2-ylamino)-6-oxo-4-(3-(pyridin-2-yl)phenylamino)-1,6-dihydropyrimidine-5-carboxamide; (R)-2-(1-amino-4-methyl-1-oxopentan-2-ylamino)-6-oxo-4-(3-(pyridin-3-yl)phenylamino)-1,6-dihydropyrimidine-5-carboxamide; (R)-2-(1-amino-4-methyl-1-oxopentan-2-ylamino)-4-(3-(2-methoxyethoxy)phenylamino)-6-oxo-1,6-dihydropyrimidine-5-carboxamide; (R)-2-(1-amino-1-oxobutan-2-ylamino)-4-(3-(2-methoxyethoxy)phenylamino)-6-oxo-1,6-dihydropyrimidine-5-carboxamide (R)-2-(4-methyl-1-(methylamino)-1-oxopentan-2-ylamino)-6-oxo-4-(3-(pyridin-2-yl)phenylamino)-1,6-dihydropyrimidine-5-carboxamide; (R)-2-(1-amino-4-methyl-1-oxopentan-2-ylamino)-4-(3-(trifluoromethoxy)phenylamino)-6-oxo-1,6-dihydropyrimidine-5-carboxamide; (R)-2-(1-amino-1-oxobutan-2-ylamino)-4-(3-(trifluoromethoxy)phenylamino)-6-oxo-1,6-dihydropyrimidine-5-carboxamide; (R)-4-(benzo[d]thiazol-6-ylamino)-2-(4-methyl-1-(methylamino)-1-oxopentan-2-ylamino)-6-oxo-1,6-dihydropyrimidine-5-carboxamide; (R)-4-(3-(1H-pyrazol-1-yl)phenylamino)-2-(4-methyl-1-(methylamino)-1-oxopentan-2-ylamino)-6-oxo-1,6-dihydropyrimidine-5-carboxamide; (R)-4-(1H-indazol-5-ylamino)-2-(4-methyl-1-(methylamino)-1-oxopentan-2-ylamino)-6-oxo-1,6-dihydropyrimidine-5-carboxamide; (R)-2-(1-amino-4-methyl-1-oxopentan-2-ylamino)-6-oxo-4-(4-(pyridin-3-yl)phenylamino)-1,6-dihydropyrimidine-5-carboxamide; (R)-2-(1-amino-4-methyl-1-oxopentan-2-ylamino)-6-oxo-4-(quinolin-7-ylamino)-1,6-dihydropyrimidine-5-carboxamide; (R)-2-(1-amino-4-methyl-1-oxopentan-2-ylamino)-6-oxo-4-(quinolin-3-ylamino)-1,6-dihydropyrimidine-5-carboxamide; (R)-2-(1-amino-1-oxobutan-2-ylamino)-6-oxo-4-(quinolin-3-ylamino)-1,6-dihydropyrimidine-5-carboxamide; (R)-2-(1-amino-1-oxobutan-2-ylamino)-4-(6-methoxypyridin-3-ylamino)-6-oxo-1,6-dihydropyrimidine-5-carboxamide; (R)-2-(1-amino-4-methyl-1-oxopentan-2-ylamino)-6-oxo-4-(4-(pyridin-4-yl)phenylamino)-1,6-dihydropyrimidine-5-carboxamide; (R)-2-(1-amino-4-methyl-1-oxopentan-2-ylamino)-6-oxo-4-(3-(pyridin-4-yl)phenylamino)-1,6-dihydropyrimidine-5-carboxamide; (R)-2-(1-amino-4-methyl-1-oxopentan-2-ylamino)-6-oxo-4-(4-(pyridin-2-yl)phenylamino)-1,6-dihydropyrimidine-5-carboxamide; (R)-2-(1-amino-4-methyl-1-oxopentan-2-ylamino)-4-(1-methyl-1H-pyrazol-4-ylamino)-6-oxo-1,6-dihydropyrimidine-5-carboxamide; (R)-2-(1-amino-4-methyl-1-oxopentan-2-ylamino)-4-(4-(1H-imidazol-1-yl)phenylamino)-6-oxo-1,6-dihydropyrimidine-5-carboxamide; (R)-2-(1-amino-1-oxobutan-2-ylamino)-4-(4-(1H-imidazol-1-yl)phenylamino)-6-oxo-1,6-dihydropyrimidine-5-carboxamide; (R)-2-(1-amino-4-methyl-1-oxopentan-2-ylamino)-4-(1-methyl-1H-pyrazol-3-ylamino)-6-oxo-1,6-dihydropyrimidine-5-carboxamide; (R)-2-(1-amino-4-methyl-1-oxopentan-2-ylamino)-4-(3-(1H-imidazol-1-yl)phenylamino)-6-oxo-1,6-dihydropyrimidine-5-carboxamide; and (R)-2-(1-amino-1-oxobutan-2-ylamino)-4-(3-(1H-imidazol-1-yl)phenylamino)-6-oxo-1,6-dihydropyrimidine-5-carboxamide.

The present invention provides in another embodiment, a compound having the structure found in the Examples.

The present invention provides in another embodiment, a compound having the structure found in the tables.

The present invention provides in any of the embodiments, the compound is not a compound selected from the group consisting of:

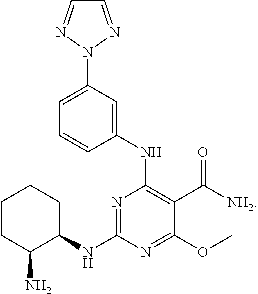 [SMILES: COc1nc(N[C@@H]2CCCC[C@@H]2N)nc(Nc2cccc(-n3nccn3)c2)c1C(N)=O]

It is understood that in another group of embodiments, any of the above embodiments may also be combined with other embodiments listed herein, to form other embodiments of the invention. Similarly, it is understood that in other embodiments, listing of groups includes embodiments wherein one or more of the elements of those groups is not included.

b. Methods of Synthesis

The compounds of the present invention may be prepared by known organic synthesis techniques, including the methods described in more detail in the Examples.

One skilled in the art will recognize that in certain embodiments of structure (I) when D1, R¹, D² or R^{3c} comprises a terminal heteroatom, it may be advantageous to use a protecting group strategy. The protecting group can be removed using methods known to those skilled in the art to yield compounds of structure (I).

The compounds of the present invention may generally be utilized as the free base. Alternatively, the compounds of this invention may be used in the form of acid addition salts as described below.

c. Inhibition of Syk Kinase

The activity of a specified compound as an inhibitor of a Syk kinase may be assessed in vitro or in vivo. In some embodiments, the activity of a specified compound can be tested in a cellular assay. Selectivity could also be ascertained in biochemical assays with isolated kinases.

d. Compositions and Methods of Administration

The present invention further provides compositions comprising one or more compounds of formula (I) or a pharmaceutically acceptable salt, ester or prodrug thereof, and a pharmaceutically acceptable carrier or diluent. It will be appreciated that the compounds of formula (I)) in this invention may be derivatized at functional groups to provide prodrug derivatives which are capable of conversion back to the parent compounds in vivo. Examples of such prodrugs include the physiologically acceptable and metabolically labile ester derivatives, such as methoxymethyl esters, methylthiomethyl esters, or pivaloyloxymethyl esters derived from a hydroxyl group of the compound or a carbamoyl moiety derived from an amino group of the compound. Additionally, any physiologically acceptable equivalents of the compounds of formula (I), similar to metabolically labile esters or carbamates, which are capable of producing the parent compounds of formula (I) in vivo, are within the scope of this invention.

As used herein, the term “pharmaceutically acceptable salts” refers to any acid or base addition salt whose counter-ions are non-toxic to the patient in pharmaceutical doses of the salts. A host of pharmaceutically acceptable salts are well known in the pharmaceutical field. If pharmaceutically acceptable salts of the compounds of this invention are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Furthermore, the basic nitrogen-containing groups may be quaternized with agents like lower alkyl halides, such as methyl, ethyl, propyl and butyl chlorides, bromides and iodides; dialkyl sulfates, such as dimethyl, diethyl, dibutyl and diamyl sulfates, long chain halides, such as decyl, lauryl, myristyl and stearyl chlorides, bromides and iodides; aralkyl halides, such as benzyl and phenethyl bromides and others. Water or oil-soluble or dispersible products are thereby obtained.

The compounds utilized in the compositions and methods of this invention may also be modified by appending appropriate functionalities to enhance selective biological properties. Such modifications are known in the art and include those which increase biological penetration into a given biological system (e.g., blood, lymphatic system, central nervous system, etc.), increase oral availability, increase solubility to allow administration by injection, alter metabolism and alter rate of excretion.

The pharmaceutical compositions of the invention can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions. Formulations may optionally contain stabilizers, pH modifiers, surfactants, bioavailability modifiers and combinations of these.

The term “unit dosage form” refers to physically discrete units suitable as unitary dosages for human subjects and other mammals, each unit containing a predetermined quantity of drug calculated to produce the desired onset, tolerability, and/or therapeutic effects, in association with a suitable pharmaceutical excipient (e.g., an ampoule). In addition, more concentrated compositions may be prepared, from which the more dilute unit dosage compositions may then be produced. The more concentrated compositions thus will contain substantially more than, e.g., at least 1, 2, 3, 4, 5, 6, 7, 8, 9, 10, or more times the amount of one or more Syk inhibitors.

Methods for preparing such dosage forms are known to those skilled in the art (see, for example, REMINGTON'S PHARMACEUTICAL SCIENCES, 18TH ED., Mack Publishing Co., Easton, Pa. (1990)). In addition, pharmaceutically acceptable salts of the Syk inhibitors of the present invention (e.g., acid addition salts) may be prepared and included in the compositions using standard procedures known to those skilled in the art of synthetic organic chemistry and described, e.g., by J. March, Advanced Organic Chemistry: Reactions, Mechanisms and Structure, 4^{th} Ed. (New York: Wiley-Interscience, 1992).

The compositions typically include a conventional pharmaceutical carrier or excipient and may additionally include other medicinal agents, carriers, adjuvants, diluents, tissue permeation enhancers, solubilizers, and the like. Preferably, the composition will contain about 0.01% to about 90%, preferably about 0.1% to about 75%, more preferably about 0.1% to 50%, still more preferably about 0.1% to 10% by weight of one or more Syk inhibitors, with the remainder consisting of suitable pharmaceutical carrier and/or excipients. Appropriate excipients can be tailored to the particular composition and route of administration by methods well known in the art, e.g., REMINGTON'S PHARMACEUTICAL SCIENCES, supra.

Pharmaceutically acceptable carriers that may be used in these compositions include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols. The compositions can additionally include lubricating agents such as talc, magnesium stearate, and mineral oil; wetting agents; emulsifying agents; suspending agents; preserving agents such as methyl-, ethyl-, and propyl-hydroxy-benzoates; pH adjusting agents such as inorganic and organic acids and bases; sweetening agents; and flavoring agents.

Administration of a composition comprising one or more Syk inhibitors with one or more suitable pharmaceutical excipients as advantageous can be carried out via any of the accepted modes of administration. Thus, administration can be, for example, oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally or intravenously. The formulations of the invention may be designed as short-acting, fast-releasing, or long-acting. Still further, compounds can be administered in a local rather than systemic means, such as administration (e.g., injection) as a sustained release formulation. According to a representative embodiment, the compositions of this invention are formulated for pharmaceutical administration to a mammal, preferably a human being.

The compositions of the present invention containing one or more Syk inhibitors can be administered repeatedly, e.g., at least 2, 3, 4, 5, 6, 7, 8, or more times, or the composition may be administered by continuous infusion. Suitable sites of administration include, but are not limited to, skin, bronchial, gastrointestinal, anal, vaginal, eye, and ear. The formulations may take the form of solid, semi-solid, lyophilized powder, or liquid dosage forms, such as, for example, tablets, pills, capsules, powders, solutions, suspensions, emulsions, suppositories, retention enemas, creams, ointments, lotions, gels, aerosols, or the like, preferably in unit dosage forms suitable for simple administration of precise dosages.

The pharmaceutical compositions of this invention may be in any orally acceptable dosage form, including tablets, capsules, cachets, emulsions, suspensions, solutions, syrups, elixirs, sprays, boluses, lozenges, powders, granules, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like. In the case of tablets for oral use, carriers that are commonly used include lactose and corn starch. Lubricating agents, such as magnesium stearate, are also typically added. For a capsule form, useful diluents include lactose and dried cornstarch. When aqueous suspensions are required for oral use, the active ingredient is combined with emulsifying and suspending agents. If desired, certain sweetening, flavoring or coloring agents may also be added.

In some embodiments, the compositions take the form of a pill, tablet, or capsule, and thus, the composition can contain, along with one or more Syk inhibitors, a diluent such as lactose, sucrose, dicalcium phosphate, and the like; a disintegrant such as starch or derivatives thereof; a lubricant such as magnesium stearate and the like; and/or a binder such a starch, gum acacia, polyvinylpyrrolidone, gelatin, cellulose and derivatives thereof. A tablet can be made by any compression or molding process known to those of skill in the art. Compressed tablets may be prepared by compressing in a suitable machine the Syk inhibitors in a free-flowing form, e.g., a powder or granules, optionally mixed with accessory ingredients, e.g., binders, lubricants, diluents, disintegrants, or dispersing agents. Molded tablets can be made by molding in a suitable machine a mixture of the powdered Syk inhibitors with any suitable carrier.

Alternatively, the pharmaceutical compositions of this invention may be in the form of suppositories for rectal administration. These may be prepared by mixing the agent with a suitable non-irritating excipient which is solid at room temperature but liquid at rectal temperature and therefore will melt in the rectum to release the drug. Such materials include cocoa butter, beeswax, polyethylene glycol (PEG), hard fat, and/or hydrogenated cocoglyceride. Compositions suitable for rectal administration may also comprise a rectal enema unit containing one or more Syk inhibitors and pharmaceutically-acceptable vehicles (e.g., 50% aqueous ethanol or an aqueous salt solution) that are physiologically compatible with the rectum and/or colon. The rectal enema unit contains an applicator tip protected by an inert cover, preferably comprised of polyethylene, lubricated with a lubricant such as white petrolatum, and preferably protected by a one-way valve to prevent back-flow of the dispensed formula. The rectal enema unit is also of sufficient length, preferably two inches, to be inserted into the colon via the anus.

Liquid compositions can be prepared by dissolving or dispersing one or more Syk inhibitors and optionally one or more pharmaceutically acceptable adjuvants in a carrier such as, for example, aqueous saline, aqueous dextrose, glycerol, ethanol, and the like, to form a solution or suspension, e.g., for oral, topical, or intravenous administration. Pharmaceutical formulations may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension formulations may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension formulations.

The pharmaceutical compositions of this invention may also be in a topical form, especially when the target of treatment includes areas or organs readily accessible by topical application, including diseases of the eye, the skin, or the lower intestinal tract. Suitable topical formulations are readily prepared for each of these areas or organs. For topical administration, the composition containing one or more Syk inhibitors can be in the form of emulsions, lotions, gels, foams, creams, jellies, solutions, suspensions, ointments, and transdermal patches.

Topical application for the lower intestinal tract may be effected in a rectal suppository formulation (see above) or in a suitable enema formulation. Topically-transdermal patches may also be used. For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions may be formulated in a suitable lotion or cream containing the active components suspended or dissolved in one or more pharmaceutically acceptable carriers. Suitable carriers include mineral oil, sorbitan monostearate, polysorbate 60, cetyl esters, wax, cetyl alcohol, 2-octyldodecanol, benzyl alcohol and water.

The pharmaceutical compositions of this invention may also be administered by nasal aerosol or inhalation. For delivery by inhalation, the compositions can be delivered as a dry powder or in liquid form via a nebulizer. Such compositions are prepared according to techniques known in the art of pharmaceutical formulation and may be prepared as solutions in saline, employing benzyl alcohol or other suitable preservatives, absorption promoters to enhance bioavailability, fluorocarbons and/or other conventional solubilizing or dispersing agents.

For ophthalmic use, the pharmaceutical compositions may be formulated as micronized suspensions in isotonic, pH adjusted sterile saline, or, preferably, as solutions in isotonic, pH adjusted sterile saline, either with our without a preservative, such as benzylalkonium chloride. Alternatively, for ophthalmic uses, the pharmaceutical compositions may be formulated in an ointment, such as petrolatum.

For parenteral administration, the compositions can be in the form of sterile injectable solutions and sterile packaged powders. Preferably, injectable solutions are formulated at a pH of about 4.5 to about 7.5.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

The compositions of the present invention can also be provided in a lyophilized form. Such compositions may include a buffer, e.g., bicarbonate, for reconstitution prior to administration, or the buffer may be included in the lyophilized composition for reconstitution with, e.g., water. The lyophilized composition may further comprise a suitable vasoconstrictor, e.g., epinephrine. The lyophilized composition can be provided in a syringe, optionally packaged in combination with the buffer for reconstitution, such that the reconstituted composition can be immediately administered to a patient.

Any of the above dosage forms containing effective amounts are within the bounds of routine experimentation and within the scope of the invention. A therapeutically effective dose may vary depending upon the route of administration and dosage form. The representative compound or compounds of the invention is a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

Besides those representative dosage forms described above, pharmaceutically acceptable excipients and carriers and dosage forms are generally known to those skilled in the art and are included in the invention. It should be understood that a specific dosage and treatment regimen for any particular patient will depend upon a variety of factors, including the activity of the specific compound employed, the age, body weight, general health, sex and diet of the patient, and the time of administration, rate of excretion, drug combination, judgment of the treating physician and severity of the particular disease being treated. The amount of active ingredient(s) will also depend upon the particular compound and other therapeutic agent, if present, in the composition.

e. Methods of Use

The invention provides methods of inhibiting or decreasing Syk activity as well as treating or ameliorating a Syk associated state, symptom, condition, disorder or disease in a patient in need thereof (e.g., human or non-human). In one embodiment, the Syk associated state, symptom, condition, disorder or disease is mediated, at least in part by Syk kinase activity. In more specific embodiments, the present invention provides a method for treating a condition or disorder mediated at least in part by Syk kinase activity is cardiovascular disease, inflammatory disease or autoimmune disease.

In one embodiment, the invention provides methods for preventing or treating a condition in a mammal mediated at least in part by syk activity comprising the step of administering to the mammal a therapeutically effective amount of a compound of the present invention. Such conditions include, but are not limited, to restenosis, acute coronary syndrome, myocardial infarction, unstable angina, refractory angina, occlusive coronary thrombosis occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolism, coagulopathy, disseminated intravascular coagulation, thrombotic thrombocytopenic purpura, thromboangiitis obliterans, thrombotic disease associated with heparin-induced thrombocytopenia, thrombotic complications associated with extracorporeal circulation, thrombotic complications associated with instrumentation such as cardiac or other intravascular catheterization, intra-aortic balloon pump, coronary stent or cardiac valve, conditions requiring the fitting of prosthetic devices, and the like.

In a further embodiment, the present invention provides a method for treating thrombosis, immune thrombocytic purura, heparin induced thrombocytopenia, dilated cardiomypathy, sickle cell disease, atherosclerosis, myocardial infarction, vacular inflammation, unstable angina or acute coronary syndromes.

In another embodiment, the present invention also provides a method for treating allergy, asthma, theumatoid arthritis, B Cell mediated disease such as Non-Hodgkin's Lymphoma, anti phospholipids syndrome, lupus, psoriasis, multiple sclerosis, end stage renal disease or chronic lymphocytic leukemia.

In another embodiment, the present invention provides a method for treating hemolytic anemia or immune thrombocytopenic purpura.

Therapy using the compounds described herein can be applied alone, or it can be applied in combination with or adjunctive to other common immunosuppressive therapies, such as, for example, the following: mercaptopurine; corticosteroids such as prednisone; methylprednisolone and prednisolone; alkylating agents such as cyclophosphamide; calcineurin inhibitors such as cyclosporine, sirolimus, and tacrolimus; inhibitors of inosine monophosphate dehydrogenase (IMPDH) such as mycophenolate, mycophenolate mofetil, and azathioprine; and agents designed to suppress cellular immunity while leaving the recipient's humoral immunologic response intact, including various antibodies (for example, antilymphocyte globulin (ALG), antithymocyte globulin (ATG), monoclonal anti-T-cell antibodies (OKT3)) and irradiation. These various agents can be used in accordance with their standard or common dosages, as specified in the prescribing information accompanying commercially available forms of the drugs (see also: the prescribing information in the 2006 Edition of The Physician's Desk Reference), the disclosures of which are incorporated herein by reference. Azathioprine is currently available from Salix Pharmaceuticals, Inc., under the brand name AZASAN; mercaptopurine is currently available from Gate Pharmaceuticals, Inc., under the brand name PURINETHOL; prednisone and prednisolone are currently available from Roxane Laboratories, Inc.; Methyl prednisolone is currently available from Pfizer; sirolimus (rapamycin) is currently available from Wyeth-Ayerst under the brand name RAPAMUNE; tacrolimus is currently available from Fujisawa under the brand name PROGRAF; cyclosporine is current available from Novartis under the brand dame SANDIMMUNE and from Abbott under the brand name GENGRAF; IMPDH inhibitors such as mycophenolate mofetil and mycophenolic acid are currently available from Roche under the brand name CELLCEPT and from Novartis under the brand name MYFORTIC; azathioprine is currently available from Glaxo Smith Kline under the brand name IMURAN; and antibodies are currently available from Ortho Biotech under the brand name ORTHOCLONE, from Novartis under the brand name SIMULECT (basiliximab), and from Roche under the brand name ZENAPAX (daclizumab).

In another embodiment, the compounds could be administered either in combination or adjunctively with an inhibitor of a Syk kinase. Syk kinase is a tyrosine kinase known to play a critical role in Fcy receptor signaling, as well as in other signaling cascades, such as those involving B-cell receptor signaling (Turner et al., (2000), Immunology Today 21:148-154) and integrins beta(1), beta (2), and beta (3) in neutrophils (Mocsai et al., (2002), Immunity 16:547-558). For example, Syk kinase plays a pivotal role in high affinity IgE receptor signaling in mast cells that leads to activation and subsequent release of multiple chemical mediators that trigger allergic attacks. However, unlike the JAK kinases, which help regulate the pathways involved in delayed or cell-mediated Type IV hypersensitivity reactions, Syk kinase helps regulate the pathways involved in immediate IgE-mediated, Type I hypersensitivity reactions. Certain compounds that affect the Syk pathway may or may not also affect the JAK pathways.

Suitable Syk inhibitory compounds are described, for example, in Ser. No. 10/355,543 filed Jan. 31, 2003 (publication no. 2004/0029902); WO 03/063794; Ser. No. 10/631,029 filed Jul. 29, 2003; WO 2004/014382; Ser. No. 10/903,263 filed Jul. 30, 2004; PCT/US2004/24716 filed Jul. 30, 2004 (WO005/016893); Ser. No. 10/903,870 filed Jul. 30, 2004; PCT/US2004/24920 filed Jul. 30, 2004; Ser. No. 60/630,808 filed Nov. 24, 2004; Ser. No. 60/645,424 filed Jan. 19, 2005; and Ser. No. 60/654,620, filed Feb. 18, 2005, the disclosures of which are incorporated herein by reference. The described herein and Syk inhibitory compounds could be used alone or in combination with one or more conventional transplant rejection treatments, as described above.

In a specific embodiment, the compounds can be used to treat or prevent these diseases in patients that are either initially non-responsive (resistant) to or that become non-responsive to treatment with a Syk inhibitory compound or one of the other current treatments for the particular disease. The compounds could also be used in combination with Syk inhibitory compounds in patients that are Syk-compound resistant or non-responsive. Suitable Syk-inhibitory compounds with which the compounds can be administered are provided infra.

In another embodiment, this invention provides a method of treating a T-cell mediated autoimmune disease, comprising administering to a patient suffering from such an autoimmune disease an amount of a compound effective to treat the autoimmune disease wherein the compound is selected from the compounds of the invention, as described herein, and the compound is administered in combination with or adjunctively to a compound that inhibits Syk kinase with an IC₅₀ in the range of at least 10 μM.

When used to treat or prevent such diseases, the compounds can be administered singly, as mixtures of one or more compounds, or in mixture or combination with other agents useful for treating such diseases and/or the symptoms associated with such diseases. The compounds may also be administered in mixture or in combination with agents useful to treat other disorders or maladies, such as steroids, membrane stabilizers, 5-lipoxygenase (5LO) inhibitors, leukotriene synthesis and receptor inhibitors, inhibitors of IgE isotype switching or IgE synthesis, IgG isotype switching or IgG synthesis, beta.-agonists, tryptase inhibitors, aspirin, cyclooxygenase (COX) inhibitors, methotrexate, anti-TNF drugs, anti CD20 antibody, PD4 inhibitors, p38 inhibitors, PDE4 inhibitors, and antihistamines, to name a few. The compounds can be administered per se in the form of prodrugs or as pharmaceutical compositions, comprising an active compound or prodrug.

Active compounds of the invention typically inhibit the Syk pathway. The activity of a specified compound as an inhibitor of a Syk kinase can be assessed in vitro or in vivo. In some embodiments, the activity of a specified compound can be tested in a cellular assay.

“Cell proliferative disorder” refers to a disorder characterized by abnormal proliferation of cells. A proliferative disorder does not imply any limitation with respect to the rate of cell growth, but merely indicates loss of normal controls that affect growth and cell division. Thus, in some embodiments, cells of a proliferative disorder can have the same cell division rates as normal cells but do not respond to signals that limit such growth. Within the ambit of “cell proliferative disorder” is neoplasm or tumor, which is an abnormal growth of tissue. Cancer refers to any of various malignant neoplasms characterized by the proliferation of cells that have the capability to invade surrounding tissue and/or metastasize to new colonization sites.

Generally, cell proliferative disorders treatable with the compounds disclosed herein relate to any disorder characterized by aberrant cell proliferation. These include various tumors and cancers, benign or malignant, metastatic or non-metastatic. Specific properties of cancers, such as tissue invasiveness or metastasis, can be targeted using the methods described herein. Cell proliferative disorders include a variety of cancers, including, among others, ovarian cancer, renal cancer, gastrointestinal cancer, kidney cancer, bladder cancer, pancreatic cancer, lung squamous carcinoma, and adenocarcinoma.

In some embodiments, the cell proliferative disorder treated is a hematopoietic neoplasm, which is aberrant growth of cells of the hematopoietic system. Hematopoietic malignancies can have its origins in pluripotent stem cells, multipotent progenitor cells, oligopotent committed progenitor cells, precursor cells, and terminally differentiated cells involved in hematopoiesis. Some hematological malignancies are believed to arise from hematopoietic stem cells, which have the ability for self renewal. For instance, cells capable of developing specific subtypes of acute myeloid leukemia (AML) (Cynthia K. Hahn, Kenneth N. Ross, Rose M. Kakoza, Steven Karr, Jinyan Du, Shao-E Ong, Todd R. Golub, Kimberly Stegmaier, Syk is a new target for AML differentiation, Blood, 2007, 110, Abstract 209) upon transplantation display the cell surface markers of hematopoietic stem cells, implicating hematopoietic stem cells as the source of leukemic cells. Blast cells that do not have a cell marker characteristic of hematopoietic stem cells appear to be incapable of establishing tumors upon transplantation (Blaire et al., 1997, Blood 89:3104-3112). The stem cell origin of certain hematological malignancies also finds support in the observation that specific chromosomal abnormalities associated with particular types of leukemia can be found in normal cells of hematopoietic lineage as well as leukemic blast cells. For instance, the reciprocal translocation t(9q34; 22q11) associated with approximately 95% of chronic myelogenous leukemia appears to be present in cells of the myeloid, erythroid, and lymphoid lineage, suggesting that the chromosomal aberration originates in hematopoietic stem cells. A subgroup of cells in certain types of CML displays the cell marker phenotype of hematopoietic stem cells.

Although hematopoietic neoplasms often originate from stem cells, committed progenitor cells or more terminally differentiated cells of a developmental lineage can also be the source of some leukemias. For example, forced expression of the fusion protein Bcr/Abl (associated with chronic myelogenous leukemia) in common myeloid progenitor or granulocyte/macrophage progenitor cells produces a leukemic-like condition. Moreover, some chromosomal aberrations associated with subtypes of leukemia are not found in the cell population with a marker phenotype of hematopoietic stem cells, but are found in a cell population displaying markers of a more differentiated state of the hematopoietic pathway (Turhan et al., 1995, Blood 85:2154-2161). Thus, while committed progenitor cells and other differentiated cells may have only a limited potential for cell division, leukemic cells may have acquired the ability to grow unregulated, in some instances mimicking the self-renewal characteristics of hematopoietic stem cells (Passegue et al., Proc. Natl. Acad. Sci. USA, 2003, 100:11842-9).

In some embodiments, the hematopoietic neoplasm treated is a lymphoid neoplasm, where the abnormal cells are derived from and/or display the characteristic phenotype of cells of the lymphoid lineage. Lymphoid neoplasms can be subdivided into B-cell neoplasms, T and NK-cell neoplasms, and Hodgkin's lymphoma. B-cell neoplasms can be further subdivided into precursor B-cell neoplasm and mature/peripheral B-cell neoplasm. Exemplary B-cell neoplasms are precursor B-lymphoblastic leukemia/lymphoma (precursor B-cell acute lymphoblastic leukemia) while exemplary mature/peripheral B-cell neoplasms are B-cell chronic lymphocytic leukemia/small lymphocytic lymphoma, B-cell prolymphocytic leukemia, lymphoplasmacytic lymphoma, splenic marginal zone B-cell lymphoma, hairy cell leukemia, plasma cell myeloma/plasmacytoma, extranodal marginal zone B-cell lymphoma of MALT type, nodal marginal zone B-cell lymphoma, follicular lymphoma, mantle-cell lymphoma, diffuse large B-cell lymphoma, mediastinal large B-cell lymphoma, primary effusion lymphoma, and Burkitt's lymphoma/Burkitt cell leukemia. T-cell and Nk-cell neoplasms are further subdivided into precursor T-cell neoplasm and mature (peripheral) T-cell neoplasms. Exemplary precursor T-cell neoplasm is precursor T-lymphoblastic lymphoma/leukemia (precursor T-cell acute lymphoblastic leukemia) while exemplary mature (peripheral) T-cell neoplasms are T-cell prolymphocytic leukemia T-cell granular lymphocytic leukemia, aggressive NK-cell leukemia, adult T-cell lymphoma/leukemia (HTLV-1), extranodal NK/T-cell lymphoma, nasal type, enteropathy-type T-cell lymphoma, hepatosplenic gamma-delta T-cell lymphoma, subcutaneous panniculitis-like T-cell lymphoma, Mycosis fungoides/Sezary syndrome, Anaplastic large-cell lymphoma, T/null cell, primary cutaneous type, Peripheral T-cell lymphoma, not otherwise characterized, Angioimmunoblastic T-cell lymphoma, Anaplastic large-cell lymphoma, T/null cell, primary systemic type. The third member of lymphoid neoplasms is Hodgkin's lymphoma, also referred to as Hodgkin's disease. Exemplary diagnosis of this class that can be treated with the compounds include, among others, nodular lymphocyte-predominant Hodgkin's lymphoma, and various classical forms of Hodgkin's disease, exemplary members of which are Nodular sclerosis Hodgkin's lymphoma (grades 1 and 2), Lymphocyte-rich classical Hodgkin's lymphoma, Mixed cellularity Hodgkin's lymphoma, and Lymphocyte depletion Hodgkin's lymphoma. In various embodiments, any of the lymphoid neoplasms that are associated with aberrant JAK activity can be treated with the Syk inhibitory compounds.

In some embodiments, the hematopoietic neoplasm treated is a myeloid neoplasm. This group comprises a large class of cell proliferative disorders involving or displaying the characteristic phenotype of the cells of the myeloid lineage. Myeloid neoplasms can be subdivided into myeloproliferative diseases, myelodysplastic/myeloproliferative diseases, myelodysplastic syndromes, and acute myeloid leukemias. Exemplary myeloproliferative diseases are chronic myelogenous leukemia (e.g., Philadelphia chromosome positive (t(9; 22)(qq34; q11)), chronic neutrophilic leukemia, chronic eosinophilic leukemia/hypereosinophilic syndrome, chronic idiopathic myelofibrosis, polycythemia vera, and essential thrombocythemia. Exemplary myelodysplastic/myeloproliferative diseases are chronic myelomonocytic leukemia, atypical chronic myelogenous leukemia, and juvenile myelomonocytic leukemia. Exemplary myelodysplastic syndromes are refractory anemia, with ringed sideroblasts and without ringed sideroblasts, refractory cytopenia (myelodysplastic syndrome) with multilineage dysplasia, refractory anemia (myelodysplastic syndrome) with excess blasts, 5q-syndrome, and myelodysplastic syndrome. In various embodiments, any of the myeloid neoplasms that are associated with aberrant Syk activity can be treated with the Syk inhibitory compounds.

In some embodiments, the compounds can be used to treat Acute myeloid leukemias (AML), which represent a large class of myeloid neoplasms having its own subdivision of disorders. These subdivisions include, among others, AMLs with recurrent cytogenetic translocations, AML with multilineage dysplasia, and other AML not otherwise categorized. Exemplary AMLs with recurrent cytogenetic translocations include, among others, AML with t(8; 21)(q22; q22), AML1(CBF-alpha)/ETO, Acute promyelocytic leukemia (AML with t(15; 17)(q22; q11-12) and variants, PML/RAR-alpha), AML with abnormal bone marrow eosinophils (inv(16)(p13q22) or t(16; 16)(p13; q11), CBFb/MYH11X), and AML with 11q23 (MLL) abnormalities. Exemplary AML with multilineage dysplasia are those that are associated with or without prior myelodysplastic syndrome. Other acute myeloid leukemias not classified within any definable group include, AML minimally differentiated, AML without maturation, AML with maturation, Acute myelomonocytic leukemia, Acute monocytic leukemia, Acute erythroid leukemia, Acute megakaryocytic leukemia, Acute basophilic leukemia, and Acute panmyelosis with myelofibrosis.

“Treating” within the context of the invention means an alleviation of symptoms associated with a disorder or disease, or halt of further progression or worsening of those symptoms, or prevention or prophylaxis of the disease or disorder.

The term “mammal” includes organisms which express Syk. Examples of mammals include mice, rats, cows, sheep, pigs, goats, horses, bears, monkeys, dogs, cats and, preferably, humans. Transgenic organisms which express Syk are also included in this definition.

The inventive methods comprise administering an effective amount of a compound or composition described herein to a mammal or non-human animal. As used herein, “effective amount” of a compound or composition of the invention includes those amounts that antagonize or inhibit Syk. An amount which antagonizes or inhibits Syk is detectable, for example, by any assay capable of determining Syk activity, including the one described below as an illustrative testing method. Effective amounts may also include those amounts which alleviate symptoms of a Syk associated disorder treatable by inhibiting Syk. Accordingly, “antagonists of Syk” include compounds which interact with the Syk, and modulate, e.g., inhibit or decrease, the ability of a second compound, e.g., another Syk ligand, to interact with the Syk. The Syk binding compounds are preferably antagonists of Syk. The language “Syk binding compound” (e.g., exhibits binding affinity to the receptor) includes those compounds which interact with Syk resulting in modulation of the activity of Syk. Syk binding compounds may be identified using an in vitro (e.g., cell and non-cell based) or in vivo method. A description of in vitro methods are provided below.

The amount of compound present in the methods and compositions described herein should be sufficient to cause a detectable decrease in the severity of the disorder, as measured by any of the assays described in the examples. The amount of Syk modulator needed will depend on the effectiveness of the modulator for the given cell type and the length of time required to treat the disorder. In certain embodiments, the compositions of this invention may further comprise another therapeutic agent. When a second agent is used, the second agent may be administered either as a separate dosage form or as part of a single dosage form with the compounds or compositions of this invention. While one or more of the inventive compounds can be used in an application of monotherapy to treat a disorder, disease or symptom, they also may be used in combination therapy, in which the use of an inventive compound or composition (therapeutic agent) is combined with the use of one or more other therapeutic agents for treating the same and/or other types of disorders, symptoms and diseases. Combination therapy includes administration of the two or more therapeutic agents concurrently or sequentially. The agents may be administered in any order. Alternatively, the multiple therapeutic agents can be combined into a single composition that can be administered to the patient. For instance, a single pharmaceutical composition could comprise the compound or pharmaceutically acceptable salt, ester or prodrug thereof according to the formula I, another therapeutic agent (e.g., methotrexate) or a tautomer thereof or a pharmaceutically acceptable salt, ester or prodrug thereof, and a pharmaceutically acceptable excipient or carrier.

The invention comprises a compound having the formula I, a method for making an inventive compound, a method for making a pharmaceutical composition from at least one inventive compound and at least one pharmaceutically acceptable carrier or excipient, and a method of using one or more inventive compounds to treat a variety of disorders, symptoms and diseases (e.g., inflammatory, autoimmune, neurological, neurodegenerative, oncology and cardiovascular), such as RA, osteoarthritis, irritable bowel disease IBD, asthma, chronic obstructive pulmonary disease COPD and MS. The inventive compounds and their pharmaceutically acceptable salts and/or neutral compositions may be formulated together with a pharmaceutically acceptable excipient or carrier and the resulting composition may be administered in vivo to mammals, such as men, women and animals, to treat a variety of disorders, symptoms and diseases. Furthermore, the inventive compounds can be used to prepare a medicament that is useful for treating a variety of disorders, symptoms and diseases.

All of the compounds of the present invention are either potent inhibitors of Syk kinases, exhibiting IC₅₀s in the respective assay in the range of less than 5 μM, with most being in the nanomolar, and several in the sub-nanomolar, range. In some embodiments, the compounds of the present invention may be “dual” Syk/JAK inhibitors in that they inhibit both Syk and JAK kinase to some degree. In other embodiments, the compounds of the present invention may selectively inhibit Syk kinase, but not appreciably inhibit one or more JAK kinases. In other embodiments, the compounds of the present invention may selectively inhibit JAK kinase, but not appreciably inhibit one or more Syk kinases.

f. Kits

Still another aspect of this invention is to provide a kit comprising separate containers in a single package, wherein the inventive pharmaceutical compounds, compositions and/or salts thereof are used in combination with pharmaceutically acceptable carriers to treat states, disorders, symptoms and diseases where Syk plays a role.

### EXAMPLES

The following examples are offered to illustrate, but not to limit, the claimed invention.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1967-2004, Volumes 1-22; Rodd's Chemistry of Carbon Compounds, Elsevier Science Publishers, 1989, Volumes 1-5 and Supplementals; and Organic Reactions, Wiley & Sons: New York, 2005, Volumes 1-65.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range from about −78° C. to about 150° C., more preferably from about 0° C. to about 125° C., and most preferably and conveniently at about room (or ambient) temperature, e.g., about 20° C. to about 75° C.

Referring to the examples that follow, compounds of the present invention were synthesized using the methods described herein, or other methods, which are well known in the art.

The compounds and/or intermediates may be characterized by high performance liquid chromatography (HPLC) using a Waters Alliance chromatography system with a 2695 Separation Module (Milford, Mass.). The analytical columns may be C-18 SpeedROD RP-18E Columns from Merck KGaA (Darmstadt, Germany). Alternately, characterization may be performed using a Waters Unity (UPLC) system with Waters Acquity UPLC BEH C-18 2.1 mm×15 mm columns. A gradient elution may be used, typically starting with 5% acetonitrile/95% water and progressing to 95% acetonitrile over a period of 5 minutes for the Alliance system and 1 minute for the Acquity system. All solvents may contain 0.1% trifluoroacetic acid (TFA). Compounds may be detected by ultraviolet light (UV) absorption at either 220 or 254 nm. HPLC solvents may be from EMD Chemicals, Inc. (Gibbstown, N.J.). In some instances, purity may be assessed by thin layer chromatography (TLC) using glass backed silica gel plates, such as, for example, EMD Silica Gel 60 2.5 cm×7.5 cm plates. TLC results may be readily detected visually under ultraviolet light, or by employing well known iodine vapor and other various staining techniques.

Mass spectrometric analysis may be performed on one of two Agilent 1100 series LCMS instruments and the Acquity system with acetonitrile/water as the mobile phase. One system may use TFA as the modifier and measure in positive ion mode [reported as MH+, (M+1) or (M+H)+] and the other may use either formic acid or ammonium acetate and measure in both positive [reported as MH⁺, (M+1) or (M+H)] and negative [reported as M−, (M−1) or (M−H)⁻] ion modes.

Nuclear magnetic resonance (NMR) analysis may be performed on some of the compounds with a Varian 400 MHz NMR (Palo Alto, Calif.). The spectral reference may be either TMS or the known chemical shift of the solvent.

The purity of some of the invention compounds may be assessed by elemental analysis (Robertson Microlit, Madison N.J.).

Melting points may be determined on a Laboratory Devices MeI-Temp apparatus (Holliston, Mass.).

Preparative separations may be carried out as needed, using either an Sql6x or an Sg100c chromatography system and prepackaged silica gel columns all purchased from Teledyne Isco, (Lincoln, Nebr.). Alternately, compounds and intermediates may be purified by flash column chromatography using silica gel (230-400 mesh) packing material, or by HPLC using a C-18 reversed phase column. Typical solvents employed for the Isco systems and flash column chromatography may be dichloromethane, methanol, ethyl acetate, hexane, acetone, aqueous hydroxyamine and triethyl amine. Typical solvents employed for the reverse phase HPLC may be varying concentrations of acetonitrile and water with 0.1% trifluoroacetic acid.

### General Methods

The following synthetic reaction schemes are merely illustrative of some methods by which the compounds of the present invention can be synthesized, and various modifications to these synthetic reaction schemes can be made and will be suggested to one skilled in the art having referred to the disclosure contained in this application.

### Example 1

### (R)-2-(1-amino-3-methyl-1-oxobutan-2-ylamino)-4-(3,5-dimethylphenylamino)-6-methoxypyrimidine-5-carboxamide

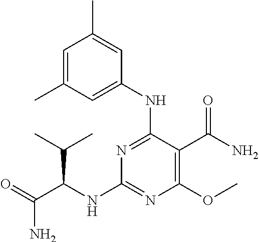 [SMILES: COc1nc(N[C@@H](C(N)=O)C(C)C)nc(Nc2cc(C)cc(C)c2)c1C(N)=O]

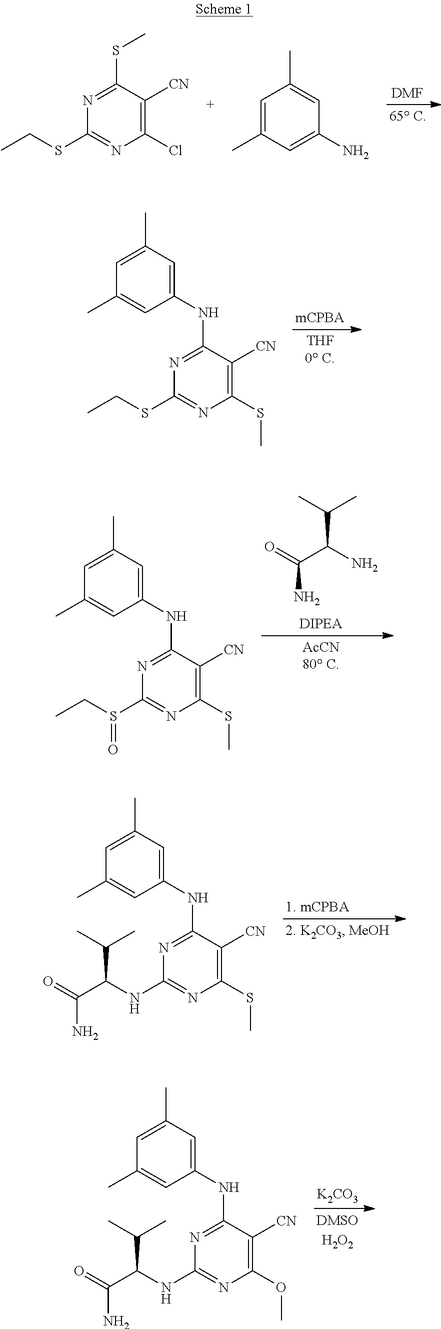

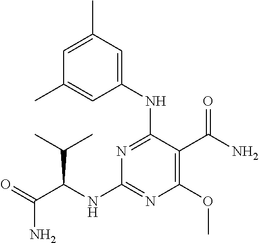 [SMILES: COc1nc(N[C@@H](C(N)=O)C(C)C)nc(Nc2cc(C)cc(C)c2)c1C(N)=O]

Step 1: To a solution of 4-chloro-2-(ethylthio)-6-(methylthio)pyrimidine-5-carbonitrile (450 mg, 1.83 mmol) in DMF (4.5 mL) was added 3,5-dimethylaniline (0.29 mL, 2.29 mmol). After heating at 65° C. for 5 h, it was diluted with EtOAc, washed with 1N HCl, Sat NaHCO₃, brine, dried and concentrated to give 4-(3,5-dimethylphenylamino)-2-(ethylthio)-6-(methylthio)pyrimidine-5-carbonitrile as crude product (430 mg).

Step 2: To a solution of 4-(3,5-dimethylphenylamino)-2-(ethylthio)-6-(methylthio)pyrimidine-5-carbonitrile (400 mg, 1.21 mmol) in THF (6 mL) at 0° C. was added mCPBA (65%, 368 mg, 1.39 mmol). After stirring for 30 min, it was diluted with EtOAc, organic layer was washed with Sat. NaHCO₃, brine, dried and concentrated to give corresponding sulfoxide.

Step 3: To a solution of the above sulfoxide in AcCN (5 mL) was added D-valinamide HCl salt (193 mg, 1.26 mmol) and DIPEA (0.64 mL, 3.63 mmol). After heating at 80° C. for 2 h, the mixture was diluted with EtOAc, the organic layer was washed with 1N HCl, Sat. NaHCO₃, Brine, dried and concentrated to give (R)-2-(5-cyano-4-(3,5-dimethylphenylamino)-6-(methylthio)pyrimidin-2-ylamino)-3-methylbutanamide (430 mg).

Step 4: To a solution of (R)-2-(5-cyano-4-(3,5-dimethylphenylamino)-6-(methylthio)pyrimidin-2-ylamino)-3-methylbutanamide (430 mg, 1.12 mmol) in THF (6 mL) was added mCPBA (65%, 420 mg, 1.58 mmol) at room temperature. After stirring for 10 min, it was diluted with EtOAc, organic layer was washed with Sat. NaHCO₃, brine, dried and concentrated to give corresponding sulfoxide. To this sulfoxide in MeOH (2 mL) was added K2CO3 (138 mg), and the mixture was heated at 45° C. for 5 min. MeOH was removed under vacuo, and the residue was added water, the resulting precipitate was collected by filtration to give (R)-2-(5-cyano-4-(3,5-dimethylphenylamino)-6-methoxypyrimidin-2-ylamino)-3-methylbutanamide (200 mg).

Step 5: To a solution of the aforementioned (R)-2-(5-cyano-4-(3,5-dimethylphenylamino)-6-methoxypyrimidin-2-ylamino)-3-methylbutanamide (200 mg) in DMSO (1.5 mL) was added K2CO3 (500 mg) and H2O2 (50%, 1.5 mL). After heating at 90° C. for 30 min, the mixture was cooled to room temperature, diluted with water, the resulting precipitate was collected by filtration and purified by preparative HPLC to give (R)-2-(1-amino-3-methyl-1-oxobutan-2-ylamino)-4-(3,5-dimethylphenylamino)pyrimidine-5-carboxamide (61 mg). MS found for C₁₉H₂₆N₆O₃ as (M+H)⁺ 387.4. λ=261.3, 288.4.

### Example 2

### (R)-2-(1-amino-3-methyl-1-oxobutan-2-ylamino)-4-(3,5-dimethylphenylamino)-6-oxo-1,6-dihydropyrimidine-5-carboxamide

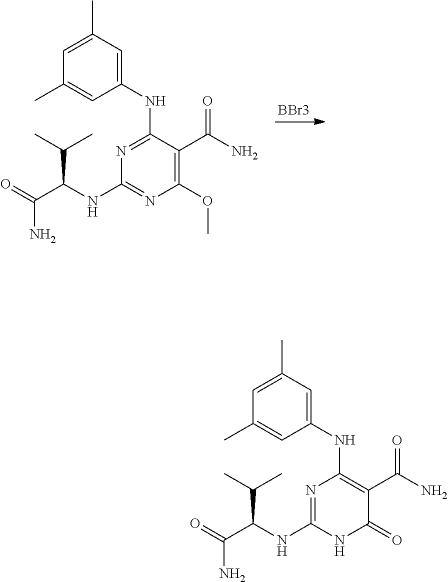 [SMILES: COc1nc(N[C@@H](C(N)=O)C(C)C)nc(Nc2cc(C)cc(C)c2)c1C(N)=O ; "BBr3" ; Cc1cc(C)cc(Nc2nc(N[C@@H](C(N)=O)C(C)C)[nH]c(=O)c2C(N)=O)c1]

To a solution of (R)-2-(1-amino-3-methyl-1-oxobutan-2-ylamino)-4-(3,5-dimethylphenylamino)-6-methoxypyrimidine-5-carboxamide (30 mg, 0.077 mmol) in DCM (2 mL) was added BBr₃ (0.05 mL) at room temperature. After stirring for 15 h at room temperature, the mixture was concentrated and purified by preparative HPLC to give (R)-2-(1-amino-3-methyl-1-oxobutan-2-ylamino)-4-(3,5-dimethylphenylamino)-6-oxo-1,6-dihydropyrimidine-5-carboxamide (21 mg). MS found for C₁₈H₂₄N₆O₃ as (M+H)⁺ 373.4. λ=259.2, 300.7.

### Example 3

### 2-((1R,2S)-2-aminocyclohexylamino)-4-(3-(2H-1,2,3-triazol-2-yl)phenylamino)-6-methoxypyrimidine-5-carboxamide

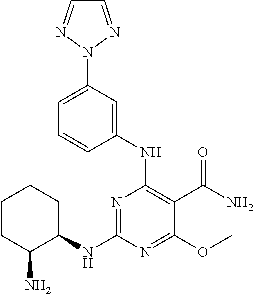 [SMILES: COc1nc(N[C@@H]2CCCC[C@@H]2N)nc(Nc2cccc(-n3nccn3)c2)c1C(N)=O]

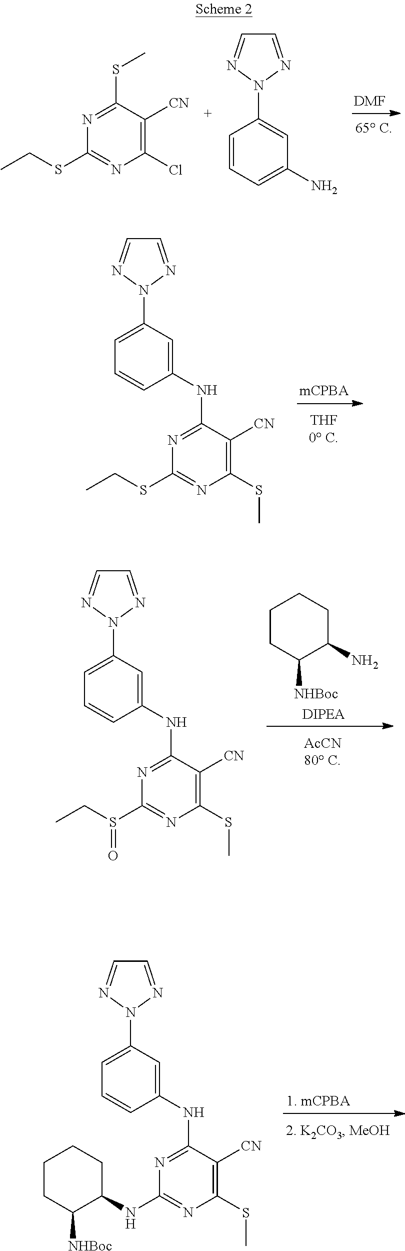

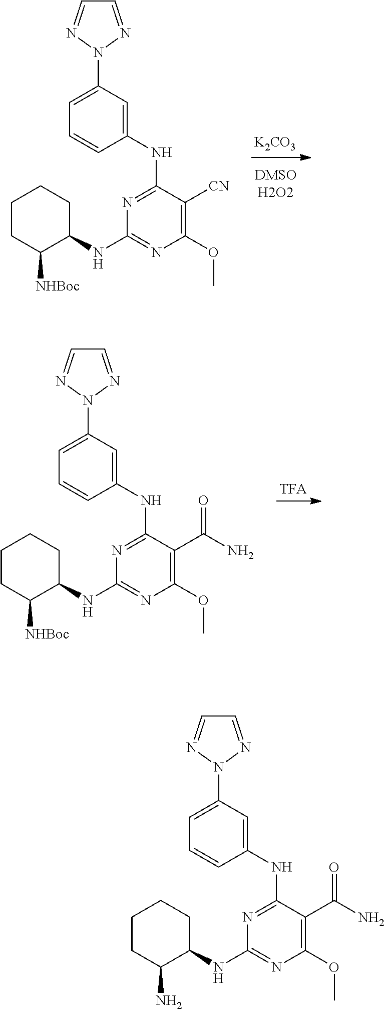 [SMILES: "TFA" ; COc1nc(N[C@@H]2CCCC[C@@H]2NC(=O)OC(C)(C)C)nc(Nc2cccc(-n3nccn3)c2)c1C#N ; "K2CO3" ; COc1nc(N[C@@H]2CCCC[C@@H]2NC(=O)OC(C)(C)C)nc(Nc2cccc(-n3nccn3)c2)c1C(N)=O ; COc1nc(N[C@@H]2CCCC[C@@H]2N)nc(Nc2cccc(-n3nccn3)c2)c1C(N)=O]

Step 1: To a solution of 4-chloro-2-(ethylthio)-6-(methylthio)pyrimidine-5-carbonitrile (930 mg, 1.83 mmol) in DMF (10 mL) was added 3-(2H-1,2,3-triazol-2-yl)aniline (750 mg, 4.69 mmol). After heating at 65° C. for 5 h, it was diluted with EtOAc, washed with 1N HCl, Sat NaHCO3, brine, dried and concentrated to give 4-(3-(2H-1,2,3-triazol-2-yl)phenylamino)-2-(ethylthio)-6-(methylthio)pyrimidine-5-carbonitrile as crude product (1.27 g).

Step 2: To a solution of 4-(3-(2H-1,2,3-triazol-2-yl)phenylamino)-2-(ethylthio)-6-(methylthio)pyrimidine-5-carbonitrile (110 mg, 0.3 mmol) in THF (1.5 mL) at 0° C. was added mCPBA (65%, 60 mg, 0.35 mmol). After stirring for 30 min at 0° C., it was diluted with EtOAc, organic layer was washed with Sat. NaHCO3, brine, dried and concentrated to give corresponding sulfoxide.

Step 3: To a solution of the above sulfoxide (120 mg) in AcCN (1.5 mL) was added tert-butyl (1S,2R) 2-aminocyclohexylcarbamate (96 mg, 0.45 mmol) and DIPEA (0.08 mL, 0.45 mmol). After heating at 75° C. for 1 h, the mixture was cooled to room temperature, water was added to the mixture, the resulting precipitate was collected by filtration to give tert-butyl (1S,2R)-2-(4-(3-(2H-1,2,3-triazol-2-yl)phenylamino)-5-cyano-6-(methylthio)pyrimidin-2-ylamino)cyclohexylcarbamate (138 mg).

Step 4: To a solution of tert-butyl (1S,2R)-2-(4-(3-(2H-1,2,3-triazol-2-yl)phenylamino)-5-cyano-6-(methylthio)pyrimidin-2-ylamino)cyclohexylcarbamate (100 mg, 0.19 mmol) in THF (2 mL) was added mCPBA (65%, 61 mg, 0.23 mmol) at room temperature. After stirring for 10 min, it was diluted with EtOAc, organic layer was washed with Sat. NaHCO₃, brine, dried and concentrated to give corresponding sulfoxide. To this sulfoxide in MeOH (2 mL) was added K2CO₃ (138 mg, excess), and the mixture was stirred at room temperature for 30 min. MeOH was removed under vacuo, and the residue was added water, the resulting precipitate was collected by filtration to give tert-butyl (1S,2R)-2-(4-(3-(2H-1,2,3-triazol-2-yl)phenylamino)-5-cyano-6-methoxypyrimidin-2-ylamino)cyclohexylcarbamate (88 mg).

Step 5: To a solution of the aforementioned tert-butyl (1S,2R)-2-(4-(3-(2H-1,2,3-triazol-2-yl)phenylamino)-5-cyano-6-methoxypyrimidin-2-ylamino)cyclohexylcarbamate (88 mg) in DMSO (1.5 mL) was added K2CO3 (160 mg) and H₂O₂ (50%, 1.5 mL). After heating at 90° C. for 30 min, the mixture was cooled to room temperature, diluted with water, the resulting precipitate was collected by filtration to give tert-butyl (1S,2R)-2-(4-(3-(2H-1,2,3-triazol-2-yl)phenylamino)-5-carbamoyl-6-methoxypyrimidin-2-ylamino)cyclohexylcarbamate (40 mg).

Step 6: To a solution of tert-butyl (1S,2R)-2-(4-(3-(2H-1,2,3-triazol-2-yl)phenylamino)-5-carbamoyl-6-methoxypyrimidin-2-ylamino)cyclohexylcarbamate (40 mg, 0.076 mmol) in DCM (1 mL) was added TFA (0.3 mL) at room temperature. After stirring for 20 min at room temperature, the mixture was concentrated and purified by preparative HPLC to give 2-((1R,2S)-2-aminocyclohexylamino)-4-(3-(2H-1,2,3-triazol-2-yl)phenylamino)-6-methoxypyrimidine-5-carboxamide (18 mg). MS found for C₁₈H₂₁N₉O₃ as (M+H)⁺

MS found for C₂₀H₂₅N₉O₂ as (M+H)⁺ 424.4. λ=262.9.

### Example 4

### (R)-2-(1-amino-3-methyl-1-oxobutan-2-ylamino)-4-(3,5-dimethylphenylamino)-6-ethoxypyrimidine-5-carboxamide

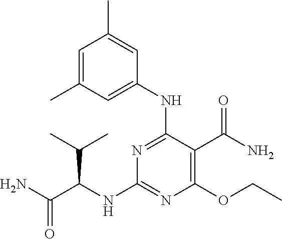 [SMILES: CCOc1nc(N[C@@H](C(N)=O)C(C)C)nc(Nc2cc(C)cc(C)c2)c1C(N)=O]

The title compound was synthesized similar to Example 1 in Scheme 1 using Ethyl alcohol to replace MeOH. MS found for C₂₀H₂₈N₆O₃ as (M+H)⁺ 401.4. λ=261.6, 290.0.

### Example 5

### 4-(3-(2H-1,2,3-triazol-2-yl)phenylamino-2-((1R,2S)-2-aminocyclohexyl)-6-oxo-1,6-dihydropyrimidine-5-carboxamide

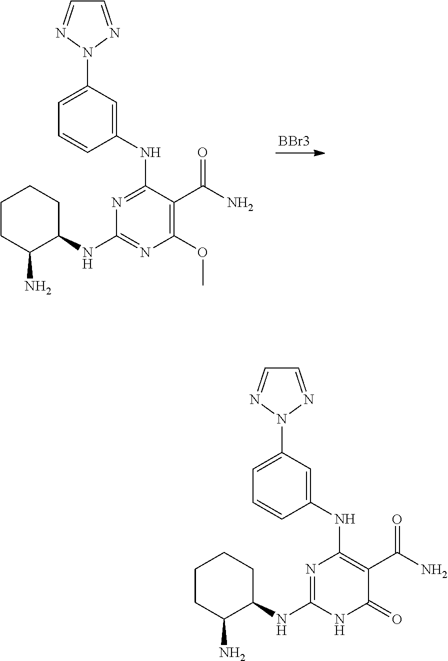 [SMILES: COc1nc(N[C@@H]2CCCC[C@@H]2N)nc(Nc2cccc(-n3nccn3)c2)c1C(N)=O ; "BBr3" ; NC(=O)c1c(Nc2cccc(-n3nccn3)c2)nc(N[C@@H]2CCCC[C@@H]2N)[nH]c1=O]

To a solution of 2-((1R,2S)-2-aminocyclohexylamino)-4-(3-(2H-1,2,3-triazol-2-yl)phenylamino)-6-methoxypyrimidine-5-carboxamide (17 mg, 0.04 mmol) in DCM (1 mL) was added BBr₃ (0.1 mL) at room temperature. After stirring for 15 h at room temperature, the mixture was concentrated and purified by preparative HPLC to give 4-(3-(2H-1,2,3-triazol-2-yl)phenylamino-2-((1R,2S)-2-aminocyclohexyl)-6-oxo-1,6-dihydropyrimidine-5-carboxamide (5 mg). MS found for C₁₉H₂₃N₉O₂ as (M+H)⁺ 410.2. λ=258.2, 299.7.

### Example 6

### (R)-4-(3-(2H-1,2,3-triazol-2-yl)phenylamino)-2-(1-amino-3-methyl-1-oxobutan-2-ylamino)-6-oxo-1,6-dihydropyrimidine-5-carboxamide

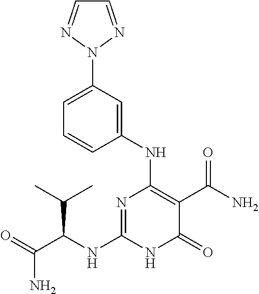 [SMILES: CC(C)[C@@H](Nc1nc(Nc2cccc(-n3nccn3)c2)c(C(N)=O)c(=O)[nH]1)C(N)=O]

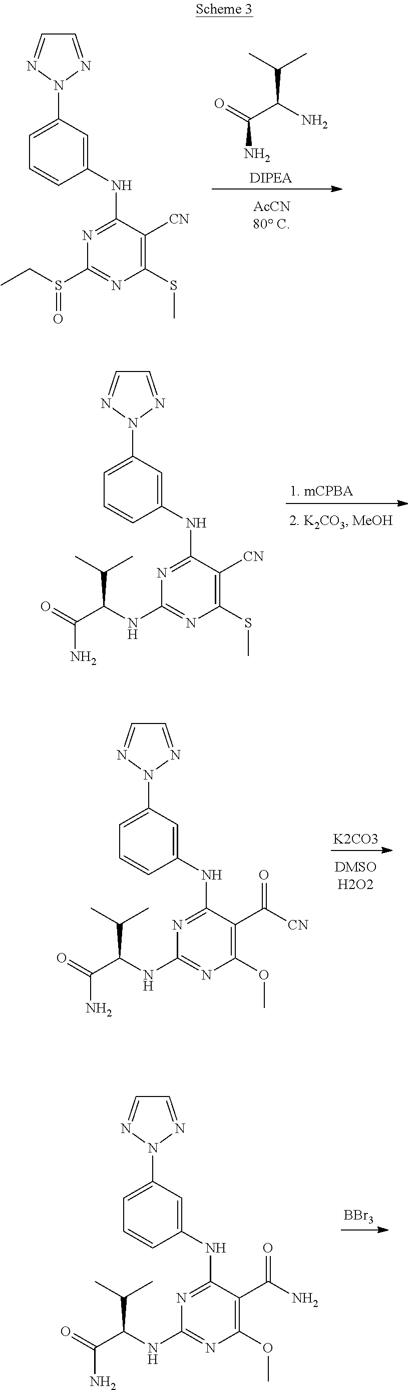

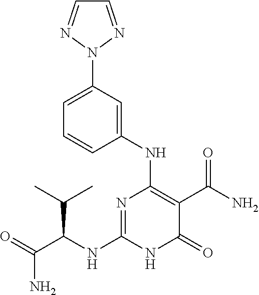 [SMILES: CC(C)[C@@H](Nc1nc(Nc2cccc(-n3nccn3)c2)c(C(N)=O)c(=O)[nH]1)C(N)=O]

Step 1: To a solution of the sulfoxide (400 mg) in AcCN (5 mL) was added D-valinamide HCl salt (240 mg, 1.56 mmol) and DIPEA (0.55 mL, 3.12 mmol). After heating at 80° C. for 2 h, the mixture was diluted with EtOAc, the organic layer was washed with 1N HCl, Sat. NaHCO₃, Brine, dried and concentrated to give (R)-2-(5-cyano-4-(3-(2H-1,2,3-triazol-2-yl)phenylamino)-6-(methylthio)pyrimidin-2-ylamino)-3-methylbutanamide (430 mg).

Step 2: To a solution of (R)-2-(5-cyano-4-(3-(2H-1,2,3-triazol-2-yl)phenylamino)-6-(methylthio)pyrimidin-2-ylamino)-3-methylbutanamide (200 mg, 0.47 mmol) in THF (2.5 mL) was added mCPBA (65%, 138 mg, 0.52 mmol) at room temperature. After stirring for 10 min, it was diluted with EtOAc, organic layer was washed with Sat. NaHCO3, brine, dried and concentrated to give corresponding sulfoxide. To this sulfoxide in MeOH (2 mL) was added K2CO3 (138 mg), and the mixture was stirred at room temperature for 5 min. MeOH was removed under vacuo, and the residue was added water, the resulting precipitate was collected by filtration to give (R)-2-(5-cyano-4-(3-(2H-1,2,3-triazol-2-yl)phenylamino)-6-methoxypyrimidin-2-ylamino)-3-methylbutanamide (160 mg).

Step 3: To a solution of the aforementioned (R)-2-(5-cyano-4-(3-(2H-1,2,3-triazol-2-yl)phenylamino)-6-methoxypyrimidin-2-ylamino)-3-methylbutanamide (160 mg) in DMSO (1.5 mL) was added K2CO3 (500 mg) and H2O2 (50%, 1.5 mL). After heating at 90° C. for 30 min, the mixture was cooled to room temperature, diluted with water, the resulting precipitate was collected by filtration to give (R)-2-(1-amino-3-methyl-1-oxobutan-2-ylamino)-4-(3-(2H-1,2,3-triazol-2-yl)phenylamino)pyrimidine-5-carboxamide (120 mg).

Step 4: To a solution of (R)-2-(1-amino-3-methyl-1-oxobutan-2-ylamino)-4-(3-(2H-1,2,3-triazol-2-yl)phenylamino)pyrimidine-5-carboxamide (100 mg, 0.24 mmol) in DCM (5 mL) was added BBr₃ (0.15 mL) at room temperature. After stirring for 15 h at room temperature, the mixture was concentrated and purified by preparative HPLC to give (R)-4-(3-(2H-1,2,3-triazol-2-yl)phenylamino)-2-(1-amino-3-methyl-1-oxobutan-2-ylamino)-6-oxo-1,6-dihydropyrimidine-5-carboxamide (18 mg). MS found for C₁₈H₂₁N₉O₃ as (M+H)⁺ 412.4. λ=260.4, 303.1.

### Example 7

### (R)-4-(3-(2H-1,2,3-triazol-2-yl)phenylamino)-2-(1-amino-4-methyl-1-oxopentan-2-ylamino)-6-oxo-1,6-dihydropyrimidine-5-carboxamide

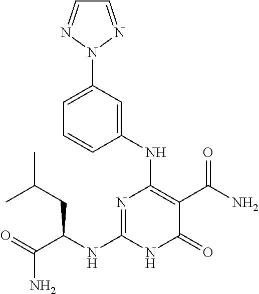 [SMILES: CC(C)C[C@@H](Nc1nc(Nc2cccc(-n3nccn3)c2)c(C(N)=O)c(=O)[nH]1)C(N)=O]

The title compound was synthesized similar to Example 6 in scheme 3 using D-leucinamide to replace D-Valinamide. MS found for C₁₇H₂₃N₉O₃ as (M+H)⁺ 426.3. λ=262.9, 308.0.

### Example 8

### (R)-4-(3-(2H-1,2,3-triazol-2-yl)phenylamino)-2-(1-amino-1-oxobutan-2-ylamino)-6-oxo-1,6-dihydropyrimidine-5-carboxamide

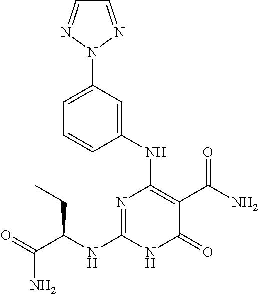 [SMILES: CC[C@@H](Nc1nc(Nc2cccc(-n3nccn3)c2)c(C(N)=O)c(=O)[nH]1)C(N)=O]

The title compound was synthesized similar to Example 6 in scheme 3 using (R)-2-aminobutanamide to replace D-Valinamide. MS found for C₁₇H₁₉N₉O₃ as (M+H)⁺ 398.3. λ=259.4, 300.9.

### Example 9

### (R)-2-(1-amino-4-methyl-1-oxopentan-2-ylamino)-6-oxo-4-(m-tolylamino)-1,6-dihydropyrimidine-5-carboxamide

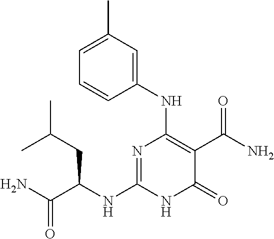 [SMILES: Cc1cccc(Nc2nc(N[C@H](CC(C)C)C(N)=O)[nH]c(=O)c2C(N)=O)c1]

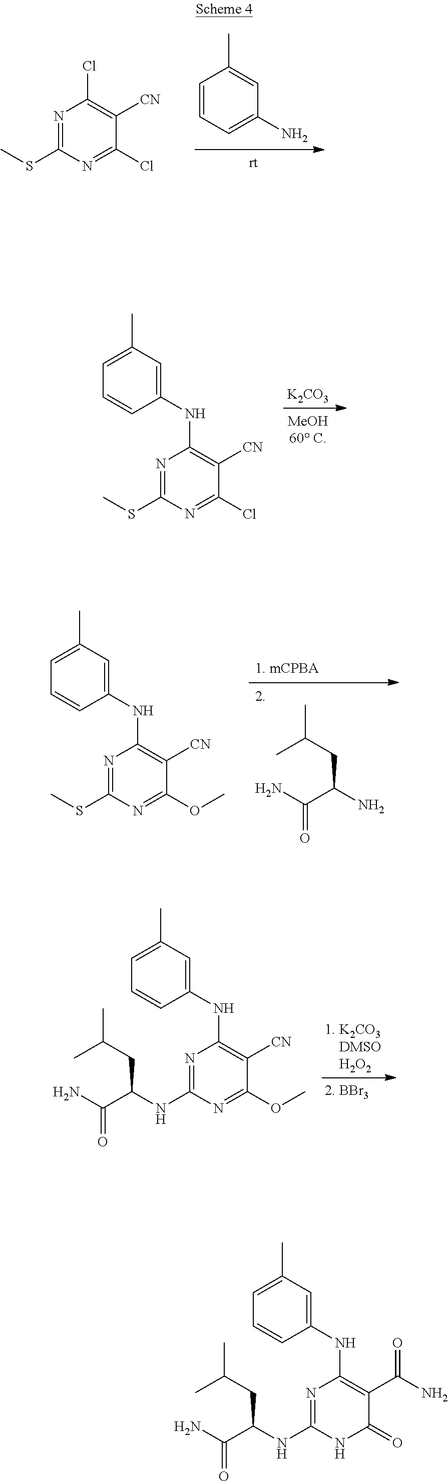

Step 1: To a suspension of 4,6-dichloro-2-(methylthio)pyrimidine-5-carbonitrile (500 mg, 2.27 mmol) in DMF (4 mL) was added m-toluidine (0.271 mL, 2.5 mmol). After stirring at room temperature for 4 h, the mixture was diluted with water, the resulting precipitate was collected by filtration to give 4-chloro-2-(methylhio)-6-(m-tolylamino)pyrimidin-5-carbonitrile (600 mg).

Step 2: To a suspension of 4-chloro-2-(methylhio)-6-(m-tolylamino)pyrimidin-5-carbonitrile (600 mg, 2.07 mmol) in MeOH (5 mL) was added K2CO3 (857 mg, 6.21 mmol), the mixture was stirred at room temperature for 4 h, MeOH was removed under vacuum, the residue was diluted with water, and the resulting precipitate was collected by filtration to give 4-methoxy-2-(methylhio)-6-(m-tolylamino)pyrimidin-5-carbonitrile (556 mg).

Step 3: To a solution of 4-methoxy-2-(methylhio)-6-(m-tolylamino)pyrimidin-5-carbonitrile (556 mg, 1.94 mmol) was added mCPBA (65%, 566 mg, 2.13 mmol). After stirring at room temperature for 30 min, it was added water and EtOAc, organic layer was separated and washed with Sat. NaHCO₃, brine, dried and concentrated to give a mixture of crude sulfoxide and sulfone. Part of the mixture (150 mg) was subsequently dissolved in AcCN (2 mL), added D-leucinamide HCl salt (103 mg, 0.62 mmol) and DIPEA (0.222 mL, 1.25 mmol), the mixture was heated at 80° C. for 2 h, cooled to room temperature, diluted with water, the precipitate was collected by filtration to give (R)-2-(5-cyano-4-methoxy-6-(m-tolylamino)pyrimidin-2-ylamino)4-methylpentamide.

Step 4: To above (R)-2-(5-cyano-4-methoxy-6-(m-tolylamino)pyrimidin-2-ylamino)4-methylpentamide in DMSO (1 mL) was added H2O2 (50%, 1 mL), K2CO3 (100 mg, 0.73 mmol), after heated at 90° C. for 1 h, it was cooled and diluted with water, the resulting precipitate was collected by filtration to give crude amide. The crude amide was diluted with DCM (1 mL) and added BBr₃ (0.1 mL), the resulting suspension was stirred overnight, and then purified by preparative HPLC to give (R)-2-(1-amino-4-methyl-1-oxopentan-2-ylamino)-6-oxo-4-(m-tolylamino)-1,6-dihydropyrimidine-5-carboxamide (37 mg). MS found for C₁₈H₂₄N₆O₃ as (M+H)⁺ 373.3. λ=257.0, 300.1.

### Example 10

### (R)-2-(1-amino-3-methyl-1-oxobutan-2-ylamino)-6-oxo-4-(m-tolylamino)-1,6-dihydropyrimidine-5-carboxamide

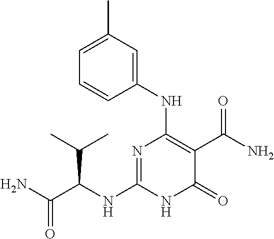 [SMILES: Cc1cccc(Nc2nc(N[C@@H](C(N)=O)C(C)C)[nH]c(=O)c2C(N)=O)c1]

The title compound was synthesized similar to example 9 in scheme 4 using D-Valinamide to replace D-leucinamide. MS found for C₁₇H₂₂N₆O₃ as (M+H)⁺ 359.2. λ=258.2, 298.5.

### Example 11

### (R)-2-(1-amino-1-oxobutan-2-ylamino)-6-oxo-4-(m-tolylamino)-1,6-dihydropyrimidine-5-carboxamide

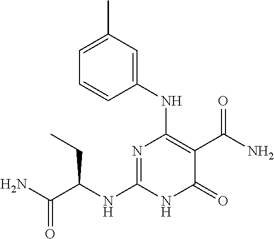 [SMILES: CC[C@@H](Nc1nc(Nc2cccc(C)c2)c(C(N)=O)c(=O)[nH]1)C(N)=O]

The title compound was synthesized similar to example 9 in scheme 4 using (R)-2-aminobutanamide to replace D-leucinamide. MS found for C₁₆H₂₀N₆O₃ as (M+H)⁺ 345.2. λ=258.2, 298.5.

### Example 12

### (R)-2-(1-amino-3-methylbutan-2-ylamino)-4-(3,5-dimethylphenylamino)-6-oxo-1,6-dihydropyrimidine-5-carboxamide

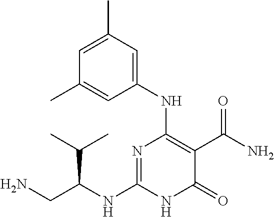 [SMILES: Cc1cc(C)cc(Nc2nc(N[C@@H](CN)C(C)C)[nH]c(=O)c2C(N)=O)c1]

The title compound was synthesized similar to example 5 using 3,5-dimethylaniline to replace 3-(2H-1,2,3-triazol-2-yl)aniline and using (R)-tert-butyl-2-amino-3-ethylbutylcarbamate to replace tert-butyl (1S,2R)-2-aminocyclohexylcarbamate. MS found for C₁₈H₂₆N₆O₂ as (M+H)⁺ 359.3. λ=257.0, 292.6.

### Example 13

### (R)-2-(1-amino-4-methyl-1-oxopentan-2-ylamino)-6-oxo-4-(quinolin-6-ylamino)-1,6-dihydropyrimidine-5-carboxamide

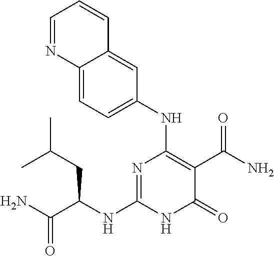 [SMILES: CC(C)C[C@@H](Nc1nc(Nc2ccc3ncccc3c2)c(C(N)=O)c(=O)[nH]1)C(N)=O]

The title compound was synthesized similar to example 9 in scheme 4 using 6-aminoquinoline to replace m-toluidine. MS found for C₂₀H₂₃N₇O₃ as (M+H)⁺ 410.3. λ=267.6, 294.9.

### Example 14

### 2-(2-(methylamino)ethylamino-6-oxo-4-(m-tolyl)-1,6-dihydropyrimidine-5-carboxamide

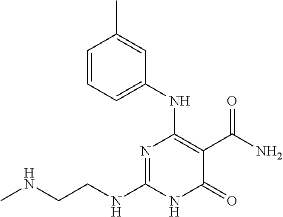 [SMILES: CNCCNc1nc(Nc2cccc(C)c2)c(C(N)=O)c(=O)[nH]1]

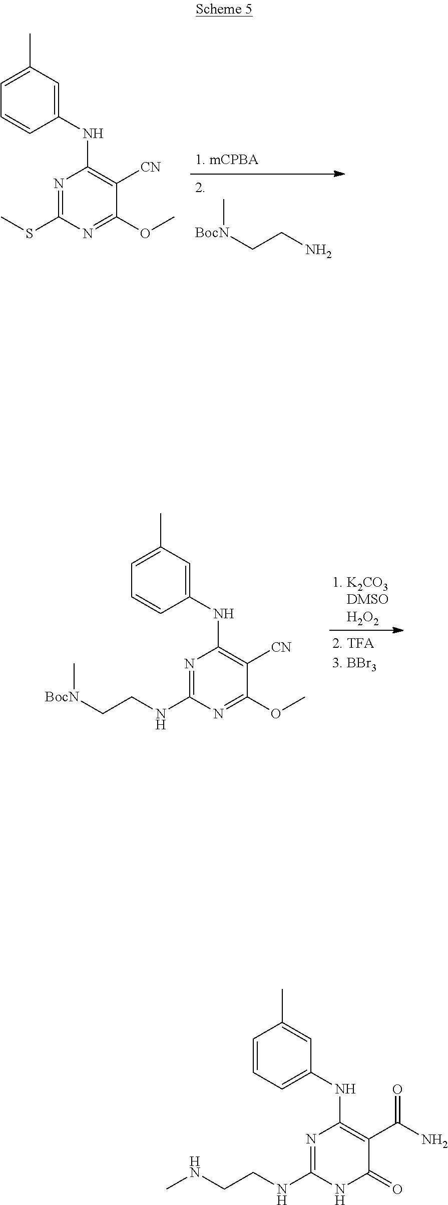 [SMILES: COc1nc(SC)nc(Nc2cccc(C)c2)c1C#N ; CN(CCN)C(=O)OC(C)(C)C ; COc1nc(NCCN(C)C(=O)OC(C)(C)C)nc(Nc2cccc(C)c2)c1C#N ; CNCCNc1nc(Nc2cccc(C)c2)c(C(N)=O)c(=O)[nH]1]

Step 1: To a solution of 4-methoxy-2-(methylhio)-6-(m-tolylamino)pyrimidin-5-carbonitrile (556 mg, 1.94 mmol) was added mCPBA (65%, 566 mg, 2.13 mmol). After stirring at room temperature for 30 min, it was added water and EtOAc, organic layer was separated and washed with Sat. NaHCO₃, brine, dried and concentrated to give a mixture of crude sulfoxide and sulfone. A portion of the mixture (150 mg) was subsequently dissolved in AcCN (1 mL), added tert-butyl 2-aminoethyl(methyl)carbamate (130 mg, 0.75 mmol) and DIPEA (0.133 mL, 0.75 mmol), the mixture was heated at 80° C. for 2 h, cooled to room temperature, diluted with water and EtOAc, organic layer was separated and washed with brine, dried and concentrated to give tert-butyl 2-(5-cyano-4-methoxy-6-(m-tolylamino)pyrimidin-2-ylamino)ethyl(methyl)carbamate as crude oil.

Step 2: To above tert-butyl 2-(5-cyano-4-methoxy-6-(m-tolylamino)pyrimidin-2-ylamino)ethyl(methyl)carbamate in DMSO (1 mL) was added H2O2 (50%, 1 mL), K2CO3 (400 mg, 2.89 mmol), after heated at 90° C. for 1 h, it was cooled and diluted with water, the resulting precipitate was collected by filtration to give crude amide (125 mg). The crude amide was diluted with DCM (1 mL) and added TFA (1 mL), 30 min later, the solution was concentrated and again diluted with DCM (1 mL), added BBr₃ (0.1 mL). After stirring at room temperature for 4 h, the mixture was concentrated and purified by preparative HPLC to give (2-(methylamino)ethylamino-6-oxo-4-(m-tolyl)-1,6-dihydropyrimidine-5-carboxamide (34 mg). MS found for C₁₅H₂₀N₆O₂ as (M+H)⁺ 317.2. λ=253.4, 291.4.

### Example 15

### (R)-2-(1-amino-3-methyl-1-oxobutan-2-ylamino)-4-methoxy-6-(quinolin-6-ylamino)pyrimidine-5-carboxamide

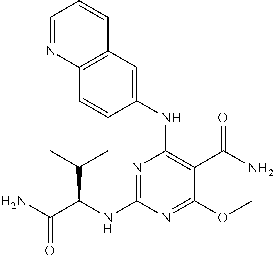 [SMILES: COc1nc(N[C@@H](C(N)=O)C(C)C)nc(Nc2ccc3ncccc3c2)c1C(N)=O]

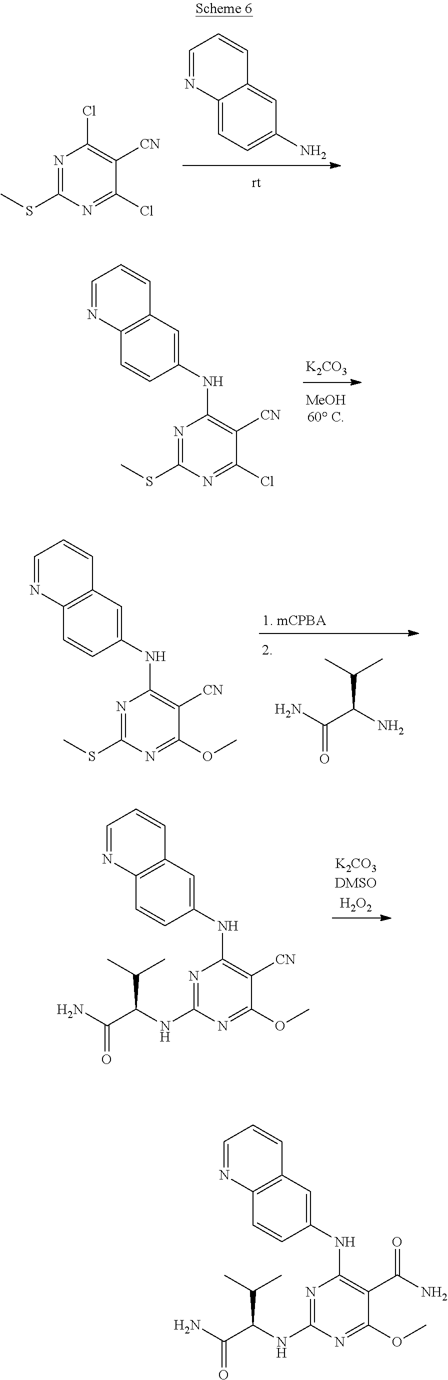

Step 1: To a suspension of 4,6-dichloro-2-(methylthio)pyrimidine-5-carbonitrile (538 mg, 2.44 mmol) in DMF (4 mL) was added 6-aminoquinoline (388 mg, 2.69 mmol). After stirring at room temperature for 20 min, the mixture was diluted with water, the resulting precipitate was collected by filtration to give 4-chloro-2-(methylhio)-6-(quinolin-6-yl)pyrimidin-5-carbonitrile (800 mg).

Step 2: To a suspension of 4-chloro-2-(methylhio)-6-(quinolin-6-yl)pyrimidin-5-carbonitrile (800 mg, 2.45 mmol) in MeOH (5 mL) was added K2CO3 (1.10 g, 7.34 mmol), the mixture was stirred at room temperature for 4 h, MeOH was removed under vacuum, the residue was diluted with water, and the resulting precipitate was collected by filtration to give 4-methoxy-2-(methylhio)-6-(quinolin-6-yl)pyrimidin-5-carbonitrile (622 mg).

Step 3: To a solution of 4-methoxy-2-(methylhio)-6-(quinolin-6-yl)pyrimidin-5-carbonitrile (200 mg, 1.94 mmol) was added mCPBA (65%, 327 mg, 1.23 mmol). After stirring at room temperature for 30 min, it was added water and EtOAc, organic layer was separated and washed with Sat. NaHCO₃, brine, dried and concentrated to give a mixture of crude sulfoxide and sulfone, which was subsequently diluted with AcCN (2 mL), added D-valinamide HCl salt (140 mg, 0.91 mmol) and DIPEA (0.38 mL, 2.1 mmol), the mixture was heated at 80° C. for 2 h, cooled to room temperature, diluted with water, the precipitate was collected by filtration to give (R)-2-(5-cyano-4-methoxy-6-(quinolin-6-yl)pyrimidin-2-ylamino)-3-methylbutanamide as crude solid (90 mg).

Step 4: To above (R)-2-(5-cyano-4-methoxy-6-(quinolin-6-yl)pyrimidin-2-ylamino)-3-methylbutanamide in DMSO (0.5 mL) was added H2O2 (50%, 1 mL), K2CO3 (300 mg, 2.2 mmol). After heating at 90° C. for 1 h, it was cooled and diluted with water, and then purified by preparative HPLC to give (R)-2-(1-amino-3-methyl-1-oxobutan-2-ylamino)-4-methoxy-6-(quinolin-6-ylamino)pyrimidine-5-carboxamide (23 mg). MS found for C₂₀H₂₃N₇O₃ as (M+H)⁺ 410.3. λ=284.2.

### Example 16

### (R)-2-(1-amino-1-oxobutan-2-ylamino)-4-methoxy-6-(quinolin-6-ylamino)pyrimidine-5-carboxamide and Example 17. (R)-6-(2-(1-amino-1-oxobutan-2-ylamino)-5-carbamoyl-6-methoxypyrimidin-4-ylamino)quinoline 1-oxide

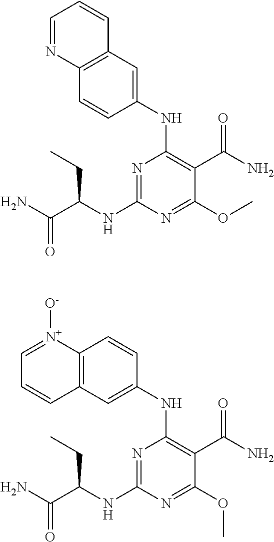 [SMILES: CC[C@@H](Nc1nc(Nc2ccc3ncccc3c2)c(C(N)=O)c(OC)n1)C(N)=O ; CC[C@@H](Nc1nc(Nc2ccc3c(ccc[n+]3[O-])c2)c(C(N)=O)c(OC)n1)C(N)=O]

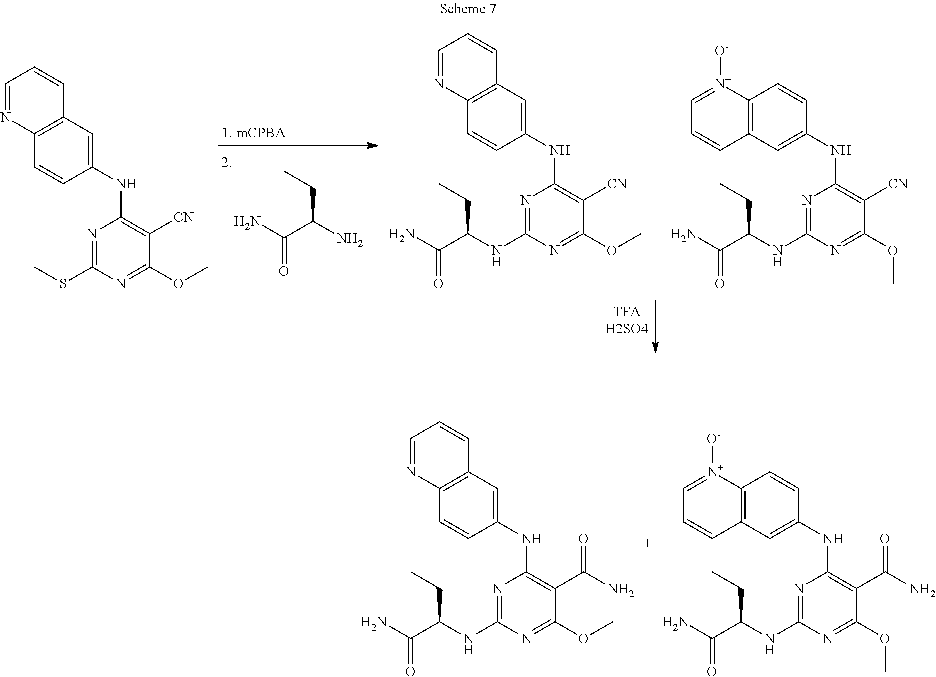

Step 1: To a solution of 4-methoxy-2-(methylhio)-6-(quinolin-6-yl)pyrimidin-5-carbonitrile (200 mg, 1.94 mmol) in AcCN (1.5 mL) was added mCPBA (65%, 327 mg, 1.23 mmol). After stirring at room temperature for 30 min, it was added water and EtOAc, organic layer was separated and washed with Sat. NaHCO3, brine, dried and concentrated to give a mixture of crude sulfoxide and sulfone, which was subsequently diluted with AcCN (2 mL), added (R)-2-aminobutanamide HCl salt (125 mg, 0.91 mmol) and DIPEA (0.38 mL, 2.1 mmol), the mixture was heated at 80° C. for 2 h, cooled to room temperature, diluted with water, the precipitate was collected by filtration to give a mixture of (R)-2-(5-cyano-4-methoxy-6-(quinolin-6-yl)pyrimidin-2-ylamino)butanamide and (R)-6-(2-(1-amino-1-oxobutan-2-ylamino)-5-cyano-6-methoxypyrimidin-4-ylamino)quinoline 1-oxide as crude solid (142 mg).

Step 4: To above mixture in TFA (3 mL) was added conc. H2SO4 (1 mL). After heating at 80° C. for 3 h, it was cooled and diluted with water, and then purified by preparative HPLC to give (R)-2-(1-amino-1-oxobutan-2-ylamino)-4-methoxy-6-(quinolin-6-ylamino)pyrimidine-5-carboxamide (40 mg, MS found for C₁₉H₂₁N₇O₃ as (M+H)⁺ 396.2. λ=283.1) and (R)-6-(2-(1-amino-1-oxobutan-2-ylamino)-5-carbamoyl-6-methoxypyrimidin-4-ylamino)quinoline 1-oxide (42 mg, MS found for C₁₉H₂₁N₇O₄ as (M+H)⁺ 412.2. λ=297.3).

### Example 18

### (R)-4-(4-(1H-pyrazol-1ylamino)phenylamino)-2-(1-amino-4-methyl-1-oxopentan-2-ylamino)-6-oxo-1,6-dihydropyrimidine-5-carboxamide

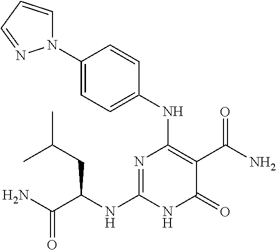 [SMILES: CC(C)C[C@@H](Nc1nc(Nc2ccc(-n3cccn3)cc2)c(C(N)=O)c(=O)[nH]1)C(N)=O]

The title compound was synthesized similar to example 9 in scheme 4 using 4-(1H-pyrazol-1-yl)aniline to replace m-toluidine. MS found for C₂₀H₂₄N₈O₃ as (M+H)⁺ 425.3. λ=276.7, 311.2.

### Example 19

### (R)-4-(4-(1H-pyrazol-1ylamino)phenylamino)-2-(1-amino-3-methyl-1-oxobutan-2-ylamino)-6-oxo-1,6-dihydropyrimidine-5-carboxamide

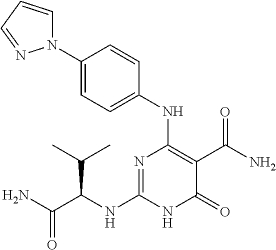 [SMILES: CC(C)[C@@H](Nc1nc(Nc2ccc(-n3cccn3)cc2)c(C(N)=O)c(=O)[nH]1)C(N)=O]

The title compound was synthesized similar to example 9 in scheme 4 using 4-(1H-pyrazol-1-yl)aniline to replace m-toluidine and using D-valinamide to replace D-leucinamide. MS found for C₁₉H₂₂N₈O₃ as (M+H)⁺ 411.3. λ=274.8, 311.6.

### Example 20

### (R)-2-(1-amino-3-methyl-1-oxobutan-2-ylamino)-6-oxo-4-(quinolin-6-ylamino)-1,6-dihydropyrimidine-5-carboxamide

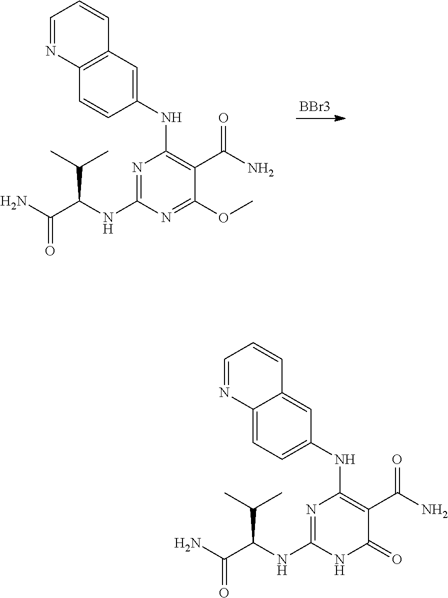 [SMILES: COc1nc(N[C@@H](C(N)=O)C(C)C)nc(Nc2ccc3ncccc3c2)c1C(N)=O ; "BBr3" ; CC(C)[C@@H](Nc1nc(Nc2ccc3ncccc3c2)c(C(N)=O)c(=O)[nH]1)C(N)=O]

To a suspension of (R)-2-(1-amino-3-methyl-1-oxobutan-2-ylamino)-4-methoxy-6-(quinolin-6-ylamino)pyrimidine-5-carboxamide (22 mg) in DCM (1.5 mL) was added BBr3 (0.15 mL). After stirring at room temperature for 15 h, the mixture was purified by preparative HPLC to give (R)-2-(1-amino-3-methyl-1-oxobutan-2-ylamino)-6-oxo-4-(quinolin-6-ylamino)-1,6-dihydropyrimidine-5-carboxamide. MS found for C₁₉H₂₁N₇O₃ as (M+H)⁺396.2. λ=267.2, 293.8.

### Example 21

### (R)-2-(1-amino-1-oxobutan-2-ylamino)-6-oxo-4-(quinolin-6-ylamino)-1,6-dihydropyrimidine-5-carboxamide

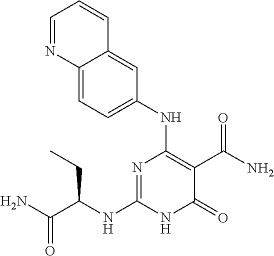 [SMILES: CC[C@@H](Nc1nc(Nc2ccc3ncccc3c2)c(C(N)=O)c(=O)[nH]1)C(N)=O]

The title compound was synthesized similar to examples 15 and 20 using (R)-2-aminobutanamide to replace D-leucinamide. MS found for C₁₈H₁₉N₇O₃ as (M+H)⁺ 382.2. λ=266.5, 293.8.

### Example 22

### (R)-4-(3-(2H-1,2,3-triazol-2-yl)phenylamino)-2-(1-amino-3-methylbutan-2-ylamino)-6-oxo-1,6-dihydropyrimidine-5-carboxamide

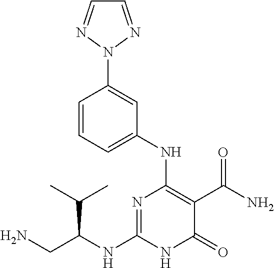 [SMILES: CC(C)[C@H](CN)Nc1nc(Nc2cccc(-n3nccn3)c2)c(C(N)=O)c(=O)[nH]1]

The title compound was synthesized similar to example 5 using (R)-tert-butyl-2-amino-3-ethylbutylcarbamate to replace tert-butyl (1S,2R)-2-aminocyclohexylcarbamate. MS found for C₁₈H₂₃N₉O₂ as (M+H)⁺ 398.3. λ=258.2, 300.9.

### Example 23

### 4-(3-(2H-1,2,3-triazol-2-yl)phenylamino)-2-(2-(methylamino)ethylamino)-6-oxo-1,6-dihydropyrimidine-5-carboxamide

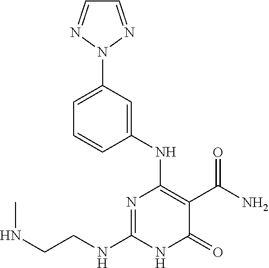 [SMILES: CNCCNc1nc(Nc2cccc(-n3nccn3)c2)c(C(N)=O)c(=O)[nH]1]

The title compound was synthesized similar to example 14 in scheme 5 using 3-(2H-1,2,3-triazol-2-yl)aniline to replace m-toluidine. MS found for C₁₆H₁₉N₉O₂ as (M+H)⁺ 370.3. λ=258.2, 298.5.

### Example 24

### (R)-4-(4-(1H-pyrazol-1ylamino)phenylamino)-2-(1-amino-1-oxobutan-2-ylamino)-6-oxo-1,6-dihydropyrimidine-5-carboxamide

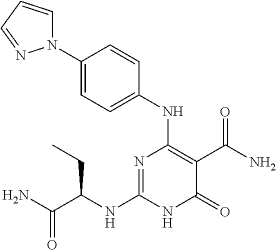 [SMILES: CC[C@@H](Nc1nc(Nc2ccc(-n3cccn3)cc2)c(C(N)=O)c(=O)[nH]1)C(N)=O]

The title compound was synthesized similar to example 9 in scheme 4 using 4-(1H-pyrazol-1-yl)aniline to replace m-toluidine and using (R)-2-aminobutanamide to replace D-leucinamide. MS found for C₁₈H₂₀N₈O₃ as (M+H)⁺ 397.4. λ=274.8, 311.6.

### Example 25

### (R)-4-(1H-indazol-5-ylamino)-2-(1-amino-4-methyl-1-oxopentan-2-ylamino)-6-oxo-1,6-dihydropyrimidine-5-carboxamide

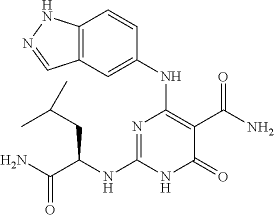 [SMILES: CC(C)C[C@@H](Nc1nc(Nc2ccc3[nH]ncc3c2)c(C(N)=O)c(=O)[nH]1)C(N)=O]

The title compound was synthesized similar to example 9 in scheme 4 using 5-aminoindazole to replace m-toluidine. MS found for C₁₈H₂₂N₈O₃ as (M+H)⁺ 399.3. λ=251.1.

### Example 26

### (R)-4-(1H-indazol-5-ylamino)-2-(1-amino-3-methyl-1-oxobutan-2-ylamino)-6-oxo-1,6-dihydropyrimidine-5-carboxamide

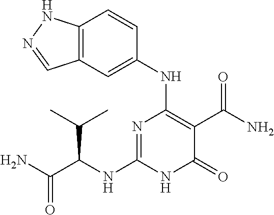 [SMILES: CC(C)[C@@H](Nc1nc(Nc2ccc3[nH]ncc3c2)c(C(N)=O)c(=O)[nH]1)C(N)=O]

The title compound was synthesized similar to example 9 in scheme 4 using 5-aminoindazole to replace m-toluidine and using D-valinamide to replace D-leucinamide. MS found for C₁₇H₂₀N₈O₃ as (M+H)⁺ 385.3. λ=249.9.

### Example 27

### (R)-2-(1-amino-4-methyl-1-oxopentan-2-ylamino)-6-oxo-4-(3-(pyrimidin-2-yl)phenylamino)-1,6-dihydropyrimidine-5-carboxamide

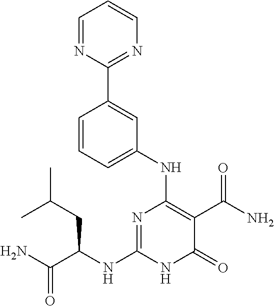 [SMILES: CC(C)C[C@@H](Nc1nc(Nc2cccc(-c3ncccn3)c2)c(C(N)=O)c(=O)[nH]1)C(N)=O]

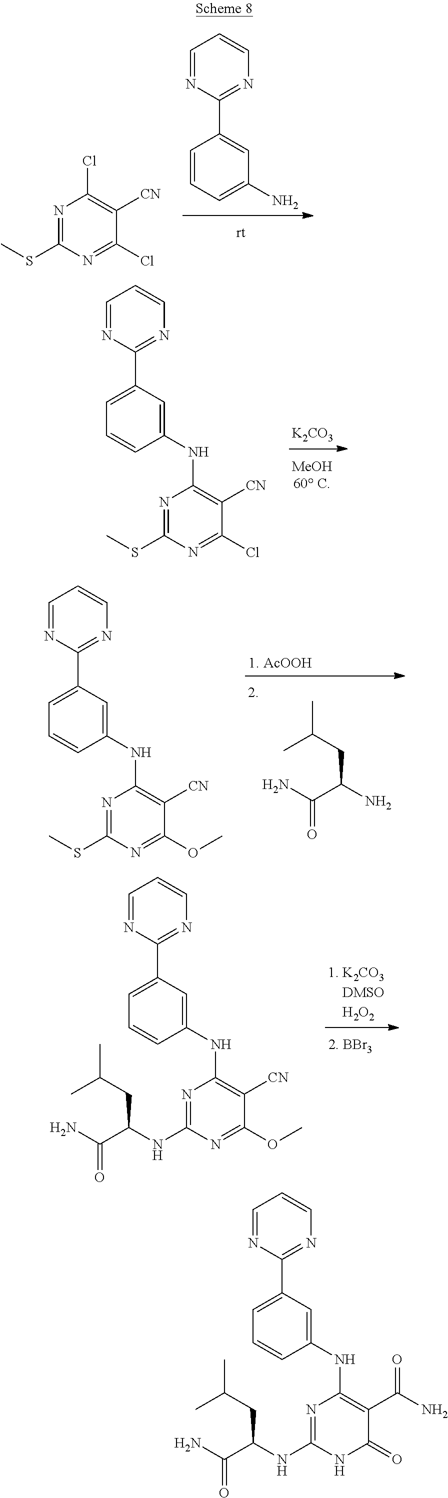

Step 1: To a solution of 4,6-dichloro-2-(methylthio)pyrimidine-5-carbonitrile (600 mg, 2.72 mmol) in DMF (3 mL) was added 3-(pyrimidin-2-yl)aniline (512 mg, 3.0 mmol). After stirring at room temperature for 3 h (DIPEA may be needed to drive the reaction to completion), the mixture was diluted with water, the resulting precipitate was collected by filtration to give 4-chloro-2-(methylthio)-6-(3-(pyrimidin-2-yl)phenylamino)pyrimidine-5-carbonitrile (970 mg).

Step 2: To a suspension of 4-chloro-2-(methylthio)-6-(3-(pyrimidin-2-yl)phenylamino)pyrimidine-5-carbonitrile (970 mg) in MeOH (10 mL) was added K2CO3 (1.13 g, 8.15 mmol), the mixture was stirred at room temperature for 2 h and heated at 60° C. for 30 min (product overlap with starting material, make sure reaction is completed by Mass spec). After cooled to room temperature, MeOH was removed under vacuo, the residue was diluted with water, the resulting precipitate was collected by filtration to give 4-methoxy-2-(methylthio)-6-(3-(pyrimidin-2-yl)phenylamino)pyrimidine-5-carbonitrile (875 mg).

Step 3: To 4-methoxy-2-(methylthio)-6-(3-(pyrimidin-2-yl)phenylamino)pyrimidine-5-carbonitrile (150 mg, 0.43 mmol) was added peracetic acid in AcOH (39%, 2 mL). After stirring at room temperature for 30 min, it was concentrated by rotavap, diluted with EtOAc, organic layer was washed with Sat. NaHCO3, brine, dried and concentrated to give crude sulfoxide. The sulfoxide was subsequently dissolved in NMP (1 mL), added L-leucinamide HCl salt (85 mg, 0.51 mmol) and DIPEA (0.4 mL, 2.2 mmol), the mixture was heated at 75° C. for 1 h, cooled to room temperature, diluted with water, the precipitate was collected by filtration to give (R)-2-(5-cyano-4-methoxy-6-(3-(pyrimidin-2-yl)phenylamino)pyrimidin-2-ylamino)-4-methylpentamide.

Step 4: To above (R)-2-(5-cyano-4-methoxy-6-(3-(pyrimidin-2-yl)phenylamino)pyrimidin-2-ylamino)-4-methylpentamide in DMSO (1 mL) was added H2O2 (50%, 1 mL), K2CO3 (300 mg, 2.2 mmol), after heated at 90° C. for 1 h, it was cooled and diluted with water, the resulting precipitate was collected by filtration to give crude amide. The crude amide was diluted with DCM (1 mL) and added BBr₃ (0.2 mL), the resulting suspension was stirred overnight, and then purified by preparative HPLC to give (R)-2-(1-amino-4-methyl-1-oxopentan-2-ylamino)-6-oxo-4-(3-(pyrimidin-2-yl)phenylamino)-1,6-dihydropyrimidine-5-carboxamide (40 mg). MS found for C₂₁H₂₄N₈O₃ as (M+H)⁺ 437.4. λ=258.2, 302.1.

### Example 28

### (R)-2-(1-amino-3-methyl-1-oxobutan-2-ylamino)-6-oxo-4-(3-(pyrimidin-2-yl)phenylamino)-1,6-dihydropyrimidine-5-carboxamide

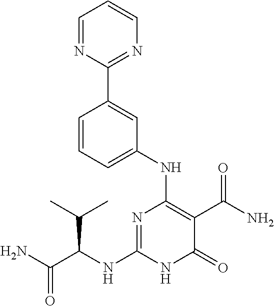 [SMILES: CC(C)[C@@H](Nc1nc(Nc2cccc(-c3ncccn3)c2)c(C(N)=O)c(=O)[nH]1)C(N)=O]

The title compound was synthesized similar to example 27 in scheme 8 using D-valinamide to replace D-leucinamide. MS found for C₂₀H₂₂N₈O₃ as (M+H)⁺ 423.3. λ=251.1, 299.7.

### Example 29

### (R)-2-(1-amino-4-methyl-1-oxopentan-2-ylamino)-6-oxo-4-(3-(pyrimidin-2-yl)phenylamino)-1,6-dihydropyrimidine-5-carboxamide

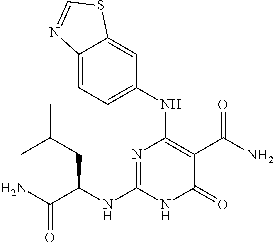 [SMILES: CC(C)C[C@@H](Nc1nc(Nc2ccc3ncsc3c2)c(C(N)=O)c(=O)[nH]1)C(N)=O]

The title compound was synthesized similar to example 9 in scheme 4 using 6-aminobenzothiazole to replace m-toluidine. MS found for C₁₈H₂₁N₇O₃S as (M+H)⁺ 416.3. λ=246.3, 278.3, 316.4.

### Example 30

### (R)-2-(1-amino-3-methyl-1-oxobutan-2-ylamino)-6-oxo-4-(3-(pyrimidin-2-yl)phenylamino)-1,6-dihydropyrimidine-5-carboxamide

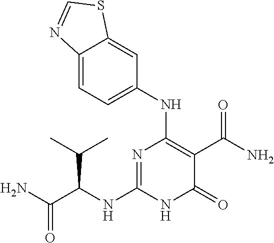 [SMILES: CC(C)[C@@H](Nc1nc(Nc2ccc3ncsc3c2)c(C(N)=O)c(=O)[nH]1)C(N)=O]

The title compound was synthesized similar to example 9 in scheme 4 using 6-aminobenzothiazole to replace m-toluidine and using D-valinamide to replace D-leucinamide. MS found for C₁₇H₁₉N₇O₃S as (M+H)⁺ 402.2. λ=246.3, 278.3, 316.4.

### Example 31

### (R)-2-(1-amino-1-oxobutan-2-ylamino)-6-oxo-4-(3-(pyrimidin-2-yl)phenylamino)-1,6-dihydropyrimidine-5-carboxamide

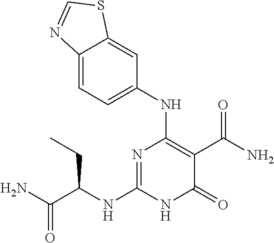 [SMILES: CC[C@@H](Nc1nc(Nc2ccc3ncsc3c2)c(C(N)=O)c(=O)[nH]1)C(N)=O]

The title compound was synthesized similar to example 9 in scheme 4 using 6-aminobenzothiazole to replace m-toluidine and using (R)-2-aminobutanamide to replace D-leucinamide. MS found for C₁₆H₁₇N₇O₃S as (M+H)⁺ 388.3. λ=246.6, 278.5, 315.5.

### Example 32

### (R)-4-(341H-pyrazol-1ylamino)phenylamino)-2-(1-amino-4-methyl-1-oxopentan-2-ylamino)-6-oxo-1,6-dihydropyrimidine-5-carboxamide

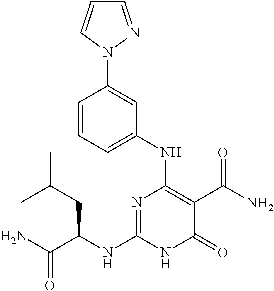 [SMILES: CC(C)C[C@@H](Nc1nc(Nc2cccc(-n3cccn3)c2)c(C(N)=O)c(=O)[nH]1)C(N)=O]

The title compound was synthesized similar to example 9 in scheme 4 using 3-(1H-pyrazol-1-yl)aniline to replace m-toluidine. MS found for C₂₀H₂₄N₈O₃ as (M+H)⁺ 425.3. λ=257.6, 301.9.

### Example 33

### (R)-4-(341H-pyrazol-1ylamino)phenylamino)-2-(1-amino-3-methyl-1-oxobutan-2-ylamino)-6-oxo-1,6-dihydropyrimidine-5-carboxamide

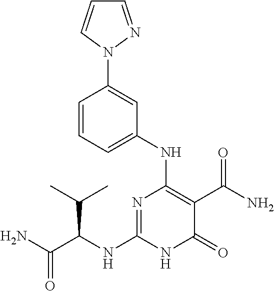 [SMILES: CC(C)[C@@H](Nc1nc(Nc2cccc(-n3cccn3)c2)c(C(N)=O)c(=O)[nH]1)C(N)=O]

The title compound was synthesized similar to example 9 in scheme 4 using 3-(1H-pyrazol-1-yl)aniline to replace m-toluidine and using D-valinamide to replace D-leucinamide. MS found for C₁₉H₂₂N₈O₃ as (M+H)⁺ 411.3. λ=255.8, 299.7.

### Example 34

### (R)-4-(341H-pyrazol-1ylamino)phenylamino)-2-(1-amino-1-oxobutan-2-ylamino)-6-oxo-1,6-dihydropyrimidine-5-carboxamide

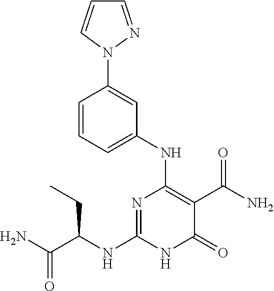 [SMILES: CC[C@@H](Nc1nc(Nc2cccc(-n3cccn3)c2)c(C(N)=O)c(=O)[nH]1)C(N)=O]

The title compound was synthesized similar to example 9 in scheme 4 using 3-(1H-pyrazol-1-yl)aniline to replace m-toluidine and using (R)-2-aminobutanamide to replace D-leucinamide. MS found for C₁₈H₂₀N₈O₃ as (M+H)⁺ 397.3. λ=259.4, 299.7.

### Example 35

### (R)-4-(1H-indazol-6-ylamino)-2-(1-amino-4-methyl-1-oxopentan-2-ylamino)-6-oxo-1,6-dihydropyrimidine-5-carboxamide

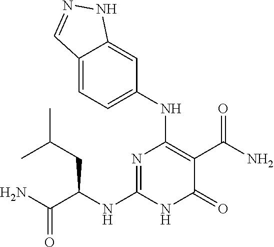 [SMILES: CC(C)C[C@@H](Nc1nc(Nc2ccc3cn[nH]c3c2)c(C(N)=O)c(=O)[nH]1)C(N)=O]

The title compound was synthesized similar to example 9 in scheme 4 using 6-aminoindazole to replace m-toluidine. MS found for C₁₈H₂₂N₈O₃ as (M+H)⁺ 399.3. λ=248.7, 277.1, 312.8.

### Example 36

### (R)-4-1H-indazol-6-ylamino)-2-(1-amino-1-oxobutan-2-ylamino)-6-oxo-1,6-dihydropyrimidine-5-carboxamide

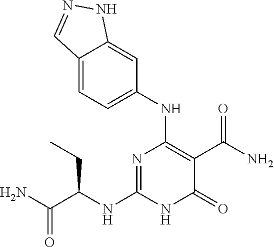 [SMILES: CC[C@@H](Nc1nc(Nc2ccc3cn[nH]c3c2)c(C(N)=O)c(=O)[nH]1)C(N)=O]

The title compound was synthesized similar to example 9 in scheme 4 using 6-aminoindazole to replace m-toluidine and using (R)-2-aminobutanamide to replace D-leucinamide. MS found for C₁₆H₁₈N₈O₃ as (M+H)⁺ 371.2. λ=248.7, 275.9, 311.6.

### Example 37

### (R)-4-(3-(2H-1,2,3-triazol-2-yl)phenylamino)-2-(1-amino-3-methylbutan-2-ylamino)-6-oxo-1,6-dihydropyrimidine-5-carboxamide

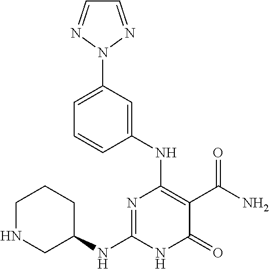 [SMILES: NC(=O)c1c(Nc2cccc(-n3nccn3)c2)nc(N[C@@H]2CCCNC2)[nH]c1=O]

The title compound was synthesized similar to example 5 using (R)-tert-butyl-3-aminopiperidine-1-carboxylate to replace tert-butyl (1S,2R)-2-aminocyclohexylcarbamate. MS found for C₁₈H₂₁N₉O₂ as (M+H)⁺ 396.3. λ=259.4, 298.5.

### Example 38

### 4-(3-(pyrimidin-2-yl)phenylamino)-2-((1R,2S)-2-aminocyclohexyl)-6-oxo-1,6-dihydropyrimidine-5-carboxamide

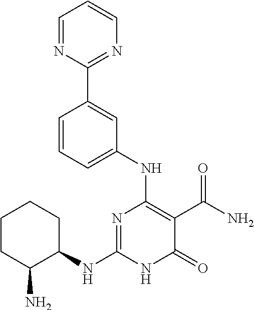 [SMILES: NC(=O)c1c(Nc2cccc(-c3ncccn3)c2)nc(N[C@@H]2CCCC[C@@H]2N)[nH]c1=O]

The title compound was synthesized similar to example 5 using 3-(pyrimidin-2-yl)aniline to replace 3-(2H-1,2,3-triazol-2-yl)aniline. MS found for C₂₁H₂₄N₈O₂ as (M+H)⁺ 421.3. λ=254.6, 298.5.

### Example 39

### (R)-2-(1-amino-1-oxobutan-2-ylamino)-4-(3-(methoxy)phenylamino)-6-oxo-1,6-dihydropyrimidine-5-carboxamide

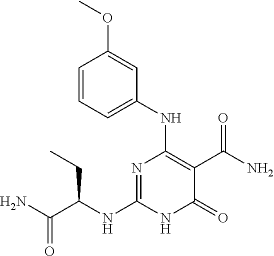 [SMILES: CC[C@@H](Nc1nc(Nc2cccc(OC)c2)c(C(N)=O)c(=O)[nH]1)C(N)=O]

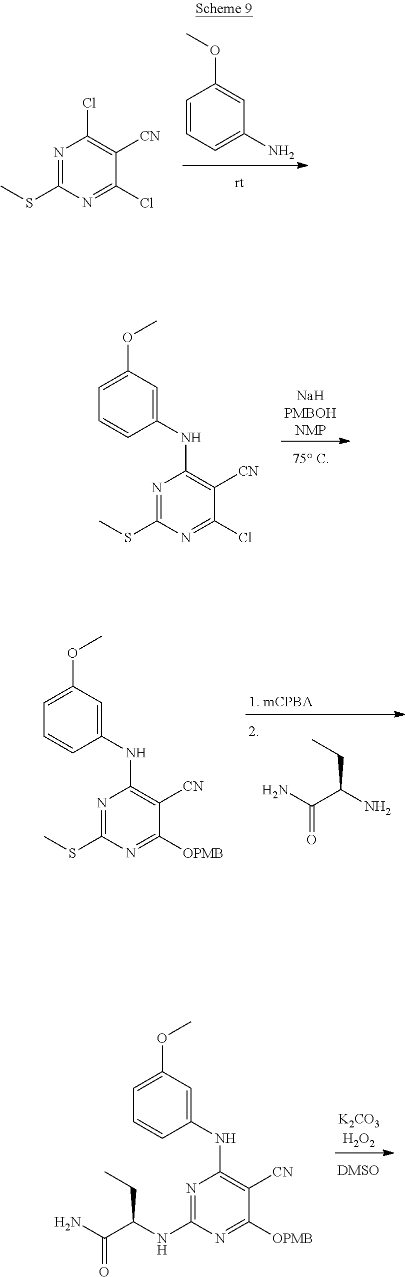

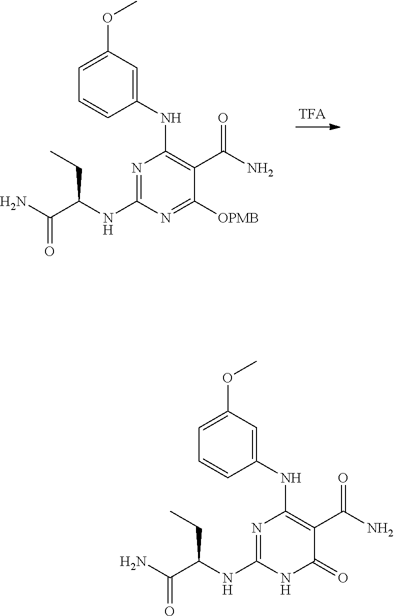

Step 1: To a suspension of 4,6-dichloro-2-(methylthio)pyrimidine-5-carbonitrile (330 mg, 1.5 mmol) in DMF (3 mL) was added m-anisidine (231 mg, 1.875 mmol). After stirring at room temperature for 4 h, the mixture was diluted with water, the resulting precipitate was collected by filtration to give 4-chloro-2-(methylhio)-6-(3-(methoxy)phenylamino)pyrimidin-5-carbonitrile (600 mg).

Step 2: To NaH (48 mg) in NMP (1 mL) was added p-methoxybenzyl alcohol (162 mg, 1.18 mmol), after stirring at room temperature for 1 h, it was added a solution of 4-chloro-2-(methylhio)-6-(3-(methoxy)phenylamino)pyrimidin-5-carbonitrile (240 mg, 0.78 mmol) in NMP (2 mL). The mixture was then heated at 60° C. for 1 h and 75° C. for 1 h, more NaH (50 mg) was added, followed by additional heating at 75° C. for 20 min. The mixture was added water, the resulting precipitate was collected by filtration to give 4-(4-methoxybenzyloxy)-2-(methylhio)-6-(3-(methoxy)phenylamino)pyrimidin-5-carbonitrile (260 mg).

Step 3: To a solution of 4-(4-methoxybenzyloxy)-2-(methylhio)-6-(3-(methoxy)phenylamino)pyrimidin-5-carbonitrile (260 mg, 0.64 mmol) was added mCPBA (65%, 204 mg, 0.77 mmol). After stirring at room temperature for 20 min, it was added water and EtOAc, organic layer was separated and washed with Sat. NaHCO3, brine, dried and concentrated to give a mixture of crude sulfoxide and sulfone. A portion of the crude sulfone and sulfoxide mixture (140 mg) was subsequently dissolved in NMP (1 mL), added (R)-2-aminobutanamide HCl salt (66 mg, 0.48 mmol) and DIPEA (0.142 mL, 0.8 mmol), the mixture was heated at 75° C. for 1 h, cooled to room temperature, diluted with water and EtOAc, organic layer was separated and washed with Sat. NaHCO₃, brine, dried and concentrated to give (R)-2-(5-cyano-4-(4-methoxybenzyloxy)-6-(3-(methoxy)phenylamino)pyrimidin-2-ylamino)butanamide as crude solid.

Step 4: To above (R)-2-(5-cyano-4-(4-methoxybenzyloxy)-6-(3-(methoxy)phenylamino)pyrimidin-2-ylamino)butanamide in DMSO (1 mL) was added H2O2 (50%, 1 mL), K2CO3 (200 mg, 1.44 mmol), after heated at 90° C. for 1 h, it was cooled and diluted with water, the resulting precipitate was collected by filtration to give crude amide (R)-2-(1-amino-1-oxobutan-2-ylamino)-4-(4-methoxybenzyloxy)-6-(3-methoxyphenylamino)pyrimidine-5-carboxamide.

Step 5: The above crude amide (R)-2-(1-amino-1-oxobutan-2-ylamino)-4-(4-methoxybenzyloxy)-6-(3-methoxyphenylamino)pyrimidine-5-carboxamide was dissolved with DCM (0.6 mL) and added TFA (0.4 mL), after 30 min the mixture was concentrated and purified by preparative HPLC to (R)-2-(1-amino-1-oxobutan-2-ylamino)-4-(3-(methoxy)phenylamino)-6-oxo-1,6-dihydropyrimidine-5-carboxamide (10 mg). MS found for C₁₆H₂₀N₆O₄ as (M+H)⁺361.3. λ=256.4, 301.9.

### Example 40

### (R)-2-(1-amino-4-methyl-1-oxopentan-2-ylamino)-6-oxo-4-(3-(pyridin-2-yl)phenylamino)-1,6-dihydropyrimidine-5-carboxamide

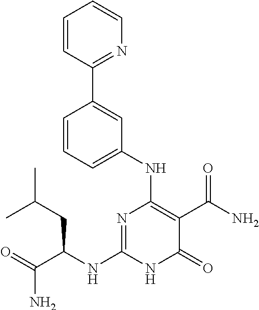 [SMILES: CC(C)C[C@@H](Nc1nc(Nc2cccc(-c3ccccn3)c2)c(C(N)=O)c(=O)[nH]1)C(N)=O]

The title compound was synthesized similar to example 27 in Scheme 8 using 3-(pyridin-2-yl)aniline to replace 3-(pyrimidin-2-yl)aniline. MS found for C₂₂H₂₅N₇O₃ as (M+H)⁺ 436.4. λ=249.9, 302.1.

### Example 41

### (R)-2-(1-amino-1-oxobutan-2-ylamino)-6-oxo-4-(3-(pyridin-2-yl)phenylamino)-1,6-dihydropyrimidine-5-carboxamide

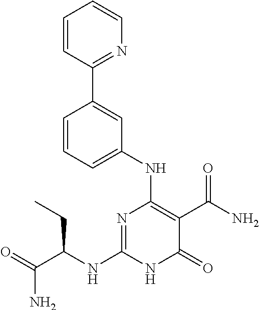 [SMILES: CC[C@@H](Nc1nc(Nc2cccc(-c3ccccn3)c2)c(C(N)=O)c(=O)[nH]1)C(N)=O]

The title compound was synthesized similar to example 27 in Scheme 8 using 3-(pyridin-2-yl)aniline to replace 3-(pyrimidin-2-yl)aniline and using (R)-2-aminobutanamide to replace D-leucinamide. MS found for C₂₀H₂₁N₇O₃ as (M+H)⁺ 408.4. λ=248.7, 300.9.

### Example 42

### Preparation of 2-(cyclopropylamino)-6-oxo-4-(4-sulfamoylphenylamino)-1,6-dihydropyrimidine-5-carboxamide

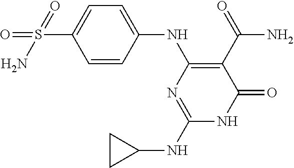 [SMILES: NC(=O)c1c(Nc2ccc(S(N)(=O)=O)cc2)nc(NC2CC2)[nH]c1=O]

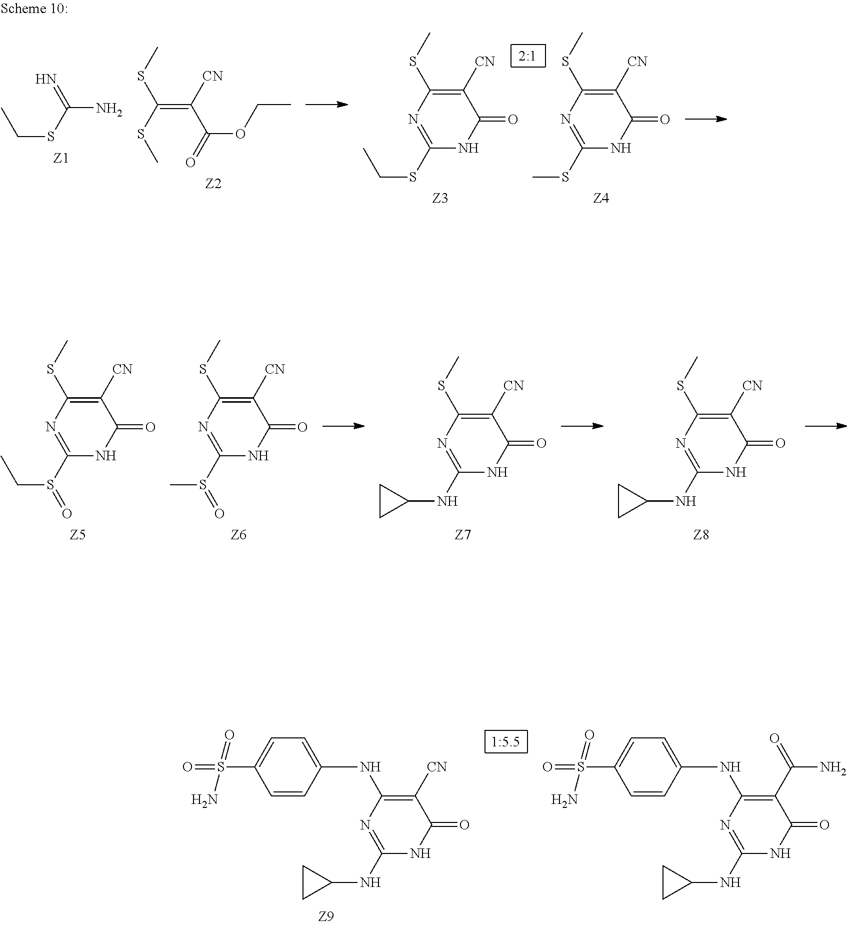

Step 1: The mixture of S-ethylisothiourea hydrobromide (Z1, 4.26 g, 23 mmol), ethyl 3,3-bis(methylthio)-2-cyanoacrylate (Z2, 5.00 g, 23 mmol), triethylamine (6.4 mL, 46 mmol) in 100 ml, acetonitrile was gently refluxed for overnight (18 h) to yield a mixture of compounds Z3 and Z4 in 2:1 ratio in >90% yield. The mixture was concentrated in vacuo and dissolved in 300 mL chloroform. It was washed with brine three times, dried, concentrated in vacuo to afford a tan-colored solid.

Step 2: The above-prepared mixture of Z3 and Z4 was dissolved in 200 mL THF. To it was added MCPBA (77% strength, 9.86 g, 44 mmol) in small portions at RT. The mixture was stirred for 2 h, and to it was poured 200 mL water. It was concentrated in vacuo at cold to remove THF. The aq. mixture was then washed with DCM three times. The aq phase was concentrated in vacuo to dryness. The solid was triturated with THF multiple times. All the THF solution was combined and concentrated in vacuo to afford a mixture of compound Z5 and Z6.

Step 3: The above prepared mixture of Z5 and Z6 (75 mg) was dissolved in 3 mL DMSO in a sealed tube. To it was added cyclopropylamine (0.15 mL). The mixture in the sealed tube was stirred at 85° C. for 3 h. The mixture was cooled to RT, diluted with water and directly subjected to revered phase HPLC to isolate the major product Z7.

Step 4: The above-prepared Z7 (20 mg, 0.084 mmol) was dissolved in 1 mL DMSO in a sealed tube. To it were added sulfanilamide (43 mg, 0.25 mmol) and PTSA (14 mg, 0.08 mmol). The mixture in the sealed tube was stirred at 120° C. for 2 h. It was an incomplete reaction with about 40% was still present. Two products (Z9 and the title compound) were found in the ratio of 1:5.5. The reaction was stopped and directly subjected to reverse phase prep HPLC to isolate the title compound. UV: 282, 310 nm. M+H found for C₁₄H₁₆N₆O₄S: 365.3. NMR (DMSO-d₆): 13.18 (1H, s), 10.54 (1H, bs), 9.09 (1H, s), 7.82 (2H, bs), 7.64 (2H, d, J=8.4 Hz), 7.14 (4H, bs), 2.66 (1H, m), 0.75 (2H, m), 0.54 (2H, m) ppm.

### Example 43

### Preparation of 2-(cyclopropylamino)-4-(4-(N-methylacetamido)phenylamino)-6-oxo-1,6-dihydropyrimidine-5-carboxamide

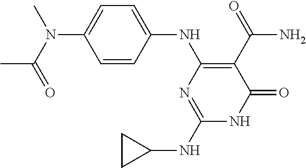 [SMILES: CC(=O)N(C)c1ccc(Nc2nc(NC3CC3)[nH]c(=O)c2C(N)=O)cc1]

The title compound was prepared using the same chemistry shown in Example 42. UV: 262, 303 nm. M+H found for C₁₇H₂₀N₆O₃: 357.3. NMR (CD₃OD): 7.75 (2H, m), 7.10 (2H, d, J=9.2 Hz), 3.12 (3H, s), 2.44 (1H, m), 1.76 (3H, s), 0.79 (2H, m), 0.55 (2H, m) ppm.

### Example 44

### Preparation of 4-(cyclopropylamino)-6-oxo-2-(4-sulfamoylphenylamino)-1,6-dihydropyrimidine-5-carboxamide

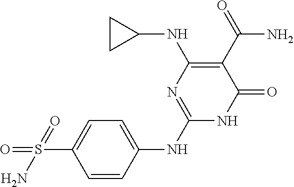 [SMILES: NC(=O)c1c(NC2CC2)nc(Nc2ccc(S(N)(=O)=O)cc2)[nH]c1=O]

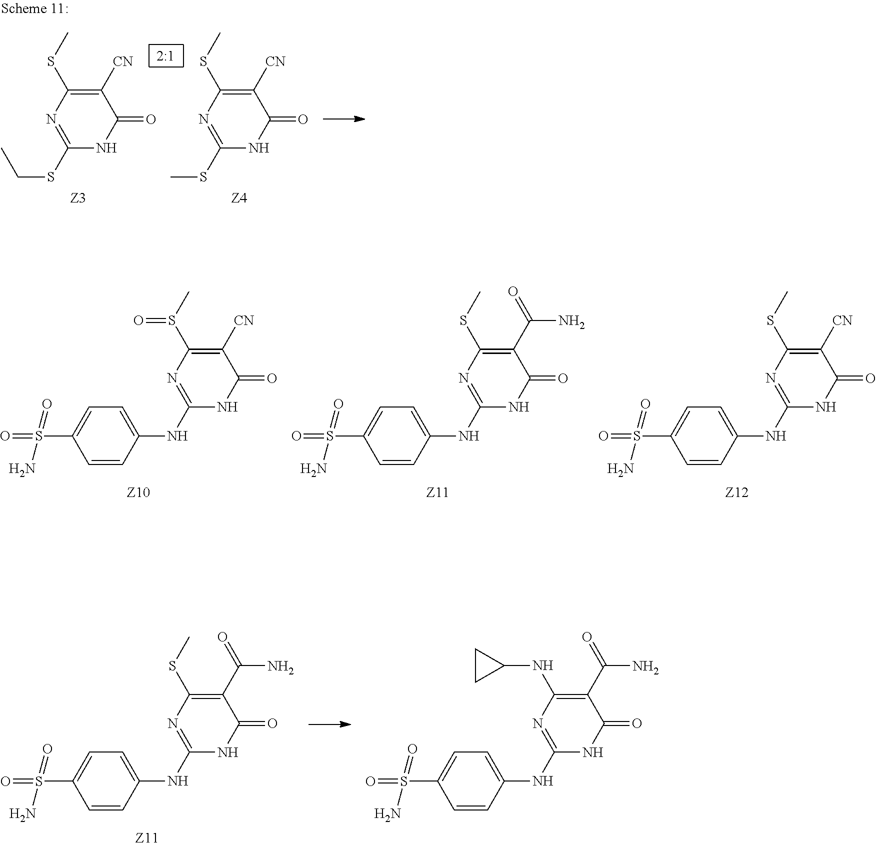

Step 1: The 2:1 mixture of compounds Z3 and Z4 (0.57 g, 2.6 mmol) was dissolved in 40 mL dioxane in a sealed tube. To it was added MCPBA (65% strength, 1.37 g, 5.2 mmol) in small portions. The mixture was stirred at RT for 30 min. Then to it was added sulfanilamide (0.90 g, 5.2 mmol). The mixture in the sealed tube was stirred at 100° C. for 1 h. Three compounds (Z10, Z11 and Z12 (major)) were found in the mixture. The mixture was subjected to reverse phase HPLC to isolate these three compounds.

Step 2: Compound Z11 (25 mg, 0.07 mmol) was dissolved in 3 mL NMP in a sealed tube. To it was added MCPBA (65% strength, 37 mg, 0.14 mmol). The mixture was stirred at RT for 20 min. To it was then added cyclopropylamine (1.0 mL). The mixture in the sealed tube was stirred at 80° C. for 1 h. The mixture was concentrated in vacuo and subjected to reverse phase HPLC to isolate the title compound. UV: 300 nm. M+H found for C₁₄H₁₆N₆O₄S: 365.3.

### Example 45

### Preparation of 4-(cyclobutylamino)-6-oxo-2-(4-sulfamoylphenylamino)-1,6-dihydropyrimidine-5-carboxamide

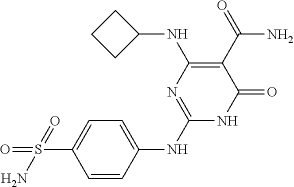 [SMILES: NC(=O)c1c(NC2CCC2)nc(Nc2ccc(S(N)(=O)=O)cc2)[nH]c1=O]

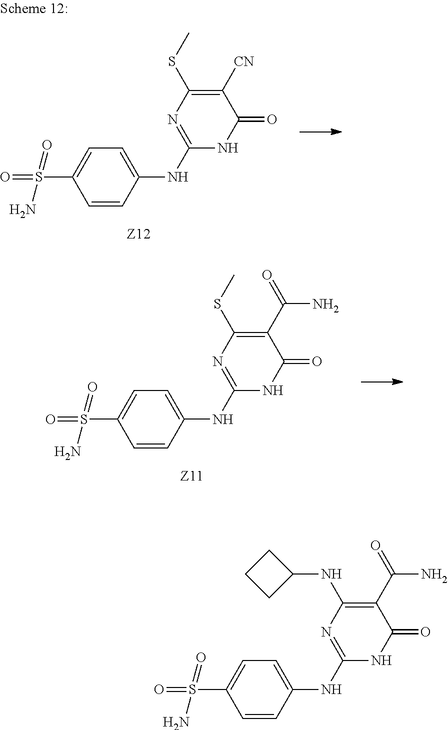 [SMILES: CSc1nc(Nc2ccc(S(N)(=O)=O)cc2)[nH]c(=O)c1C#N ; CSc1nc(Nc2ccc(S(N)(=O)=O)cc2)[nH]c(=O)c1C(N)=O ; NC(=O)c1c(NC2CCC2)nc(Nc2ccc(S(N)(=O)=O)cc2)[nH]c1=O]

Step 1: Compound Z12 (100 mg) was treated with 5 mL TFA and 1.2 mL concentrated H₂SO₄ at 80° C. for 1 h. The mixture was cooled to RT. To it was added 5 mL water. The mixture was directly subjected to reverse phase HPLC to isolate compound Z11.

Step 2: Compound Z11 (12 mg, 0.034 mmol) was dissolved in 3 mL NMP in a sealed tube. To it was added MCPBA (65% strength, 18 mg, 0.068 mmol). The mixture was stirred at RT for 15 min. To it was then added cyclobutylamine (0.5 mL). The mixture in the sealed tube was stirred at 80° C. for 40 min. The mixture was concentrated in vacuo and subjected to reverse phase HPLC to isolate the title compound. UV: 299 nm. M+H found for C₁₅H₁₈N₆O₄S: 379.3.

### Example 46

### Preparation of 2,4-bis(4-(4-acetylpiperazin-1-yl)phenylamino)-6-oxo-1,6-dihydropyrimidine-5-carboxamide

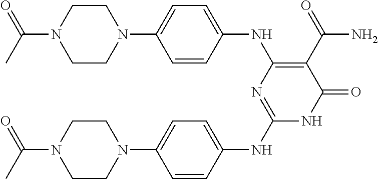 [SMILES: CC(=O)N1CCN(c2ccc(Nc3nc(Nc4ccc(N5CCN(C(C)=O)CC5)cc4)c(C(N)=O)c(=O)[nH]3)cc2)CC1]

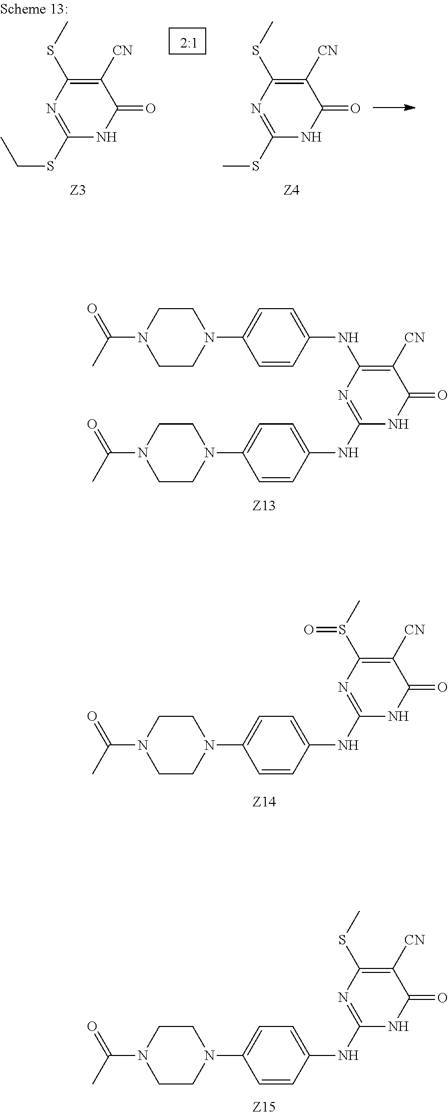 [SMILES: CCSc1nc(SC)c(C#N)c(=O)[nH]1 ; CSc1nc(SC)c(C#N)c(=O)[nH]1 ; CC(=O)N1CCN(c2ccc(Nc3nc(Nc4ccc(N5CCN(C(C)=O)CC5)cc4)c(C#N)c(=O)[nH]3)cc2)CC1 ; CC(=O)N1CCN(c2ccc(Nc3nc(S(C)=O)c(C#N)c(=O)[nH]3)cc2)CC1 ; CSc1nc(Nc2ccc(N3CCN(C(C)=O)CC3)cc2)[nH]c(=O)c1C#N]

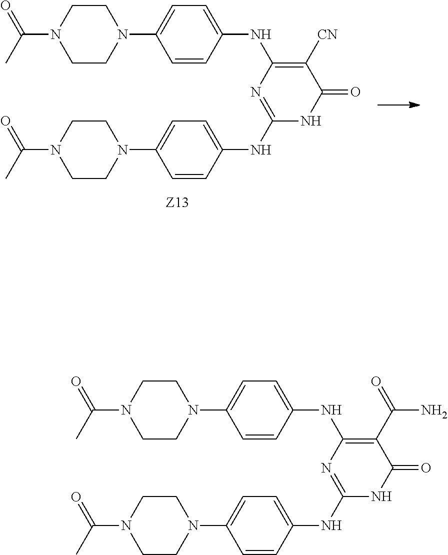 [SMILES: CC(=O)N1CCN(c2ccc(Nc3nc(Nc4ccc(N5CCN(C(C)=O)CC5)cc4)c(C#N)c(=O)[nH]3)cc2)CC1 ; CC(=O)N1CCN(c2ccc(Nc3nc(Nc4ccc(N5CCN(C(C)=O)CC5)cc4)c(C(N)=O)c(=O)[nH]3)cc2)CC1]

Step 1: The 2:1 mixture of compounds Z3 and Z4 (0.29 g, 1.3 mmol) was dissolved in 30 mL dioxane in a sealed tube. To it was added MCPBA (65% strength, 0.69 g, 2.6 mmol) in small portions. The mixture was stirred at RT for 30 min. Then to it was added 1-(4-(4-aminophenyl)piperazin-1-yl)ethanone (0.57 g, 2.6 mmol). The mixture in the sealed tube was stirred at 100° C. for 1.5 h. Three compounds (Z13, Z14 and Z15 (major)) were found in the mixture. The mixture was subjected to reverse phase HPLC to isolate these three compounds.

Step 2: Compound Z13 (70 mg) was treated with 6 mL TFA and 2 mL concentrated H₂SO₄ at 80° C. for 90 min. The mixture was cooled to RT. To it was added 5 mL water. The mixture was directly subjected to reverse phase HPLC to isolate the title compound. UV: 249, 302 nm. M+H found for C₂₉H₃₅N₉O₄: 574.3.

### Example 47

### Preparation of 2-(4-(4-acetylpiperazin-1-yl)phenylamino)-4-(cyclobutylamino)-6-oxo-1,6-dihydropyrimidine-5-carboxamide

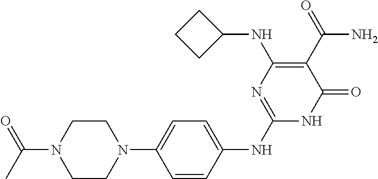 [SMILES: CC(=O)N1CCN(c2ccc(Nc3nc(NC4CCC4)c(C(N)=O)c(=O)[nH]3)cc2)CC1]

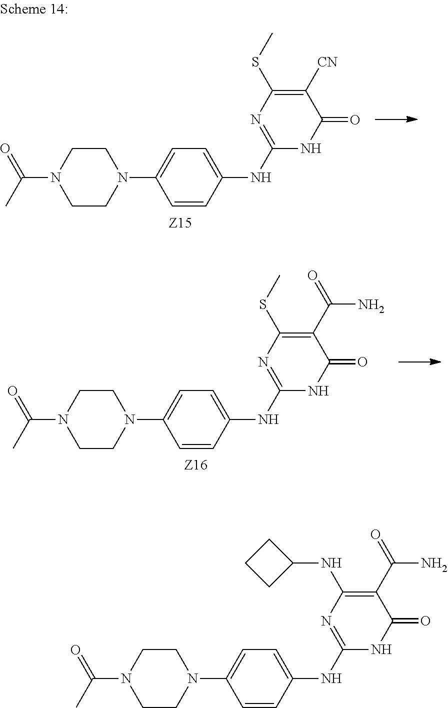 [SMILES: CSc1nc(Nc2ccc(N3CCN(C(C)=O)CC3)cc2)[nH]c(=O)c1C#N ; CSc1nc(Nc2ccc(N3CCN(C(C)=O)CC3)cc2)[nH]c(=O)c1C(N)=O ; CC(=O)N1CCN(c2ccc(Nc3nc(NC4CCC4)c(C(N)=O)c(=O)[nH]3)cc2)CC1]

Step 1: Compound Z15 (190 mg) was treated with 5 mL TFA and 1.2 mL concentrated H₂SO₄ at 85° C. for 1 h. The mixture was cooled to RT. To it was added 5 mL water. The mixture was directly subjected to reverse phase HPLC to isolate compound Z16.

Step 2: Compound Z16 (30 mg, 0.075 mmol) was dissolved in 3 mL NMP in a sealed tube. To it was added MCPBA (65% strength, 40 mg, 0.15 mmol). The mixture was stirred at RT for 15 min. To it was then added cyclobutylamine (0.5 mL). The mixture in the sealed tube was stirred at 80° C. for 40 min. The mixture was concentrated in vacuo and subjected to reverse phase HPLC to isolate the title compound. UV: 295 nm. M+H found for C₂₁H₂₇N₇O₃: 426.2.

### Example 48

### 2-(2-aminocyclohexylamino)-4-(cyclopropylamino)-6-hydroxypyrimidine-5-carboxamide

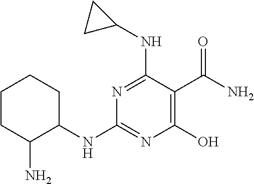 [SMILES: NC(=O)c1c(O)nc(NC2CCCCC2N)nc1NC1CC1]

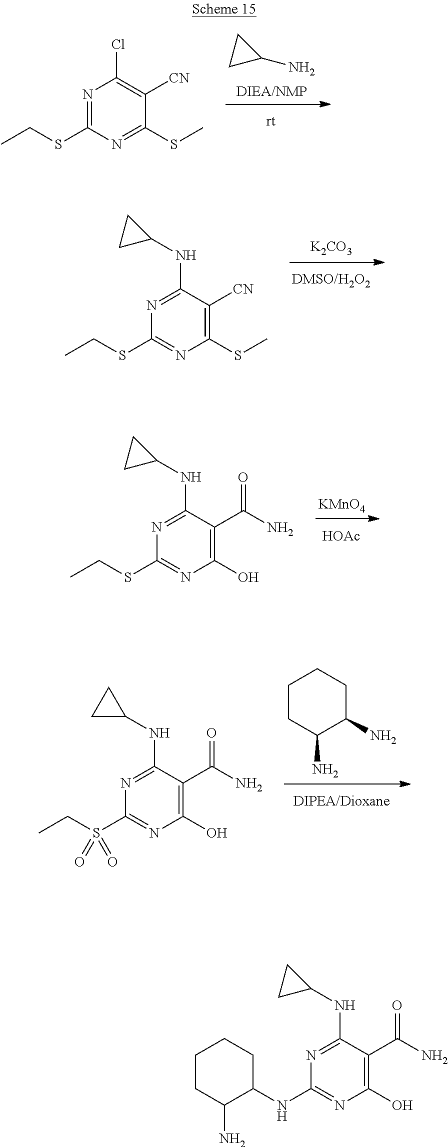

Step 1: To a solution of 4-chloro-2-(ethylthio)-6-(methylthio)pyrimidine-5-carbonitrile (2.45 g, 10 mmol) and DIEA (10 mmol) in NMP (25 mL) was added cyclopropanamine (20 mmol). Stirring at rt for 30 min, the reaction solution was diluted with EtOAc, washed with 1N HCl, Sat NaHCO₃, brine, dried and concentrated to give 2-(2-amino-2-oxoethylamino)-4-(cyclopropylamino)-6-hydroxypyrimidine-5-carboxamide (2.32 g) ready for the next step.

Step 2: To a solution of 2-(2-amino-2-oxoethylamino)-4-(cyclopropylamino)-6-hydroxypyrimidine-5-carboxamide (1.61 g, 6.1 mmol) in DMSO (100 mL) was added H₂O₂ (50%, 40 mL) and K₂CO₃ (200 mg, 1.44 mmol). Stirring for at 70° C. for 5 h, the reaction solution was diluted with EtOAc, washed with 1N HCl, Sat NaHCO₃, brine, dried and concentrated to give 4-(cyclopropylamino)-2-(ethylthio)-6-hydroxypyrimidine-5-carboxamide.

Step 3: A mixture of 4-(cyclopropylamino)-2-(ethylthio)-6-hydroxypyrimidine-5-carboxamide (20 mg) and KMnO4 (24 mg) in HOAc (2 mL) was stirred at rt for 2 h.

The reaction solution was diluted with EtOAc, washed with Sat NaHCO₃, brine, dried and concentrated to give 4-(cyclopropylamino)-2-(ethylsulfonyl)-6-hydroxypyrimidine-5-carboxamide.

Step 4: A mixture of 4-(cyclopropylamino)-2-(ethylsulfonyl)-6-hydroxypyrimidine-5-carboxamide (14 mg), glycinamide hydrochloride (33 mg) and DIEA (100 uL) Dioxane (3 mL) was heated at 150° C. under microwave for 10 min. the product 2-(2-aminocyclohexylamino)-4-(cyclopropylamino)-6-hydroxypyrimidine-5-carboxamide was purified with HPLC. MS found for C₁₄H₂₂N₆O₂ as (M+H)⁺ 307.2. λ=237.9, 279.2.

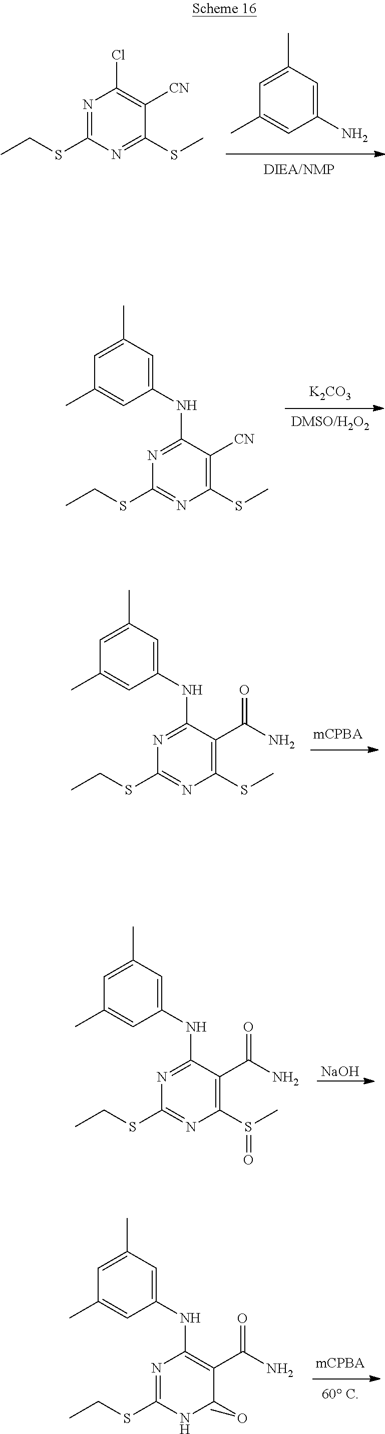

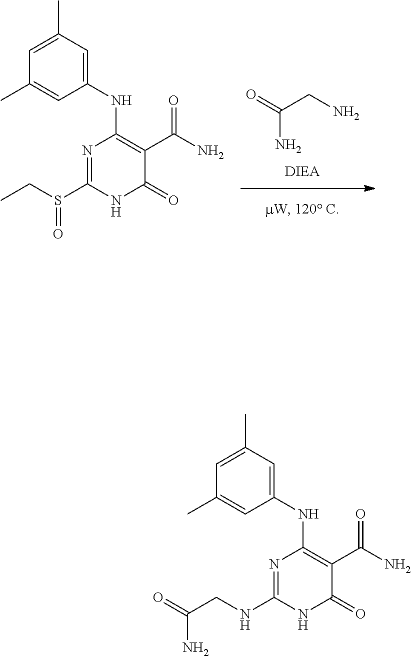 [SMILES: CCS(=O)c1nc(Nc2cc(C)cc(C)c2)c(C(N)=O)c(=O)[nH]1 ; NCC(N)=O ; Cc1cc(C)cc(Nc2nc(NCC(N)=O)[nH]c(=O)c2C(N)=O)c1]

### Example 49

### 2-(2-amino-2-oxoethylamino)-4-(3,5-dimethylphenylamino)-6-oxo-1,6-dihydropyrimidine-5-carboxamide

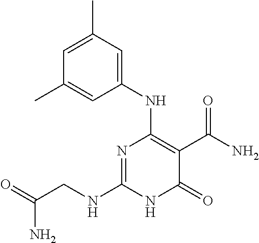 [SMILES: Cc1cc(C)cc(Nc2nc(NCC(N)=O)[nH]c(=O)c2C(N)=O)c1]

Step 1: To a solution of 4-chloro-2-(ethylthio)-6-(methylthio)pyrimidine-5-carbonitrile (3.45 g, 14 mmol) and DIEA (28 mmol) in NMP (80 mL) was added 3,5-dimethylaniline (28 mmol). Stirring at r.t. for 2 h, the reaction solution was diluted with EtOAc, washed with 1N HCl, Sat NaHCO₃, brine, dried and concentrated to give 4-(3,5-dimethylphenylamino)-2-(ethylthio)-6-(methylthio)pyrimidine-5-carbonitrile (3.76 g) ready for the next step.

Step 2: To a solution of 4-(3,5-dimethylphenylamino)-2-(ethylthio)-6-(methylthio)pyrimidine-5-carbonitrile (1.65 g, 5.0 mmol) in DMSO/THF (80/80 mL) was added H₂O₂ (50%, 32 mL) and K₂CO₃ (138 mg, 1 mmol). Stirring for at 70° C. for 2 h, the reaction solution was diluted with EtOAc, washed with 1N HCl, Sat NaHCO₃, brine, dried and concentrated to give 4-(3,5-dimethylphenylamino)-2-(ethylthio)-6-(methylthio)pyrimidine-5-carboxamide (1.63 g).

Step 3: A mixture of 4-(3,5-dimethylphenylamino)-2-(ethylthio)-6-(methylthio)pyrimidine-5-carboxamide (1.63 g) and mCPBA (77%, 1.15 g) in THF (110 mL) was stirred at 0° C. for l h and then at r.t. overnight. The reaction solution was concentrated and then extracted with EtOAc, washed with Sat NaHCO₃, brine, dried and concentrated to give 4-(3,5-dimethylphenylamino)-2-(ethylthio)-6-(methylsulfinyl)pyrimidine-5-carboxamide.

Step 4: A mixture of 4-(3,5-dimethylphenylamino)-2-(ethylthio)-6-(methylsulfinyl)pyrimidine-5-carboxamide from step 3 was dissolved in THF (100 mL) was treated with 2N NaOH (15 mL) at 60° C. for 90 minutes. The reaction solution was concentrated and then extracted with EtOAc, washed with 1N HCl, brine. After concentration and drying, 4-(3,5-dimethylphenylamino)-2-(ethylthio)-6-methoxy-1,6-dihydropyrimidine-5-carboxamide was obtained.

Step 5: A mixture of 4-(3,5-dimethylphenylamino)-2-(ethylthio)-6-methoxy-1,6-dihydropyrimidine-5-carboxamide (439 m g) and mCPBA (77%, 1.6 g) in THF (100 mL) was stirred at r.t. overnight. The reaction solution was concentrated and then extracted with EtOAc, washed with Sat NaHCO₃, brine, dried and concentrated to 4-(3,5-dimethylphenylamino)-2-(ethylsulfinyl)-6-oxo-1,6-dihydropyrimidine-5-carboxamide.

Step 6: A mixture of 4-(3,5-dimethylphenylamino)-2-(ethylsulfinyl)-6-oxo-1,6-dihydropyrimidine-5-carboxamide (35 mg), glycinamide hydrochloride (55 mg) and DIEA (110 uL) Dioxane (3 mL) was heated at 150° C. under microwave for 25 min. the product 2-(2-amino-2-oxoethylamino)-4-(3,5-dimethylphenylamino)-6-oxo-1,6-dihydropyrimidine-5-carboxamide2-(2-amino-2-oxoethylamino)-4-(3,5-dimethylphenylamino)-6-oxo-1,6-dihydropyrimidine-5-carboxamide was purified with HPLC. MS found for C₁₅H₁₈N₆O₃ as (M+H)⁺ 331.0, λ=257.9, 301.6.

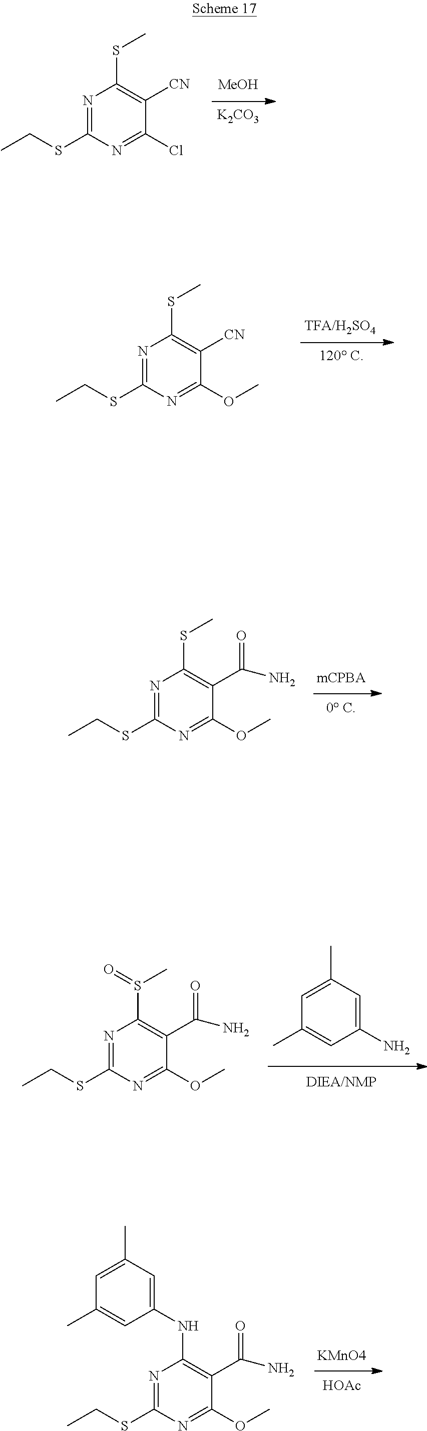

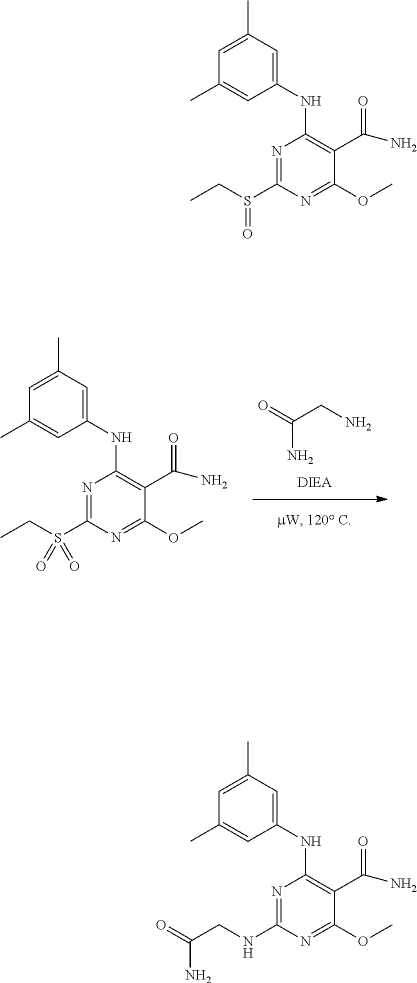 [SMILES: CCS(=O)c1nc(Nc2cc(C)cc(C)c2)c(C(N)=O)c(OC)n1 ; CCS(=O)(=O)c1nc(Nc2cc(C)cc(C)c2)c(C(N)=O)c(OC)n1 ; NCC(N)=O ; COc1nc(NCC(N)=O)nc(Nc2cc(C)cc(C)c2)c1C(N)=O]

### Example 50

### 2-(2-amino-2-oxoethylamino)-4-(3,5-dimethylphenylamino)-6-methoxypyrimidine-5-carboxamide

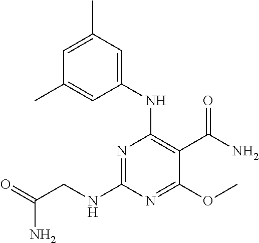 [SMILES: COc1nc(NCC(N)=O)nc(Nc2cc(C)cc(C)c2)c1C(N)=O]

Step 1: To a solution of 4-chloro-2-(ethylthio)-6-(methylthio)pyrimidine-5-carbonitrile (1.17 g, 4.78 mmol) in MeOH (50 mL) was added K₂CO₃ (10 mmol). Stirring at r.t. for 1 h, the reaction solution was diluted with EtOAc, washed with 1N HCl, brine, dried and concentrated to give 2-(ethylthio)-4-methoxy-6-(methylthio)pyrimidine-5-carbonitrile (1.086 g).

Step 2: 2-(ethylthio)-4-methoxy-6-(methylthio)pyrimidine-5-carbonitrile (735 mg, 3 mmol) was treated with TFA/H₂SO₄ (15/5 mL) at 85° C. for 3 h. The solution was concentrated and poured to ice water. The precipitate was collected.

Step 3: To 2-(ethylthio)-4-methoxy-6-(methylthio)pyrimidine-5-carboxamide (3 mmol) in THF (60 mL), was added mCPBA (77%, 3.6 mmol) at 0° C. The reaction solution was stirred at 0° C. for 2 h and then concentrated for the next step.

Step 4: To 2-(ethylthio)-4-methoxy-6-(methylsulfinyl)pyrimidine-5-carboxamide (3 mmol) in NMP (60 mL), was added 3,5-dimethylaniline (15 mmol) and DIEA (15 mmol). The mixture was stirred at 100° C. for 2 h. EtOAc and water was added. The organic layer was washed with aqueous citric acid and brine. After concentrated and dried, the product 4-(3,5-dimethylphenylamino)-2-(ethylthio)-6-methoxypyrimidine-5-carboxamide was obtained.

Step 5: To 4-(3,5-dimethylphenylamino)-2-(ethylthio)-6-methoxypyrimidine-5-carboxamide (0.5 mmol) in HOAc (12 mL), was added KMnO₄ (1 mmol). After extraction with EtOAc, the product mixture of 4-(3,5-dimethylphenylamino)-2-(ethylsulfinyl)-6-methoxypyrimidine-5-carboxamide and 4-(3,5-dimethylphenylamino)-2-(ethylsulfonyl)-6-methoxypyrimidine-5-carboxamide were obtained.

Step 6: To the mixture of 4-(3,5-dimethylphenylamino)-2-(ethylsulfinyl)-6-methoxypyrimidine-5-carboxamide and 4-(3,5-dimethylphenylamino)-2-(ethylsulfonyl)-6-methoxypyrimidine-5-carboxamide (0.5 mmol)), 2-aminoacetamide hydrochloride (2 mmol)) and DIEA (2 mmol) Dioxane (2 mL) was heated at 150° C. under microwave for 25 min. the product 2-(2-amino-2-oxo ethylamino)-4-(3,5-dimethylphenylamino)-6-methoxypyrimidine-5-carboxamide was purified with HPLC. MS found for C16H20N6O3 as (M+H)⁺ 345.2. λ=246.1.

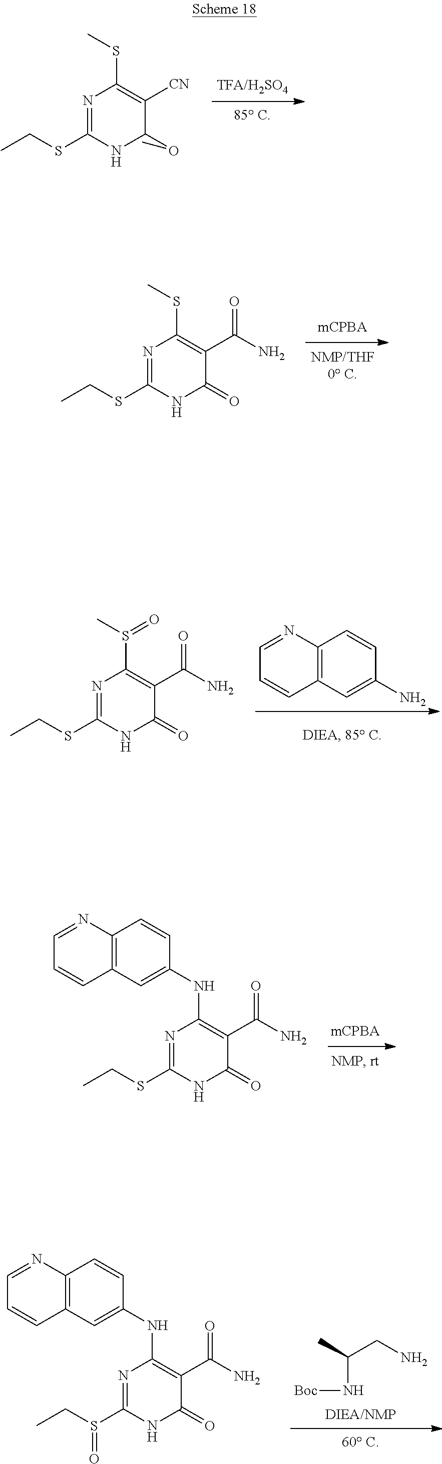

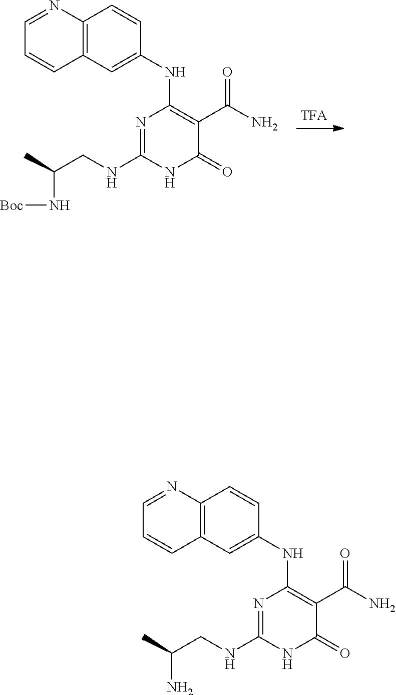 [SMILES: C[C@@H](CNc1nc(Nc2ccc3ncccc3c2)c(C(N)=O)c(=O)[nH]1)NC(=O)OC(C)(C)C ; C[C@H](N)CNc1nc(Nc2ccc3ncccc3c2)c(C(N)=O)c(=O)[nH]1]

### Example 51

### (S)-2-(2-aminopropylamino)-6-oxo-4-(quinolin-6-ylamino)-1,6-dihydropyrimidine-5-carboxamide

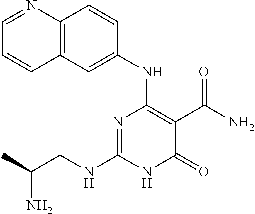 [SMILES: C[C@H](N)CNc1nc(Nc2ccc3ncccc3c2)c(C(N)=O)c(=O)[nH]1]

Step 1: 2-(ethylthio)-6-methoxy-4-(methylthio)-1,6-dihydropyrimidine-5-carbonitrile (3.4 g, 15 mmol) was treated with TFA/H₂SO₄ (75/25 mL) at 85° C. for 4 h. The solution was concentrated and poured to ice water. The precipitate was collected and dried. 2-(ethylthio)-4-(methylthio)-6-oxo-1,6-dihydropyrimidine-5-carboxamide (3.25 g) was obtained.

Step 2: To 2-(ethylthio)-4-(methylthio)-6-oxo-1,6-dihydropyrimidine-5-carboxamide (255 mg, 1.0 mmol) in NMP/THF (15/3 mL), was added mCPBA (65%, 1.2 mmol) at 0° C. The reaction solution was stirred at 0° C. for 2 h and then concentrated for the next step.

Step 3: To 2-(ethylthio)-4-(methylsulfinyl)-6-oxo-1,6-dihydropyrimidine-5-carboxamide (1 mmol), quinolin-6-amine was added 3,5-dimethylaniline (6 mmol) and DIEA (6 mmol). The mixture was stirred at 70° C. for 20 h. EtOAc and water was added. The precipitate was collected and dried, the product 2-(ethylthio)-6-oxo-4-(quinolin-6-ylamino)-1,6-dihydropyrimidine-5-carboxamide was obtained.

Step 4: To 2-(ethylthio)-6-oxo-4-(quinolin-6-ylamino)-1,6-dihydropyrimidine-5-carboxamide (0.1 mmol) in NMP (2 mL), was added mCPBA (65%, 0.1 mmol) at rt. The reaction solution was stirred at rt for 3 h and then used for the next step.

Step 5: To the above solution, was added (5)-tert-butyl 1-aminopropan-2-ylcarbamate (0.2 mmol) and DIEA (0.5 mmol). The reaction solution was stirred at 70° C. for 15 h and then diluted with ethyl acetate and water. The organic layers were washed with citric acid aq. and brine. After concentrated and dried, treatment with TFA/DCM (1/3) gave desired product (S)-2-(2-aminopropylamino)-6-oxo-4-(quinolin-6-ylamino)-1,6-dihydropyrimidine-5-carboxamide. MS found for C17H19N7O2 as (M+H)⁺ 354.3, λ=266.8, 292.9.

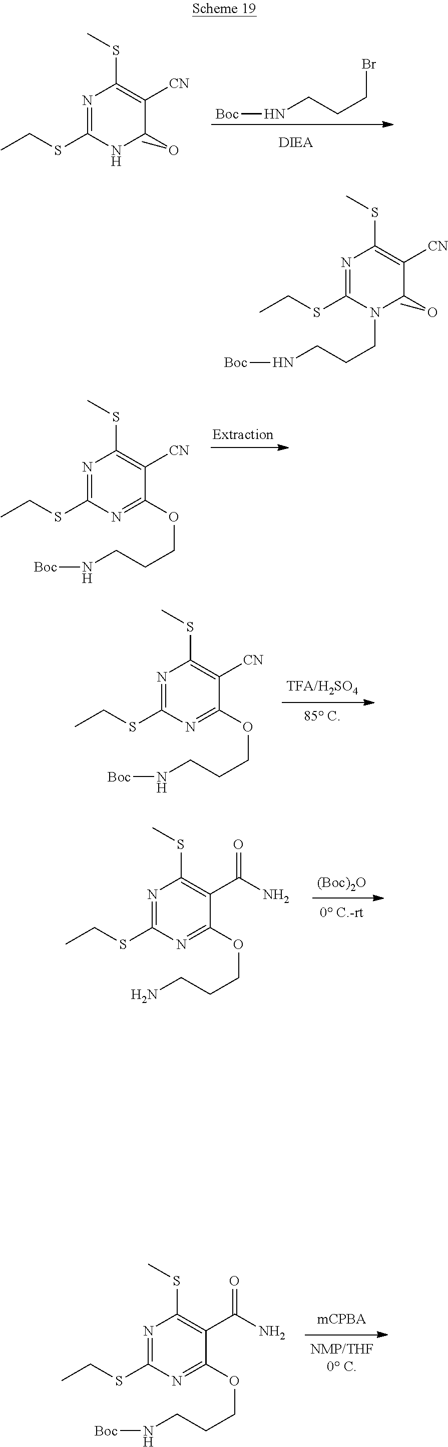

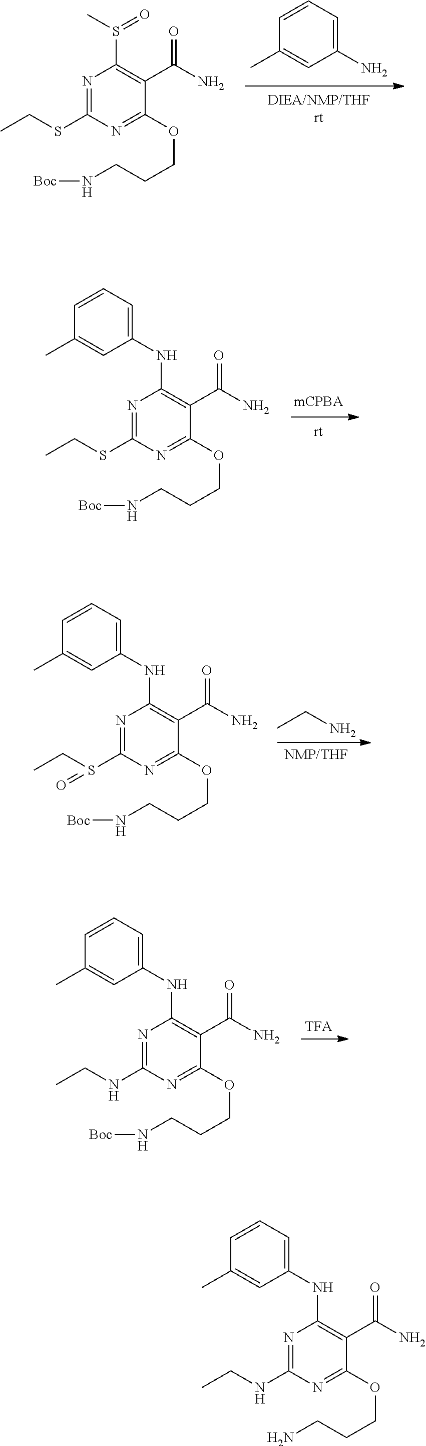

### Example 52

### 4-(m-toluidino)-6-(3-aminopropoxy)-2-(ethylamino)pyrimidine-5-carboxamide

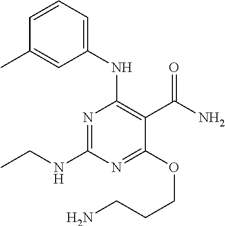 [SMILES: CCNc1nc(Nc2cccc(C)c2)c(C(N)=O)c(OCCCN)n1]

Step 1: A mixture of 2-(ethylthio)-6-methoxy-4-(methylthio)-1,6-dihydropyrimidine-S-carbonitrile (11.1 mmol), DIEA(28 mmol) and tert-butyl 3-bromopropylcarbamate (22.2 mmol) in DMF (20 mL) was stirred at 45° C. for 2 days. Extraction with hexane gave tert-butyl 3-(5-cyano-2-(ethylthio)-6-(methylthio)pyrimidin-4-yloxy)propylcarbamate.

Step 2: Tert-butyl 3-(5-cyano-2-(ethylthio)-6-(methylthio)pyrimidin-4-yloxy)propylcarbamate (10 mmol) was treated with TFA/H₂SO₄ (50/17 mL) at 85° C. for 2 h. The solution was concentrated and poured to ice water. Extraction with ButOH gave 4-(3-aminopropoxy)-2-(ethylthio)-6-(methylthio)pyrimidine-5-carboxamide.

Step 3: To 4-(3-aminopropoxy)-2-(ethylthio)-6-(methylthio)pyrimidine-5-carboxamide (˜10 mmol) in ButOH, was added (Boc)2O (9 mmol) at 0° C. After stirred at rt for 20 minutes, washed with brine and concentrated, tert-butyl 3-(5-carbamoyl-2-(ethylthio)-6-(methylthio)pyrimidin-4-yloxy)propylcarbamate was obtained.

Step 4: To tert-butyl 3-(5-carbamoyl-2-(ethylthio)-6-(methylthio)pyrimidin-4-yloxy)propylcarbamate (3 mmol) in NMP/THF (50/10 mL), was added mCPBA (3 mmol) at 0° C. and then stirred for 8 h.

Step 5: To the above solution, was added m-toluidine (18 mmol) and DIEA (18 mmol). After stirred at rt over night, extraction with ethyl acetate gave tert-butyl 3-(6-(m-toluidino)-5-carbamoyl-2-(ethylthio)pyrimidin-4-yloxy)propylcarbamate as brown oil (900 mg).

Step 6: To tert-butyl 3-(6-(m-toluidino)-5-carbamoyl-2-(ethylthio)pyrimidin-4-yloxy)propylcarbamate (2 mmol) in NMP (25 mL), was added mCPBA (65%, 6.6 mmol) at rt. The reaction solution was stirred at rt for 3 h and then used for the next step.

Step 7: To the above solution (0.6 mmol), was added ethylamine solution (2M in THF, 10 mmol). After stirred at rt over night, concentration, extraction with ethyl acetate and treated with TFA/DCM gave 4-(m-toluidino)-6-(3-aminopropoxy)-2-(ethylamino)pyrimidine-5-carboxamide. MS found for C17H24N6O2 as (M+H)⁺ 345.2, λ=265, 292.

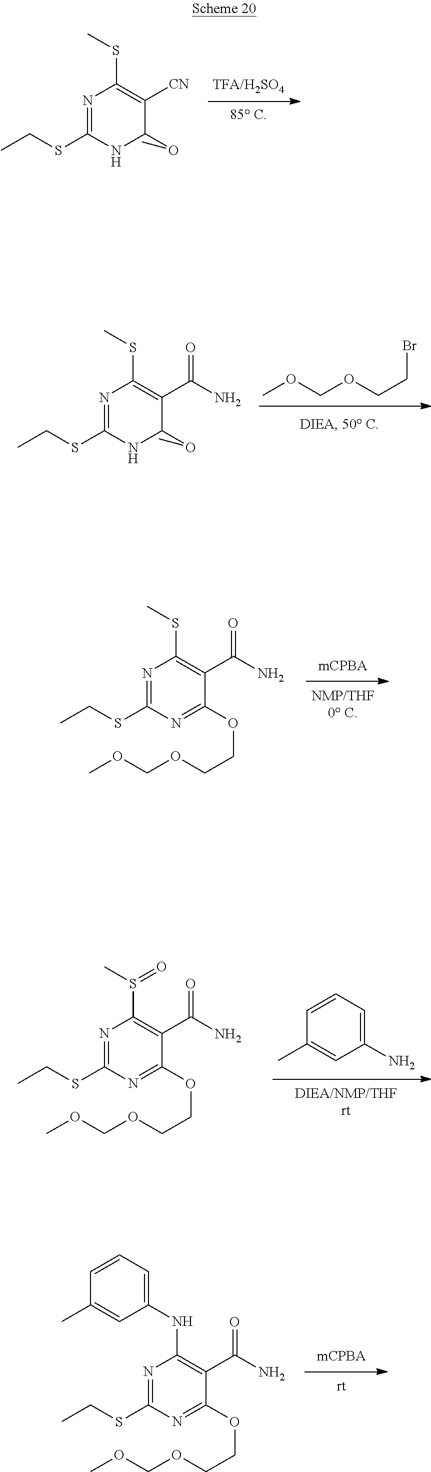

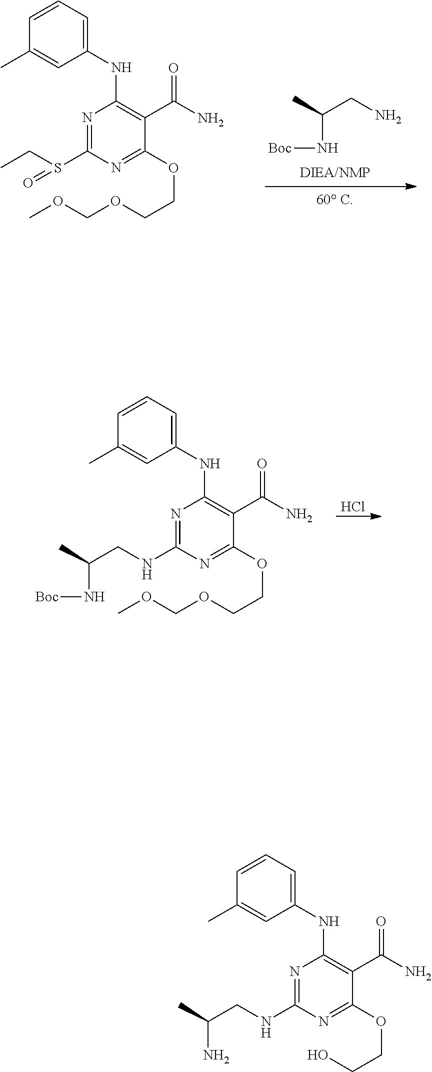 [SMILES: CCS(=O)c1nc(Nc2cccc(C)c2)c(C(N)=O)c(OCCOCOC)n1 ; C[C@@H](CN)NC(=O)OC(C)(C)C ; COCOCCOc1nc(NC[C@H](C)NC(=O)OC(C)(C)C)nc(Nc2cccc(C)c2)c1C(N)=O ; Cl ; Cc1cccc(Nc2nc(NC[C@H](C)N)nc(OCCO)c2C(N)=O)c1]

### Example 53

### (S)-4-(m-toluidino)-2-(2-aminopropylamino)-6-(2-hydroxyethoxy)pyrimidine-5-carboxamide

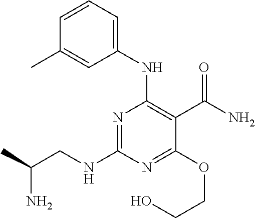 [SMILES: Cc1cccc(Nc2nc(NC[C@H](C)N)nc(OCCO)c2C(N)=O)c1]

Step 1: 2-(ethylthio)-4-(methylthio)-6-oxo-1,6-dihydropyrimidine-5-carbonitrile (15 mmol) was treated with TFA/H₂SO₄ (75/25 mL) at 85° C. for 4 h. The solution was concentrated and poured to ice water and DCM. The precipitate 2-(ethylthio)-4-(methylthio)-6-oxo-1,6-dihydropyrimidine-5-carboxamide was collected and dried for the next step.

Step 2: To 2-(ethylthio)-4-(methylthio)-6-oxo-1,6-dihydropyrimidine-5-carboxamide (2 mmol) in DMF (20 mL), were added 1-bromo-2-(methoxymethoxy)ethane (5 mmol) and DIEA (8 mmol). After stirred at 50° C. for 36 h, the reaction solution was diluted with ethyl acetate and water. The organic layer was concentrate and then recrystallized from ethyl acetate. The product 2-(ethylthio)-4-(2-(methoxymethoxy)ethoxy)-6-(methylthio)pyrimidine-5-carboxamide was obtained.

Step 3: 2-(ethylthio)-4-(2-(methoxymethoxy)ethoxy)-6-(methylthio)pyrimidine-5-carboxamide (1 mmol) in NMP/THF (10/3 mL), was added mCPBA (1.2 mmol) at 0° C. for 2 h.

Step 4: To the above solution, was added m-toluidine (6 mmol) and DIEA (6 mmol). After stirred at rt over night, extraction with ethyl acetate gave 4-(m-toluidino)-2-(ethylthio)-6-(2-(methoxymethoxy)ethoxy)pyrimidine-5-carboxamide.

Step 5: To 4-(m-toluidino)-2-(ethylthio)-6-(2-(methoxymethoxy)ethoxy)pyrimidine-5-carboxamide (1 mmol) in NMP (10 mL), was added mCPBA (65%, 2.2 mmol) at rt. The reaction solution was stirred at rt for 12 h and then used for the next step.

Step 7: To the above solution (0.3 mmol), was added (5)-tert-butyl 1-aminopropan-2-ylcarbamate (0.5 mmol) and DIEA (2 mmol). After stirred at 60° C. for 16 h, concentration, extraction with ethyl acetate and treated with HCl in dioxane gave (S)-4-(m-toluidino)-2-(2-aminopropylamino)-6-(2-hydroxyethoxy)pyrimidine-5-carboxamide. MS found for C17H24N6O3 as (M+H)⁺ 361.3, λ=260.9.

### Example 54

### (S)-2-(2-aminopropylamino)-4-(3,5-dimethylphenylamino)-6-oxo-1,6-dihydropyrimidine-5-carboxamide

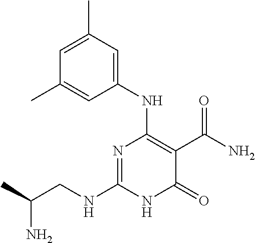 [SMILES: Cc1cc(C)cc(Nc2nc(NC[C@H](C)N)[nH]c(=O)c2C(N)=O)c1]

The title compound was synthesized similar to example 49 in scheme 16.

### Example 55

### (R)-2-(1-amino-4-methyl-1-oxopentan-2-ylamino)-4-(3,5-dimethylphenylamino)-6-oxo-1,6-dihydropyrimidine-5-carboxamide

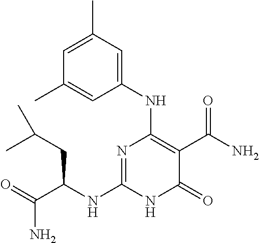 [SMILES: Cc1cc(C)cc(Nc2nc(N[C@H](CC(C)C)C(N)=O)[nH]c(=O)c2C(N)=O)c1]

The title compound was synthesized similar to example 49 in scheme 16.

### Example 56

### (R)-2-(1-amino-1-oxopropan-2-ylamino)-4-(3,5-dimethylphenylamino)-6-oxo-1,6-dihydropyrimidine-5-carboxamide

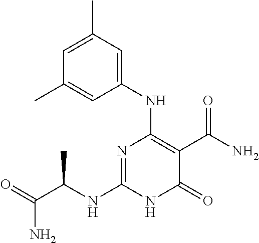 [SMILES: Cc1cc(C)cc(Nc2nc(N[C@H](C)C(N)=O)[nH]c(=O)c2C(N)=O)c1]

The title compound was synthesized similar to example 49 in scheme 16.

### Example 57

### 2-(2-aminocyclohexylamino)-4-(3,5-dimethylphenylamino)-6-oxo-1,6-dihydropyrimidine-5-carboxamide

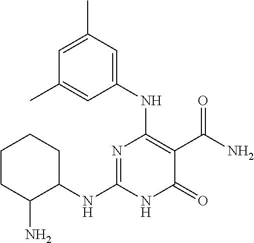 [SMILES: Cc1cc(C)cc(Nc2nc(NC3CCCCC3N)[nH]c(=O)c2C(N)=O)c1]

The title compound was synthesized similar to example 49 in scheme 16.

### Example 58

### 4-(cyclopropylamino)-2-(4-(N-methylacetamido)phenylamino)-6-oxo-1,6-dihydropyrimidine-5-carboxamide

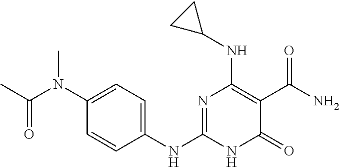 [SMILES: CC(=O)N(C)c1ccc(Nc2nc(NC3CC3)c(C(N)=O)c(=O)[nH]2)cc1]

The title compound was synthesized similar to example 49 in scheme 16.

### Example 59

### 2-(4-(4-acetylpiperazin-1-yl)phenylamino)-4-(3,5-dimethylphenylamino)-6-oxo-1,6-dihydropyrimidine-5-carboxamide

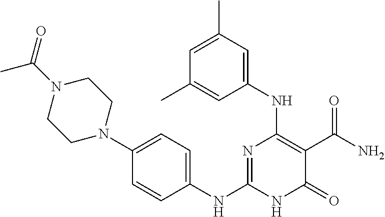 [SMILES: CC(=O)N1CCN(c2ccc(Nc3nc(Nc4cc(C)cc(C)c4)c(C(N)=O)c(=O)[nH]3)cc2)CC1]

The title compound was synthesized similar to example 49 in scheme 16.

### Example 60

### 2-(cyclopropylamino)-4-(3,5-dimethylphenylamino)-6-oxo-1,6-dihydropyrimidine-5-carboxamide

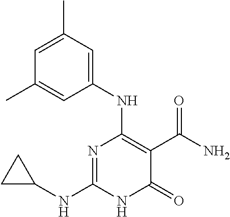 [SMILES: Cc1cc(C)cc(Nc2nc(NC3CC3)[nH]c(=O)c2C(N)=O)c1]

The title compound was synthesized similar to example 49 in scheme 16.

### Example 61

### 4-(4-(3,5-dimethylphenylamino)-5-carboxamide-6-oxo-1,6-dihydropyrimidin-2-ylamino)benzenesulfonamide

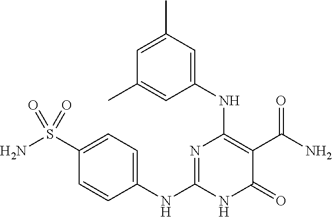 [SMILES: Cc1cc(C)cc(Nc2nc(Nc3ccc(S(N)(=O)=O)cc3)[nH]c(=O)c2C(N)=O)c1]

The title compound was synthesized similar to example 49 in scheme 16.

### Example 62

### 2-(4-(4-acetylpiperazin-1-yl)phenylamino)-4-(cyclopropylamino)-6-oxo-1,6-dihydropyrimidine-5-carboxamide

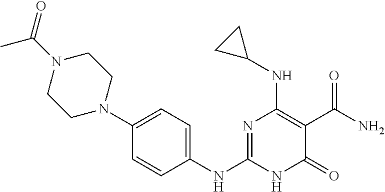 [SMILES: CC(=O)N1CCN(c2ccc(Nc3nc(NC4CC4)c(C(N)=O)c(=O)[nH]3)cc2)CC1]

The title compound was synthesized similar to example 49 in scheme 16.

### Example 63

### 4-(4-(3-methylphenylamino)-5-carboxamide-6-oxo-1,6-dihydropyrimidin-2-ylamino)benzenesulfonamide

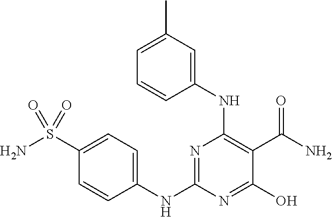 [SMILES: Cc1cccc(Nc2nc(Nc3ccc(S(N)(=O)=O)cc3)nc(O)c2C(N)=O)c1]

The title compound was synthesized similar to example 48 in scheme 15.

### Example 64

### 4-(m-toluidino)-2-(4-(4-acetylpiperazin-1-yl)phenylamino)-6-hydroxypyrimidine-5-carboxamide

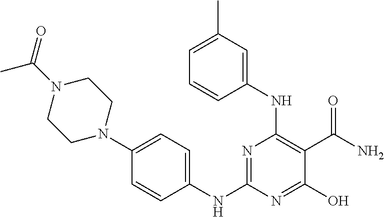 [SMILES: CC(=O)N1CCN(c2ccc(Nc3nc(O)c(C(N)=O)c(Nc4cccc(C)c4)n3)cc2)CC1]

The title compound was synthesized similar to example 48 in scheme 15.

### Example 65

### 2-(2-aminocyclohexylamino)-4-(3,5-dimethylphenylamino)-6-methoxypyrimidine-5-carboxamide

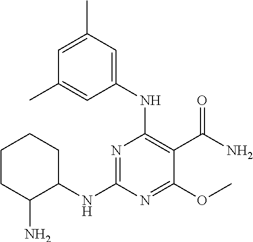 [SMILES: COc1nc(NC2CCCCC2N)nc(Nc2cc(C)cc(C)c2)c1C(N)=O]

The title compound was synthesized similar to example 50 in scheme 17.

### Example 66

### (R)-2-(1-amino-1-oxopropan-2-ylamino)-4-(3,5-dimethylphenylamino)-6-methoxypyrimidine-5-carboxamide

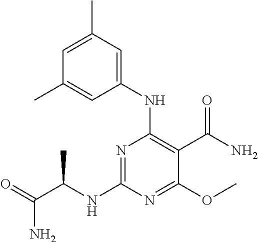 [SMILES: COc1nc(N[C@H](C)C(N)=O)nc(Nc2cc(C)cc(C)c2)c1C(N)=O]

The title compound was synthesized similar to example 50 in scheme 17.

### Example 67

### (R)-2-(1-amino-4-methyl-1-oxopentan-2-ylamino)-4-(3,5-dimethylphenylamino)-6-methoxypyrimidine-5-carboxamide

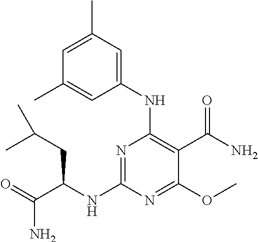 [SMILES: COc1nc(N[C@H](CC(C)C)C(N)=O)nc(Nc2cc(C)cc(C)c2)c1C(N)=O]

The title compound was synthesized similar to example 50 in scheme 17.

### Example 68

### (S)-2-(2-aminopropylamino)-4-(3,5-dimethylphenylamino)-6-methoxypyrimidine-5-carboxamide

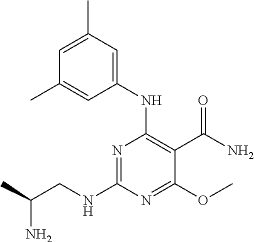 [SMILES: COc1nc(NC[C@H](C)N)nc(Nc2cc(C)cc(C)c2)c1C(N)=O]

The title compound was synthesized similar to example 50 in scheme 17.

### Example 69

### 2-(4-(4-acetylpiperazin-1-yl)phenylamino)-4-(cyclopropylamino)-6-methoxypyrimidine-5-carboxamide

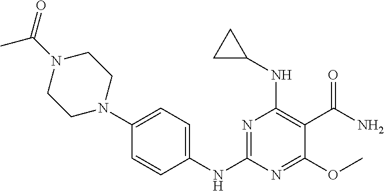 [SMILES: COc1nc(Nc2ccc(N3CCN(C(C)=O)CC3)cc2)nc(NC2CC2)c1C(N)=O]

The title compound was synthesized similar to example 50 in scheme 17.

### Example 70

### 4-(m-toluidino)-6-methoxy-2-(4-morpholinophenylamino)pyrimidine-5-carboxamide

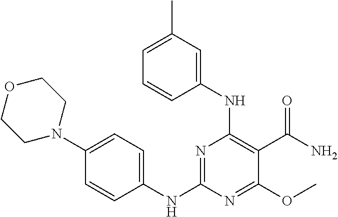 [SMILES: COc1nc(Nc2ccc(N3CCOCC3)cc2)nc(Nc2cccc(C)c2)c1C(N)=O]

The title compound was synthesized similar to example 50 in scheme 17.

### Example 71

### (R)-4-(m-toluidino)-2-(2-amino-2-oxo-1-phenylethylamino)-6-methoxypyrimidine-5-carboxamide

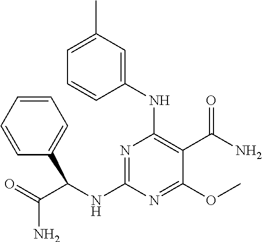 [SMILES: COc1nc(N[C@@H](C(N)=O)c2ccccc2)nc(Nc2cccc(C)c2)c1C(N)=O]

The title compound was synthesized similar to example 50 in scheme 17.

### Example 72

### (R)-2-(2-amino-2-oxo-1-phenylethylamino)-4-(3,5-dimethylphenylamino)-6-methoxypyrimidine-5-carboxamide

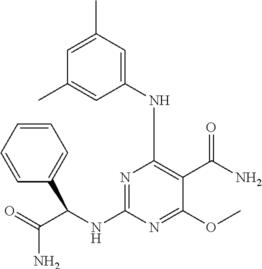 [SMILES: COc1nc(N[C@@H](C(N)=O)c2ccccc2)nc(Nc2cc(C)cc(C)c2)c1C(N)=O]

The title compound was synthesized similar to example 50 in scheme 17.

### Example 73

### 4-(cyclopropylamino)-6-methoxy-2-(4-morpholinophenylamino)pyrimidine-5-carboxamide

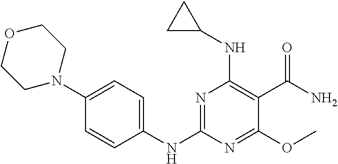 [SMILES: COc1nc(Nc2ccc(N3CCOCC3)cc2)nc(NC2CC2)c1C(N)=O]

The title compound was synthesized similar to example 50 in scheme 17.

### Example 74

### 4-(m-toluidino)-2-(4-(4-acetylpiperazin-1-yl)phenylamino)-6-methoxypyrimidine-5-carboxamide

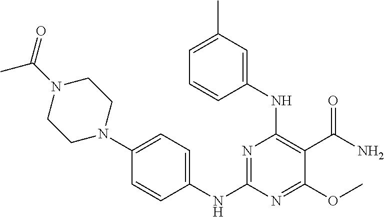 [SMILES: COc1nc(Nc2ccc(N3CCN(C(C)=O)CC3)cc2)nc(Nc2cccc(C)c2)c1C(N)=O]

The title compound was synthesized similar to example 50 in scheme 17.

### Example 75

### 4-(m-toluidino)-2-(ethylamino)-6-(2-hydroxyethoxy)pyrimidine-5-carboxamide

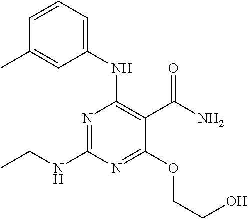 [SMILES: CCNc1nc(Nc2cccc(C)c2)c(C(N)=O)c(OCCO)n1]

The title compound was synthesized similar to example 53 in scheme 20.

### Example 76

### (R)-2-(1-amino-1-oxobutan-2-ylamino)-4-(benzo[d][1,3]dioxol-5-ylamino)-6-oxo-1,6-dihydropyrimidine-5-carboxamide

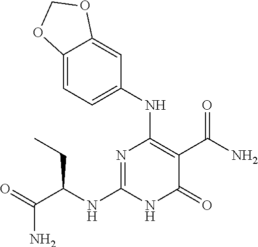 [SMILES: CC[C@@H](Nc1nc(Nc2ccc3c(c2)OCO3)c(C(N)=O)c(=O)[nH]1)C(N)=O]

The title compound was synthesized similar to example 39 in Scheme 9 using benzo[d][1,3]dioxol-5-amine to replace 3-methoxyaniline. MS found for C₁₆H₁₈N₆O₅ as (M+H)⁺375.2. λ=265.3, 299.7.

### Example 77

### (R)-2-(1-amino-4-methyl-1-oxopentan-2-ylamino)-4-(3-methoxyphenylamino)-6-oxo-1,6-dihydropyrimidine-5-carboxamide

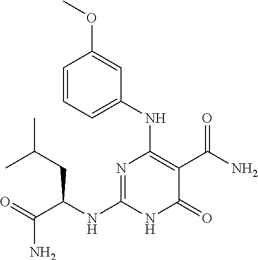 [SMILES: COc1cccc(Nc2nc(N[C@H](CC(C)C)C(N)=O)[nH]c(=O)c2C(N)=O)c1]

The title compound was synthesized similar to example 39 in Scheme 9 using D-leucinamide to replace (R)-2-aminobutanamide. MS found for C₁₈H₂₄N₆O₄ as (M+H)⁺ 389.4. λ=256.4, 301.3.

### Example 78

### (R)-2-(1-amino-4-methyl-1-oxopentan-2-ylamino)-4-(2,3-dihydrobenzo[b][1,4]dioxin-6-ylamino)-6-oxo-1,6-dihydropyrimidine-5-carboxamide

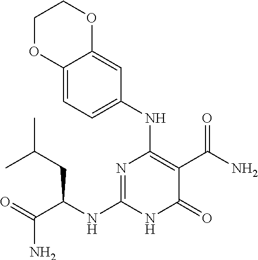 [SMILES: CC(C)C[C@@H](Nc1nc(Nc2ccc3c(c2)OCCO3)c(C(N)=O)c(=O)[nH]1)C(N)=O]

The title compound was synthesized similar to example 39 in Scheme 9 using 2,3-dihydrobenzo[b][1,4]dioxin-6-amine to replace 3-methoxyaniline and using D-leucinamide to replace (R)-2-aminobutanamide. MS found for C₁₉H₂₄N₆O₅ as (M+H)⁺ 417.3. λ=261.7, 297.3.

### Example 79

### (R)-2-(1-amino-4-methyl-1-oxopentan-2-ylamino)-4-(benzo[d][1,3]-dioxol-5-ylamino)-6-oxo-1,6-dihydropyrimidine-5-carboxamide

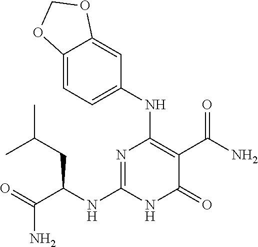 [SMILES: CC(C)C[C@@H](Nc1nc(Nc2ccc3c(c2)OCO3)c(C(N)=O)c(=O)[nH]1)C(N)=O]

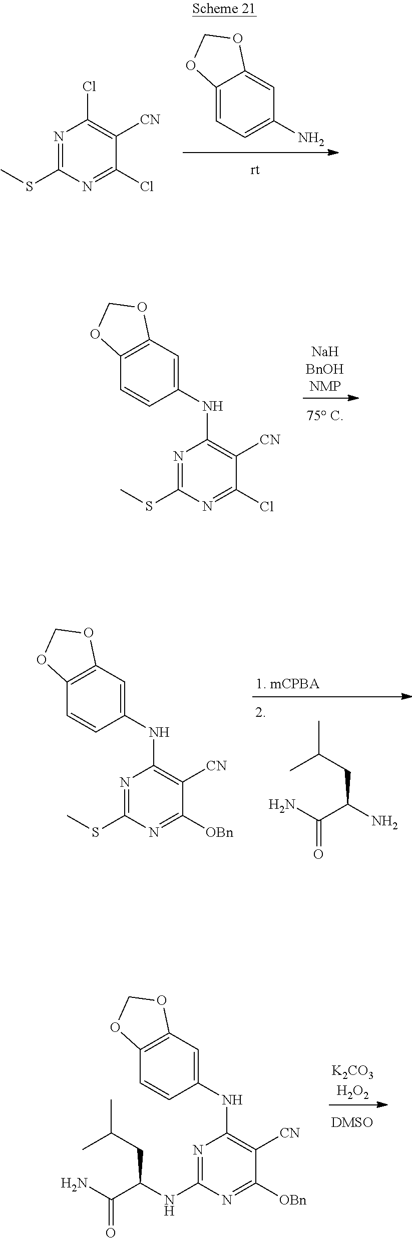

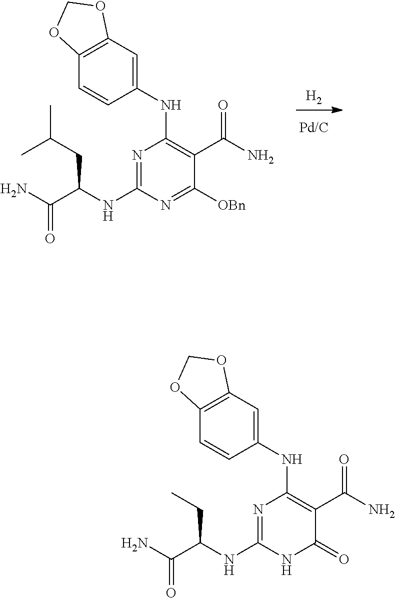 [SMILES: CC(C)C[C@@H](Nc1nc(Nc2ccc3c(c2)OCO3)c(C(N)=O)c(OCc2ccccc2)n1)C(N)=O ; "H2" ; CC[C@@H](Nc1nc(Nc2ccc3c(c2)OCO3)c(C(N)=O)c(=O)[nH]1)C(N)=O]

Step 1: To a suspension of 4,6-dichloro-2-(methylthio)pyrimidine-5-carbonitrile (176 mg, 0.8 mmol) in DMF (2 mL) was added benzo[d][1,3]dioxol-5-amine (137.1 mg, 1 mmol). After stirring at room temperature for 15 h, the mixture was diluted with water, the resulting precipitate was collected by filtration to give 4-(benzo[d][2,3]dioxol-5-ylamino)-6-chloro-2-(methylthio)pyrimidin-5-carbonitrile (273 mg).

Step 2: To NaH (89 mg) in NMP (1.2 mL) was added benzyl alcohol (130 mg, 1.2 mmol), after stirring at room temperature for 30 min, it was added a solution of 4-(benzo[d][2,3]dioxol-5-ylamino)-6-chloro-2-(methylthio)pyrimidin-5-carbonitrile (240 mg, 0.78 mmol) in NMP (1.5 mL). The mixture was then heated at 75° C. for 2 h, more NaH (89 mg) was added, followed by additional heating at 75° C. for 30 min. The mixture was added ice water, the resulting precipitate was collected by filtration to give 4-(benzo[d][2,3]dioxol-5-ylamino)-6-(benzyloxy)-2-(methylthio)pyrimidin-5-carbonitrile (322 mg).

Step 3: To a solution of 4-(benzo[d][2,3]dioxol-5-ylamino)-6-(benzyloxy)-2-(methylthio)pyrimidin-5-carbonitrile (322 mg, 0.8 mmol) was added mCPBA (65%, 425 mg, 1.6 mmol) and NaHCO₃ (202 mg, 2.4 mmol). After stirring at room temperature for 20 min, it was added water and EtOAc, organic layer was separated and washed with Sat. NaHCO₃, brine, dried and concentrated to give a mixture of crude sulfoxide and sulfone, which was subsequently dissolved in NMP (2 mL), added (R)-leucinamide HCl salt (133 mg, 0.96 mmol) and DIPEA (0.285 mL, 1.6 mmol), the mixture was heated at 60° C. for 1 h, cooled to room temperature, diluted with water and EtOAc, organic layer was separated and washed with Sat. NaHCO₃, brine, dried and concentrated to give (R)-2-(4-(benzo[d][2,3]dioxol-5-ylamino)-6-(benzyloxy)-5-cyanopyrimidine-2-ylamino)-4-methylpentanamide as crude solid (390 mg).

Step 4: To above (R)-2-(4-(benzo[d][2,3]dioxol-5-ylamino)-6-(benzyloxy)-5-cyanopyrimidine-2-ylamino)-4-methylpentanamide as crude solid (390 mg) in DMSO (2 mL) was added H₂O₂ (50%, 1 mL), K₂CO₃ (332 mg, 2.4 mmol), after heated at 90° C. for 1 h, it was cooled and diluted with water, the resulting precipitate was collected by filtration to give crude amide (R)-2-(1-amino-4-methyl-1-oxopentan-2-ylamino)-4-(benzo[d][2,3]dioxol-5-ylamino)-6-(benzyloxy)pyrimidine-5-carboxamide.

Step 5: To a solution of the above crude amide (R)-2-(1-amino-4-methyl-1-oxopentan-2-ylamino)-4-(benzo[d][2,3]dioxol-5-ylamino)-6-(benzyloxy)pyrimidine-5-carboxamide in THF (0.8 mL) and EtOH (0.8 mL) was added Pd/C (40 mg), charged with H₂ (1 atm). After stirred at room temperature for 2 h, Pd/C was filtered off, the filtrate was concentrated and purified by preparative HPLC to give (R)-2-(1-amino-4-methyl-1-oxopentan-2-ylamino)-4-(benzo[d][1,3]dioxol-5-ylamino)-6-oxo-1,6-dihydropyrimidine-5-carboxamide (27 mg). MS found for C₁₆H₁₈N₆O₅ as (M+H)⁺ 403.3. λ=264.1, 299.7.

### Example 80

### (R)-2-(1-amino-4-methyl-1-oxopentan-2-ylamino)-6-oxo-4-(3-(3-fluoropyridin-2-yl)phenylamino)-1,6-dihydropyrimidine-5-carboxamide

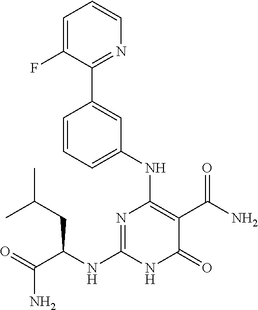 [SMILES: CC(C)C[C@@H](Nc1nc(Nc2cccc(-c3ncccc3F)c2)c(C(N)=O)c(=O)[nH]1)C(N)=O]

The title compound was synthesized similar to example 79 in Scheme 21 using 3-(3-fluoropyridin-2-yl)aniline to replace benzo[d][1,3]dioxol-5-amine. MS found for C₂₂H₂₄FN₇O₃ as (M+H)⁺ 454.4. λ=257.0, 300.9.

### Example 81

### (R)-2-(1-amino-1-oxobutan-2-ylamino)-6-oxo-4-(3-(3-fluoropyridin-2-yl)phenylamino)-1,6-dihydropyrimidine-5-carboxamide and Example 82 (R)-2-(1-amino-1-oxobutan-2-ylamino)-6-oxo-4-(3-(3-hydroxypyridin-2-yl)phenylamino)-1,6-dihydropyrimidine-5-carboxamide

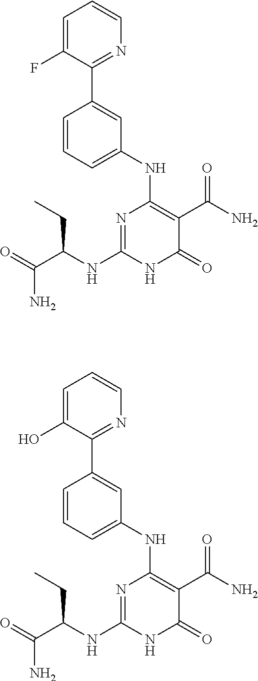 [SMILES: CC[C@@H](Nc1nc(Nc2cccc(-c3ncccc3F)c2)c(C(N)=O)c(=O)[nH]1)C(N)=O ; CC[C@@H](Nc1nc(Nc2cccc(-c3ncccc3O)c2)c(C(N)=O)c(=O)[nH]1)C(N)=O]

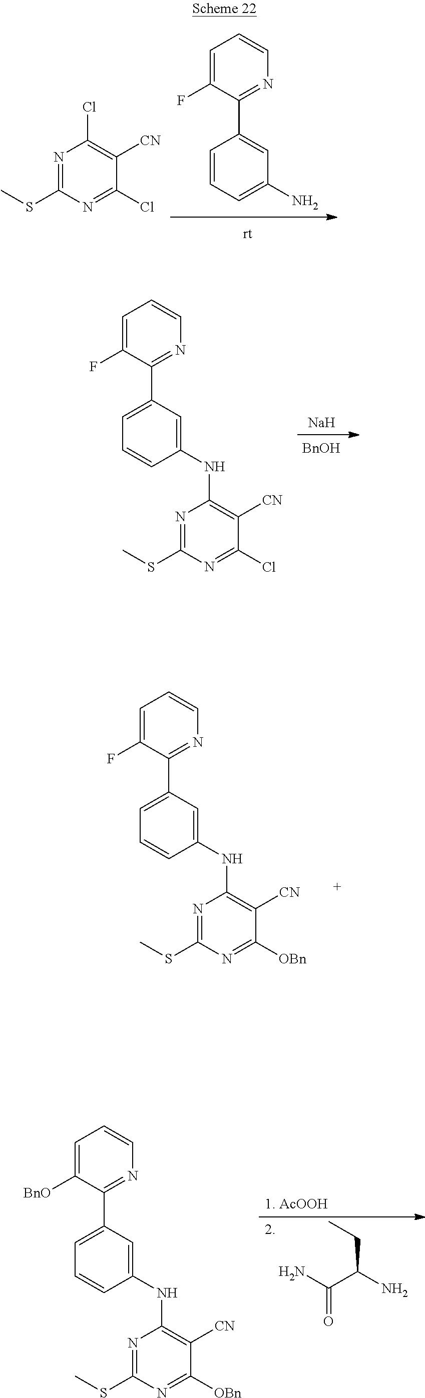

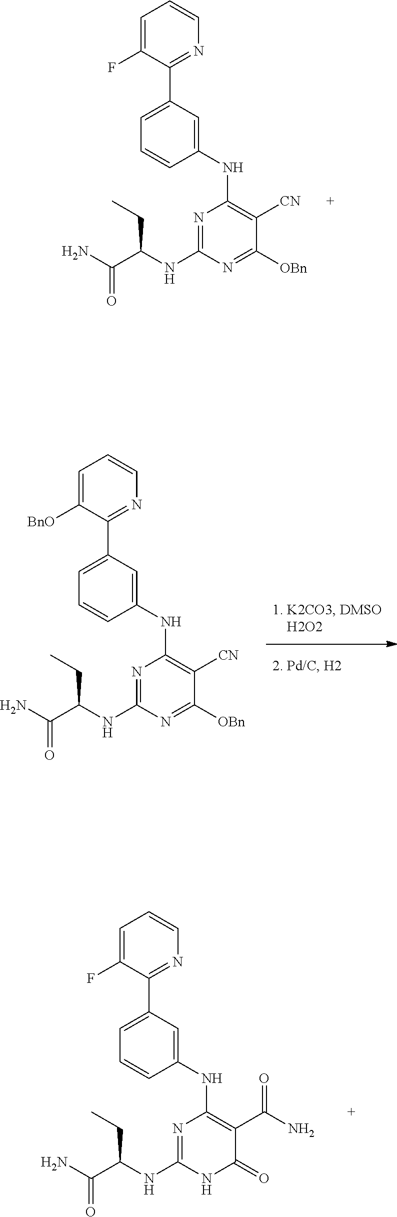 [SMILES: CC[C@@H](Nc1nc(Nc2cccc(-c3ncccc3F)c2)c(C#N)c(OCc2ccccc2)n1)C(N)=O ; CC[C@@H](Nc1nc(Nc2cccc(-c3ncccc3OCc3ccccc3)c2)c(C#N)c(OCc2ccccc2)n1)C(N)=O ; CC[C@@H](Nc1nc(Nc2cccc(-c3ncccc3F)c2)c(C(N)=O)c(=O)[nH]1)C(N)=O]

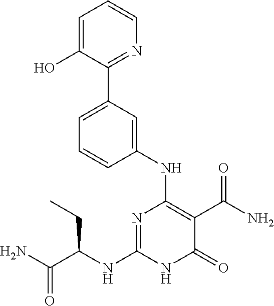 [SMILES: CC[C@@H](Nc1nc(Nc2cccc(-c3ncccc3O)c2)c(C(N)=O)c(=O)[nH]1)C(N)=O]

Step 1: To a suspension of 4,6-dichloro-2-(methylthio)pyrimidine-5-carbonitrile (330 mg, 1.5 mmol) in DMF (3 mL) was added 3-(3-fluoropyridin-2-yl)aniline (352 mg, 1.875 mmol). After stirring at room temperature for 15 h, the mixture was diluted with water, the resulting precipitate was collected by filtration to give 4-chloro-6-(3-(3-fluoropyridin-2-yl)phenylamino)-2-(methylthio)pyrimidin-5-carbonitrile (455 mg).

Step 2: To NaH (135 mg) in NMP (1.7 mL) was added benzyl alcohol (184 mg, 1.71 mmol), after stirring at room temperature for 30 min, it was added a solution of 4-chloro-6-(3-(3-fluoropyridin-2-yl)phenylamino)-2-(methylthio)pyrimidin-5-carbonitrile (455 mg) in NMP (2 mL). The mixture was then heated at 75° C. for 2 h, more NaH (150 mg) was added, followed by additional heating at 75° C. for 30 min. The mixture was added ice water, the resulting precipitate was collected by filtration to give a mixture of 4-(benzyloxy)-6-(3-(3-fluoropyridin-2-yl)phenylamino)-2-(methylthio)pyrimidin-5-carbonitrile and 4-(benzyloxy)-6-(3-(3-benzoxypyridin-2-yl)phenylamino)-2-(methylthio)pyrimidin-5-carbonitrile (540 mg total).

Step 3: To a solution of above mixture in AcCN (4 mL) was added AcOOH (solution in AcOH, 0.4 mL). After stirring at room temperature for 1 h, AcCN was removed and the residue was diluted with water, the precipitate was collected by filtration to give corresponding sulfoxide (570 mg). A portion of the sulfoxide (270 mg) in NMP (2 mL) was added (R)-2-aminobutanamide HCl salt (100 mg, 1.25 mmol) and DIPEA (0.206 mL, 1.16 mmol), the mixture was heated at 60° C. for 1 h, cooled to room temperature, diluted with water, the precipitate was collected by filtration to give a mixture of (R)-2-(4-(benzyloxy)-5-cyano-6-(3-(3-fluoropyridin-2-yl)phenylpyrimidine-2-ylamino)butanamide and (R)-2-(4-(benzyloxy)-5-cyano-6-(3-(3-benzoxypyridin-2-yl)phenylpyrimidine-2-ylamino)butanamide

Step 4: To above mixture in DMSO (2.5 mL) was added H₂O₂ (50%, 1.5 mL), K₂CO₃ (400 mg), after heated at 90° C. for 1 h, it was cooled and diluted with water, the resulting precipitate was collected by filtration to give mixture of (R)-2-(1-amino-1-oxobutan-2-ylamino)-4-(benzyloxy)-6-(3-(3-fluoropyridin-2-yl)phenylamino)-1,6-dihydropyrimidine-5-carboxamide and (R)-2-(1-amino-1-oxobutan-2-ylamino)-4-(benzyloxy)-6-(3-(3-benzyoxypyridin-2-yl)phenylamino)-1,6-dihydropyrimidine-5-carboxamide (224 mg total).

Step 5: To a solution of the above mixture of crude amides in THF (2 mL) and EtOH (2 mL) was added Pd/C (150 mg), charged with H₂ (1 atm). After stirred at room temperature for 2 h, Pd/C was filtered off, the filtrate was concentrated and purified by preparative HPLC to give (R)-2-(1-amino-1-oxobutan-2-ylamino)-6-oxo-4-(3-(3-fluoropyridin-2-yl)phenylamino)-1,6-dihydropyrimidine-5-carboxamide (85 mg, MS found for C₂₀H₂₀FN₇₀₃ as (M+H)⁺ 426.3. λ=257.0, 299.7) and (R)-2-(1-amino-1-oxobutan-2-ylamino)-6-oxo-4-(3-(3-hydroxypyridin-2-yl)phenylamino)-1,6-dihydropyrimidine-5-carboxamide 15 mg, MS found for C₂₀H₂₁N₇O₄ as (M+H)⁺ 424.3. λ=252.3, 309.2).

### Example 83

### (R)-2-(1-amino-4-methyl-1-oxopentan-2-ylamino)-4-(3,4-dimethoxyphenylamino)-6-oxo-1,6-dihydropyrimidine-5-carboxamide

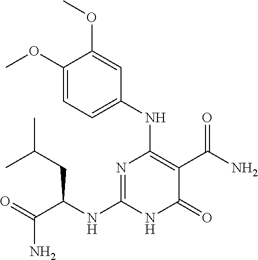 [SMILES: COc1ccc(Nc2nc(N[C@H](CC(C)C)C(N)=O)[nH]c(=O)c2C(N)=O)cc1OC]

The title compound was synthesized similarly to example 79 in Scheme 21 using 3,4-dimethoxyaniline to replace benzo[d][1,3]dioxol-5-amine. MS found for C₁₉H₂₆N₆O₅ as (M+H)⁺419.3. λ=262.9, 299.7.

### Example 86

### (R)-2-(1-amino-1-oxobutan-2-ylamino)-4-(2,3-dihydrobenzo[b][1,4]dioxin-6-ylamino)-6-oxo-1,6-dihydropyrimidine-5-carboxamide

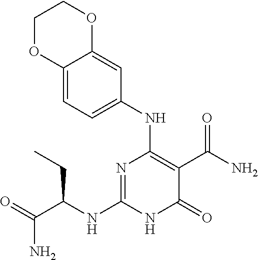 [SMILES: CC[C@@H](Nc1nc(Nc2ccc3c(c2)OCCO3)c(C(N)=O)c(=O)[nH]1)C(N)=O]

The title compound was synthesized similarly to example 79 in Scheme 21 using 2,3-dihydrobenzo[b][1,4]dioxin-6-amine to replace benzo[d][1,3]dioxol-5-amine and using (R)-2-aminobutanamide to replace D-leucinamide. MS found for C₁₇H₂₀N₆O₅ as (M+H)⁺ 389.2. λ=260.5, 306.8.

### Example 87

### (R)-2-(1-amino-4-methyl-1-oxopentan-2-ylamino)-4-(3-(difluoromethoxy)phenylamino)-6-oxo-1,6-dihydropyrimidine-5-carboxamide

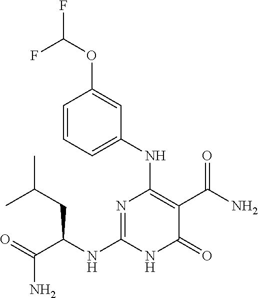 [SMILES: CC(C)C[C@@H](Nc1nc(Nc2cccc(OC(F)F)c2)c(C(N)=O)c(=O)[nH]1)C(N)=O]

The title compound was synthesized similarly to example 79 in Scheme 21 using 3-(difluoromethoxy)aniline to replace benzo[d][1,3]dioxol-5-amine. MS found for C₁₈H₂₂F₂N₆O₄ as (M+H)⁺ 425.3. λ=255.8, 300.9.

### Example 88

### (R)-2-(1-amino-1-oxobutan-2-ylamino)-4-(3-(difluoromethoxy)phenylamino)-6-oxo-1,6-dihydropyrimidine-5-carboxamide

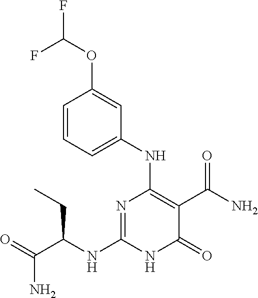 [SMILES: CC[C@@H](Nc1nc(Nc2cccc(OC(F)F)c2)c(C(N)=O)c(=O)[nH]1)C(N)=O]

The title compound was synthesized similarly to example 79 in Scheme 21 using 3-(difluoromethoxy)aniline to replace benzo[d][1,3]dioxol-5-amine and using (R)-2-aminobutanamide to replace D-leucinamide. MS found for C₁₆H₁₈F₂N₆O₄ as (M+H)⁺ 397.3. λ=255.8, 300.9.

### Example 89

### (R)-2-(1-amino-4-methyl-1-oxopentan-2-ylamino)-4-(4-methoxyphenylamino)-6-oxo-1,6-dihydropyrimidine-5-carboxamide

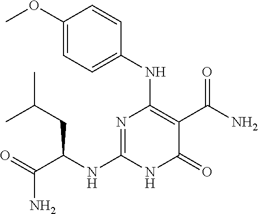 [SMILES: COc1ccc(Nc2nc(N[C@H](CC(C)C)C(N)=O)[nH]c(=O)c2C(N)=O)cc1]

The title compound was synthesized similarly to example 79 in Scheme 21 using 4-methoxyaniline to replace benzo[d][1,3]dioxol-5-amine. MS found for C₁₈H₂₄N₆O₄ as (M+H)⁺389.3. λ=260.5, 297.3.

### Example 90

### (R)-2-(1-amino-1-oxobutan-2-ylamino)-4-(4-methoxyphenylamino)-6-oxo-1,6-dihydropyrimidine-5-carboxamide

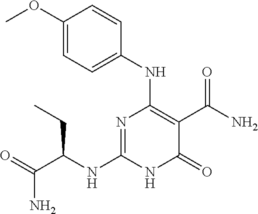 [SMILES: CC[C@@H](Nc1nc(Nc2ccc(OC)cc2)c(C(N)=O)c(=O)[nH]1)C(N)=O]

The title compound was synthesized similarly to example 79 in Scheme 21 using 4-methoxyaniline to replace benzo[d][1,3]dioxol-5-amine and using (R)-2-aminobutanamide to replace D-leucinamide. MS found for C₁₆H₂₀N₆O₄ as (M+H)⁺ 361.3. λ=260.5, 296.1.

### Example 91

### (R)-2-(1-amino-4-methyl-1-oxopentan-2-ylamino)-4-(4-(trifluoromethoxy)phenylamino)-6-oxo-1,6-dihydropyrimidine-5-carboxamide

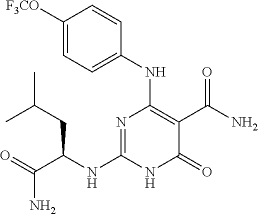 [SMILES: CC(C)C[C@@H](Nc1nc(Nc2ccc(OC(F)(F)F)cc2)c(C(N)=O)c(=O)[nH]1)C(N)=O]

The title compound was synthesized similarly to example 79 in Scheme 21 using 4-(trifluoromethoxy)aniline to replace benzo[d][1,3]dioxol-5-amine. MS found for C₁₈H₂₁F₃N₆O₄ as (M+H)⁺ 443.3. λ=255.8, 298.5.

### Example 92

### (R)-2-(1-amino-1-oxobutan-2-ylamino)-4-(4-(trifluoromethoxy)phenylamino)-6-oxo-1,6-dihydropyrimidine-5-carboxamide

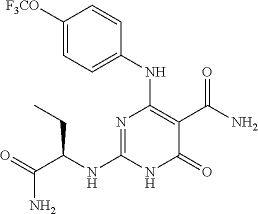 [SMILES: CC[C@@H](Nc1nc(Nc2ccc(OC(F)(F)F)cc2)c(C(N)=O)c(=O)[nH]1)C(N)=O]

The title compound was synthesized similarly to example 79 in Scheme 21 using 4-(trifluoromethoxy)aniline to replace benzo[d][1,3]dioxol-5-amine and using (R)-2-aminobutanamide to replace D-leucinamide. MS found for C₁₆H₁₇F₃N₆O₄ as (M+H)⁺ 415.2. λ=258.2, 300.9.

### Example 93

### (R)-2-(1-amino-4-methyl-1-oxopentan-2-ylamino)-4-(4-(2-methoxyethoxy)phenylamino)-6-oxo-1,6-dihydropyrimidine-5-carboxamide

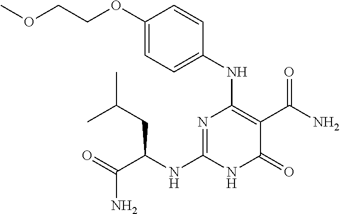 [SMILES: COCCOc1ccc(Nc2nc(N[C@H](CC(C)C)C(N)=O)[nH]c(=O)c2C(N)=O)cc1]

The title compound was synthesized similarly to example 79 in Scheme 21 using 4-(2-methoxyethoxy)aniline to replace benzo[d][1,3]dioxol-5-amine. MS found for C₂₀H₂₈N₆O₅ as (M+H)⁺ 433.3. λ=259.4, 297.3.

### Example 94

### (R)-2-(1-amino-1-oxobutan-2-ylamino)-4-(4-(2-methoxyethoxy)phenylamino)-6-oxo-1,6-dihydropyrimidine-5-carboxamide

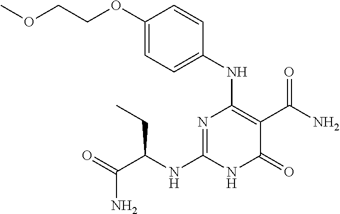 [SMILES: CC[C@@H](Nc1nc(Nc2ccc(OCCOC)cc2)c(C(N)=O)c(=O)[nH]1)C(N)=O]

The title compound was synthesized similarly to example 79 in Scheme 21 using 4-(2-methoxyethoxy)aniline to replace benzo[d][1,3]dioxol-5-amine and using (R)-2-aminobutanamide to replace D-leucinamide. MS found for C₁₈H₂₄N₆O₅ as (M+H)⁺ 405.3. λ=259.4, 296.1.

### Example 95

### (R)-2-(1-amino-3-cyclopropyl-1-oxopropan-2-ylamino)-6-oxo-4-(3-(pyridin-2-yl)phenylamino)-1,6-dihydropyrimidine-5-carboxamide

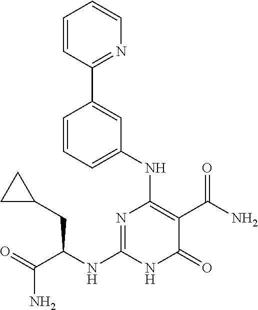 [SMILES: NC(=O)c1c(Nc2cccc(-c3ccccn3)c2)nc(N[C@H](CC2CC2)C(N)=O)[nH]c1=O]

The title compound was synthesized similar to example 79 in Scheme 21 using 3-(pyridin-2-yl)aniline to replace benzo[d][1,3]dioxol-5-amine and using (R)-2-amino-3-cyclopropylpropanamide to replace D-leucinamide. MS found for C₂₂H₂₃N₇O₃ as (M+H)⁺ 434.2. λ=249.9, 302.1.

### Example 96

### (R)-2-(1-amino-3-methyl-1-oxobuan-2-ylamino)-6-oxo-4-(3-(pyridin-2-yl)phenylamino)-1,6-dihydropyrimidine-5-carboxamide

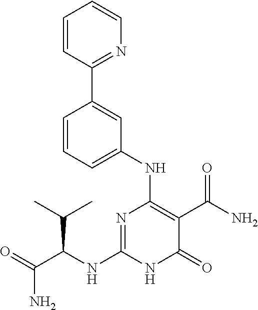 [SMILES: CC(C)[C@@H](Nc1nc(Nc2cccc(-c3ccccn3)c2)c(C(N)=O)c(=O)[nH]1)C(N)=O]

The title compound was synthesized similar to example 79 in Scheme 21 using 3-(pyridin-2-yl)aniline to replace benzo[d][1,3]dioxol-5-amine and using D-valinamide to replace D-leucinamide. MS found for C₂₁H₂₃N₇O₃ as (M+H)⁺ 422.2. λ=249.9, 302.1.

### Example 97

### (R)-2-(1-amino-4-methyl-1-oxopentan-2-ylamino)-6-oxo-4-(3-(pyridin-3-yl)phenylamino)-1,6-dihydropyrimidine-5-carboxamide

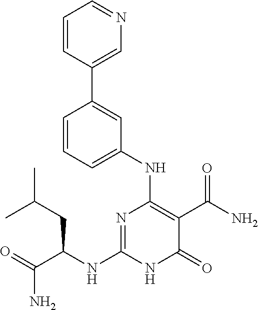 [SMILES: CC(C)C[C@@H](Nc1nc(Nc2cccc(-c3cccnc3)c2)c(C(N)=O)c(=O)[nH]1)C(N)=O]

The title compound was synthesized similar to example 79 in Scheme 21 using 3-(pyridin-3-yl)aniline to replace benzo[d][1,3]dioxol-5-amine. MS found for C₂₂H₂₅N₇O₃ as (M+H)⁺ 436.6. λ=255.8, 297.6.

### Example 98

### (R)-2-(1-amino-4-methyl-1-oxopentan-2-ylamino)-4-(3-(2-methoxyethoxy)phenylamino)-6-oxo-1,6-dihydropyrimidine-5-carboxamide

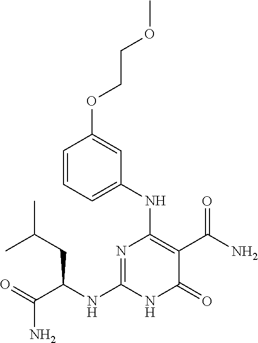 [SMILES: COCCOc1cccc(Nc2nc(N[C@H](CC(C)C)C(N)=O)[nH]c(=O)c2C(N)=O)c1]

The title compound was synthesized similarly to example 79 in Scheme 21 using 3-(2-methoxyethoxy)aniline to replace benzo[d][1,3]dioxol-5-amine. MS found for C₂₀H₂₈N₆O₅ as (M+H)⁺ 433.5. λ=256.4, 302.5.

### Example 99

### (R)-2-(1-amino-1-oxobutan-2-ylamino)-4-(3-(2-methoxyethoxy)phenylamino)-6-oxo-1,6-dihydropyrimidine-5-carboxamide

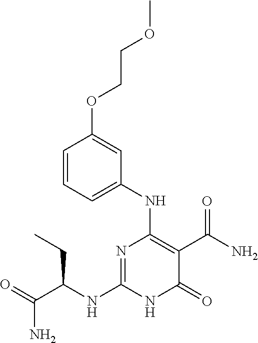 [SMILES: CC[C@@H](Nc1nc(Nc2cccc(OCCOC)c2)c(C(N)=O)c(=O)[nH]1)C(N)=O]

The title compound was synthesized similarly to example 79 in Scheme 21 using 3-(2-methoxyethoxy)aniline to replace benzo[d][1,3]dioxol-5-amine and using (R)-2-aminobutanamide to replace D-leucinamide. MS found for C₁₈H₂₄N₆O₅ as (M+H)⁺ 405.5. λ=256.4, 301.9.

### Example 100

### (R)-2-(4-methyl-1-(methylamino)-1-oxopentan-2-ylamino)-6-oxo-4-(3-(pyridin-2-yl)phenylamino)-1,6-dihydropyrimidine-5-carboxamide

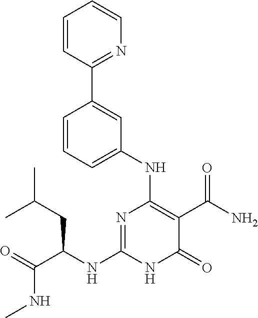 [SMILES: CNC(=O)[C@@H](CC(C)C)Nc1nc(Nc2cccc(-c3ccccn3)c2)c(C(N)=O)c(=O)[nH]1]

The title compound was synthesized similar to example 79 in Scheme 21 using 3-(pyridin-2-yl)aniline to replace benzo[d][1,3]dioxol-5-amine and using (R)-2-amino-N,4-dimethylpentanamide to replace D-leucinamide MS found for C₂₃H₂₇N₇O₃ as (M+H)⁺ 450.6. λ=249.9, 300.9.

### Example 101

### (R)-2-(1-amino-4-methyl-1-oxopentan-2-ylamino)-4-(3-(trifluoromethoxy)phenylamino)-6-oxo-1,6-dihydropyrimidine-5-carboxamide

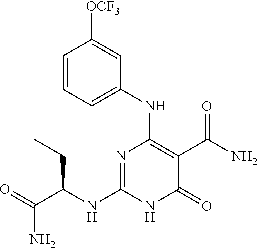 [SMILES: CC[C@@H](Nc1nc(Nc2cccc(OC(F)(F)F)c2)c(C(N)=O)c(=O)[nH]1)C(N)=O]

The title compound was synthesized similarly to example 79 in Scheme 21 using 3-(trifluoromethoxy)aniline to replace benzo[d][1,3]dioxol-5-amine. MS found for C₁₈H₂₁F₃N₆O₄ as (M+H)⁺ 443.5. λ=255.8, 300.9.

### Example 102

### (R)-2-(1-amino-1-oxobutan-2-ylamino)-4-(3-(trifluoromethoxy)phenylamino)-6-oxo-1,6-dihydropyrimidine-5-carboxamide

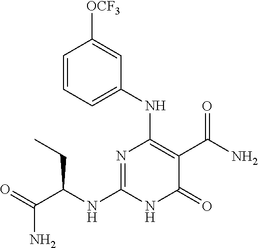 [SMILES: CC[C@@H](Nc1nc(Nc2cccc(OC(F)(F)F)c2)c(C(N)=O)c(=O)[nH]1)C(N)=O]

The title compound was synthesized similarly to example 79 in Scheme 21 using 3-(trifluoromethoxy)aniline to replace benzo[d][1,3]dioxol-5-amine and using (R)-2-aminobutanamide to replace D-leucinamide. MS found for C₁₆H₁₇F₃N₆O₄ as (M+H)⁺ 415.4. λ=255.8, 300.9.

### Example 103

### (R)-4-(benzo[d]thiazol-6-ylamino)-2-(4-methyl-1-(methylamino)-1-oxopentan-2-ylamino)-6-oxo-1,6-dihydropyrimidine-5-carboxamide

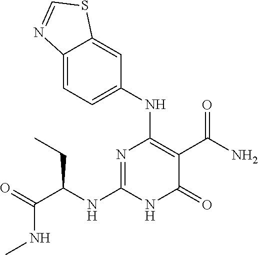 [SMILES: CC[C@@H](Nc1nc(Nc2ccc3ncsc3c2)c(C(N)=O)c(=O)[nH]1)C(=O)NC]

The title compound was synthesized similar to example 27 in Scheme 8 using 6-aminobenzothiazole to replace 3-(pyrimidin-2-yl)aniline and using (R)-2-amino-N,4-dimethylpentanamide to replace D-leucinamide MS found for C₁₉H₂₃N₇O₃S as (M+H)⁺ 430.5. λ=246.6, 279.1, 316.1.

### Example 104

### (R)-4-(3-(1H-pyrazol-1-yl)phenylamino)-2-(4-methyl-1-(methylamino)-1-oxopentan-2-ylamino)-6-oxo-1,6-dihydropyrimidine-5-carboxamide

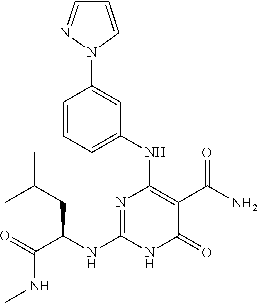 [SMILES: CNC(=O)[C@@H](CC(C)C)Nc1nc(Nc2cccc(-n3cccn3)c2)c(C(N)=O)c(=O)[nH]1]

The title compound was synthesized similar to example 27 in Scheme 8 using 3-(1H-pyrazol-1-yl)aniline to replace 3-(pyrimidin-2-yl)aniline and using (R)-2-amino-N,4-dimethylpentanamide to replace D-leucinamide MS found for C₂₁H₂₆N₈O₃ as (M+H)⁺ 439.6. λ=258.2, 300.9.

### Example 105

### (R)-4-(1H-indazol-5-ylamino)-2-(4-methyl-1-(methylamino)-1-oxopentan-2-ylamino)-6-oxo-1,6-dihydropyrimidine-5-carboxamide

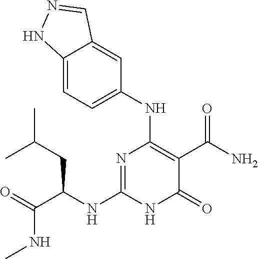 [SMILES: CNC(=O)[C@@H](CC(C)C)Nc1nc(Nc2ccc3[nH]ncc3c2)c(C(N)=O)c(=O)[nH]1]

The title compound was synthesized similar to example 27 in Scheme 8 using 5-aminoindazole to replace 3-(pyrimidin-2-yl)aniline and using (R)-2-amino-N,4-dimethylpentanamide to replace D-leucinamide MS found for C₁₉H₂₄N₈O₃ as (M+H)⁺ 413.5. λ=251.1.

### Example 106

### (R)-2-(1-amino-4-methyl-1-oxopentan-2-ylamino)-6-oxo-4-(4-(pyridin-3-yl)phenylamino)-1,6-dihydropyrimidine-5-carboxamide

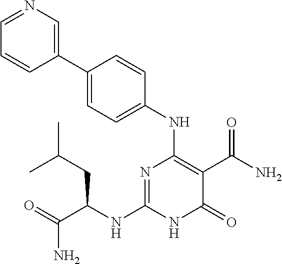 [SMILES: CC(C)C[C@@H](Nc1nc(Nc2ccc(-c3cccnc3)cc2)c(C(N)=O)c(=O)[nH]1)C(N)=O]

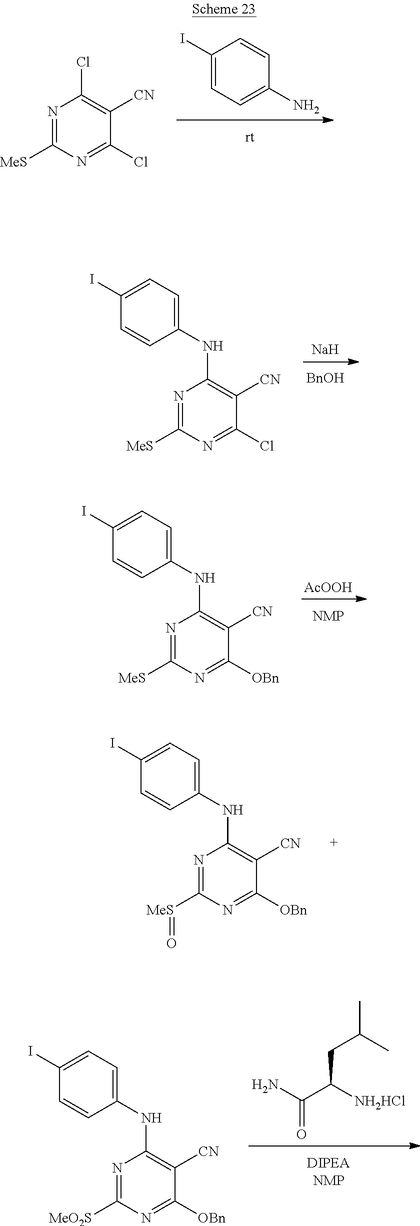

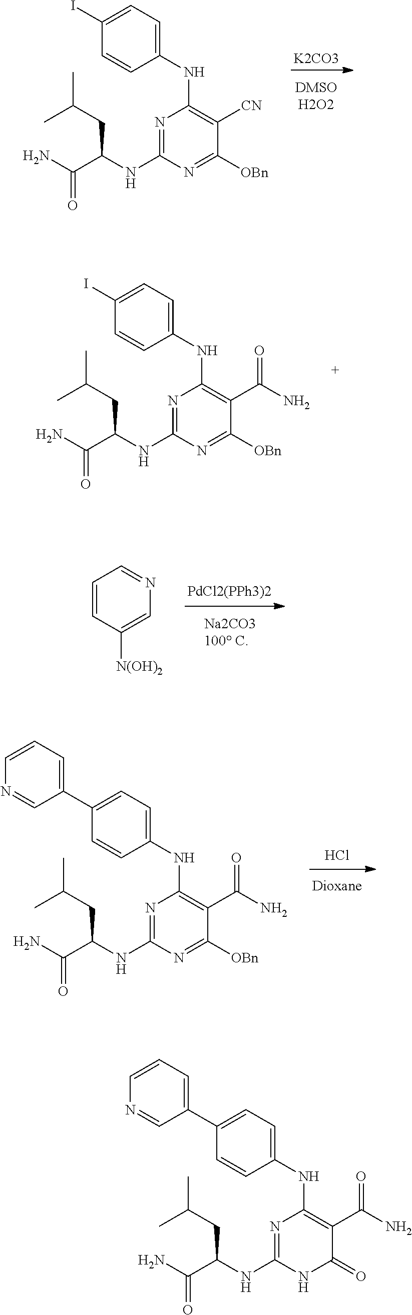

Step 1: To a suspension of 4,6-dichloro-2-(methylthio)pyrimidine-5-carbonitrile (330 mg, 1.5 mmol) in DMF (3 mL) was added 4-iodoaniline (361 mg, 1.65 mmol). After stirring at room temperature for 3 h, the mixture was diluted with water, the resulting precipitate was collected by filtration to give 4-chloro-6-(4-iodophenylamino)-2-(methylthio)pyrimidin-5-carbonitrile (541 mg).

Step 2: To benzyl alcohol (174 mg, 1.61 mmol) in NMP (2 mL) was added NaH (180 mg), after stirring at room temperature for 30 min, it was added a solution of 4-chloro-6-(4-iodophenylamino)-2-(methylthio)pyrimidin-5-carbonitrile (541 mg) in NMP (2 mL). The mixture was then heated at 75° C. for 1 h, more NaH (100 mg) was added, followed by additional heating at 75° C. for 30 min. The mixture was added ice water, the resulting precipitate was collected by filtration to give 4-(benzyloxy)-6-(4-iodophenylamino)-2-(methylthio)pyrimidin-5-carbonitrile (650 mg).

Step 3: To a solution of 4-(benzyloxy)-6-(4-iodophenylamino)-2-(methylthio)pyrimidin-5-carbonitrile (650 mg) in NMP (3 mL) was added AcOOH (35% in AcOH, 0.3 mL). After stirring at room temperature for 15 h, it was added water and the precipitates were isolated by filtration to give a mixture of corresponding sulfoxide and sulfone (610 mg), which was dissolved in NMP (2.5 mL), added (R)-leucinamide HCl salt (228 mg, 1.37 mmol) and DIPEA (0.486 mL, 2.73 mmol), the mixture was heated at 60° C. for 1 h, cooled to room temperature, diluted with water, the resulting precipitates were collected by filtration to give (R)-2-(4-(benzyloxy)-5-cyano-6-(4-iodophenylaminopyrimidine-2-ylamino)-4-methylpentanamide as crude solid.

Step 4: To above (R)-2-(4-(benzyloxy)-5-cyano-6-(4-iodophenylaminopyrimidine-2-ylamino)-4-methylpentanamide in DMSO (2.5 mL) was added H₂O₂ (50%, 1.5 mL), K₂CO₃ (471 mg, 3.41 mmol), after heated at 70° C. for 1 h, it was cooled and diluted with water, the resulting precipitate was collected by filtration to give crude amide (R)-2-(1-amino-4-methyl-1-oxopentan-2-ylamino)-4-(benzyloxy)-6-(iodophenylamino)pyrimidine-5-carboxamide.

Step 5: To a suspension of (R)-2-(1-amino-4-methyl-1-oxopentan-2-ylamino)-4-(benzyloxy)-6-(iodophenylamino)pyrimidine-5-carboxamide (185 mg, 0.32 mmol), pyridin-3-ylboronic acid (50 mg, 0.40 mmol) and PdCl₂(PPh₃)₂ (45 mg, 0.064 mmol) in 1M Na₂CO₃ (0.4 mL) was added a mixture of DME/EtOH/H₂O (7:2:3 vol/vol/vol, 3.6 mL). The mixture was heated at 90° C. for 1 h, cooled and added water and EtOAc, the organic layer was separated, dried and concentrated to give (R)-2-(1-amino-4-methyl-1-oxopentan-2-ylamino)-4-(benzyloxy)-6-(4-(pyridine-3-yl)phenylamino)-pyrimidine-5-carboxamide.

Step 5: To a solution of the above crude amide (R)-2-(1-amino-4-methyl-1-oxopentan-2-ylamino)-4-(benzyloxy)-6-(4-(pyridine-3-yl)phenylamino)-pyrimidine-5-carboxamide (200 mg) in THF (1 mL) was added 4M HCl in Dioxane (1 mL), after stirred at room temperature for 2 h, it was concentrated and purified by preparative HPLC to give (R)-2-(1-amino-4-methyl-1-oxopentan-2-ylamino)-6-oxo-4-(4-(pyridin-3-yl)phenylamino)-1,6-dihydropyrimidine-5-carboxamide (14 mg). MS found for C₂₂H₂₅N₇O₃ as (M+H)⁺ 436.6. λ=245.2, 281.9, 325.9.

### Example 107

### (R)-2-(1-amino-4-methyl-1-oxopentan-2-ylamino)-6-oxo-4-(quinolin-7-ylamino)-1,6-dihydropyrimidine-5-carboxamide

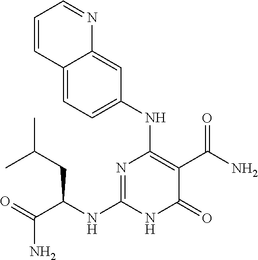 [SMILES: CC(C)C[C@@H](Nc1nc(Nc2ccc3cccnc3c2)c(C(N)=O)c(=O)[nH]1)C(N)=O]

The title compound was synthesized similarly to example 79 in Scheme 21 using 7-aminoquinoline to replace benzo[d][1,3]dioxol-5-amine. MS found for C₂₀H₂₃N₇O₃ as (M+H)⁺410.5. λ=265.3, 287.8.

### Example 108

### (R)-2-(1-amino-4-methyl-1-oxopentan-2-ylamino)-6-oxo-4-(quinolin-3-ylamino)-1,6-dihydropyrimidine-5-carboxamide

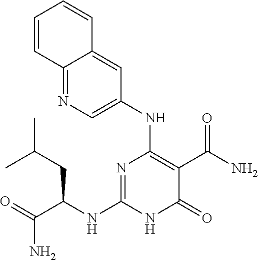 [SMILES: CC(C)C[C@@H](Nc1nc(Nc2cnc3ccccc3c2)c(C(N)=O)c(=O)[nH]1)C(N)=O]

The title compound was synthesized similarly to example 79 in Scheme 21 using 3-aminoquinoline to replace benzo[d][1,3]dioxol-5-amine. MS found for C₂₀H₂₃N₇O₃ as (M+H)⁺ 410.5. λ=219.2, 252.3, 289.0.

### Example 109

### (R)-2-(1-amino-1-oxobutan-2-ylamino)-6-oxo-4-(quinolin-3-ylamino)-1,6-dihydropyrimidine-5-carboxamide

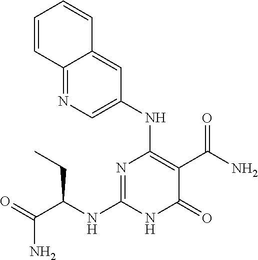 [SMILES: CC[C@@H](Nc1nc(Nc2cnc3ccccc3c2)c(C(N)=O)c(=O)[nH]1)C(N)=O]

The title compound was synthesized similarly to example 79 in Scheme 21 using 3-aminoquinoline to replace benzo[d][1,3]dioxol-5-amine and using (R)-2-aminobutanamide to replace D-leucinamide. MS found for C₁₈H₁₉N₇O₃ as (M+H)⁺ 382.5. λ=219.2, 252.3, 290.2.

### Example 110

### (R)-2-(1-amino-1-oxobutan-2-ylamino)-4-(6-methoxypyridin-3-ylamino)-6-oxo-1,6-dihydropyrimidine-5-carboxamide

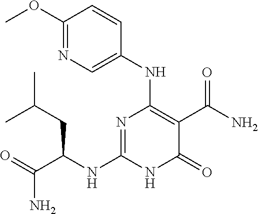 [SMILES: COc1ccc(Nc2nc(N[C@H](CC(C)C)C(N)=O)[nH]c(=O)c2C(N)=O)cn1]

The title compound was synthesized similarly to example 79 in Scheme 21 using 6-methoxy-3-aminopyridine to replace benzo[d][1,3]dioxol-5-amine. MS found for C₁₇H₂₃N₇O₄ as (M+H)⁺390.5. λ=255.8, 293.8.

### Example 111

### (R)-2-(1-amino-4-methyl-1-oxopentan-2-ylamino)-6-oxo-4-(4-(pyridin-4-yl)phenylamino)-1,6-dihydropyrimidine-5-carboxamide

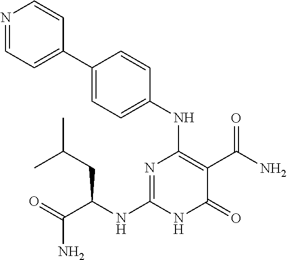 [SMILES: CC(C)C[C@@H](Nc1nc(Nc2ccc(-c3ccncc3)cc2)c(C(N)=O)c(=O)[nH]1)C(N)=O]

The title compound was synthesized similarly to example 106 in Scheme 23 using pyridin-4-ylboronic acid to replace pyridin-3-ylboronic acid. MS found for C₂₂H₂₅N₇O₃ as (M+H)⁺ 436.6. λ=249.7, 277.9.

### Example 112

### (R)-2-(1-amino-4-methyl-1-oxopentan-2-ylamino)-6-oxo-4-(3-(pyridin-4-yl)phenylamino)-1,6-dihydropyrimidine-5-carboxamide

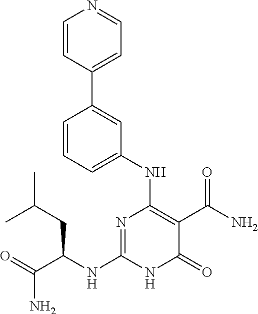 [SMILES: CC(C)C[C@@H](Nc1nc(Nc2cccc(-c3ccncc3)c2)c(C(N)=O)c(=O)[nH]1)C(N)=O]

The title compound was synthesized similarly to example 106 in Scheme 23 using 3-iodoaniline to replace 4-iodoaniline and using pyridin-4-ylboronic acid to replace pyridin-3-ylboronic acid. MS found for C₂₂H₂₅N₇O₃ as (M+H)⁺ 436.5. λ=261.7, 296.1.

### Example 113

### (R)-2-(1-amino-4-methyl-1-oxopentan-2-ylamino)-6-oxo-4-(4-(pyridin-2-yl)phenylamino)-1,6-dihydropyrimidine-5-carboxamide

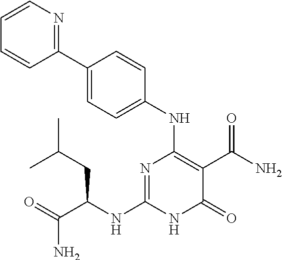 [SMILES: CC(C)C[C@@H](Nc1nc(Nc2ccc(-c3ccccn3)cc2)c(C(N)=O)c(=O)[nH]1)C(N)=O]

The title compound was synthesized similarly to example 79 in Scheme 21 using 4-(pyridin-2-yl)aniline to replace benzo[d][1,3]dioxol-5-amine. MS found for C₂₂H₂₅N₇O₃ as (M+H)⁺ 436.5. λ=242.8, 277.1.

### Example 114

### (R)-2-(1-amino-4-methyl-1-oxopentan-2-ylamino)-4-(1-methyl-1H-pyrazol-4-ylamino)-6-oxo-1,6-dihydropyrimidine-5-carboxamide

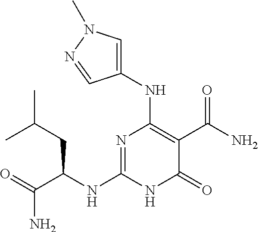 [SMILES: CC(C)C[C@@H](Nc1nc(Nc2cnn(C)c2)c(C(N)=O)c(=O)[nH]1)C(N)=O]

The title compound was synthesized similarly to example 79 in Scheme 21 using 1-methyl-1H-pyrazol-4-amine to replace benzo[d][1,3]dioxol-5-amine. MS found for C₁₅H₂₂N₈O₃ as (M+H)⁺ 363.3. λ=249.9, 293.8.

### Example 115

### (R)-2-(1-amino-4-methyl-1-oxopentan-2-ylamino)-4-(4-(1H-imidazol-1-yl)phenylamino)-6-oxo-1,6-dihydropyrimidine-5-carboxamide

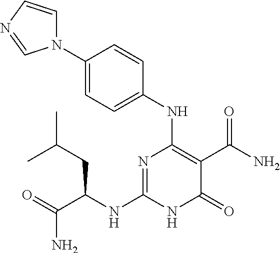 [SMILES: CC(C)C[C@@H](Nc1nc(Nc2ccc(-n3ccnc3)cc2)c(C(N)=O)c(=O)[nH]1)C(N)=O]

The title compound was synthesized similarly to example 79 in Scheme 21 using 4-(1H-imidazol-1-yl)aniline to replace benzo[d][1,3]dioxol-5-amine. MS found for C₂₀H₂₄N₈O₃ as (M+H)⁺ 425.5. λ=273.6, 306.8.

### Example 116

### (R)-2-(1-amino-1-oxobutan-2-ylamino)-4-(4-(1H-imidazol-1-yl)phenylamino)-6-oxo-1,6-dihydropyrimidine-5-carboxamide

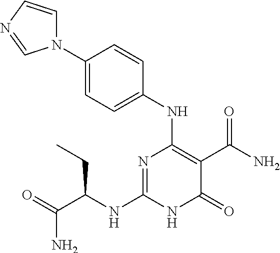 [SMILES: CC[C@@H](Nc1nc(Nc2ccc(-n3ccnc3)cc2)c(C(N)=O)c(=O)[nH]1)C(N)=O]

The title compound was synthesized similarly to example 79 in Scheme 21 using 4-(1H-imidazol-1-yl)aniline to replace benzo[d][1,3]dioxol-5-amine and using (R)-2-aminobutanamide to replace D-leucinamide. MS found for C₁₈H₂₀N₈O₃ as (M+H)⁺ 397.5. λ=271.2, 305.6.

### Example 117

### (R)-2-(1-amino-4-methyl-1-oxopentan-2-ylamino)-4-(1-methyl-1H-pyrazol-3-ylamino)-6-oxo-1,6-dihydropyrimidine-5-carboxamide

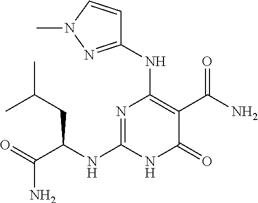 [SMILES: CC(C)C[C@@H](Nc1nc(Nc2ccn(C)n2)c(C(N)=O)c(=O)[nH]1)C(N)=O]

The title compound was synthesized similarly to example 79 in Scheme 21 using 1-methyl-1H-pyrazol-3-amine to replace benzo[d][1,3]dioxol-5-amine. MS found for C₁₅H₂₂N₈O₃ as (M+H)⁺ 363.4. λ=254.6, 294.9.

### Example 118

### (R)-2-(1-amino-4-methyl-1-oxopentan-2-ylamino)-4-(3-(1H-imidazol-1-yl)phenylamino)-6-oxo-1,6-dihydropyrimidine-5-carboxamide

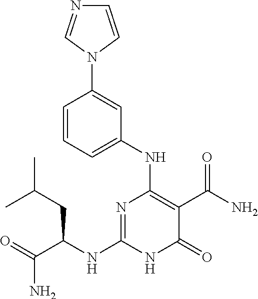 [SMILES: CC(C)C[C@@H](Nc1nc(Nc2cccc(-n3ccnc3)c2)c(C(N)=O)c(=O)[nH]1)C(N)=O]

The title compound was synthesized similarly to example 79 in Scheme 21 using 3-(1H-imidazol-1-yl)aniline to replace benzo[d][1,3]dioxol-5-amine. MS found for C₂₀H₂₄N₈O₃ as (M+H)⁺ 425.6. λ=257.0, 300.9.

### Example 119

### (R)-2-(1-amino-1-oxobutan-2-ylamino)-4-(3-(1H-imidazol-1-yl)phenylamino)-6-oxo-1,6-dihydropyrimidine-5-carboxamide

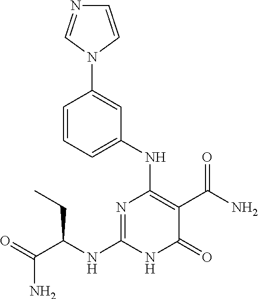 [SMILES: CC[C@@H](Nc1nc(Nc2cccc(-n3ccnc3)c2)c(C(N)=O)c(=O)[nH]1)C(N)=O]

The title compound was synthesized similarly to example 79 in Scheme 21 using 3-(1H-imidazol-1-yl)aniline to replace benzo[d][1,3]dioxol-5-amine and using (R)-2-aminobutanamide to replace D-leucinamide. MS found for C₁₈H₂₀N₈O₃ as (M+H)⁺ 397.5. λ=257.0, 299.7.

### Example 120

This example illustrates methods for evaluating the compounds of the invention, along with results obtained for such assays. The in vitro and in vivo human Syk activities of the inventive compounds can be determined by various procedures known in the art, such as a test for their ability to inhibit the activity of human plasma Syk. The potent affinities for human Syk inhibition exhibited by the inventive compounds can be measured by an IC₅ₒ value (in nM). The IC₅₀ value is the concentration (in nM) of the compound required to provide 50% inhibition of human Syk proteolytic activity. The smaller the IC₅₀ value, the more active (potent) is a compound for inhibiting Syk activity.

Assays for detecting and measuring inhibition activity against Syk is as follows:

### Inhibition of Syk Tyrosine Phosphorylation Activity

Potency of candidate molecules for inhibiting Syk tyrosine phosphorylation activity is assessed by measuring the ability of a test compound to inhibit Syk-mediated tyrosine phosphorylation of a Syk-specific substrate.

SYK tyrosine phosphorylation activity is measured using the LANCE™ Technology developed by Perkin Elmer Life and Analytical Sciences (Boston, Mass.). LANCE™ refers to homogeneous time resolved fluorometry applications using techniques such as time-resolved fluorescence resonance energy transfer assay (TR-FRET) (see generally for procedures in Perkin Elmer Application Note—How to Optimize a Tyrosine Kinase Assay Using Time Resolved Fluorescence-Based LANCE Detection, wwww.perkinelmer.com/lifesciences). The assay principle involves detection of a phosphorylated substrate using energy transfer from a phosphospecific europium-labeled antibody to streptavidin-allophycocyanin as an acceptor.

To test the ability of candidate molecules to inhibit SYK tyrosine phosphorylation activity, molecules are reconstituted in 30% DMSO and serially diluted 1:3 with the final dilution containing DMSO in the absence of the candidate molecule. The final DMSO concentration in the assay is 3%. Kinase assays are performed as a two part reaction. The first reaction is a kinase reaction and which comprises of a candidate molecule, full length active recombinant SYK enzyme (Millipore, Calif.) and biotin-labeled SYK-specific substrate biotin-DEEDYESP-OH. The second reaction involves termination of the kinase reaction and the simultaneous addition of the detection reagents-europium-labeled anti-phosphotyrosine reagent (Eu-W1024-PY100, Perkin Elmer, Boston, Mass.) and Streptavidin-Allophycocyanin detection reagent (SA-APC, Prozyme, Calif.). The kinase reaction is performed in a black U-bottom 96-well microtitre plate. The final reaction volume is 50 μL and contains a final concentration of 1 nM active SYK enzyme, 550 nM SYK-substrate, and 100 μM ATP diluted in a buffer containing 50 mM Tris pH 7.5, 5 mM MgCl₂, and 1 mM DTT. The reaction is allowed to proceed for 1 hour at room temperature. The quench buffer contains 100 mM Tris pH 7.5, 300 mM NaCl₂, 20 mM EDTA, 0.02% Brij35, and 0.5% BSA. The detection reagents are added to the reaction mixture at the following dilutions-1:500 for Eu-W1024-PY100 and 1:250 for SA-APC. The kinase reaction is terminated by the addition of 50 μL quench buffer containing the detection reagents. The detection is allowed to proceed for 1 hr at room temperature. Detection of the phosphorlated substrate in the absence and presence of inhibitors is measured in the TR-FRET instrument, Analyst HT (Molecular Probes, Sunnyvale, Calif.) and the condition for measurements are set up using CriterionHost Release 2.0 (Molecular Probes, Sunnyvale, Calif.). The settings used are a follows: excitation 360 nm, emission 665-7.5 nm, beam splitter 350 nm 50/50, flash 100 pulses, delay 60 us, integration 400 us, z-height 2 mm. Inhibition of SYK-tyrosine kinase activity is calculated as the maximum response observed in the presence of inhibitor, compared to that in the absence of inhibitor. IC₅₀s were derived by non-linear regression analysis.

Intracellular phospho-flow cytometry was used to test compound inhibition of Syk activity in intact non-Hodgkin's lymphoma cell lines Ramos and SUDHL-6. 10×10⁶ cells in log phase growth were aliqoted; Syk kinase is activated by incubating cells for 10 minutes with 3 μg/ml antibody specific to the B cell receptor. Directly following, cells are fixed in 1% paraformaldehyde for 5 minutes at room temperature, washed in phosphate buffered saline, and then permeablized by incubation for 2 hours in ice cold methanol. Cells are again washed in phosphate buffered saline, then incubated for 30 minutes with antibody specific for phosphorylated Erk (Y²⁰⁴) and BLNK (Y⁸⁴), which are indicators of Syk kinase activity, and phosphorylated Syk (Y³⁵²), a measure of Src family kinase activity. All antibodies used are purchased from BD Pharmingen (San Jose, Calif.). After incubation with antibodies, cells are again washed and subjected to flow cytometry. Representative data detailing inhibition of B cell receptor signaling by compounds are shown in Table 1 as IC₅₀ ranges.

The anti-proliferative effects of compounds on non-Hodgkin's lymphoma B cell lines SUDHL-4, SUDHL-6, and Toledo was also assessed. SUDHL-4 and SUDHL-6 require B cell receptor signaling for growth and survival, while the Toledo cell line (serving here as a negative control) does not. Cells were aliquoted into each well of a 96-well plate and incubated with increasing concentrations of compound for 72 hours, after which cell survival and proliferation was determined using the MTT assay (Chemicon International, Inc., Temecula, Calif.) following protocols supplied by the manufacturer. Data are detailed in the Tables and Figures herein as IC₅₀ values plus or minus standard deviations from 5 or 6 independent experiments.

Induction of apoptosis in non-Hodgkin's lymphoma B cell lines SUDHL-4, SUDHL-6, and Toledo was assessed by measuring the apoptotis marker Caspase 3. Cells were incubated with 1, 3, or 10 μM compound for 24, 48, and 72 hours. At the conclusion of each time point, cells were processed for flow cytometry analysis using the Monoclonal Rabbit Anti-Active Caspase-3 Antibody Kit and related protocols (BD Pharmingen). Data from two independent experiments are presented in Tables 7A and 7B, representing the percent of total cells undergoing apoptosis following incubation with compounds under the indicated conditions.

Syk activity is not only required for B cell signaling, proliferation, and survival, as shown, but is also critical for cellular activation upon cross-linking of the B cell receptor. B cell activation leads to increased cell surface expression of several proteins involved in cell signaling, antigen presentation, and adhesion. Among these, CD80, CD86, and CD69 are commonly measured to determine B cell activation status. Therefore, primary mouse B cells isolated from spleen were aliquoted and incubated with increasing concentrations of compound (0.05 to 2 μM) in the presence of goat anti-mouse IgD (eBiosciences, Inc., San Diego, Calif.) for 20 hours to cross-link the B cell receptor. Following, cells were washed and incubated for 30 minutes on ice with antibodies specific for the CD80, CD86, and CD69 B cell activation markers. B cells were identified from the pooled population by staining with the B cell marker CD45RO. All antibodies were purchased from BD Pharmingen. Table 8 depicts the IC₅₀ range in which these compounds inhibited B cell receptor induced activation of mouse primary B cells

In the table below, activity in the Syk assays is provided as follows: +++++=IC₅₀<0.0010 μM; ++++=0.0010 μM<IC₅₀<0.010 μM, +++=0.010 μM<IC₅₀<0.10 μM, ++=0.10 μM<IC₅₀<1 μM, +=IC₅₀>1 μM.

| Example No. | SYK IC50 |
|---|---|
| 1 | +++ |
| 2 | ++++ |
| 3 | +++ |
| 4 | ++ |
| 5 | ++++ |
| 6 | ++++ |
| 7 | +++ |
| 8 | +++ |
| | ++++ |
| 9 | ++++ |
| 10 | ++++ |
| 11 | ++++ |
| 12 | ++++ |
| 13 | +++++ |
| 14 | +++ |
| 15 | +++ |
| 16 | +++ |
| 17 | +++ |
| 18 | ++++ |
| 19 | ++++ |
| 20 | ++++ |
| 21 | +++++ |
| 22 | ++++ |
| 23 | +++ |
| 24 | ++++ |
| 25 | +++++ |
| 26 | +++++ |
| 27 | ++++ |
| 28 | +++ |
| 29 | +++++ |
| 30 | ++++++ |
| 31 | +++++ |
| 32 | ++++ |
| 33 | ++++ |
| 34 | ++++ |
| 35 | +++++ |
| 36 | +++++ |
| 37 | +++++ |
| 38 | ++++ |
| 39 | ++++ |
| 40 | ++++ |
| 41 | ++++ |
| 42 | +++ |
| 43 | + |
| 44 | + |
| 45 | + |
| 46 | + |
| 47 | + |
| 48 | + |
| 49 | +++ |
| 50 | ++ |
| 51 | ++++ |
| 52 | + |
| 53 | + |
| 54 | + |
| 55 | + |
| 56 | + |
| 57 | ++ |
| 58 | + |
| 59 | + |
| 60 | + |
| 61 | ++ |
| 62 | + |
| 63 | ++ |
| 64 | + |
| 65 | +++ |
| 66 | ++ |
| 67 | ++ |
| 68 | ++ |
| 69 | + |
| 70 | + |
| 71 | ++ |
| 72 | ++ |
| 73 | + |
| 74 | + |
| 75 | + |
| 76 | ++++ |
| 77 | ++++ |
| 78 | ++++ |
| 79 | +++++ |
| 80 | ++++ |
| 81 | ++++ |
| | +++ |
| 83 | +++++ |
| 86 | ++++ |
| 87 | ++++ |
| 88 | ++++ |
| 89 | ++++ |
| 90 | ++++ |
| 91 | ++++ |
| 92 | ++++ |
| 93 | ++++ |
| 94 | ++++ |
| 95 | ++++ |
| 96 | ++++ |
| 97 | ++++ |
| 98 | ++++ |
| 99 | ++++ |
| 100 | + |
| 101 | ++++ |
| 102 | ++++ |
| 103 | +++ |
| 104 | + |
| 105 | ++ |
| 106 | ++++ |
| 107 | ++++ |
| 108 | +++++ |
| 109 | +++++ |
| 110 | ++++ |
| 111 | ++++ |
| 112 | ++++ |
| 113 | ++++ |
| 114 | ++++ |
| 115 | ++++ |
| 116 | ++++ |
| 117 | ++ |
| 118 | ++++ |
| 119 | ++++ |
| | +++ |
| | ++++ |
| | ++++ |
| | ++++ |
| | ++++ |
| | ++++ |
| | ++ |
| | + |

As detailed herein, Syk has been implicated experimentally in B cell development, proliferation, and survival. Moreover, Syk is implicated as an oncogene. Expression of constitutively active Syk in adoptively transferred bone marrow cells induces leukemia in mice, and over-activity of Syk is associated with a variety of lymphomas in humans Given the role of Syk in B cell biology, its selective inhibition may be sufficient to provide clinical benefit in B cell proliferative disorders, while reducing toxicities that may arise due to suppression of other off-target kinases.

The present invention provides a number of embodiments. It is apparent that the examples may be altered to provide other embodiments of this invention. Therefore, it will be appreciated that the scope of this invention is to be defined by the appended claims rather than by the specific embodiments, which have been represented by way of example.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety. From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A compound having formula (I): 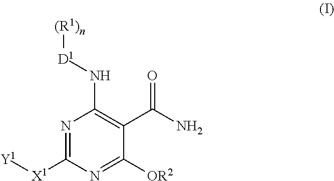
or a tautomer thereof or a pharmaceutically acceptable salt or hydrate thereof,
wherein:
D¹ is selected from the group consisting of:
(a) aryl substituted with a group, R⁵,
R³ is selected from the group consisting of:
(i) heteroaryl;
(ii) C₁₋₈alkyl;
(iii) C₁₋₈alkoxy;
(iv) C₁₋₈alkylcarbonylamino;
(v) aminosulfonyl;
(vi) heterocyclyl;
(vii) halo;
(viii) haloalkoxy;
each R⁵ is optionally further substituted with from 1 to 3 substituents independently selected from the group consisting of C₁₋₈alkyl, C₂₋₈ alkenyl, C₂₋₈alkynyl, hydroxyl, C₁₋₈alkoxy, oxo, halo, C₁₋₈alkylcarbonyl, aminocarbonyl, C₁₋₈ alkylcarbonylamino; C₁₋₈ alkylaminocarbonyl; aminosulfonyl, heteroaryl and heterocyclyl;
(b) C₃₋₈cycloalkyl;
(c) heteroaryl; and
each R¹ is independently selected from the group consisting of C₁₋₈alkyl, C₂₋₈ alkenyl, C₂₋₈alkynyl, hydroxyl, C₁₋₈alkoxy, oxo, halo, C₁₋₈alkylcarbonyl, aminocarbonyl, C₁₋₈ alkylcarbonylamino; C₁₋₈ alkylaminocarbonyl; aminosulfonyl, heteroaryl and heterocyclyl;
X¹ is NH or S;
Y¹ is selected from the group consisting of: 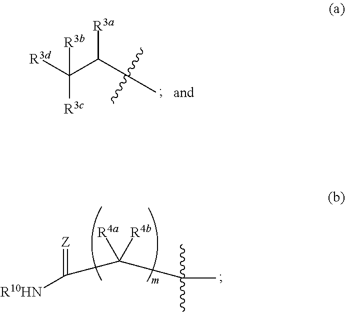
Z is O or S; and
(c) phenyl; substituted with a group, R^{6a}, selected from the group consisting of:
(i) heterocyclyl;
(ii) C₁₋₈alkylcarbonylamino;
(iii) C₁₋₈alkoxy;
(iv) heteroaryl;
(v) aminosulfonyl;
each R^{6a} is optionally further substituted with from 1 to 2 substituents independently selected from the group consisting of C₁₋₈alkyl, C₂₋₈ alkenyl, C₂₋₈alkynyl, hydroxyl, C₁₋₈alkoxy, oxo, halo, C₁₋₈alkylcarbonyl, aminocarbonyl, C₁₋₈ alkylcarbonylamino; C₁₋₈ alkylaminocarbonyl; aminosulfonyl, heteroaryl and heterocyclyl;
R² is H or C₁₋₈alkyl, optionally substituted with from 1 to 2 hydroxy, C₁₋₈ alkoxy or amino groups;
each R^{3a}, R^{4a} and R^{4b} is independently selected from the group consisting of: H, C₁₋₈alkyl, hydroxyC₁₋₈alkyl, C₁₋₈haloalkyl, amino, C₁₋₈alkylamino, C₁₋₈alkoxycarbonylaminoC₁₋₈alkylene, C₃₋₈cycloalkyl, heteroaryl, C₁₋₈ alkylC₃₋₈cycloalkyl, C₁₋₈alkylthioC₁₋₈ alkyl, C₁₋₈alkylsulfonylC₁₋₈ alkylene, aminocarbonyl, C₁₋₈alkoxyC₁₋₈alkyl, haloC₁₋₈alkyl, aryl and heterocyclyl; wherein the aryl is optionally substituted by hydroxyl. C₁₋₈alkoxy, halo or haloC₁₋₈alkyl; or taken together with R^{3b} and the atoms to which they are attached to form a C₃₋₈ cycloalkyl or heterocycloalkyl ring;
R^{3b} is selected from the group consisting of H, C₁₋₈alkyl, C₁₋₈alkylamino, amino aminoC₁₋₈alkyl, carboxy, C₁₋₈alkylaminoC₁₋₈alkyl, C₁₋₈alkoxyC₁₋₈alkyl, hydroxyC₁₋₈alkyl; carboxyC₁₋₈alkyl, C₃₋₈cycloalkylC₁₋₈alkyl, aryloxyC₁₋₈alkyl, arylC₁₋₈alkyl, heteroarylC₁₋₈alkyl, and hydroxyC₁₋₈alkoxy and hydroxyC₁₋₈alkoxy; or may be combined with R^{3a} or R^{3d} and the atoms to which they are attached to form a C₃₋₈ cycloalkyl or heterocyclyl ring;
R^{3c} is selected from the group consisting of H, amino, C₁₋₈alkylamino, hydroxycarbonylamino C₁₋₈alkoxycarbonylamino, arylC₁₋₈alkoxycarbonylamino and hydroxyl;
R^{3d} is H or alkyl or may be combined with R^{3b} and the atoms to which they are attached to form a C₃₋₈ cycloalkyl or heterocyclyl ring;
R¹⁰ is H or C₁₋₈alkyl;
the subscript n is 0, 1, 2, 3 or 4; and
the subscript m is an integer of 1, 2 or 3; and the wavy line indicates the point of attachment to the rest of the molecule.

2. The compound of claim 1 wherein R² is H.

3. The compound of claim 1 wherein R² is C₁₋₈alkyl.

4. The compound of claim 1 wherein Y¹ is:

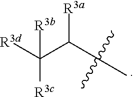

5. The compound of claim 1 wherein Y¹ is:

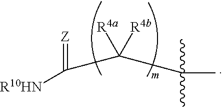

6.-7. (canceled)

8. The compound of claim 1 wherein Y¹ is phenyl.

9. The compound of claim 1 wherein D¹ is aryl.

10.-18. (canceled)

19. The compound of claim 1 wherein D¹ is C₃₋₈cycloalkyl.

20. (canceled)

21. The compound of claim 1 wherein D¹ is heteroaryl.

22. The compound of claim 1 wherein X¹ is NH.

23. The compound of claim 1 wherein X¹ is S.

24.-33. (canceled)

34. The compound of claim 1, wherein each R¹ is independently selected from the group consisting of R¹ is independently selected from the group consisting of C₁₋₈alkyl, C₂₋₈ alkenyl, C₂₋₈alkynyl, hydroxyl, C₁₋₈alkoxy, oxo, halo, C₁₋₈alkylcarbonyl, aminocarbonyl, C₁₋₈ alkylcarbonylamino; C₁₋₈ alkylaminocarbonyl; aminosulfonyl, heteroaryl and heterocyclyl.

35.-44. (canceled)

45. The compound of claim 5, wherein the moiety: 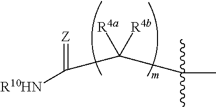
is 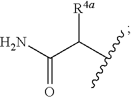
and the wavy line indicates the point of attachment to the rest of the molecule.

46.-53. (canceled)

54. The compound of claim 4, wherein the moiety: 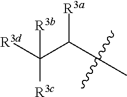
is selected from the group consisting of

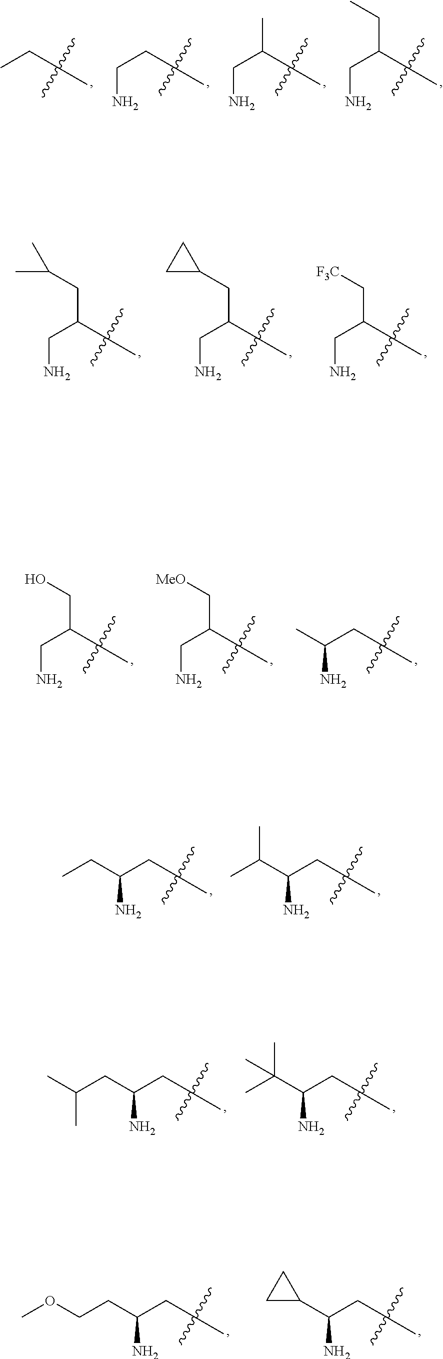

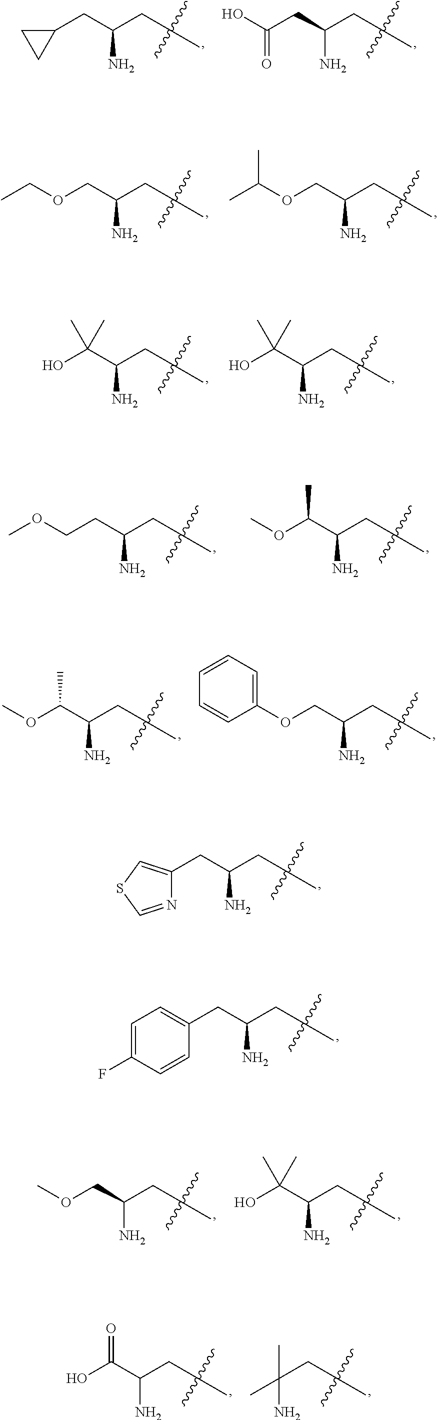

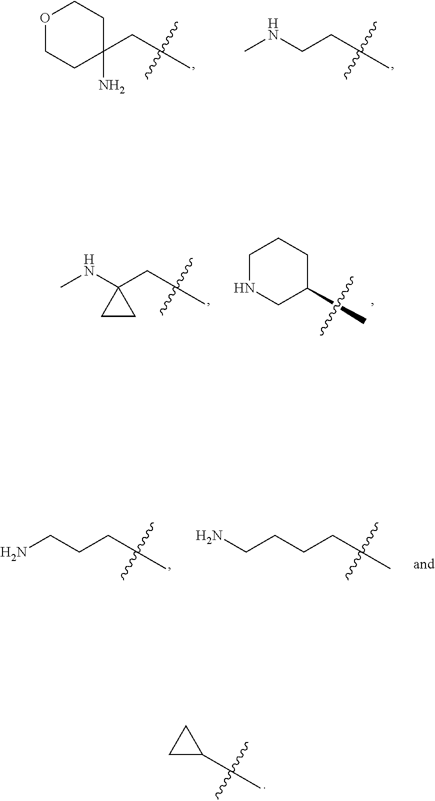

55. (canceled)

56. A compound of claim 1 selected from the group consisting of (R)-2-(1-amino-3-methyl-1-oxobutan-2-ylamino)-4-(3,5-dimethylphenylamino)-6-methoxypyrimidine-5-carboxamide; (R)-2-(1-amino-3-methyl-1-oxobutan-2-ylamino)-4-(3,5-dimethylphenylamino)-6-oxo-1,6-dihydropyrimidine-5-carboxamide; 2-((1R,2S)-2-aminocyclohexylamino)-4-(3-(2H-1,2,3-triazol-2-yl)phenylamino)-6-methoxypyrimidine-5-carboxamide; (R)-2-(1-amino-3-methyl-1-oxobutan-2-ylamino)-4-(3,5-dimethylphenylamino)-6-ethoxypyrimidine-5-carboxamide; 4-(3-(2H-1,2,3-triazol-2-yl)phenylamino-2-((1R,2S)-2-amino cyclohexyl)-6-oxo-1,6-dihydropyrimidine-5-carboxamide; (R)-4-(3-(2H-1,2,3-triazol-2-yl)phenyl amino)-2-(1-amino-3-methyl-1-oxobutan-2-ylamino)-6-oxo-1,6-dihydropyrimidine-5-carboxamide; (R)-4-(3-(2H-1,2,3-triazol-2-yl)phenylamino)-2-(1-amino-4-methyl-1-oxopentan-2-ylamino)-6-oxo-1,6-dihydropyrimidine-5-carboxamide; (R)-4-(3-(2H-1,2,3-triazol-2-yl)phenylamino)-2-(1-amino-1-oxobutan-2-ylamino)-6-oxo-1,6-dihydropyrimidine-5-carboxamide; (R)-2-(1-amino-4-methyl-1-oxopentan-2-ylamino)-6-oxo-4-(m-tolylamino)-1,6-dihydropyrimidine-5-carboxamide; (R)-2-(1-amino-3-methyl-1-oxobutan-2-ylamino)-6-oxo-4-(m-tolylamino)-1,6-dihydropyrimidine-5-carboxamide; (R)-2-(1-amino-1-oxobutan-2-ylamino)-6-oxo-4-(m-tolylamino)-1,6-dihydropyrimidine-5-carboxamide; (R)-2-(1-amino-3-methylbutan-2-ylamino)-4-(3,5-dimethylphenylamino)-6-oxo-1,6-dihydropyrimidine-5-carboxamide; (R)-2-(1-amino-4-methyl-1-oxopentan-2-ylamino)-6-oxo-4-(quinolin-6-ylamino)-1,6-dihydropyrimidine-5-carboxamide; 2-(2-(methylamino)ethylamino-6-oxo-4-(m-tolyl)-1,6-dihydropyrimidine-5-carboxamide; (R)-2-(1-amino-3-methyl-1-oxobutan-2-ylamino)-4-methoxy-6-(quinolin-6-ylamino)pyrimidine-5-carboxamide; (R)-2-(1-amino-1-oxobutan-2-ylamino)-4-methoxy-6-(quinolin-6-ylamino)pyrimidine-5-carboxamide; (R)-6-(2-(1-amino-1-oxobutan-2-ylamino)-5-carbamoyl-6-methoxypyrimidin-4-ylamino)quinoline 1-oxide; (R)-4-(4-(1H-pyrazol-1ylamino)phenylamino)-2-(1-amino-4-methyl-1-oxopentan-2-ylamino)-6-oxo-1,6-dihydropyrimidine-5-carboxamide; (R)-4-(4-(1H-pyrazol-1ylamino)phenylamino)-2-(1-amino-3-methyl-1-oxobutan-2-ylamino)-6-oxo-1,6-dihydropyrimidine-5-carboxamide; (R)-2-(1-amino-3-methyl-1-oxobutan-2-ylamino)-6-oxo-4-(quinolin-6-ylamino)-1,6-dihydropyrimidine-5-carboxamide; (R)-2-(1-amino-1-oxobutan-2-ylamino)-6-oxo-4-(quinolin-6-ylamino)-1,6-dihydropyrimidine-5-carboxamide; (R)-4-(3-(2H-1,2,3-triazol-2-yl)phenyl amino)-2-(1-amino-3-methylbutan-2-ylamino)-6-oxo-1,6-dihydropyrimidine-5-carboxamide; 4-(3-(2H-1,2,3-triazol-2-yl)phenylamino)-2-(2-(methylamino)ethylamino)-6-oxo-1,6-dihydropyrimidine-5-carboxamide; (R)-4-(4-(1H-pyrazol-1ylamino)phenylamino)-2-(1-amino-1-oxobutan-2-ylamino)-6-oxo-1,6-dihydropyrimidine-5-carboxamide; (R)-4-(1H-indazol-5-ylamino)-2-(1-amino-4-methyl-1-oxopentan-2-ylamino)-6-oxo-1,6-dihydropyrimidine-5-carboxamide; (R)-4-(1H-indazol-5-ylamino)-2-(1-amino-3-methyl-1-oxobutan-2-ylamino)-6-oxo-1,6-dihydropyrimidine-5-carboxamide; (R-2-(1-amino-4-methyl-1-oxopentan-2-ylamino)-6-oxo-4-(3-(pyrimidin-2-yl)phenylamino)-1,6-dihydropyrimidine-5-carboxamide; (R)-2-(1-amino-3-methyl-1-oxobutan-2-ylamino)-6-oxo-4-(3-(pyrimidin-2-yl)phenylamino)-1,6-dihydropyrimidine-5-carboxamide; (R)-2-(1-amino-4-methyl-1-oxopentan-2-ylamino)-6-oxo-4-(3-(pyrimidin-2-yl)phenylamino)-1,6-dihydropyrimidine-5-carboxamide; (R)-2-(1-amino-3-methyl-1-oxobutan-2-ylamino)-6-oxo-4-(3-(pyrimidin-2-yl)phenylamino)-1,6-dihydropyrimidine-5-carboxamide; (R)-2-(1-amino-1-oxobutan-2-ylamino)-6-oxo-4-(3-(pyrimidin-2-yl)phenylamino)-1,6-dihydropyrimidine-5-carboxamide; (R)-4-(3-(1H-pyrazol-1-ylamino)phenylamino)-2-(1-amino-4-methyl-1-oxopentan-2-ylamino)-6-oxo-1,6-dihydropyrimidine-5-carboxamide; (R)-4-(3-(1H-pyrazol-1-ylamino)phenylamino)-2-(1-amino-3-methyl-1-oxobutan-2-ylamino)-6-oxo-1,6-dihydropyrimidine-5-carboxamide; (R)-4-(3-(1H-pyrazol-1ylamino)phenylamino)-2-(1-amino-1-oxobutan-2-ylamino)-6-oxo-1,6-dihydropyrimidine-5-carboxamide; (R)-4-(1H-indazol-6-yl amino)-2-(1-amino-4-methyl-1-oxopentan-2-yl amino)-6-oxo-1,6-dihydropyrimidine-5-carboxamide; (R)-4-(1H-indazol-6-ylamino)-2-(1-amino-1-oxobutan-2-yl amino)-6-oxo-1,6-dihydropyrimidine-5-carboxamide; (R)-4-(3-(2H-1,2,3-triazol-2-yl)phenyl amino)-2-(1-amino-3-methyl butan-2-ylamino)-6-oxo-1,6-dihydropyrimidine-5-carboxamide; 4-(3-(pyrimidin-2-yl)phenylamino)-2-(1R,2S)-2-aminocyclohexyl)-6-oxo-1,6-dihydropyrimidine-5-carboxamide; (R)-2-(1-amino-1-oxobutan-2-ylamino)-4-(3-(methoxy)phenylamino)-6-oxo-1,6-dihydropyrimidine-5-carboxamide; (R)-2-(1-amino-4-methyl-1-oxopentan-2-ylamino)-6-oxo-4-(3-(pyridin-2-yl)phenylamino)-1,6-dihydropyrimidine-5-carboxamide; (R)-2-(1-amino-1-oxobutan-2-ylamino)-6-oxo-4-(3-(pyridin-2-yl)phenylamino)-1,6-dihydropyrimidine-5-carboxamide; 2-(cyclopropylamino)-6-oxo-4-(4-sulfamoylphenylamino)-1,6-dihydropyrimidine-5-carboxamide; 2-(cyclopropylamino)-4-(4-(N-methylacetamido)phenylamino)-6-oxo-1,6-dihydropyrimidine-5-carboxamide; 4-(cyclopropylamino)-6-oxo-2-(4-sulfamoylphenylamino)-1,6-dihydropyrimidine-5-carboxamide; 4-(cyclobutylamino)-6-oxo-2-(4-sulfamoylphenylamino)-1,6-dihydropyrimidine-5-carboxamide; 2,4-bis(4-(4-acetylpiperazin-1-yl)phenyl amino)-6-oxo-1,6-dihydropyrimidine-5-carboxamide; 2-(4-(4-acetylpiperazin-1-yl)phenylamino)-4-(cyclobutylamino)-6-oxo-1,6-dihydropyrimidine-5-carboxamide and 2-(2-aminocyclohexylamino)-4-(cyclopropylamino)-6-hydroxypyrimidine-5-carboxamide; 2-(2-amino-2-oxoethylamino)-4-(3,5-dimethylphenylamino)-6-oxo-1,6-dihydropyrimidine-5-carboxamide; 2-(2-amino-2-oxoethylamino)-4-(3,5-dimethylphenylamino)-6-methoxypyrimidine-5-carboxamide; (S)-2-(2-aminopropylthio)-6-oxo-4-(quinolin-6-ylamino)-1,6-dihydropyrimidine-5-carboxamide; 4-(m-toluidino)-6-(3-aminopropoxy)-2-(ethylamino)pyrimidine-5-carboxamide; (S)-4-(m-toluidine)-2-(2-aminopropylamino)-6-(2-hydroxyethoxy)pyrimidine-5-carboxamide; (S)-2-(2-aminopropylamino)-4-(3,5-dimethylphenylamino)-6-oxo-1,6-dihydropyrimidine-5-carboxamide; (R)-2-(1-amino-4-methyl-1-oxopentan-2-ylamino)-4-(3,5-dimethylphenylamino)-6-oxo-1,6-dihydropyrimidine-5-carboxamide; (R)-2-(1-amino-1-oxopropan-2-ylamino)-4-(3,5-dimethylphenylamino)-6-oxo-1,6-dihydropyrimidine-5-carboxamide; 2-(2-aminocyclohexylamino)-4-(3,5-dimethylphenylamino)-6-oxo-1,6-dihydropyrimidine-5-carboxamide; 4-(cyclopropylamino)-2-(4-(N-methylacetamido)phenylamino)-6-oxo-1,6-dihydropyrimidine-5-carboxamide; 2-(4-(4-acetylpiperazin-1-yl)phenylamino)-4-(3,5-dimethylphenylamino)-6-oxo-1,6-dihydropyrimidine-5-carboxamide; 2-(cyclopropylamino)-4-(3,5-dimethylphenylamino)-6-oxo-1,6-dihydropyrimidine-5-carboxamide; 4-(4-(3,5-dimethylphenylamino)-5-carboxamide-6-oxo-1,6-dihydropyrimidin-2-ylamino)benzenesulfonamide; 2-(4-(4-acetylpiperazin-1-yl)phenylamino)-4-(cyclopropylamino)-6-oxo-1,6-dihydropyrimidine-5-carboxamide; 4-(4-(3-methylphenylamino)-5-carboxamide-6-oxo-1,6-dihydropyrimidin-2-ylamino)benzenesulfonamide; 4-(m-toluidino)-2-(4-(4-acetylpiperazin-1-yl)phenylamino)-6-hydroxypyrimidine-5-carboxamide; 2-(2-aminocyclohexylamino)-4-(3,5-dimethylphenylamino)-6-methoxypyrimidine-5-carboxamide; (R)-2-(1-amino-1-oxopropan-2-ylamino)-4-(3,5-dimethylphenylamino)-6-methoxypyrimidine-5-carboxamide; (R)-2-(1-amino-4-methyl-1-oxopentan-2-yl amino)-4-(3,5-dimethylphenylamino)-6-methoxypyrimidine-5-carboxamide; (S)-2-(2-aminopropylamino)-4-(3,5-dimethylphenylamino)-6-methoxypyrimidine-5-carboxamide, 2-(4-(4-acetylpiperazin-1-yl)phenylamino)-4-(cyclopropylamino)-6-methoxypyrimidine-5-carboxamide; 4-(m-toluidino)-6-methoxy-2-(4-morpholinophenylamino)pyrimidine-5-carboxamide; (R)-4-(m-toluidino)-2-(2-amino-2-oxo-1-phenylethylamino)-6-methoxypyrimidine-5-carboxamide; (R)-2-(2-amino-2-oxo-1-phenylethylamino)-4-(3,5-dimethylphenyl amino)-6-methoxypyrimidine-5-carboxamide; 4-(cyclopropylamino)-6-methoxy-2-(4-morpholinophenylamino)pyrimidine-5-carboxamide; 4-(m-toluidino)-2-(4-(4-acetylpiperazin-1-yl)phenylamino)-6-methoxypyrimidine-5-carboxamide; 4-(m-toluidino)-2-(ethylamino)-6-(2-hydroxyethoxy)pyrimidine-5-carboxamide: (R)-2-(1-amino-1-oxobutan-2-ylamino)-4-(benzo[d][1,3]dioxol-5-ylamino)-6-oxo-1,6-dihydropyrimidine-5-carboxamide; (R)-2-(1-amino-4-methyl-1-oxopentan-2-ylamino)-4-(3-methoxyphenylamino)-6-oxo-1,6-dihydropyrimidine-5-carboxamide; (R)-2-(1-amino-4-methyl-1-oxopentan-2-ylamino)-4-(2,3-dihydrobenzo[b][1,4]dioxin-6-ylamino)-6-oxo-1,6-dihydropyrimidine-5-carboxamide; (R)-2-(1-amino-4-methyl-1-oxopentan-2-ylamino)-4-(benzo[d][1,3]dioxol-5-ylamino)-6-oxo-1,6-dihydropyrimidine-5-carboxamide; (R)-2-(1-amino-4-methyl-1-oxopentan-2-ylamino)-6-oxo-4-(3-(3-fluoropyridin-2-yl)phenylamino)-1,6-dihydropyrimidine-5-carboxamide; (R)-2-(1-amino-1-oxobutan-2-ylamino)-6-oxo-4-(3-(3-fluoropyridin-2-yl)phenylamino)-1,6-dihydropyrimidine-5-carboxamide; (R)-2-(1-amino-1-oxobutan-2-ylamino)-6-oxo-4-(3-(3-hydroxypyridin-2-yl)phenylamino)-1,6-dihydropyrimidine-5-carboxamide; (R)-2-(1-amino-4-methyl-1-oxopentan-2-yl amino)-4-(3,4-dimethoxyphenyl amino)-6-oxo-1,6-dihydropyrimidine-5-carboxamide; (R)-2-(1-amino-1-oxobutan-2-ylamino)-4-(2,3-dihydrobenzo[b][1,4]dioxin-6-ylamino)-6-oxo-1,6-dihydropyrimidine-5-carboxamide (R)-2-(1-amino-4-methyl-1-oxopentan-2-ylamino)-4-(3-(difluoromethoxy)phenylamino)-6-oxo-1,6-dihydropyrimidine-5-carboxamide; (R)-2-(1-amino-1-oxobutan-2-ylamino)-4-(3-(difluoromethoxy)phenylamino)-6-oxo-1,6-dihydropyrimidine-5-carboxamide; (R)-2-(1-amino-4-methyl-1-oxopentan-2-ylamino)-4-(4-methoxyphenylamino)-6-oxo-1,6-dihydropyrimidine-5-carboxamide; (R)-2-(1-amino-1-oxobutan-2-ylamino)-4-(4-methoxyphenylamino)-6-oxo-1,6-dihydropyrimidine-5-carboxamide; (R)-2-(1-amino-4-methyl-1-oxopentan-2-ylamino)-4-(4-(trifluoromethoxy)phenylamino)-6-oxo-1,6-dihydropyrimidine-5-carboxamide; (R)-2-(1-amino-1-oxobutan-2-ylamino)-4-(4-(trifluoromethoxy)phenylamino)-6-oxo-1,6-dihydropyrimidine-5-carboxamide; (R)-2-(1-amino-4-methyl-1-oxopentan-2-ylamino)-4-(4-(2-methoxyethoxy)phenylamino)-6-oxo-1,6-dihydropyrimidine-5-carboxamide; (R)-2-(1-amino-1-oxobutan-2-ylamino)-4-(4-(2-methoxyethoxy)phenylamino)-6-oxo-1,6-dihydropyrimidine-5-carboxamide; (R)-2-(1-amino-3-cyclopropyl-1-oxopropan-2-ylamino)-6-oxo-4-(3-(pyridin-2-yl)phenylamino)-1,6-dihydropyrimidine-5-carboxamide; (R)-2-(1-amino-3-methyl-1-oxobuan-2-ylamino)-6-oxo-4-(3-(pyridin-2-yl)phenylamino)-1,6-dihydropyrimidine-5-carboxamide; (R)-2-(1-amino-4-methyl-1-oxopentan-2-ylamino)-6-oxo-4-(3-(pyridin-3-yl)phenylamino)-1,6-dihydropyrimidine-5-carboxamide; (R)-2-(1-amino-4-methyl-1-oxopentan-2-ylamino)-4-(3-(2-methoxyethoxy)phenylamino)-6-oxo-1,6-dihydropyrimidine-5-carboxamide; (R)-2-(1-amino-1-oxobutan-2-ylamino)-4-(3-(2-methoxyethoxy)phenylamino)-6-oxo-1,6-dihydropyrimidine-5-carboxamide (R)-2-(4-methyl-1-(methylamino)-1-oxopentan-2-ylamino)-6-oxo-4-(3-(pyridin-2-yl)phenylamino)-1,6-dihydropyrimidine-5-carboxamide; (R)-2-(1-amino-4-methyl-1-oxopentan-2-ylamino)-4-(3-(trifluoromethoxy)phenylamino)-6-oxo-1,6-dihydropyrimidine-5-carboxamide; (R)-2-(1-amino-1-oxobutan-2-ylamino)-4-(3-(trifluoromethoxy)phenylamino)-6-oxo-1,6-dihydropyrimidine-5-carboxamide; (R)-4-(benzo[d]thiazol-6-ylamino)-2-(4-methyl-1-(methylamino)-1-oxopentan-2-ylamino)-6-oxo-1,6-dihydropyrimidine-5-carboxamide; (R)-4-(3-(1H-pyrazol-1-yl)phenylamino)-2-(4-methyl-1-(methylamino)-1-oxopentan-2-ylamino)-6-oxo-1,6-dihydropyrimidine-5-carboxamide; (R)-4-(1H-indazol-5-ylamino)-2-(4-methyl-1-(methylamino)-1-oxopentan-2-ylamino)-6-oxo-1,6-dihydropyrimidine-5-carboxamide; (R)-2-(1-amino-4-methyl-1-oxopentan-2-ylamino)-6-oxo-4-(4-(pyridin-3-yl)phenylamino)-1,6-dihydropyrimidine-5-carboxamide; (R)-2-(1-amino-4-methyl-1-oxopentan-2-ylamino)-6-oxo-4-(quinolin-7-ylamino)-1,6-dihydropyrimidine-5-carboxamide; (R)-2-(1-amino-4-methyl-1-oxopentan-2-ylamino)-6-oxo-4-(quinolin-3-ylamino)-1,6-dihydropyrimidine-5-carboxamide; (R)-2-(1-amino-1-oxobutan-2-ylamino)-6-oxo-4-(quinolin-3-ylamino)-1,6-dihydropyrirnidine-5-carboxamide; (R)-2-(1-amino-1-oxobutan-2-ylamino)-4-(6-methoxypyridin-3-ylamino)-6-oxo-1,6-dihydropyrimidine-5-carboxamide; (R)-2-(1-amino-4-methyl-1-oxopentan-2-ylamino)-6-oxo-4-(4-(pyridin-4-yl)phenylamino)-1,6-dihydropyrimidine-5-carboxamide; (R)-2-(1-amino-4-methyl-1-oxopentan-2-ylamino)-6-oxo-4-(3-(pyridin-4-yl)phenylamino)-1,6-dihydropyrimidine-5-carboxamide; (R)-2-(1-amino-4-methyl-1-oxopentan-2-ylamino)-6-oxo-4-(4-(pyridin-2-yl)phenylamino)-1,6-dihydropyrimidine-5-carboxamide; (R)-2-(1-amino-4-methyl-1-oxopentan-2-ylamino)-4-(1-methyl-1H-pyrazol-4-ylamino)-6-oxo-1,6-dihydropyrimidine-5-carboxamide; (R)-2-(1-amino-4-methyl-1-oxopentan-2-ylamino)-4-(4-(1H-imidazol-1-yl)phenylamino)-6-oxo-1,6-dihydropyrimidine-5-carboxamide; (R)-2-(1-amino-1-oxobutan-2-ylamino)-4-(4-(1H-imidazol-1-yl)phenylamino)-6-oxo-1,6-dihydropyrirnidine-5-carboxamide; (R)-2-(1-amino-4-methyl-1-oxopentan-2-ylamino)-4-(1-methyl-1H-pyrazol-3-ylamino)-6-oxo-1,6-dihydropyrimidine-5-carboxamide; (R)-2-(1-amino-4-methyl-1-oxopentan-2-ylamino)-4-(3-(1H-imidazol-1-yl)phenylamino)-6-oxo-1,6-dihydropyrimidine-5-carboxamide; and (R)-2-(1-amino-1-oxobutan-2-ylamino)-4-(3-(1H-imidazol-1-yl)phenylamino)-6-oxo-1,6-dihydropyrimidine-5-carboxamide.

57.-58. (canceled)

59. A composition comprising a compound of claim 1 in combination with a pharmaceutically acceptable carrier or diluent.

60. A method for inhibiting Syk kinase or a signal transduction pathway mediated at least in part by Syk kinase activity comprising the step of contacting a cell with a compound of claim 1.

61. A method for treating a condition or disorder mediated at least in part by Syk kinase activity in a subject comprising the step of administering to a subject in need of such treatment a therapeutically effective amount of a composition of claim 59.

62.-71. (canceled)

72. A kit comprising a composition of claim 59, packaging and instructions for use.
